SEQUENCE LISTING (94 sequences)

SEQ ID NO: 1 - protein, 414 aa, Homo sapiens
  misc_feature: Incyte ID No 3356677CD1
MSNVSGILETAGVPLVSANWPQPSPPPAVPAGPQMDHMGNSSQGAPWLFLTSALARGVSGIFVWTALVLTCHQIYLHLRSYTVPQEQRYIIRLLLIVPIYAFDSWLSLLLLGDHQYYVYFDSVRDCYEAFVIYSFLSLCFQYLGGEGAIMAEIRGKPIKSSCLYGTCCLRGMTYSIGFLRFCKQATLQFCLVKPVMAVTTIILQAFGKYHDGDFNVRSVYLYVTLIYNASVSLALYALFLFYFTTRELLRPFQPVLKFLTIKAVIFLSFWQGLLLAILERCGVIPEVETSGGNKLGAGTLAAGYQNFIICVEMLFASVALRYAFPCQVYAEKKENSPAPPAPMQSISSGIRETVSPQDIVQDAIHNFSPAYQHYTQQATHEAPRPGTHPGGGGSGGSRKSRSLEKRMLIPSEDL

SEQ ID NO: 2 - protein, 836 aa, Homo sapiens
  misc_feature: Incyte ID No 7481665CD1
MNPFYFHAVNIILHCLVTLVLMYTCDKTVFKNRGLAFVTALLFAVHPIHTEAVAGIVGRADVLACLLFLLAFLSYNRSLDQGCVGGSFPSTVSPFFLLLSLFLGTCAMLVKETGITVFGVCLVYDLFSLSNKQDKSSNGALCPRSPQQPGSPQPSSLPGHPHRENGKQQRFPHKGAWGGCHSPLPPEPKSSGFPVSPRAVWSMMRYLRASSNRNFLLTMRPFLKRAILVLSYVLVILYFRLWIMGGSMPLFSEQDNPASFSPYILTRFLTYSYLLAFNVWLLLAPVTLCYDWQVGSIPLVETIWDMRNLATIFLAVVMALLSLHCLAAFKRLEHKEVLVGLLFLVFPFIPASNLFFRVGFVVAERVLYMPSMGYCILFVHGLSKLCTWLNRCGATTLIVSTVLLLLLFSWKTVKQNEIWLSRESLFRSGVQTLPHNAKVHYNYANFLKDQGRNKEAIYHYRTALKLYPRHASALNNLGTLTRDTAEAKMYYQRALQLHPQHNRALFNLGNLLKSQEKKEEAITLLKDSIKYGPEFADAYSSLASLLAEQERFKEAEEIYQTGIKNCPDSSDLHNNYGVFLVDTGLPEKAVAHYQQAIKLSPSHHVAMVNLGRLYRSLGENSMAEEWYKRALQVAHKAEILSPLGALYYNTGRYEEALQIYQEAAALQPSQRELRLALAQVLAVMGQTKEAEKMTNHIVSEETGCLECYRLLSAIYSKQENHDKALDAIDKALQLKPKDPKVISELFFTKGNQLREQNLLDKAFESYRVAVQLNPDQAQAWMNMGGIQHIKGKYVSARAYYERALQLVPDSKLLKENLAKLDRLEKRLQEVREKDQT

SEQ ID NO: 3 - protein, 401 aa, Homo sapiens
  misc_feature: Incyte ID No 3563859CD1
MDKDSPEQLALSQELEHGRSQGHRTNRKGHRLLSGHILPHTRGQDPADVADLHPGPLLPPAPLPHLPAANRALPCVCHTCHCLSLSRGGPISPSSAAQQSKQLVGRGGNMERGPVVGAGLGAGARIQALLGCLLKVLLWVASALLYFGSEQAARLLGSPCLRRLYHAWLAAVVIFGPLLQFHVNPRTIFASHGNFFNIKFVNSAWGWTCTFLGGFVLLVVFLATRRVAVTARHLSRLVVGAAVWRGAGRAFLLIEDLTGSCFEPLPQGLLLHELPDRRSCLAAGHQWRGYTVSSHTFLLTFCCLLMAEEAAVFAKYLAHGLPAGAPLRLVFLLNVLLLGLWNFLLLCTVIYFHQYTHKVVGAAVGTFAWYLTYGSWYHQPWSPGSPGHGLFPRPHSSRKHN

SEQ ID NO: 4 - protein, 181 aa, Homo sapiens
  misc_feature: Incyte ID No 2588884CD1
MTFDDLKIQTVKDQPDEKSNGKKAKGLQFLYSPWWCLAAATLGVLCLGLVVTIMVLGMQLSQVSDLLTQEQANLTHQKKKLEGQISARQQAEEASQESENELKEMIETLARKLNEKSKEQMELHHQNLNLQETLKRVANCSGLHPASNFLFQFSILDGAVSEEPQLPMALGGRFSFDAPLI

SEQ ID NO: 5 - protein, 249 aa, Homo sapiens
  misc_feature: Incyte ID No 7503422CD1
MTATEALLRVLLLLLAFGHSTYGAECFPACNPQNGFCEDDNVCRCQPGWQGPLCDQCVTSPGCLHGLCGEPGQCICTDGWDGELCDRDVRACSSAPCANNGYSGKDCQKKDGPCVINGSPCQHGGTCVDDEGRASHASCLCPPGFSGNFCEIVASPCQNGGTCLQHTQAICFTILGVLTSLVVLGTVGIVFLNKCETWVSNLRYNHMLRKKKKNLLLQYNSGEDLAVNIIFPEKIDMTTFSKEAGDEEI

SEQ ID NO: 6 - protein, 289 aa, Homo sapiens
  misc_feature: Incyte ID No 7503424CD1
MTATEALLRVLLLLLAFGHSTYGAECFPACNPQNGFCEDDNVCRCQPGWQGPLCDQCVTSPGCLHGLCGEPGQCICTDGWDGELCDRDVRACSSAPCANNGTCVSLDDGLYECSCAPGYSGKDCQKKDGPCVINGSPCQHGGTCVDDEGRASHASCLCPPGFSGNFCEIVASPCQNGGTCLQHTQPEHRILKVSMKELNKKTPLLTEGQAICFTILGVLTSLVVLGTVGIVFLNKCETWVSNLRYNHMLRKKKNLLLQYNSGEDLAVNIIFPEKIDMTTFSKEAGDEEI

SEQ ID NO: 7 - protein, 170 aa, Homo sapiens
  misc_feature: Incyte ID No 7503571CD1
MCTGKCARCVGLSLITLCLVCIVANALLLVPNGETSWTNTNHLSLQVWLMGGFIGGGLMMLRSVFSSAFGVLGAIYCLSVSGAGLRNGPRCLMNGEWGYHFEDTAGAYLLNRTLWDRCEAPPRVVPWNVTLFSLLVAASCLEIVLCGIQLVNATIGVFCGDCRKKQDTPH

SEQ ID NO: 8 - protein, 328 aa, Homo sapiens
  misc_feature: Incyte ID No 7505722CD1
MEKSIWLLACLAWVLPTGSFVRTKIDTTENLLNTEVHSSPAQRWSMQVPPEVSAEAGDAAVLPCTFTHPHRHYDGPLTAIWRAGEPYAGPQVFRCAAARGSELCQTALSLHGRFRLLGNPRRNDLSLRVERLALADDRRYFCRVEFAGDVHDRYESRHGVRLHVTAAPRIVNISVLPSPAHAFRALCTAEGEPPPALAWSGPALGNSLAAVRSPREGHGHLVTAELPALTHDGRYTCTAANSLGRSEASVYLFRFHGASGASTVALLLGALGFKALLLLGVLAARAARRRPEHLDTPDTPPRSQAQESNYENLSQMNPRSPPATMCSP

SEQ ID NO: 9 - protein, 287 aa, Homo sapiens
  misc_feature: Incyte ID No 7505798CD1
MASADELTFHEFEEATNLLADTPDAATTSRSDQLTPQGHVAVAVGSGGSYGAEDEVEEESDKAAVLDRIKGSLLPRPGHNFVRHHLRNRPDLYGPFWICATLAFVLAVTGNLTLVLAQRRDPSIHYSPQFHKVTVAGISIYCYAWLVPLALWGFLRWRKGVQERMGPYTFLETVCIYGYSLFVFIPMVVLWLIPVPWLQWLFGALALGLSAAGLVFTLWPVVREDTRLVATVLLSVVVLLHALLAMGCKLYFFQSLPPENVAPPPQITSLPSNIALSPTLPQSLAPS

SEQ ID NO: 10 - protein, 300 aa, Homo sapiens
  misc_feature: Incyte ID No 7505847CD1
MAAACGPGAAGYCLLLGLHLFLLTAGPALGWNDPDRMLLRDVKALTLHYDRYTTSRRLDPIPQLKCVGGTAGCDSYTPKVIQCQNKGWDGYDVQWECKTDLDIAYKFGKTVVSCEGYESSEDQYVLRGSCGLEYNLDYTELGLQKLKESGKQHGFASFSDYYYKWSSADSCNMSGLITIVVLLGIAFVVYKLFLSDGQYSPPPYSEYPPFSHRYQRFTNSAGPPPPGLGTGGILGYLFGSNRAATPFSDSWYYPSYPPSYPGTWNRAYSPLHGGSGSYSVCSNSDTKTRTASGYGGTRRR

SEQ ID NO: 11 - protein, 297 aa, Homo sapiens
  misc_feature: Incyte ID No 7505862CD1
MAALIAENFRFLSLFFKSKDVMIFNGLVALGTVGSQELFSVVAFHCPCSPARNYLYGLAAIGVPALVLFIIGIILLLSSILGRAAVAPVTWSVISLLRGEAYVCALSEFVDPSSLTAREEHFPSAHATEILARFPCKENPDNLSDFREEVSRRLRYESQLFGWLLIGVVAILVFLTKCLKHYCSPLSYRQEAYWAQYRANEDQLFQRTAEVHSRVLAANNVRRFFGFVALNKDDEELIANFPVEGTQPRPQWNAITGVYLYRENQGLPLYSRLHKWAQGLAGNGAAPDNVEMALLPS

SEQ ID NO: 12 - protein, 200 aa, Homo sapiens
  misc_feature: Incyte ID No 7762537CD1
MPPDPYLQETRFEGPLPPPRRRAAAPPPPAPAQTAQAPGFVVPTHAGTVGTLPLGGYVAPGYPLQLQPCTAYVPVYPVGTPYAGGTPGGTGVTSTLPPPPQGPGLALLEPRRPPHDYMPIAVLTTICCFWPTGIIAIFKAVQVRTALARGDMVSAEIASREARNFSFISLAVGIAAMVLCTILTVVIIIAAQHHENYWDP

SEQ ID NO: 13 - protein, 282 aa, Homo sapiens [sequence omitted]

SEQ ID NO: 14 - protein, 805 aa, Homo sapiens
  misc_feature: Incyte ID No 1644869CD1
MAQPLAFILDVPETPGDQGQGPSPYDESEVHDSFQQLIQEQSQCTAQEGLELQQREREVTGSSQQTLWRPEGTQSTATLRILASMPSRTIGRSRGAIISQYYNRTVQLRCRSSRPLLGNFVRSAWPSLRLYDLELDPTALEEEEKQSLLVKELQSLAVAQRDHMLRGMPLSLAEKRSLREKSRTPRGKWRGQPGSGGVCSCCGRLRYACVLALHSLGLALLSALQALMPWRYALKRIGGQFGSSVLSYFLFLKTLLAFNALLLLLLVAFIMGPQVAFPPALPGPAPVCTGLELLTGAGCFTHTVMYYGHYSNATLNQPCGSPLDGSQCTPRVGGLPYNMPLAYLSTVGVSFFITCITLVYSMAHSFGESYRVGSTSGIHAITVFCSWDYKVTQKRASRLQQDNIRTRLKELLAEWQLRHSPRSVCGRLRQAAVLGLVWLLCLGTALGCAVAVHVFSEFMIQSPEAAGQEAVLLVLPLVVGLLNLGAPYLCRVLAALEPHDSPVLEVYVAICRNLILKLAILGTLCYHWLGRRVGVLQGQCWEDFVGQELYRFLVMDFVLMLLDTLFGELVWRIISEKKLKRRRKPEFDIARNVLELIYGQTLTWLGVLFSPLLPAVQIIKLLLVFYVKKTSLLANCQAPRRPWLASHMSTVFLTLLCFPAFLGAAVFLCYAVWQVKPSSTCGPFRTLDTMYEAGRVWVRHLEAAGPRVSWLPWVHRYLMENTFFVFLVSALLLAVIYLNIQVVRGQRKVICLLKEQISNEGEDKIFLINKLHSIYERKEREERSRVGTTEEAAAPPALLTDEQDA

SEQ ID NO: 15 - protein, 96 aa, Homo sapiens
  misc_feature: Incyte ID No 6288712CD1
MFPFLNSYDEDQGSKLIQKAKEAPFVLVGIEGFAAIVAYGLYKLKSRGNTKMSLHLIHMGVAAQGFVVGAMTVVISCIENSGQNLSLRRRDAVLLL

SEQ ID NO: 16 - protein, 244 aa, Homo sapiens
  misc_feature: Incyte ID No 71830156CD1
MAEIHTPYSSLKKLLSLLNGFVAVSGIILVGLGIGGKCGGASLTNVLGLSSAYLLHVGNLCLVMGCITVLLGCAGWYGATKESRGTLLFCILSMVIVLIMEVTAATVVLLFFPIVGDVALEHTFVTLRKNYRGYNEPDDYSTQWNLVMEKLKCCGVNNYTDFSGSSFEMTTGHTYPRSCCKSIGSVSCDGRDVSPNVIHQKGCFHKLLKITKTQSFTLSGSSLGAAVIQLPGILATLLLFIKLG

SEQ ID NO: 17 - protein, 237 aa, Homo sapiens
  misc_feature: Incyte ID No 7505044CD1
MPLLLLLPLLWADLTHRPKILIPGTLEPGHSKNLTCSVSWACEQGTPPIFSWLSAAPTSLGPRTTHSSVLIITPRPQDHGTNLTCQVKFAGAGVTTERTIQLNVTYVPQNPTTGIFPGDGSGKQETRAGVVHGAIGGAGVTALLALCLCLIFFIVKTHRRKAARTAVGRNDTHPTTGSASPKHQKKSKLHGPTETSSCSGAAPTVEMDEELHYASLNFHGMNPSKDTSTEYSEVRTQ

SEQ ID NO: 18 - protein, 790 aa, Homo sapiens
  misc_feature: Incyte ID No 7505086CD1
MANCQIAILYQRFQRVVFGISQLLCFSALISELTNQKEVAAWTYHYSTKAYSWNISRKYCQNRYTDLVAIQNKNEIDYLNKVLPYYSSYYWIGIRKNNKTWTWVGTKKALTNEAENWADNEPNNKRNNEDCVEIYIKSPSAPGKWNDEHCLKKKHALCYTASCQDMSCSKQGECLETIGNYTCSCYPGFYGPECEYVRECGELELPQHVLMNCSHPLGNFSFNSQCSFHCTDGYQVNGPSKLECLASGIWTNKPPQCLAAQCPPLKIPERGNMTCLHSAKAFQHQSSCSFSCEEGFALVGPEVVQCTASGVWTAPAPVCKAVQCQHLEAPSEGTMDCVHPLTAFAYGSSCKFECQPGYRVRGLDMLRCIDSGHWSAPLPTCEAISCEPLESPVHGSMDCSPSLRAFQYDTNCSFRCAEGFMLRGADIVRCDNLGQWTAPAPVCQALQCQDLPVPNEARVNCSHPFGAFRYQSVCSFTCNEGLLLVGASVLQCLATGNWNSVPPECQAIPCTPLLSPQNGTMTCVQPLGSSSYKSTCQFICDEGYSLSGPERLDCTRSGRWTDSPPMCEAIKCPELFAPEQGSLDCSDTRGEFNVGSTCHFSCDNGFKLEGPNNVECTTSGRWSATPPTCKGIASLPTPGVQCPALTTPGQGTMYCRHHPGTFGFNTTCYFGCNAGFTLIGDSTLSCRPSGQWTAVTPACRAVKCSELHVNKPIAMNCSNLWGNFSYGSICSFHCLEGQLLNGSAQTACQENGHWSTTVPTCQDDGKCPLNPHSHLGTYGVFTNAAFDPSP

SEQ ID NO: 19 - protein, 172 aa, Homo sapiens
  misc_feature: Incyte ID No 7505784CD1
MQCFSFIKTMMILFNLLIFFFFILLLIFIAEVAAAVVALVYTTMAEHFLTLLVVPAIKKDYGSQEDFTQVWNTTMKGLKCCGFTNYTDFEDSPYFKENSAFPPFCCNDNVTNTANETCTKQKAHDQKVEGCFNQLLYDIRTNAVTVGGVAAGIGGLELAAMIVSMYLYCNLQ

SEQ ID NO: 20 - protein, 253 aa, Homo sapiens
  misc_feature: Incyte ID No 7505813CD1
MSAQESCLSLIKYFLFVFNLFFFVLGSLIFCFGIWILIDKTSFVSFVGLAFVPLQIWSKVLAISGIFTMGIALLGCVGALKELRCLLGLYFGMLLLLFATQITLGILISTQRAQLERSLRDVVEKTIQKYGTNPEETAAEESWDYVQFQLRCCGWHYPQDWFQVLILRGNGSEAHRVPCSCYNLSATNDSTILDKVILPQLSRLGHLARSRHSADICAVPAESHIYRELGFMTLSIFLCRNLDHVYNRLARYR

SEQ ID NO: 21 - protein, 431 aa, Homo sapiens
  misc_feature: Incyte ID No 7505873CD1
MLLFFTLGLLIHFVFFASIFDIYFTSPLVHGMTPQFTPLPPPARRLVLFVADGLRADALYELDENGNSRAPFIRNIIMHEGSWGISHTRVPTESRPGHVALIAGFYEDVSAVAKGWKENPVEFDSLFNESKYTWSWGSPDILPMFAKGASGDHVYTYSYDAKREDFGAQDATKLDTWVFDNVKDFFHHARNNQSLFSKINEEKIVFFLHLLGIDTNGHAHRPSSRDYKDNIKKVDDGVKEIVSMFNHFYGNDGKTTFIFTSDHGMTDWGSHGAGHPSETLTPLVTWGAGIKYPQRVSAQQFDDAFLKEWRLENWKRLDVNQADIAPLMTSLIGVPFPLNSVGILPVDYLNNTDLFKAESMFTNAVQILEQFKVKMTQKKEVTLPFLFTPFNISHYVIVMSMTIFLVFLNGLAQLLTTKKLRLCGKPKSHFM

SEQ ID NO: 22 - protein, 206 aa, Homo sapiens
  misc_feature: Incyte ID No 7505881CD1
MLLQSQTMGVSHSFTPKGITIPQREKPGHMYQNEDYLQNGLPTETTVLGTVQILCCLLISSLGAILVFAPYPSHFNPAISTTLMSGYPFLGALCFGITGSLSIISGKQSTKPFDLSSLTSNAVSSVTAGAGLFLLADSMVALRTASQHCGSEMDYLSSLPYSEYYYPIYEIKDCLLTSVSLTSSFSSTQSQDHIQQVKKSSSRSWI

SEQ ID NO: 23 - protein, 694 aa, Homo sapiens
  misc_feature: Incyte ID No 7503510CD1
MARPVRGGLGAPRRSPCLLLLWLLLLRLEPVTAAAGPRAPCAAACTCAGDSLDCGGRGLAALPGDLPSWTRSLNLSYNKLSEIDPAGFEDLPNLQEVYLNNNELTAVPSLGAASSHVVSLFLQHNKIRSVEGSQLKAYLSLEVLDLSLNNITEVRNTCFPHGPPIKELNLAGNRIGTLELGAFDGLSRSLLTLRLSKNRITQLPVRAFKLPRLTQLDLNRNRIRLIEGLTFQGLNSLEVLKLQRNNISKLTDGAFWGLSKMHVLHLEYNSLVEVNSGSLYGLTALHQLHLSNNSIARIHRKGWSFCQKLHELVLSFNNLTRLDEESLAELSSLSVLRLSHNSISHIAEGAFKGLRSLRVLDLDHNEISGTIEDTSGAFSGLDSLSKLTLFGNKIKSVAKRAFSGLEGLEHLNLGGNAIRSVQFDAFVKMKNLKELHISSDSFLCDCQLKWLPPWLIGRMLQAFVTATCAHPESLKGQSIFSVPPESFVCDDFLKPQIITQPETTMAMVGKDIRFTCSAASSSSSPMTFAWKKDNEVLTNADMENFVHVHAQDGEVMEYTTILHLRQVTFGHEGRYQCVITNHFGSTYSHKARLTVNVLPSFTKTPHDITIRTTTMARLECAATGHPNPQIAWQKDGGTDFPAAQTPSLVVPLEDRVVSVGVTKSILHITSAAGLPLGPAPSAKGRSTPVTTIEC

SEQ ID NO: 24 - protein, 228 aa, Homo sapiens
  misc_feature: Incyte ID No 7714715CD1
MCHVIVTCRSMLWTLLSIVVAFAELIAFMSADWLIGKARSRGGVEPAGPGGGSPEPYHPTLGIYARCIRNPGVQHFQRDTLCGPYAESFGEIASGFWQATAIFLAVGIFILCMVALVSVFTMCVQSIMKKSIFNVCGLLQGIAGLFLILGLILYPAGWGCQKAIDYCGHYASAYKPGDCSLGWAFYTAIGGTVLTFICAVFSAQAEIATSSDKVQEEIEEGKNLICLL

SEQ ID NO: 25 - protein, 216 aa, Homo sapiens
  misc_feature: Incyte ID No 7506032CD1
MTAAVFFGCAFIAFGPALALYVFTIATEPLRIIFLIAGAFFWLVSLLISSLVWFMARVIIDNKDGPTQKYLLIFGAFVSVYIQEMFRFAYYKLLKKASEGLKSINPGETAPSMRLLAYAFMTLVIILLHVFWGIVFFDGCEKKKWGILLIVLLTHLLVSAQTFISSYYGINLASAFIILVLMGTWAFLAAGGSCRSLKLCLLCQDKNFLLYNQRSR

SEQ ID NO: 26 - protein, 359 aa, Homo sapiens
  misc_feature: Incyte ID No 7506034CD1
MGTFCSVIKFENLQELKRLCHWGPIIALGVIAICSTMAMIDSVLWYWPLHTTGGSVNFIMLINWTVMILYNYFNAMFVGPGFVPLGWKPEISQDTMYLQYCKVCQAYKAPRSHHCRKCNRCVMKMDHHCPWINNCCGYQNHASFTLFLLLAPLGCIHAAFIFVMTMYTQLYHRMKIILRNKTSIESWIEEKAKDRIQYYQLDEVFVFPYDMGSRWRNFKQVFTWSGVPEGDGLEWPVREGCHQYSLTIEQLKQKADKRVRSVRYKVIEDYSGACCPLNKGIKTFFTSPCTEEPRIQLQKGEFILATRGLRYWLYGDKILDDSFIEGVSRIRGWFPRKCVEKCPCDAETDQAPEGEKKNR

SEQ ID NO: 27 - protein, 115 aa, Homo sapiens
  misc_feature: Incyte ID No 7506100CD1
MEVKDANSALLSNYETLKYISKTPCRHQSPEIVREFLTALKSHKLTKAEKLQLLNHRPVTAVEIQLMVEESEERLTEEQIEALLHTVTSILPAEPEAEQKKNTNSNVAMDEEDPA

SEQ ID NO: 28 - protein, 454 aa, Homo sapiens
  misc_feature: Incyte ID No 1743113CD1
MGRTYIVEETVGQYLSNINLQGKAFVSGLLIGQCSSQKDYVILATRTPPKEEQSENLKHPKAKLDNLDEEWATEHACQVSRMLPGGLLVLGVFIITTLELANDFQNALRRLMFAVEKSINRKRLWNFTEEEVSERVTLHICASTKKIFCRTYDIHDPKSSARPADWKYQSGLSSSWLSLECTVHINIHIPLSATSVSYTLEKNTKNGLTRWAKEIENGVYLINGQVKDEDCDLLEGQKKSSRGNTQATSHSFDVRVLTQLLLNSDHRSTATVQICSGSVNLKGAVKCRAYIHSSKPKVKDAVQAVKRDILNTVADRCEMLFEDLLLNEIPEKKDSEKEFHVLPYRVFVPLPGSTVMLCDYKFDDESAEEIRDHFMEMLDHTIQIEDLEIAEETNTACMSSSMNSQASLDNTDDEQPKQPIKTTMLLKIQQNIGVIAAFTVAVLAAGISFHYFSD

SEQ ID NO: 29 - protein, 251 aa, Homo sapiens
  misc_feature: Incyte ID No 7505144CD1
MLPAATEVQLRLQGQKDMVWKWMPLLLLLVCVATMCSAQDRTDLLNVCMDAKHHKTKPGPEDKLHDQCSPWKKNACCTASTSQELHKDTSRLYNFNWDHCGKMEPACKRHFIQDTCLYECSPNLGPWIQQVNQSWRKERFLDVPLCKEDCQRWWEDCHTSHTCKSNWHRGWDWTSAALCEGLWSHSYKVSNYSRGSGRCIQMWFDSAQGNPNEEVARFYAAAMHVNAGEMLHGTGGLLLSLALMLQLWLLG

SEQ ID NO: 30 - protein, 193 aa, Homo sapiens
  misc_feature: Incyte ID No 7506132CD1
MGRFRGGLRCIKYLLLGFNLLFWLAGSAVIAFGLWFRFGGAIKELSSEDKSPEYFYVGLYVLVGAGALMMAVGFFGCCGAMRESQCVLGSFFTCLLVIFAAEVTTGVFAFIGKGVAIRHVQTMYEEAYNDYLKDRGKGNGTLITFHSTFQCCGKESSEQVQPTCPKELLGHKIFGMIFSMVLCCAIRNSRDVI

SEQ ID NO: 31 - protein, 529 aa, Homo sapiens
  misc_feature: Incyte ID No 8142016CD1
MQYSNKNIALDGARTAKMKAIIHLTLLALLSVNTATNQGNSADAVTTTETATSGPTVAAADTTETNFPETASTTANTPSFPTATSPAPPIISTHSSSTIPTPAPPIISTHSSSTIPIPTAADSESTTNVNSLATSDIITASSPNDGLITMVPSETQSNNEMSPTTEDNQSSGPPTGTALLETSTLNSTGPSNPCQDDPCADNSLCVKLHNTSFCLCLEGYYYNSSTCKKGKVFPGKISVTVSETFDPEEKHSMAYQDLHSEITSLFKDVFGTSVYGQTVILTVSTSLSPRSEMRADDKFVNVTIVTILAETTSDNEKTVTEKINKAIRSSSSNFLNYDLTLRCDYYGCNQTADDCLNGLACDCKSDLQRPNPQSPFCVASSLKCPDACNAQHKQCLIKKSGGAPECACVPGYQEDANGNCQKCAFGYSGLDCKDKFQLILTIVGTIAGIVILSMIIALIVTARSNNKTKHIEEENLIDEDFQNLKLRSTGFTNLGAEGSVFPKVRITASRDSQMQNPYSSHSSMPRPDY

SEQ ID NO: 32 - protein, 573 aa, Homo sapiens
  misc_feature: Incyte ID No 7506135CD1
MMPSPSDSSRSLTSRPSTRGLTHLRLHRPWLQALLTLGLVQVLLGILVVTFSMVASSVTTTESIKRSCPSWAGFSNLLFSVCGLTICAAIICTLSAIVCCIQIFSLDLVHTQLAPERSVSGPLGPLGCTSPPPAPLLHTMLDLEEFVPPVPPPPYYPPEYTCSSETDAQSITYNGSMDSPVPLYPTDCPPSYEAVMGLRGDSQATLFDPQLHDGSCICERVASIVDVSMDSGSLVLSAIGDLPGGSSPSEDSCLLELQGSVRSVDYVLFRSIQRSRAGYCLSLDCGLRGPFEESPLPRRPPRAARSYSCSAPEAPPPLGAPTAARSCHRLEGWPPWVGPCFPELRRRVPRGGGRPAAAPPTRAPTRRFSDSSGSLTPPGHRPPHPASPPPLLLPRSHSDPGITTSSDTADFRDLYTKVLEEEAASVSSADTGLCSEACLFRLARCPSPKLLRARSAEKRRPVPTFQKVPLPSGPAPAHSLGDLKGSWPGRGLVTRFLQISRKAPDPSGTGAHGHKQVPRSLWGRPGRESLHLRSCGDLSSSSSLRRLLSGRRLERGTRPHSLSLNGGSRETGL

SEQ ID NO: 33 - protein, 232 aa, Homo sapiens
  misc_feature: Incyte ID No 90086301CD1
MALNDCFLLNLEVDHFMHCNISSHSADLPVNDDWSHPGILYVIPAVYGVIILIGLIGNITLIKIFCTVKSMRNVPNLFISSLALGDLLLLITCAPVDASRYLADRWLFGRIGCKLIPFIQLTSVGVSVFTLTALSADREKNRKESEGMNCKDAAAGNSSLEFQSRPSSPSTERESSFKSQVALLRGSSVFPIQSHCPANGYPGLSCPDEDLPQSRLYLDHLHAAGHSRGRVF

SEQ ID NO: 34 - protein, 312 aa, Homo sapiens
  misc_feature: Incyte ID No 7487373CD1
MGLNKSASTFQLTGFPGMEKAHHWIFIPLLAAYISILLGNGTLLFLIRNDHNLHEPMYYFLAMLAATDLGVTLTTMPTVLGVLWLDHRETGHGACFSQAYFIHTLSVMESGVLLAMAYDCFIAIHNPLRYISILTNTQVMKIGVGVLTRAGLSIMPIVVRLHWFPYCRAHVFSHAFCLHQDVIKLACADITLNRLYPVVVLFAMVLLDFLIIFFSYILILKTVMGIGSGGERAKALNTCVSHICCILVFYVTVVCLTFIHRFGKHVPHVVHITMRYIHFLFPPFMNPFIYSIKTKQIQSGILRLFSLPHSRA

SEQ ID NO: 35 - protein, 379 aa, Homo sapiens
  misc_feature: Incyte ID No 7506228CD1
MFFTCGPNEAMVVSGFCRSPPVMVAGGRVFVLPCIQQIQRISLNTLTLNVKSEKVYTRHGVPISVTGIAQEIYKDRQKFSEQVFKVASSDLVNMGISVVSYTLKDIHDDQDYLHSLGKARTAQVQKDARIGEAEAKRDAGIREAKAKQEKVSAQYLSEIEMAKAQRDYELKKAAYDIEVNTRRAQADLAYQLQVAKTKQQIEEQRVQVQVVERAQQVAVQEQEIARREKELEARVRKPAEAERYKLERLAEAEKSQLIMQAEAEAASVRMRGEAEAFAIGARARAEAEQMAKKAEAFQLYQEAAQLDMLLEKLPQVAEEISGPLTSANKITLVSSGSGTMGAAKVTGEVLDILTRLPESVERLTGVSISQVNHKPLRTA

SEQ ID NO: 36 - protein, 453 aa, Homo sapiens
  misc_feature: Incyte ID No 7506194CD1
MSWFSGLLVPKVDERKTAWGERNGQKRSRRRGTRAGGFCTPRYMSCLRDAEPPSPTPAGPPRCPWQDDAFIRRGGPGKGKELGLRAVALGFEDTEVTTTAGGTAEVAPDAVPRSGRSCWRRLVQVFQSKQFRSAKLERLYQRYFFQMNQSSLTLLMAVLVLLTAVLLAFHAAPARPQPAYVALLACAAALFVGLMVVCNRHSFRQDSMWVVSYVVLGILAAVQVGGALAADPRSPSAGLWCPVFFVYIAYTLLPIRMRAAVLSGLGLSTLHLILAWQLNRGDAFLWKQLGANVLLFLCTNVIGICTHYPAEVSQRQAFQETRGYIQARLHLQHENRQQERLLLSVLPQHVAMEMKEDINEHSFNNFQMKIGLNMGPVVAGVIGARKPQYDIWGNTVNVSSRMDSTGVPDRIQVTTDLYQVLAAKGYQLECRGVVKVKGKGEMTTYFLNGGPSS

SEQ ID NO: 37 - protein, 36 aa, Homo sapiens
  misc_feature: Incyte ID No 7506434CD1
MWGLAGGRLFGIFSAPVLVAVVCCAQSVNDPRSAWG

SEQ ID NO: 38 - protein, 398 aa, Homo sapiens
  misc_feature: Incyte ID No 7490974CD1
MDRSLRNVLVVSFGFLLLFTAYGGLQSLQLQRRLPGQQACGWRFLGAVAASSLGDTEDLLVPHPAVSILCSDRAGFVPTEQPVQRGGPGCHSAQHPLWRHAPVLHVPPTAPHREAGLQGDHHPLHVWLRGLLRGQLLRQLVYTLLGIYTGSGVLAVLMIAAFLQPIRDVQRESEGEKKSVPFWSTLLSTFKLYRDKRLCLLILLPLYSGLQQGFLSSEYTRSYVTCTLGIQFVGYVMICFSATDALCSVLYGKVSQYTGRAVLYVLGAVTHVSCMIALLLWRPRADHLAVFFVFSGLWGVADAVWQTQNNALYGVLFEKSKEAAFANYRLWEALGFVIAFGYSTFLCVHVKLYILLGVLSLTMVAYGLVECVESKNPIRPHAPGQVNQAEDEEIQTKM

SEQ ID NO: 39 - protein, 750 aa, Homo sapiens
  misc_feature: Incyte ID No 7506224CD1
MTPEFDEEVVFENSPLYQYLQDLGHTDFEICSSLSPKTEKCTTEGQQKPPTRVLPKDIGFRLDSLHTILQQEVLLQEDVELIELLDPSILSAGQSQQQENGHLPTLCSLATPNIWDLSMLFAFISLLVMLPTWWIVSSWLVWGVILFVYLVIRALRLWRTAKLQVTLKKYSVHLEDMATNSRAFTNLVRKALRLIQETEVISRGFTLVSAACPFNKAGQHPSQHLIGLRKAVYRTLRANFQAARLATLYMLKNYPLNSESDNVTNYICVVPFKELGLGLSEEQISEEEAHNFTDGFSLPALKVLFQLWVAQSSEFFRRLALLLSTANSPPGPLLTPALLPHRILSDVTQGLPHAHSACLEELKRSYEFYRYFETQHQSVPQCLSKTQQKSRELNNVHTAVRSLQLHLKALLNEVIILEDELEKLVCTKETQELVSEAYPILEQKLKLIQPHVQASNNCWEEAISQVDKLLRRNTDKKGKPEIACENPHCTVVPLKQPTLHIADKDPIPEEQELEAYVDDIDIDSDFRKDDFYYLSQEDKERQKREHEESKRVLQELKSVLGFKASEAERQKWKQLLFSDHEAVLKSLSPVDPVEPISNSEPSMNSDMGKVSKNDTEEESNKSATTDNEISRTEYLCENALEGKNKDNSSNEVFPQGAEERMCYQCESEDEPQADGSGLTTAPPTPRDSLQPSIKQRLARLQLSPDFTFTAGLAAEVAARSLSFTTMQEQTFGDEEEEQIIEENKNEIEEK

SEQ ID NO: 40 - protein, 162 aa, Homo sapiens
  misc_feature: Incyte ID No 7506280CD1
MDVNIAPLRAWDDFFPGSDRFARPDFRDISIVGFLSPFNMILGGIVVVLVFTGFVWAAHNKDVLRRMKKRYPTTFVMVVMLASYFLISMFGGVMVFVFGITFPLLLMFIHASLRLRNLKNKLENKMEGIGLKRTPMGIVLDALEQQEEGINRLTDYISKVKE

SEQ ID NO: 41 - protein, 417 aa, Homo sapiens
  misc_feature: Incyte ID No 7508326CD1
MSTEKVDQKEEAGEKEVCGDQIKGPDKEEEPPAAASHGQGWRPGGRAARNARPEPGARHPALPAMVNDPPVPALLWAQEVGQVLAGRARRLLLQFGVLFCTILLLLWVSVFLYGSFYYSYMPTVSHLSPVHFYYRTDCDSSTTSLCSFPVANVSLTKGGRDRVLMYGQPYRVTLELELPESPVNQDLGMFLVTISCYTRGGRIISTSSRSYVPTTGAIIEIHSKRIQLYGAYLRIHAHFTGLRYLLYNFPMTCAFIGVASNFTFLSVIVLFSYMQWVWGGIWPRHRFSLQVNIRKRDNSRKEVQRRISAHQPGPEGQEESTPQSDVTEDGESPEDPSGTEGQLSEEEKPDQQPLSGEEELEPEASDGSGSWEDAALLTEANLPAPAPASASAPVLETLGSSEPAGGALRQRPTCSSS

SEQ ID NO: 42 - protein, 176 aa, Homo sapiens
  misc_feature: Incyte ID No 7506370CD1
MNGLPSAEAPGGAGCALAGLPPLPRGLSGLLNASGGSWRELERVYSQRSRIHDELSRAARAPDGPRHAAGAANAGPAAGPRRPVNLDSALAALRKEMLWGLYESIQDYKHLCQDLSFCQDLSSSLHSDSSYPPDAGLSDDEEPPDASLPPDPPPLTVPQTHNARDQWLQDAFHISL

SEQ ID NO: 43 - protein, 579 aa, Homo sapiens
  misc_feature: Incyte ID No 6312989CD1
MSRCPRVSSSPRARCPSSARGGEGALRGPSVTPLAWPPPQLASLGHAPGDPARGWALERVGWRGERTGPWAPLRCSQVPGRAERGAGNRAAGMMPSPSDSSRSLTSRPSTRGLTHLRLHRPWLQALLTLGLVQVLLGILVVTFSMVASSVTTTESIKRSCPSWAGFSLAFSGVVGIVSWKRPFTLVISFFSLLSVLCVMLSMAGSVLSCKNAQLARDFQQCSLEGKVCVCCPSVPLLRPCPESGQELKVAPNSTCDEARGALKNLLFSVCGLTICAAIICTLSAIVCCIQIFSLDLVHTQLAPERSVSGPLGPLGCTSPPPAPLLHTMLDLEEFVPPVPPPPYYPPEYTCSSETDAQSITYNGSMDSPVPLYPTDCPPSYEAVMGLRGDSQATLFDPQLHDGSCICERVASIVDADFRDLYTKVLEEEAASVSSADTGLCSEACLFRLARCPSPKLLRARSAEKRRPVPTFQKVPLPSGPAPAHSLGDLKGSWPGRGLVTRFLQISRKAPDPSGTGAHGHKQVPRSLWGRPGRESLHLRSCGDLSSSSSLRRLLSGRRLERGTRPHSLSLNGGSRETGL

SEQ ID NO: 44 - protein, 357 aa, Homo sapiens
  misc_feature: Incyte ID No 7501108CD1
MRGSVECTWGWGHCAPSPLLLWTLLLFAAPFGLLGEKTRQVSLEVIPNWLGPLQNLLHIRAVGTNSTLHYVWSSLGPLAVVMVATNTPHSTLSVNWSLLLSPEPDGGLMVLPKDSIQFSSALVFTRLLEFDSTNVSDTAAKPLGRPYPPYSLADFSWNNITDSLDPATLSATFQGHPMNDPTRTFANGSLAFRVQAFSRSSRPAQPPRLLHTADTCQLEVALIGASPRGNRSLFGLEVATLGPGPDCPSMQEQHSIDDEYAPAVFQSPIVRAFFGSQNNFCAFNLTFGASTGPGYWDQHYLSWSMLLGVGFPPVDGLSPLVLGIMAVALGAPGLMLLGGGLVLLLHHKKYSEYQSIN

SEQ ID NO: 45 - protein, 301 aa, Homo sapiens
  misc_feature: Incyte ID No 7507581CD1
MHPEPAPPPSRSSPELPPSGGSTTSGSRRSRRRSGDGEPPGAPPPPPSAVTYPDWIGQSYSEVMSLNEHSMQALSWRKLYLSRAKLKASSRTSALLSGFAMVAMVEVQLDADHDYPPGLLIAFSACTTVLVAVHLFALMISTCILPNIEAVSNVHNLNSVKESPHERMHRHIELAWAFSTVIGTLLFLAEVVLLCWVKFLPLKKQPGQPRPTSKPPASGAAANVSTSGITPGQAAAIASTTIMVPFGLIFIVFAVHFYRSLVSHKTDRQFQELNELAEFARLQDQLDHRGDHPLTPGSHYA

SEQ ID NO: 46 - protein, 562 aa, Homo sapiens
  misc_feature: Incyte ID No 7506361CD1
MEGFGGVGGRGTRGFAAKGVWRGRAEEGPVLGAAERGFMVSTGSRRRVFEGPGGGGLRWTPGKGTGRQRGAWGPRAEDGVRRRTLGMPRGSRRDVRAPCGPAGSWGARGGRRRDGPSRRRRGSATAAARHHVPPAPGGPFGPRAPAGSTRVPARAGGAVEPTGAAAVARLARPAGGALPTAGAQGAGPARGRSGEGSEWARRGKGRPGPYQSPLGPAVAEGQELKDKSRLRYPINGFQALVLTALLVGLGMSAGLPLGALPEMLLPLAFVATLTAFIFSLFLYMKAQVAPVSALAPGGNSGNPIYDFFLGRELNPRICFFDFKYFCELRPGLIGWVLINLALLMKEAELRGSPSLAMWLVNGFQLLYVGDALWHEEAVLTTMDITHDGFGFMLAFGDMAWVPFTYSLQAQFLLHHPQPLGLPMASVICLINGLETISTATGRKLLVSGWWGMVRHPNYLGDLIMALAWSLPCGVSHLLPYFYLLYFTALLVHREARDERSACRSTAWPGRSTAGVCLTASCPTSTEAAPPPQVGHVPTHPPAHPGPGASTHLGLKGLHPTQP

SEQ ID NO: 47 - protein, 651 aa, Homo sapiens
  misc_feature: Incyte ID No 7509211CD1
MMPSPSDSSRSLTSRPSTRGLTHLRLHRPWLQALLTLGLVQVLLGILVVTFSMVASSVTTTESIKRSCPSWAGFSISFFSLLSVLCVMLSMAGSVLSCKNAQLARDFQQCSLEGKVCVCCPSVPLLRPCPESGQELKVAPNSTCDEARGALKNLLFSVCGLTICAAIICTLSAIVCCIQIFSLDLVHTQLAPERSVSGPLGPLGCTSPPPAPLLHTMLDLEEFVPPVPPPPYYPPEYTCSSETDAQSITYNGSMDSPVPLYPTDCPPSYEAVMGLRGDSQATLFDPQLHDGSCICERVASIVDVSMDSGSLVLSAIGDLPGGSSPSEDSCLLELQGSVRSVDYVLFRSIQRSRAGYCLSLDCGLRGPFEESPLPRRPPRAARSYSCSAPEAPPPLGAPTAARSCHRLEGWPPWVGPCFPELRRRVPRGGGRPAAAPPTRAPTRRFSDSSGSLTPPGHRPPHPASPPPLLLPRSHSDPGITTSSDTADFRDLYTKVLEEEAASVSSADTGLCSEACLFRLARCPSPKLLRARSAEKRRPVPTFQKVPLPSGPAPAHSLGDLKGSWPKVGALVTRFLQISRKAPDPSGTGAHGHKQVPRSLWGRPGRESLHLRSCGDLSSSSSLRRLLSGRRLERGTRPHSLSLNGGSRETGL

SEQ ID NO: 48 - DNA, 2061 bp, Homo sapiens
  misc_feature: Incyte ID No 3356677CB1
agaatgaccagctaggcttccctggaggccaggcctccctggactgggccaggtgcttcctgccagcaaagatgagtaatgtctcagggatcctggagacagccggcgtccccctggtgtcagcgaactggccgcagcccagccccccaccggctgtgccagctgggccgcagatggaccacatggggaacagctcccagggggccccctggctcttcctcacctccgcactggcccgaggcgtctcggggatcttcgtgtggactgccctggtgctcacctgccaccagatctatctgcacctgcgctcctacaccgtgccacaggagcaacgttacatcatccgcctgctcctcatcgtgcccatctacgccttcgactcctggctcagcctcctcctcctcggagaccaccagtactacgtctacttcgactctgtgcgggactgctacgaagcctttgtcatttacagcttcctgagcctgtgtttccagtacctgggaggcgagggcgccatcatggctgagattcgtggaaagcccatcaagtccagctgcttgtacggcacctgctgcctccggggcatgacctactccatcgggttcctgcgcttctgtaagcaggccactctgcagttctgcctggtgaagcccgtcatggccgtcaccaccatcatcctccaggcatttggcaaataccacgacggggacttcaatgtccgcagcgtctacctctatgtgaccctcatctacaacgcctccgtcagcctcgccctctacgccctgttcctcttctacttcaccaccagggagctcctgcggcccttccagcccgtcctcaagttcctcaccatcaaagccgtcatcttcctgtcgttctggcaagggctgctgctggccatcctggagcggtgcggggtcatcccggaggtggagaccagcggcgggaacaagctgggggctggcacgctggccgccggctaccagaacttcatcatctgcgtggagatgctgttcgcctccgtggccctgcgttatgccttcccctgccaggtgtacgcagagaagaaggagaattcaccagcccccccggcacccatgcagagcatctccagcggcatcagggagacagtgagcccccaggacatcgtgcaggacgccatccacaacttctcccccgcctaccagcactacacgcagcaggccacgcacgaggcgcccaggcccggcacccaccccggcggcggcggctccggcgggagcaggaagagccggagcctggagaagcggatgctgatcccctcggaggacctgtaggggggcctgggctgccagtgctgtagggacccaggctgcccaggcctctggggaagaacagggtccccccacccaccaactcctgccaaaggtggggcctctcctgagagcccacctgtgaggccctcggagcccacttcccatcctccctccagccagggggtcagggcacctgatggccctggcaggcacccaggtgggcccgccaccgcaggagagggcacctgagccaatcggaagagcctggggaccccctgggatcacccagccatcagccccaggagccactgtggggcggagagtgagtgtggctgcggggccttggctgcacggaccccatgggagctgcgagtgggtcagactccctggttcaggagacagacagcggacggatcccaggctgggcagctggagggaggggcgccggggcgctgggcagccgggctctgacacagtcagcagctccgggcgccgcaggccggcggggtccacacaggctggccggggctgggcctccttggagcctgctacggccctcgtgggcacgtggagaagggcccacgtgtctccacacgccagccacaggggagccctggccaggcgcccagccaggggagcgtgtgcctgggatgggtcacagaaccagcgggcacctgtgaggctggccagcaccgtggggctgtgggaatcgctcttatttatatttaaacaccttggattttcaaaaaaaaaaaaaaggtggcgg

SEQ ID NO: 49 - DNA, 2649 bp, Homo sapiens
  misc_feature: Incyte ID No 7481665CB1
gagagcgtctctccgggcgccgctgctgttgtcccgggtgctgaaaagccgggaacttcccgggaagcagcggcacttttctccctggaaggctaaacatatttttgactggtatgaacccattctactttcatgcagtaaatataattttacactgcttagtgactcttgtgctgatgtacacctgtgataaaactgtcttcaagaatcgtggacttgcttttgtaacggcattgctttttgctgtacatcctattcatactgaggcggtggctgggatcgttggcagagcggacgtgttagcgtgtctgctgtttctattggcctttctctcgtacaacaggagtctggatcagggctgtgttgggggaagtttcccttccacggtgtctcccttcttcttgctgctcagtttgtttctggggacctgtgcgatgctggtgaaagagacaggcatcacggtgtttggagtgtgcttggtttatgacctcttttccctttccaacaagcaagacaagtcgagcaatggggccctctgtccacgcagcccacagcagcccgggagcccccagccctcctcactgccaggccatcctcaccgggagaatgggaagcagcagcggttccctcacaaaggagcttggggtggctgccactctccactgccaccagaacccaagagcagtggattcccagtgtccccacgagctgtgtggtccatgatgaggtatctgagagcaagcagcaatagaaatttcctgcttaccatgagaccatttttaaaaagggccattttggtcctaagttatgtgcttgtcattttgtacttccgtctgtggataatgggaggatctatgcccctcttttcagagcaggataatccagcttcattttcgccttacattcttacgagattcctcacctattcctacctcttggccttcaatgtgtggcttctgcttgcacccgtgaccctgtgctatgactggcaggtcggcagtattcctctggtagagaccatatgggacatgcggaacttagccaccatctttctggcggttgtgatggccttattgagcctgcactgcttagcagcctttaagagactggagcacaaggaggttttagtcggcttgttgttcctggtgttcccgttcattccagccagcaacctcttcttcagggtgggttttgtggtggcggagagagtcctttacatgcctagcatgggctactgcatcctttttgtgcatggactgagcaagctctgcacttggctgaatcgatgtggggccaccaccctgattgtgtccactgtgttgctgctgttgcttttctcttggaaaactgtgaaacagaatgaaatttggctgtcaagagagtccctattcaggtctggagttcaaactctgccccacaatgccaaggttcactacaactatgccaatttcctgaaggaccaaggtcggaacaaggaagcgatctaccactacagaacagctctcaagttgtatccacgccatgcaagtgcgctcaacaaccttggaacactgacgagagacacagcagaggcaaagatgtactatcagagggctctccagctccatccacagcataaccgggctcttttcaatctggggaatctcctcaagtcccaggagaaaaaggaagaagctatcaccttactgaaggattccatcaaatatggtccagagtttgcagatgcatattcaagcttagcttcgttattggctgagcaggagcggtttaaagaagctgaagaaatataccaaactggaataaagaactgtccagacagctcagatttacacaacaactatggggttttcttagttgatactggcttaccagaaaaggcagtggcccattaccagcaggccatcaaacttagccccagtcatcacgtggccatggtgaacttgggaagactctacaggtcactgggagagaacagcatggctgaagaatggtacaagcgcgccctgcaggtggcacacaaagctgagatattgtcacctttgggagcactgtattacaacactggccgatacgaagaggctttgcagatttaccaggaagctgcagcacttcagccttctcagagggagctccgcttggcactggctcaggttttggccgtgatgggtcagacaaaagaagctgaaaagatgaccaatcacattgtgtcagaggagaccggatgccttgaatgctatcgcctcttgtcagccatctatagcaagcaggagaaccacgacaaggcacttgatgctatagacaaggctctccagctgaaaccaaaggacccaaaagtcatttctgaactttttttcacaaaaggaaaccaattaagagagcagaaccttctcgacaaagcttttgagagctatagagtggctgtgcaactaaacccagaccaagcacaggcctggatgaacatgggtggcatccaacacatcaagggaaaatatgtgtctgcaagagcttattatgagagagccttacagctggttccagacagcaaactgctgaaggaaaatcttgccaaattggatcgcctagaaaaacgattacaagaagttcgagaaaaggatcaaacatagcaccaccgtctgacccaactcatag

SEQ ID NO: 50 - DNA, 1528 bp, Homo sapiens
  misc_feature: Incyte ID No 3563859CB1
cacagggcactgaagtggatacccatgctgatctgcctcccaaggcctatgatggccacagtgctgtgcacagccaccttgctgcatcctgctgttggtgggctcttgtgctcagaaccccaggagaagggctagatcacactgataagactggcagctttttttaaaaaacgactttcaaggtttggttatttttatctccatgacctctgatttccctgtggagcaaatggtaaagtaggggtgggtggagagggactttggcagactatggacaaggacagccctgaacagctggccctgtcacaggaactggaacatggtcggagccaaggacacaggactaacaggaaaggacacagactgctgtcaggacacatactaccacacacccgaggccaggaccctgctgacgtggcagacctccaccctggccccttactgccccccgcccctctcccccacctgccagctgccaacagggctctgccttgtgtctgccacacctgtcactgcctatccttgtccagggggggccccatcagcccctcctcagctgcccagcaaagcaagcagttagtggggaggggagggaacatggagcgggggccggtggtgggggcaggactgggggccggggcccgaatccaggcactgctgggctgcctgctcaaggtgctgctctgggtggcctctgccttgctgtactttggaagcgaacaggccgcccgccttctgggcagcccctgcttacggcgcctctaccatgcctggctggcagcagtggtcatctttgggccgcttctgcagttccatgtcaaccctcggactatcttcgccagccacggcaacttcttcaacataaaatttgtgaattcagcctggggctggacatgcactttcttagggggctttgtgttgctggtggtgttcctggctacacggcgcgtggcagtaactgccagacacctgagccgactggtagtaggggcagccgtgtggcggggagccggccgggccttcctgctcatcgaggacctgactggctcctgcttcgagccactgccccagggtctgctgctccacgagctgcctgaccgccgcagctgcctggcagccggccaccagtggcgaggctacaccgtctcctcccacaccttcctgctcaccttttgctgcctgctcatggcagaggaagcagctgtgttcgccaagtacctggcccatgggcttcctgccggcgccccactgcgccttgtcttcctgctgaacgtgctgctgctgggcctctggaacttcttgctgctctgtaccgtcatctatttccaccagtacactcacaaggtggtgggcgccgcagtgggcacctttgcctggtacctcacctatggcagctggtatcatcagccctggtctccagggagcccaggccatgggctcttcccccgtccccactccagccgcaagcataactgaaagaaataaaaaccatcgggcctgaaaaaaaaaaaaaaaaaaaaaaaaaaaa

SEQ ID NO: 51 - DNA, 1469 bp, Homo sapiens
  misc_feature: Incyte ID No 2588884CB1
gagggcggggttgggggaggggtgtgtcgggattgtgcctttgtctgtttgtcgttgttggtcggtcgtgtggttgttctgtcggtttcgatgtcgtgcagcgcctcaggtctgttgggtagtatttcgtcgtgatgtgtgtctcggctgcctgcttgtgctgtctctgctcagttgtgtttgcggcgatgcgtgcgttgacgcgttccgcgcccgtctggttttggtcctcccccctgattaacgcgctgtcctccccgttgtcgcttaagagtggggagtttgtttgggcacccgaaatcagacgggaacttttccgaaaaggtcggtaacaaactccgcgcccagttgacgccaaattggccgggtaggcggtgtaacggtgggaggtctatataagcagagctggtttagtgaaccgtcagatccgctagccgcaattactgtgagttagctcactcattaggcaccccaggctttacacttggacacctgctgctccgtatattgtgtggaagaattcggctcgaggactgcttcactctctcattcttagcttgaatttggaaatgacttttgatgacctaaagatccagactgtgaaggaccagcctgatgagaagtcaaatggaaaaaaagctaaaggtcttcagtttctttactctccatggtggtgcctggctgctgcgactctaggggtcctttgcctgggattagtagtgaccattatggtgctgggcatgcaattatcccaggtgtctgacctcctaacacaagagcaagcaaacctaactcaccagaaaaagaaactggagggacagatctcagcccggcaacaagcagaagaagcttcacaggagtcagaaaacgaactcaaggaaatgatagaaacccttgctcggaagctgaatgagaaatccaaagagcaaatggaacttcaccaccagaatctgaatctccaagaaacactgaagagagtagcaaattgttcaggacttcatccagcaagcaatttcctattccagttttccattctggatggggctgtctcggaggaaccccagctacccatggctctgggaggacggttctcctttgatgccccacttatttagagtccgaggcgctgtctcccagacatacccttcaggtacctgtgcatatatacaacgaggagctgtttatgcggaaaactgcattttagctgccttcagtatatgtcagaagaaggcaaacctaagagcacagtgaatttgaaggctctggaagaaaagaaaaaagtctttgagttttattctggaatttaagctattctttgtcacttgggtgccaaacatgagagcccagaaaactgtcatttagctggctgcagaactcctttgcagaaactggggttccaggtgcctggcacctttatgtcaacatttttgattctagctacctgtattatttcacctagcttgtcccaa

SEQ ID NO: 52 - DNA, 1343 bp, Homo sapiens
  misc_feature: Incyte ID No 7503422CB1
gcggcggtggagagcgcagcgcgcagcccggtgcagccctggctttcccctcgctgcgcgcccgcgccccctttcgcgtccgcaaccagaagcccagtgcggcgccaggagccggacccgcgcccgcaccgctcccgggaccgcgaccccggccgcccagagatgaccgcgaccgaagccctcctgcgcgtcctcttgctcctgctggctttcggccacagcacctatggggctgaatgcttcccggcctgcaacccccaaaatggattctgcgaggatgacaatgtttgcaggtgccagcctggctggcagggtcccctttgtgaccagtgcgtgacctctcccggctgccttcacggactctgtggagaacccgggcagtgcatttgcaccgacggctgggacggggagctctgtgatagagatgttcgggcctgctcctcggccccctgtgccaacaacgggtactcgggaaaggactgccagaaaaaggacgggccctgtgtgatcaacggctccccctgccagcacggaggcacctgcgtggatgatgagggccgggcctcccatgcctcctgcctgtgcccccctggcttctcaggcaatttctgcgagatcgtggccagcccgtgccagaacgggggcacctgcctgcagcacacccaggccatctgcttcaccatcctgggcgtgctcaccagcctggtggtgctgggcactgtgggtatcgtcttcctcaacaagtgcgagacctgggtgtccaacctgcgctacaaccacatgctgcggaagaagaagaagaacctgctgcttcagtacaacagcggggaggacctggccgtcaacatcatcttccccgagaagatcgacatgaccaccttcagcaaggaggccggcgacgaggagatctaagcagcgttcccacagccccctctagattcttggagttccgcagagcttactatacgcggtctgtcctaatctttgtggtgttcgctatctcttgtgtcaaatctggtgaacgctacgcttacatatattgtctttgtgctgctgtgtgacaaacgcaatgcaaaaacaatcctctttctctctcttaatgcatgatacagaataataataagaatttcatctttaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaccggatatcgagaaatcgcgtaaattgcagagaatcccaaaaggggggccccttttaggagcccccgaatttt

SEQ ID NO: 53 - DNA, 1464 bp, Homo sapiens
  misc_feature: Incyte ID No 7503424CB1
ggcggcggtggagagcgcagcgcgcagcccggtgcagccctggctttcccctcgctgcgcgcccgcgccccctttcgcgtccgcaaccagaagcccagtgcggcgccaggagccggacccgcgcccgcaccgctcccgggaccgcgaccccggccgcccagagatgaccgcgaccgaagccctcctgcgcgtcctcttgctcctgctggctttcggccacagcacctatggggctgaatgcttcccggcctgcaacccccaaaatggattctgcgaggatgacaatgtttgcaggtgccagcctggctggcagggtcccctttgtgaccagtgcgtgacctctcccggctgccttcacggactctgtggagaacccgggcagtgcatttgcaccgacggctgggacggggagctctgtgatagagatgttcgggcctgctcctcggccccctgtgccaacaacgggacctgcgtgagcctggacgatggcctctatgaatgctcctgtgcccccgggtactcgggaaaggactgccagaaaaaggacgggccctgtgtgatcaacggctccccctgccagcacggaggcacctgcgtggatgatgagggccgggcctcccatgcctcctgcctgtgcccccctggcttctcaggcaatttctgcgagatcgtggccagcccgtgccagaacgggggcacctgcctgcagcacacccagccggagcaccgcatcctgaaggtgtccatgaaagagctcaacaagaaaacccctctcctcaccgagggccaggccatctgcttcaccatcctgggcgtgctcaccagcctggtggtgctgggcactgtgggtatcgtcttcctcaacaagtgcgagacctgggtgtccaacctgcgctacaaccacatgctgcggaagaagaagaacctgctgcttcagtacaacagcggggaggacctggccgtcaacatcatcttccccgagaagatcgacatgaccaccttcagcaaggaggccggcgacgaggagatctaagcagcgttcccacagccccctctagattcttggagttccgcagagcttactatacgcggtctgtcctaatctttgtggtgttcgctatctcttgtgtcaaatctggtgaacgctacgcttacatatattgtctttgtgctgctgtgtgacaaacgcaatgcaaaaacaatcctctttctctctcttaatgcatgatacagaataataataagaatttcatctttaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaccggatatcgagaaatcgcgtaaattgcagagaatcccaaaaggggggccccttttaggagcccccgaatttt

SEQ ID NO: 54 - DNA, 657 bp, Homo sapiens
  misc_feature: Incyte ID No 7503571CB1
cttcctgacacctcaccatgtgtacgggaaaatgtgcccgctgtgtggggctctccctcattaccctctgcctcgtctgcattgtggccaacgccctcctgctggtacctaatggggagacctcctggaccaacaccaaccatctcagcttgcaagtctggctcatgggcggcttcattggcgggggcctaatgatgctgcgctcggtcttctcctcggcgttcggggtgcttggtgccatctactgcctctcggtgtctggagctgggctccgaaatggacccagatgcttaatgaacggcgagtggggctaccacttcgaagacaccgcgggagcttacttgctcaaccgcactctatgggatcggtgcgaggcgccccctcgcgtggtcccctggaatgtgacgctcttctcgctgctggtggccgcctcctgcctggagatagtactgtgtgggatccagctggtgaacgcgaccattggtgtcttctgcggcgattgcaggaaaaaacaggacacacctcactgaggctccactgaccgccgggttacacctgctccttcctggacgcctacctggctcgctcactcccttgctcgctagaataaactgctttgcgctctctaaaaaaaaaaaaaaaaaaaaaaaaaaaaaa

SEQ ID NO: 55 - DNA, 1513 bp, Homo sapiens
  misc_feature: Incyte ID No 7505722CB1
cagatgctcacagcatggaaaagtccatctggctgctggcctgcttggcgtgggttctcccgacaggctcatttgtgagaactaaaatagatactacggagaacttgctcaacacagaggtgcacagctcgccagcgcagcgctggtccatgcaggtgccacccgaggtgagcgcggaggcaggcgacgcggcagtgctgccctgcaccttcacgcacccgcaccgccactacgacgggccgctgacggccatctggcgcgcgggcgagccctatgcgggcccgcaggtgttccgctgcgctgcggcgcggggcagcgagctctgccagacggcgctgagcctgcacggccgcttccggctgctgggcaacccgcgccgcaacgacctctcgctgcgcgtcgagcgcctcgccctggctgacgaccgccgctacttctgccgcgtcgagttcgccggcgacgtccatgaccgctacgagagccgccacggcgtccggctgcacgtgacagccgcgccgcggatcgtcaacatctcggtgctgcccagtccggctcacgccttccgcgcgctctgcactgccgaaggggagccgccgcccgccctcgcctggtccggcccggccctgggcaacagcttggcagccgtgcggagcccgcgtgagggtcacggccacctagtgaccgccgaactgcccgcactgacccatgacggccgctacacgtgtacggccgccaacagcctgggccgctccgaggccagcgtctacctgttccgcttccatggcgccagcggggcctcgacggtcgccctcctgctcggcgctctcggcttcaaggcgctgctgctgctcggggtcctggccgcccgcgctgcccgccgccgcccagagcatctggacaccccggacaccccaccacggtcccaggcccaggagtccaattatgaaaatttgagccagatgaacccccggagcccaccagccaccatgtgctcaccgtgaggagtccctcagccaccaacatccatttcagcactgtaaagaacaaaggccagtgcgaggcttggctggcacagccagtcctggttctcgggcaccttggcagcccccagctgggtggctcctcccctgctcaaggtcaagaccctgctcataggaggctcatctggcctcctatgtggacaaccatttcggagctccctgatatttttgccagcatttcgtaaatgtgcatacgtctgtgtgtgtgtgtgtgtgtgagagagagagagagagagtacacgcattagcttgagcgtgaaacttccagaaatgttcccttgccctttcttacctagaacacctgctatagtaaacgcagacaggaaactgtttacagggcctggaggcccagtcttgtcctcctctgtccccgacttgctgtgtggacctgggacactctcttcacttctctgggtctcattcatttactgttgaacctttccagcacactggcgccgtanttcagcgatgca

SEQ ID NO: 56 - DNA, 1026 bp, Homo sapiens
  misc_feature: Incyte ID No 7505798CB1
gcgagacgcgcaggcgcagagagccccagccacgccggcccaggtggcctcaggtgagggggggcggagcgcacctgtggggacgggacgacgagttcaagcctccgtgggtgcagttggtcgccagcgagggatgcggagacgcccctgaacgaccatggcatcggccgacgagctgaccttccatgaattcgaggaggccactaatcttctggctgacaccccagatgcagccaccaccagcagaagcgatcagctgaccccacaagggcacgtggctgtggccgtgggctcaggtggcagctatggagccgaggatgaggtggaggaggagagtgacaaggccgcggtcctggaccggatcaaaggctcactgctgccccggcctggccacaactttgtgcggcaccatctgcggaatcggccggatctgtatggccccttctggatctgtgccacgttggcctttgtcctggccgtcactggcaacctgacgctggtgctggcccagaggagggacccctccatccactacagcccccagttccacaaggtgaccgtggcaggcatcagcatctactgctatgcgtggctggtgcccctggccctgtggggcttcctgcggtggcgcaagggtgtccaggagcgcatggggccctacaccttcctggagactgtgtgcatctacggctactccctctttgtcttcatccccatggtggtcctgtggctcatccctgtgccttggctgcagtggctctttggggcgctggccctgggcctgtcagccgccgggctggtattcaccctctggcccgtggtccgtgaggacaccaggctggtggccacagtgctgctgtccgtggtcgtgctgctccacgccctcctggccatgggctgtaagttgtacttcttccagtcgctgcctccggagaacgtggctcctccaccccaaatcacatctctgccctcaaacatcgcgctgtcccctaccttgccgcagtccctggccccctcctaggaagg

SEQ ID NO: 57 - DNA, 1895 bp, Homo sapiens
  misc_feature: Incyte ID No 7505847CB1
acgggccgcagcggcagtgacgtagggttggcgcacggatccgttgcggctgcagctctgcagtcgggccgttccttcgccgccgccaggggtagcggtgtagctgcgcagcgtcgcgcgcgctaccgcacccaggttcggcccgtaggcgtctggcagcccggcgccatcttcatcgagcgccatggccgcagcctgcgggccgggagcggccgggtactgcttgctcctcggcttgcatttgtttctgctgaccgcgggccctgccctgggctggaacgaccctgacagaatgttgctgcgggatgtaaaagctcttaccctccactatgaccgctataccacctcccgcaggctggatcccatcccacagttgaaatgtgttggaggcacagctggttgtgattcttataccccaaaagtcatacagtgtcagaacaaaggctgggatgggtatgatgtacagtgggaatgtaagacggacttagatattgcatacaaatttggaaaaactgtggtgagctgtgaaggctatgagtcctctgaagaccagtatgtactaagaggttcttgtggcttggagtataatttagattatacagaacttggcctgcagaaactgaaggagtctggaaagcagcacggctttgcctctttctctgattattattataagtggtcctcggcggattcctgtaacatgagtggattgattaccatcgtggtactccttgggatcgcctttgtagtctataagctgttcctgagtgacgggcagtattctcctccaccgtactctgagtatcctccattttcccaccgttaccagagattcaccaactcagcaggacctcctcccccaggcttgggaactggtggaatactaggatatttgtttggcagcaatagagcggcaacacccttctcagactcgtggtactacccgtcctatcctccctcctaccctggcacgtggaatagggcttactcaccccttcatggaggctcgggcagctattcggtatgttcaaactcagacacgaaaaccagaactgcatcaggatatggtggtaccaggagacgataaagtagaaagttggagtcaaacactggatgcagaaattttggatttttcatcactttctctttagaaaaaaaaagtactacctgttaacaattgggaaaaggggatattcaaaagttctgtggtgttatgtccagtgtagctttttgtattctattatttgaggctaaaagttgatgtgtgacaaaatacttatgtgttgtatgtcagtgtaacatgcagatgtatattgcagtttttgaaagtgatcattactgtggaatgctaaaaatacattaatttctaaaacctgtgatgccctaagaagcattaagaatgaaggtgttgtactaatagaaactaagtacagaaaatttcagttttaggtggttgtagctgatgagttattacctcatagagactataatattctatttggtattatattatttgatgtttgctgttcttcaaacatttaaatcaagctttggactaattatgctaatttgtgagttctgatcacttttgagctctgaagctttgaatcattcagtggtggagatggccttctggtaactgaatattaccttctgtaggaaaaggtagaaaataagcatctagaaggttgttgtgaatgactctgtgctggcaaaaatgcttgaaacctctatatttctttcgttcataagaggtaaaggtcaaatttttcaacaaaagtcttttaataacaaaagcatgcagttctctgtgaaatctcaaatattgttgtaatagtctgtttcaatcttaaaaagaatcaataaaaacaaacaaggggaaaaaaaaaaaaaaa

SEQ ID NO: 58 - DNA, 1723 bp, Homo sapiens
  misc_feature: Incyte ID No 7505862CB1
ggtcacccaagcttgaatctcagtctgcctctcatctgtgaccctggcgagccactcgtcctcaggaagccttcactttccctagtgcacggcgggcacacagctcaacgtgggactgtgaggatgggaaatgaggggtgccatgcaccctggaggaactcagtgaacagtggcaactgtcacttccctggggccctatggtccttcctttctccccagcctgtccacactagcatcttcctcaactcctggttttcagagggaaacacttatcggtcatctgctccacaggaaacaccaggccaaccacagctggggataaaatagcacaaccacaccctgccgtccagcgcctcccagcctgtgccccttcctagtaccaccagcaaccatcaatcccgtctcctcctgcctcctctcctgcaatccaccccgccacgactatcgccatggcagccctgatcgcagagaacttccgcttcctgtcacttttcttcaagagcaaggatgtgatgattttcaacggcctggtggcactgggcacggtgggcagccaggagctgttctctgtggtggccttccactgcccctgctcgccggcccggaactacctgtacgggctggcggccatcggcgtgcccgccctggtgctcttcatcattggcatcatcctccttctaagctccatcctgggacgtgcggctgtggcccctgtcacctggtctgtcatctccctgctgcgtggtgaggcttatgtctgtgctctcagtgagttcgtggacccttcctcactcacggccagggaagagcacttcccatcagcccacgccactgaaatcctggccaggttcccctgcaaggagaaccctgacaacctgtcagacttccgggaggaggtcagccgcaggctcaggtatgagtcccagctctttggatggctgctcatcggcgtggtggccatcctggtgttcctgaccaagtgcctcaagcattactgctcaccactcagctaccgccaggaggcctactgggcgcagtaccgcgccaatgaggaccagctgttccagcgcacggccgaggtgcactctcgggtgctcgctgccaacaatgtgcgccgcttctttggctttgtggcgctcaacaaggatgatgaggaactgattgccaacttcccagtggaaggcacgcagccacggccacagtggaatgccatcaccggcgtctacttgtaccgtgagaaccagggcctcccactctacagccgcctgcacaagtgggcccagggtctggcaggcaacggcgcggcccctgacaacgtggagatggccctgctcccctcctaaggaggtgcttcccatgctctttgtaaatggcactacttggtcccaaactgaaccccactgcttgctcacatccatatcagaaggggatttttaaaaaactgttatcttcttggccaggggaaaggaccacaaggcaatctggggtgtggacagacccagtagacaatggaagccccagccagcagggccaggtgacagtgaagctcaccagtgggctcctttatggtactctatgcagttaacatgtatctagctgcatagggacacccagcgcagcagtgcaccactgggaagtggcctccagtgcagcctctggccttattttatatatttaaatttttgataaagtttttcttactaaaaggaaaaaaaaaaaaagg

SEQ ID NO: 59 - DNA, 1714 bp, Homo sapiens
  misc_feature: Incyte ID No 7762537CB1
ggtgagacccccagacccacgtccacaggcagccccttcctcacaccatcaccaccaccaccactaccatcagtctggcaccgccaccctcccgcgcttaggggcagggggcctggcctcttccgcggccaccgctcagcgcggtccctcctcctctgccacgctgccgaggcccccccaccacgcccctcccggccctgctgccggggcacccccacccggctgcgctaccttgccccgcatgccacccgacccttacctgcaggagactcgcttcgagggcccacttcccccgccgcggcgccgtgccgccgccccgcccccgccggcgccagcccagactgcccaggcccctggcttcgtggtgcccacgcacgcggggactgtgggcacgctgccgctggggggctacgtagcgcccggataccccctgcagctgcagccttgcactgcttacgtgccggtctacccggtgggcacgccatatgcaggcgggaccccggggggaacaggagtgacctccactctccccccgccgccccagggcccagggctggccctactggagccgaggcgcccgccacacgactacatgcccatcgcggtgctgaccaccatctgttgcttctggcctactggcatcattgccatcttcaaggccgtgcaggtgcgcacggccttggcccgcggagacatggtgtcggccgagatcgcttcacgcgaggcccggaacttctccttcatctccctggccgtgggcatcgcggccatggtgctctgtaccatcctcaccgtagtcatcatcatcgccgcgcagcaccacgagaactactgggatccctaaaaacgcccctggtccggccccactctgcgcccctcgatctcccaggctctttctgcagtcataccgcggacccaatgggcgccctgcacacccgtttctggggccgtcagacttggatacatcgtaaactccgcctccacggaacgtctcgccttgcgagcaagctcggaatccagttcctcaggaacccctccaaaacccacacccccagggacgccgctttccgggatcccggccaaacgccggaccctcagtcgctccaggccccctcaccctcaaagtgtagcgcccccaaccgagcaacctcggtttggtccctaaaaccccgcctcctctataagcaccgccccagctctgacaaaaccccgcctccaggtcggcaggctccgccttcttttcttctccgcggggtgattcagtccagtgattgggtttgtggctccaggcctcgcccacagacggacagacccctccctttcttccggcaaaaggaccgagccctggggtagtaaggcccccacactcctgttttttgcaagtacatttttgtccctcctccacccaggtatctgcctattttcttgctaatcccagaacctttccttttgctttttttaaggacatttgggaagttcctggtgtaggacccttctccctgggataagaaacctgcctgtaaacgctctgtaaatactcccttccacccatcccagcccctgggcagccgggcagaagggaatccaggctatggacctcccaagtccccgctccccgctcccctcggcggccccgccttgttctgatctgtgtgtgagtgtgtgtgaacttctgaaagacaatattaaagagacttagttgatttaaaaaaaa

SEQ ID NO: 60 - DNA, 1199 bp, Homo sapiens
  misc_feature: Incyte ID No 90033462CB1
gtgaatgccccttccatcccccgcccctggtgtctcggtctggtctgaggagacggggacccttctcacaccggccgctgccgccgccgcgctccggaacagatccagtccttctgtggaacttctgaacatcttttattagtggaaatattttctacacaatgaagtcaacaacttaatttaaaccagtgtttgtgcggttctgattcatctgctgtggttcccgaagcttgagatctaaggagtacagggtcttttgtgatgacaatatgactaatagtaaaggaagatctattaccgataaaacaagtggtggtccaagtagtgggggaggttttgtagattggactttacgtttaaacacaattcaatccgacaagtttttaaatttactcttgagtatggttccagtgatttaccagaaaaaccaagaagacaggcacaaaaaagcaaacggcatttggcaagatggattatcaactgcagtacagacttttagtaatagatctgagcaacacatggagtatcacagtttctcagagcagtcttttcatgccaataatgggcacgcatcatcaagctgcagccaaaagtatgatgactatgccaattataattactgtgatggaagggagacttcagaaaccactgccatgttacaagatgaagatatatctagtgatggtgatgaagatgctattgtagaagtgaccccaaaattaccaaaggaatccagtggcatcatggcattgcaaatacttgtgccctttttgctagctggttttggaacagtttcagctggcatggtactggatatagtacagcactgggaggtgttcagaaaagttacagaagttttcattttagtccctgcacttcttggtctcaaagggaacttggaaatgacattggcatccagattatccactgcagtatttaccttgctgtggattgctgactggatggtccatcacttctggaggaaaggaaaggacccggatagtttctccatcccctacctaacagcattgggtgatctgctcgggacagctctgttagccttaagttttcattttctttggcttattggagatcgagatggagatgttggagactaataaattctacaaactgctcttaagttaccaaggaagaaaatacacgacaaccacttatggctctttttcaaaactcttaaa

SEQ ID NO: 61 - DNA, 4572 bp, Homo sapiens
  misc_feature: Incyte ID No 1644869CB1
tcgccccctggggccctttgccactcccttggcaaggagagccgagacctcagttcccggcggctcttgcggggcacaggtgagccctggctgcgcgcgcggcccctcctccccggcgcctcccaggtgagcggccgcgatcccggtcccgggtcccgccagccccagctgcttctcctatgcggggacagcggcaatcccccctgaattcctttcggcctctggggccatttggcagccgagttccctccccgggtgctcctggagctcccagggcttgaccctccggctcggacagagctgggagcggcaggggcggggaggacagtgcggggagcaaaatccgcagcccccacccaggtcccccagccgagcgccggggcacggggaggggaggggagggggtgtggccgggatgggaaggggcgggggacagggaggccggctggacgggttcggtggccccagagttgcagccgctctgggctctggggccaggttgcggttctccctgggtagcggctgctcccgggaagctcgctgccggctggggtgggggttcctggaaacgggcgccacccagcctctccccacctagctcaggaatggtgcgcctggaaactccgctggacttgcctgggcctcggggcggagtgctggggcgctgggtgcccgagtccccgggttccctgagcttagaagggcctgtctggaggaagggattccagctctagggactgggtggggcgtcctcagcccctctttcggcagctgtcccgtgttaggctgggtggggccgcctcggacccagctctgggctggttttcccacgtctttgctcctgctggagggagggggtggtgtggggcctgggcccgaagatggcacccaggacactgagggagggcgccgtccggtcctgtgactggcacccctgcaaggagctgctctgtcggggcgcccccgggctttgcctgtccgaggaaagcgcccttcaacccggccgactttgagccctgagttcctgagggtgagcgcaggaggagtccagggcagaggccggcaggactcgctggtggctgggagctgagtcctggctccactctcacagatgaacgggccccgagcccagggccccagacaaggctgctctggttccagttgtgccgggtgacggcagagccaggacaaaggaggcctgttgaggtcctggccacccccagccggccgccctgggatgatggaatgcagggcagtgggtcttctgagaacatgaggaccttggcgccctgacggcggtggcatggagtgacctctgtgtgtgggggaggagccccctgtgctgcttccgcctggggctggttgggggtgagggctccagcagctaatttcctgctagaggaagttctttgaggtcacagggcagacgcagagaaggcctacagcctactctgacctttctctccaaaaaggaggtgtctctgtgccagaaccctgggcatggagatcctgaaaggggctgtgtctacaggcaccctacctcttccccatttcccagggactggcgacatcgaggaaacagatgtggaactgcagaccccacacccttccttcctctctcccctcccctgacactggagggggccccccattccattctggcatgaggcttattcggtgcctcaccatttatgtacctggggtgataggggggtgggcgggactgtctgggctgtgagtggcacctcctgtggttcccccaggaatgggtggtgtggacagctgccaggcgttgaagaccacccctggctctgtgcccctgtctcatcagatgggggctccggaggtggcgcccaggctctgagctaccctaggtctgcagactagcgggcattggccagagacatggcccagccactggccttcatcctcgatgtccctgagaccccaggggaccagggccagggccccagcccctatgatgaaagcgaagtgcacgactccttccagcagctcatccaggagcagagccagtgcacggcccaggaggggctggagctgcagcagagagagcgggaggtgacaggaagtagccagcagacactctggcggcccgagggcacccagagcacggccacactccgcatcctggccagcatgcccagccgcaccattggccgcagccgaggtgccatcatctcccagtactacaaccgcacggtgcagcttcggtgcaggagcagccggcccctgctcgggaactttgtccgctccgcctggcccagcctccgcctgtacgacctggagctggaccccacggccctggaggaggaggagaagcagagcctcctggtgaaggagctccagagcctggcagtggcacagcgggaccacatgcttcgcgggatgcccttaagcctggctgagaaacgcagcctgcgagagaagagcaggaccccgagggggaagtggaggggccagccgggcagcggcggggtctgctcctgctgtggccggctcagatatgcctgcgtgctggccttgcacagcctgggcctggcgctgctctccgccctgcaggccctgatgccgtggcgctacgccctgaagcgcatcgggggccagttcggctccagcgtgctctcctacttcctctttctcaagaccctgctggctttcaatgccctcctgctgctgctgctggtggccttcatcatgggccctcaggtcgccttcccacccgccctgccgggccctgcccccgtctgcacaggcctggagctcctcacaggcgcgggttgcttcacccacaccgtcatgtactacggccactacagtaacgccacgctgaaccagccgtgtggcagccccctggatggcagccagtgcacacccagggtgggtggcctgccctacaacatgcccctggcctacctctccactgtgggcgtgagcttctttatcacctgcatcaccctggtgtacagcatggctcactctttcggggagagctaccgggtgggcagcacctctggcatccacgccatcaccgtcttctgctcctgggactacaaggtgacgcagaagcgggcctcccgcctccagcaggacaatattcgcacccggctgaaggagctgctggccgagtggcagctgcggcacagccccaggagcgtgtgcgggaggctgcggcaggcggctgtgctggggcttgtgtggctgctgtgtctggggaccgcgctgggctgcgccgtggccgtccacgtcttctcggagttcatgatccagagtccagaggctgctggccaggaggctgtgctgctggtcctgcccctggtggttggcctcctcaacctgggggccccctacctgtgccgtgtcctggccgccctggagccgcatgactccccggtactggaggtgtacgtggccatctgcaggaacctcatcctcaagctggccatcctggggacactgtgctaccactggctgggccgcagggtgggcgtcctgcagggccagtgctgggaggattttgtgggccaggagctgtaccggttcctggtgatggacttcgtcctcatgttgctggacacgctttttggggaactggtgtggaggattatctccgagaagaagctgaagaggaggcggaagccggagtttgacattgcccggaatgtcctggagctgatttatgggcagactctgacctggctgggggtgctcttctcgcccctcctccccgccgtgcagatcatcaagctgctgctcgtcttctatgtcaagaagaccagccttctggccaactgccaggcgccgcgccggccctggctggcctcacacatgagcaccgtcttcctcacgctgctctgcttccccgccttcctgggcgccgctgtcttcctctgctacgccgtctggcaggtgaagccctcgagcacctgcggccccttccggaccctggacaccatgtacgaggccggcagggtgtgggtgcgccacctggaggcggcaggccccagggtctcctggctgccctgggtgcaccggtacctgatggaaaacaccttctttgtcttcctggtgtcagccctgctgctggccgtgatctacctcaacatccaggtggtgcggggccagcgcaaggtcatctgcctgctcaaggagcagatcagcaatgagggtgaggacaaaatcttcttaatcaacaagcttcactccatctacgagaggaaggagagggaggagaggagcagggttgggacaaccgaggaggctgcggcaccccctgccctgctcacagatgaacaggatgcctagggggacggcgatgggcctcacgggcccgcccagcaccctgagaccacactgttgcctcccagtgaccctgctgggacaccaggacaaggaagacagtttcgcctctcgaaagccgcagctgcgcctaggctggagctggaagggtgggtgaatccggcttgggcatccccaatgaactctgccctgcctgggactctatttattctgattaaaggggttttgcaaatggaaaaaaaaaaaaaaaaaaaa

SEQ ID NO: 62 - DNA, 2044 bp, Homo sapiens
  misc_feature: Incyte ID No 6288712CB1
actgcagcctctaactcctggtctcaagtgatctcctgcttcagcctcagaccctattctaagctgtctcattggttgatactctacaagaagcacagaataaacaagagagaaaaacaaatagaagtacaggtccattcagttaagttctttttactaaatacctggtagaagtgggacactttgccacaggatccagatcagtgattctaacccatgaaatcttgctgtgtttgtgagttagttataaaacgaatgaattgtcactgaagtatcaaaaattaacatgatgatttacttataaattaactagttaagatttagcatgatgactataatagtttttgcaaattatttacttttaaattaaccagttaagatttagtgtaatgcctatagtattttagctttaatgaatttgtgttctctatggtttcttgagcaggagagtcatagatctacagcgattttagttggggaaattgtgtgtgtctatggtttttcatatcatcaagtatgtattgtgatggaaactgatagagaattcattattctgggggttagagagatgaatgcgacatctgttctaggcccttccaggtctttctgtccagacgaatggatgagacttcagatacctatgatacaagaaagtaagatgatgtcatgacaaacttagaatgaaactatgagaatagagagaagaatgacagtgaaaatgtgtgcctgctacatagaaatggacctcttcaggttggcgtgtgcgagattttcttgctctagggagattgtttaagcaatcactatgtcgacaaacacagatgtttccctttctgaattcatatgatgaagatcagggatccaaacttattcaaaaagctaaagaggcaccattcgtacttgttggaatagaaggttttgcagcaattgttgcatatggattatacaaattgaagagcaggggaaatactaaaatgtcccttcatctgatccacatgggtgtggcagcccaaggctttgttgtaggagcaatgactgttgttatatcatgtatcgagaattctgggcaaaacctaagccttagaagaagagacgctgtcttgctcttgtaggaggagcttgttttagttagacgtctcattattgaagttacgtattattgttgaaaataaactaatttgtatgggtttagatggtaacacggcattttgaatattggcttcctttcttgcaggcttgatttgcctgttgactgaattactagtgactagtttactaactaggtcattcaaggaagtcaagttaacttaaaagaaacatgtcacctaaatgcacttgatggtgttgaatgtccaccttcttaaattgttaagatgaacttagttctaaggaagataacaggccaaccctgaagtactcccagtttgctgcagaatctcacatattttggatgttatagaagagtcctatttgccctagttaatttaactttttttttgcctgttttgtggactggctggctcttttagaactctgtccaaaaagtgcatggactataacttgtaaagcttcccacaactgacaatatatatatgtgcatgtatttaaaccaaatctggaaaacttacaatacagctgcataatggtagtatttattaaagaatcacaattgtaaacatgagaataacttatggattctagtttagttctttagtaattgcaaattatatttttgctgttgttatattagaataatttttgaatgtcatcttgaaatagaaatatgtattttaagcactcatgcaaaggtaaatgaacactgtttaaatgtgtgctttgcttattttttctgtaacaatcgtaaacattaaactgaacaaattacctacagtagttttgattaatgacctaagagcaagctggtttggccagacagtgtacccaaacttttatataccatcgaatgttattacacttgtgaaattctcctgtctaacctgaatttacattccatggtggttacatggtatatgtattattatta

SEQ ID NO: 63 - DNA, 1300 bp, Homo sapiens
  misc_feature: Incyte ID No 71830156CB1
gcagagcaagtcagcattggcgccccttcctcagatccctatcatcttgggaaacagtagcccagaggttcaggaagatgttaacttaaatgttcagggtgccccagtctgttcagcatggctgaaatccacactccgtattcttccttgaagaaactgttatctttactcaatggcttcgtggctgtgtctggcatcatcctagttggcctgggcattggtggtaaatgtggaggggcctctctgacgaatgtcctcgggctgtcctccgcatacctccttcacgttggcaacctgtgcctggtgatgggatgcatcacggtactgcttggctgtgccgggtggtatggagcgactaaagagagcagaggcacgctcttgttttgcatcctgtcaatggttattgtcctcatcatggaagttacagctgccacagtggtccttcttttctttccaattgttggagatgtggccttggaacacaccttcgtgaccctgaggaagaattacagaggttacaacgagccagacgactattctacacagtggaacttggtcatggagaagctaaagtgctgtggggtgaataactacacagatttttctggctcttccttcgaaatgacaacgggccacacctaccccaggagttgctgtaaatccatcggaagtgtgtcctgtgacggacgcgatgtgtctccaaacgtcatccaccagaagggctgtttccataaactcctaaaaatcaccaagactcagagcttcaccctgagtgggagctctctgggagctgcagtgatacagttgccaggaattcttgccactttgctgctgtttatcaagctgggctgacacccaggcctggagaagatgagacacctgggcccatctggctgctggagattcagtctcagttttatttctctgtggcactcactgcttctggaggggagactgttaataaaagatttgggaaacccctaaaaaaaaaaaaaaaaaaaaaaaaaaaaaaaaaaaaaaaaaaaaaaaaaaaaaaaaaaaaaacaaacaaaaaaaacaacaacaagaaaaaaaaaaacaaacacagacggggcgggcacacaattatagcggcagctggagctaaggacgagagatataattagggcgggcgcagagagaaccctgccagttactattcaacgaaagaggtaattatagaaggagcaaaaatactatacatggacaacataaagagatatgttttagcaaaacaaggagagatattatctccgaacccgactacaacgagcagaactaa

SEQ ID NO: 64 - DNA, 1081 bp, Homo sapiens
  misc_feature: Incyte ID No 7505044CB1
ctggaagctgcttcctcagacatgccgctgctgctactgctgcccctgctgtgggcagacttgacccacaggcccaaaatcctcatccctggcactctagaacccggccactccaaaaacctgacctgctctgtgtcctgggcctgtgagcagggaacacccccgatcttctcctggttgtcagctgcccccacctccctgggccccaggactactcactcctcggtgctcataatcaccccacggccccaggaccacggcaccaacctgacctgtcaggtgaagttcgctggagctggtgtgactacggagagaaccatccagctcaacgtcacctatgttccacagaacccaacaactggtatctttccaggagatggctcagggaaacaagagaccagagcaggagtggttcatggggccattggaggagctggtgttacagccctgctcgctctttgtctctgcctcatcttcttcatagtgaagacccacaggaggaaagcagccaggacagcagtgggcaggaatgacacccaccctaccacagggtcagcctccccgaaacaccagaagaagtccaagttacatggccccactgaaacctcaagctgttcaggtgccgcccctactgtggagatggatgaggagctgcattatgcttccctcaactttcatgggatgaatccttccaaggacacctccaccgaatactcagaggtcaggacccagtgaggaacccacaagagcatcaggctcagctagaagatccacatcctctacaggtcggggaccaaaggctgattcttggagatttaacaccccacaggcaatgggtttatagacattatgtgagtttcctgctatattaacatcatcttagactttgcaagcagagagtcgtggaatcaaatctgtgctctttcatttgctaagtgtatgatgtcacacaagctccttaaccttccatgtctccattttcttctctgtgaagtaggtataagaagtcctatctcatagggatgctgtgagcattaaataaaggtacacatggaaaacaccaaaaaaaaaaaaaaggggg

SEQ ID NO: 65 - DNA, 2899 bp, Homo sapiens
  misc_feature: Incyte ID No 7505086CB1
cagaaggcagaaaaccagcagagtcacagaggagatggccaactgccaaatagccatcttgtaccagagattccagagagtggtctttggaatttcccaactcctttgcttcagtgccctgatctctgaactaacaaaccagaaagaagtggcagcatggacttatcattacagcacaaaagcatactcatggaatatttcccgtaaatactgccagaatcgctacacagacttagtggccatccagaataaaaatgaaattgattacctcaataaggtcctaccctactacagctcctactactggattgggatccgaaagaacaataagacatggacatgggtgggaaccaaaaaggctctcaccaacgaggctgagaactgggctgataatgaacctaacaacaaaaggaacaacgaggactgcgtggagatatacatcaagagtccgtcagcccctggcaagtggaatgatgagcactgcttgaagaaaaagcacgcattgtgttacacagcctcctgccaggacatgtcctgcagcaaacaaggagagtgcctcgagaccatcgggaactacacctgctcctgttaccctggattctatgggccagaatgtgaatacgtgagagagtgtggagaacttgagctccctcaacacgtgctcatgaactgcagccaccctctgggaaacttctcttttaactcgcagtgcagcttccactgcactgacgggtaccaagtaaatgggcccagcaagctggaatgcttggcttctggaatctggacaaataagcctccacagtgtttagctgcccagtgcccacccctgaagattcctgaacgaggaaacatgacctgccttcattctgcaaaagcattccagcatcagtctagctgcagcttcagttgtgaagagggatttgcattagttggaccggaagtggtgcaatgcacagcctcgggggtatggacagccccagccccagtgtgtaaagctgtgcagtgtcagcacctggaagcccccagtgaaggaaccatggactgtgttcatccgctcactgcttttgcctatggctccagctgtaaatttgagtgccagcccggctacagagtgaggggcttggacatgctccgctgcattgactctggacactggtctgcacccttgccaacctgtgaggctatttcgtgtgagccgctggagagtcctgtccacggaagcatggattgctctccatccttgagagcgtttcagtatgacaccaactgtagcttccgctgtgctgaaggtttcatgctgagaggagccgatatagttcggtgtgataacttgggacagtggacagcaccagccccagtctgtcaagctttgcagtgccaggatctcccagttccaaatgaggcccgggtgaactgctcccaccccttcggtgcctttaggtaccagtcagtctgcagcttcacctgcaatgaaggcttgctcctggtgggagcaagtgtgctacagtgcttggctactggaaactggaattctgttcctccagaatgccaagccattccctgcacacctttgctaagccctcagaatggaacaatgacctgtgttcaacctcttggaagttccagttataaatccacatgtcaattcatctgtgacgagggatattctttgtctggaccagaaagattggattgtactcgatcgggacgctggacagactccccaccaatgtgtgaagccatcaagtgcccagaactctttgccccagagcagggcagcctggattgttctgacactcgtggagaattcaatgttggctccacctgccatttctcttgtgacaacggctttaagctggaggggcccaataatgtggaatgcacaacttctggaagatggtcagctactccaccaacctgcaaaggcatagcatcacttcctactccaggggtgcaatgtccagccctcaccactcctgggcagggaaccatgtactgtaggcatcatccgggaacctttggttttaataccacttgttactttggctgcaacgctggattcacactcataggagacagcactctcagctgcagaccttcaggacaatggacagcagtaactccagcatgcagagctgtgaaatgctcagaactacatgttaataagccaatagcgatgaactgctccaacctctggggaaacttcagttatggatcaatctgctctttccattgtctagagggccagttacttaatggctctgcacaaacagcatgccaagagaatggccactggtcaactaccgtgccaacctgccaagatgatgggaaatgccccttgaatcctcacagccacctaggaacatatggagtttttacaaacgctgcatttgacccgagtccttaaggtttccataaacacccatgaatcaaagacatggaattaccttagattagctctggaccagcctgttggacccgctctggaccaaccctgtttcctgagtttgggattgtggtacaatctcaaattctcaacctaccaccccttcctgtcccacctcttctcttcctgtaacacaagccacagaagccaggagcaaatgtttctgcagtagtctctgtgctttgactcacctgttacttgaaataccagtgaaccaaagagactggagcatctgactcacaagaagaccagactgtggagaaataaaaatacctctttattttttgattgaaggaaggttttctccactttgttggaaagcaggtggcatctctaattggaagaaattcctgtagcatcttctggagtctccagtggttgctgttgatgaggcctcttggacctctgctctgaggcttccagagagtcctctggatggcaccagaggctgca

SEQ ID NO: 66 - DNA, 880 bp, Homo sapiens
  misc_feature: Incyte ID No 7505784CB1
ggtgagcagtggtgtgtgagagccaggcgtccctctgcctgcccactcagtggcaacacccgggagctgttttgtcctttgtggagcctcagcagttccctctttcagaactcactgccaagagccctgaacaggagccaccatgcagtgcttcagcttcattaagaccatgatgatcctcttcaatttgctcatctttttcttcttcatcctcctcctcatcttcattgctgaggttgcagctgctgtggtcgccttggtgtacaccacaatggctgagcacttcctgacgttgctggtagtgcctgccatcaagaaagattatggttcccaggaagacttcactcaagtgtggaacaccaccatgaaagggctcaagtgctgtggcttcaccaactatacggattttgaggactcaccctacttcaaagagaacagtgcctttcccccattctgttgcaatgacaacgtcaccaacacagccaatgaaacctgcaccaagcaaaaggctcacgaccaaaaagtagagggttgcttcaatcagcttttgtatgacatccgaactaatgcagtcaccgtgggtggtgtggcagctggaattgggggcctcgagctggctgccatgattgtgtccatgtatctgtactgcaatctacaataagtccacttctgcctctgccactactgctgccacatgggaactgtgaagaggcaccctggcaagcagcagtgattgggggaggggacaggatctaacaatgtcacttgggccagaatggactgccctttctgctccagacttggggctagatagggaccactccttttaggcgatgcctgactttccttccattggtgggtggatgggatgggggcattt

SEQ ID NO: 67 - DNA, 1172 bp, Homo sapiens
  misc_feature: Incyte ID No 7505813CB1
tttctctctcagctctccgtctctctttctctctcagcctctttctttctccctgtctcccccactgtcagcacctcttctgtgtggtgagtggaccgcttaccccactaggtgaagatgtcagcccaggagagctgcctcagcctcatcaagtacttcctcttcgttttcaacctcttcttcttcgtcctcggcagcctgatcttctgcttcggcatctggatcctcatcgacaagaccagcttcgtgtcctttgtgggcttggccttcgtgcctctgcagatctggtccaaagtcctggccatctcaggaatcttcaccatgggcatcgccctcctgggttgtgtgggggccctcaaggagctccgctgcctcctgggcctgtattttgggatgctgctgctcctgtttgccacacagatcaccctgggaatcctcatctccactcagcgggcccagctggagcgaagcttgcgggacgtcgtagagaaaaccatccaaaagtacggcaccaaccccgaggagaccgcggccgaggagagctgggactatgtgcagttccagctgcgctgctgcggctggcactacccgcaggactggttccaagtcctcatcctgagaggtaacgggtcggaggcgcaccgcgtgccctgctcctgctacaacttgtcggcgaccaacgactccacaatcctagataaggtgatcttgccccagctcagcaggcttggacacctggcgcggtccagacacagtgcagacatctgcgctgtccctgcagagagccacatctaccgcgagctcgggttcatgacgctctcgatattcctgtgcagaaacctggaccacgtctacaaccggctcgctcgataccgttaggccccgccctccccaaagtcccgccccgcccccgtcacgtgcgctgggcacttccctgctgcctgtaaatatttgtttaatccccagttcgcctggagccctccgccttcacattcccctggggacccacgtggctgcgtgcccctgctgctgtcacctctcccacgggacctggggctttcgtccacagcttcctgtccccatctgtcggcctaccaccacccacaagattatttttcacccaaacctcaaataaatcccctgcgtttttggtaaaaaaaaaaaaaaaaaaa

SEQ ID NO: 68 - DNA, 1998 bp, Homo sapiens
  misc_feature: Incyte ID No 7505873CB1
taggaataccctcccggctatcgcttcagtgggtagaggggaccactgcccgagagctttaatggagctgggtcctgccttcgcgctgaggagccctcgttttcgagatcaggcctgaccgggataagctccagctgttcttgaagactaattagttacactggatatgctgctgttctttactttgggattgcttatacattttgtgttcttcgcctccatctttgacatttattttacatctcctttggttcatggaatgactcctcagtttacaccattgcctcctccagcgagaagattagtgttgtttgttgctgatggccttcgagcagatgcactttacgaattagatgaaaatggaaactctagagcaccgtttattaggaatatcataatgcatgaaggcagctggggcatatctcatacacgtgtgccaacagaatctcggccaggtcatgtagctctgatagctgggttttatgaagatgtcagtgcagttgccaaaggatggaaggaaaatcctgtagagtttgattctctttttaatgaaagtaaatacacatggagctggggaagcccagatatcctgcctatgtttgccaaaggtgctagtggagaccacgtttatacatatagttatgatgctaaaagagaggattttggtgctcaagatgcaacaaaactggatacgtgggtttttgataatgttaaggacttctttcatcatgccagaaacaaccagtctttgttttctaaaataaatgaagagaaaatagtttttttcttacatttattaggaatagatacaaacggacatgctcatcgaccatcctcgagagactacaaggacaatattaaaaaagttgatgatggagttaaagaaatcgtgtctatgtttaaccatttctatggaaatgatgggaaaacaacatttatctttacctctgaccatggaatgacagactggggttcccatggggctggtcatccttcagagactttaactcctttagtcacttggggagctggaatcaagtatccccaaagagtatcagctcagcaatttgatgatgcatttttgaaagagtggagattggagaattggaagaggctagatgtcaatcaggctgatattgcaccattgatgacttcccttattggagttccctttcctcttaactcagtgggaatccttcctgtggattatcttaacaacactgatctcttcaaagcagagagcatgtttacaaatgcagtacagattcttgaacagttcaaggtgaaaatgactcagaagaaagaagttactttaccatttttgtttacaccatttaacatcagccactatgtgattgtcatgtccatgaccatctttttggtgttcctcaatggcctggcccagctgctcacaacgaagaaactcagactatgtggcaaacccaaaagtcacttcatgtgaggttgctgaagcaccattcagcatctggatcctgattctccttttaagctaaaatctcatcaaggcttcaataagaagatggatatggatatatagtatattctactcctgtaaggaaaatggtatttggaattccgaattgacaggttatctggaacaaaggagcttcttttttttttctaggttttgcaggcatgaaatagtgattatatctgtggaaaagcataggaaggcattctcctttttcatttttttcctttggctggcagctcttcccagtgatgttgagagcacctgcagcaatctggtccccagtcgcacaacttcccacatacccagaggagagcatatgcctgtgggggcagtgctgatggcatccagagtcattgctgtggctgagctggaaggaaatcaccaggtgccaccgtcaatatttatcagctttcagcactggttttgttagacagtcagggtgtattatttcaagagcttcaataaaaaaagtgtttgcaaaaaaaaaaaaaaaaaaaaaattg

SEQ ID NO: 69 - DNA, 919 bp, Homo sapiens
  misc_feature: Incyte ID No 7505881CB1
gtgtaatcataaatactgctctaagaaagggacaggaagtctcagaggctggagagcagagcaccaagatcgttctggcaggaacagccagtgggaggttccagctgagcgctccccagaggtgagctgatccccagccacagcacacaggaccaggctgcgagaacagcatcatcagcatcatgctattacaatcccaaaccatgggggtttctcacagctttacaccaaagggcatcactatccctcaaagagagaaacctggacacatgtaccaaaacgaagattacctgcagaacgggctgccaacagaaaccaccgttcttgggacagtccagatcctgtgttgcctgttgatttcaagtctgggggccatcttggtttttgctccctacccctcccacttcaatccagcaatttccaccactttgatgtctgggtacccatttttaggagctctgtgttttggcattactggatccctctcaattatctctggaaaacaatcaactaagccctttgacctgagcagcttgacctcaaatgcagtgagttctgttactgcaggagcaggcctcttcctccttgctgacagcatggtagccctgaggactgcctctcaacattgtggctcagaaatggattatctatcctcattgccttattcggagtactattatccaatatatgaaatcaaagattgtctcctgaccagtgtcagtttaacaagttcattttcctcgacccagtcacaagatcatatccaacaggtcaaaaagagttcttcacggtcttggatataagtaactcttggcctcagaggaaggaaaagcaactcaacactcatggtcaagtgtgattagactttcctgaaatctctgccattttagatactgtgaaacaaactaaaaaaaaaaaa

SEQ ID NO: 70 - DNA, 3858 bp, Homo sapiens
  misc_feature: Incyte ID No 7503510CB1
atggcgcggccggtccggggagggctcggggccccgcgccgctcgccttgccttctccttctctggctgcttttgcttcggctggagccggtgaccgccgcggccggcccgcgggcgccctgcgcggccgcctgcacttgcgctggggactcgctggactgcggtgggcgcgggctggctgcgttgcccggggacctgccctcctggacgcggagcctaaacctgagttacaacaaactctctgagattgaccctgctggttttgaggacttgccgaacctacaggaagtgtacctcaataataatgagttgacagcggtaccatccctgggcgctgcttcatcacatgtcgtctctctctttctgcagcacaacaagattcgcagcgtggaggggagccagctgaaggcctacctttccttagaagtgttagatctgagtttgaacaacatcacggaagtgcggaacacctgctttccacacggaccgcctataaaggagctcaacctggcaggcaatcggattggcaccctggagttgggagcatttgatggtctgtcacggtcgctgctaactcttcgcctgagcaaaaacaggatcacccagcttcctgtaagagcattcaagctacccaggctgacacaactggacctcaatcggaacaggattcggctgatagagggcctcaccttccaggggctcaacagcttggaggtgctgaagcttcagcgaaacaacatcagcaaactgacagatggggccttctggggactgtccaagatgcatgtgctgcacctggagtacaacagcctggtagaagtgaacagcggctcgctctacggcctcacggccctgcatcagctccacctcagcaacaattccatcgctcgcattcaccgcaagggctggagcttctgccagaagctgcatgagttggtcctgtccttcaacaacctgacacggctggacgaggagagcctggccgagctgagcagcctgagtgtcctgcgtctcagccacaattccatcagccacattgcggagggtgccttcaagggactcaggagcctgcgagtcttggatctggaccataacgagatttcgggcacaatagaggacacgagcggcgccttctcagggctcgacagcctcagcaagctgactctgtttggaaacaagatcaagtctgtggctaagagagcattctcggggctggaaggcctggagcacctgaaccttggagggaatgcgatcagatctgtccagtttgatgcctttgtgaagatgaagaatcttaaagagctccatatcagcagcgacagcttcctgtgtgactgccagctgaagtggctgcccccgtggctaattggcaggatgctgcaggcctttgtgacagccacctgtgcccacccagaatcactgaagggtcagagcattttctctgtgccaccagagagtttcgtgtgcgatgacttcctgaagccacagatcatcacccagccagaaaccaccatggctatggtgggcaaggacatccggtttacatgctcagcagccagcagcagcagctcccccatgacctttgcctggaagaaagacaatgaagtcctgaccaatgcagacatggagaactttgtccacgtccacgcgcaggacggggaagtgatggagtacaccaccatcctgcacctccgtcaggtcactttcgggcacgagggccgctaccaatgtgtcatcaccaaccactttggctccacctattcacataaggccaggctcaccgtgaatgtgttgccatcattcaccaaaacgccccacgacataaccatccggaccaccaccatggcccgcctcgaatgtgctgccacaggtcacccaaaccctcagattgcctggcagaaggatggaggcacggatttccccgctgcccagaccccatccttggtggtccccttggaagaccgtgtggtatctgtgggagtgaccaagagcattctccacatcaccagtgcagcaggactgccgctgggtcctgccccgagtgccaagggtcgctctaccccagtaaccacgatagaatgctgacggctgtgaagaaaaagccaatggcatctctagatgggaaaggggattcttcctggactttagcaaggttgtatcacccggactccacagagctacagcctgcatcttcattaacttcaggcagtccagagcgcgcggaagccccgtacttgcttgtttccaatggccacctccccaaagcatgtgacgccagtcccgagtccacgccactgacaggacagctccccgggaaacagagggtgccactgctgttggcaccaaaaagctaggttttgtctacctcagttcttgtcataccaatctctacgggaaagagaggtaggagaggctgcgaggaagcttgggttcaagcgtcactcatctgtacatagttgtaactcccatgtggagtatcagtcgctcacaggacttggatctgaagcacagtaaacgcaagaggggatttgtgtacaaaaggcaaaaaaagtatttgatatcattgtacataagagttttcagagatttcatatatatcttttacagaggctattttaatctttagtgcatggttaacagaaaaaaattatacaattttgacaatattatttttcgtatcaggttgctgtttaattttggagggggtggggaaatagttctggtgccttaacgcatggctggaatttatagaggctacaaccacatttgttcacaggagtttttggtgcggggtgggaaggatggaaggccttggatttatattgcacttcatagacccctaggctgctgtgcggtgggactccacatgcgccggaaggagcttcaggtgagcactgctcatgtgtggatgcccctgcaacaggcttccctgtctgtagagccaggggtgcaagtgccatccacacttgcagtgaatggcttttccttttaggtttaagtcctgtctgtctgtaaggcgtagaatctgtccgtctgtaaggcgtagaatgagggttgttaatccatcacaagcaaaaggtcagaacagttaaacactgcctttcctcctcctcttattttatgataaaagcaaatgtggccttctcagtatcattcgattgctatttgagacttttaaattaaggtaaaggctgctggtgttggtacctgtggatttttctatactgatgttttcgttttgccaatataatgagtattacattggccttgggggacagaaaggaggaagttctgacttttcagggctaccttatttctactaaggacccagagcaggcctgtccatgccattccttcgcacagatgaaactgagctgggactggaaaggacagcccttgacctgggttctgggtataatttgcacttttgagactggtagctaaccatcttatgagtgccaatgtgtcatttagtaaaacttaaatagaaacaaggtccttcaaatgttcctttggccaaaagctgaagggagttactgagaaaatagttaacaattactgtcaggtgtcatcactgttcaaaaggtaagcacatttagaattttgttcttgacagttaactgactaatcttacttccacaaaatatgtgaatttgctgcttctgagaggcaatgtgaaagagggagtattacttttatgtacaaagttatttatttatagaaattttggtacagtgtacattgaaaaccatgtaaaatattgaagtgtctaacaaatggcattgaagtgtctttaataaaggttcatttataaatgtcaaaaaaaaaaaaaaaaaaaaaaaaaaaaaaagatcggtc

SEQ ID NO: 71 - DNA, 4992 bp, Homo sapiens
  misc_feature: Incyte ID No 7714715CB1
ttgtacggcgccagtgtgctggaaaggcgctctctcgcctcgctgcctcctcccccgcgccgcggaccctctcccctcccttgcgttgcccccctctcccgccgcctcctcccgctctcctctcgctctcctcccgcctccttcttccaggcgcggccagcgggggcaagagcgaggtggctgcgaggaggcgtcactgaaagccctgacttggggaaggctgcgggctcggagccggagacgccgagctggggccgggtgagttgggaatcagactcttgagttgacagctggcctatggtttctgcattttgttaagaagcacctcatgcaagcgagggacctgggactgctcacagccaggtggcatttttctgaagttgttcattaagacttcccagcattcctacagatataaataggtgaggccgcaggcggtgctctgggtccgggagcgctgtccccagcatgaacgcggccggcggcgggagtgaatgactgcagctgcgacttccttcccgggccgcccgagcctccttccccaccgactttcttgttttgattaactccgtggactcctgactctttcttcgcccggaacatcaatatgtgtcatgtcattgtcacctgtcgctcgatgctctggaccttgctgagtattgtggtggcttttgccgagctcattgccttcatgagtgcagactggctgatcgggaaagcgaggagccgcggcggcgtggagccggcgggcccgggcgggggctccccggagccctaccaccccaccctgggcatctacgcccgctgcatccggaacccaggggtgcagcacttccagcgggacacgctgtgcgggccctacgccgagagcttcggcgagatcgccagcggcttctggcaggccacagctattttcctggctgtgggaatctttattctctgcatggtggccttggtgtccgtcttcaccatgtgtgtacagagcatcatgaagaaaagcatcttcaatgtctgtgggctgttgcaaggaattgcaggtctattccttatcctcggtttgatactctaccctgctggctggggttgccagaaggccatagactactgtggacattatgcatctgcctacaaacctggagactgctccttgggctgggccttttataccgccattgggggcacagtcctcactttcatctgtgctgtcttctctgcacaagcagaaattgcaacctctagtgacaaagtacaggaagaaattgaagaggggaaaaacctgatctgcctcctttagtttggaagagacaatgccattttctcccttgagtaatcttgtgaaacagtccacagtttcatcatttgagtcaagtggagaactaacctttacctaccaaagccacgttccacggcccgaggcttaaacaggaccaatgagaggccacatccagctacgcaaagttactggacatgcggtctgcagtgcacattataaggaatggaacatgaaaatagtatataatcctagacctggagttgccaagttctgtcagactccatctcccccaggttcaatgaaggataataatctaaatcattagggcagcagtttctctggtaacggaagagaccgtccgccagatctgcaggctgtttctgctccaacactgcttgcttgtgagcatctctgcctcagaatggggttttgggttggagttcttgttttcctctgttctttcaagttgtctccaacgaacagaaaactataaacttactggggacaggatgtgtgctaaagggcacagcaagacactgtcttttgcttagctgaccaaaggggtcagcagggatggcgtggagtcatgctgtggaacttattctaggctgaatcctagggtaaggtggatcaactgaactgtcactccagagattttagaaatttgagtaaagaaacaataaggacctatacaatcatatgagaacaaaaatatgaaatcttgctagtgaagacgtattttttcttcttcccagcagccaggctagcaccagttctggcccagtctcctcttcttctggagatcacatgtttttcttctaaggttaggattgtgctttgactgcgaaaggaaacctcactgtttcctccttccagggactgagggtctccaagctagctgtggcttatgcagatgttcactgggaggacctgccagaatctcggcacttggggggagacctttactcccagtttggtgaccatgctgtagtcagctctatttccaatcccgacagtagcagaatggcattctacaacaaaaagaagctagttatgggagttaagtttttgtagttactggtgttgatcctgaaagcagactgagataacattaaattgctgcaactgaagaactgcagccaagaccttaattccaggaaagcacagaggacaaagttaattcaaaaagaggcgctagatcaaggtcacagcactgcctacacctgtttacaaaaagaatcaaataccactatgaataaggattcaggggtttttaatctactttccataaattaccaatatcactgattcaggaagatagtatctcagaatgaccagagcagcacagaaacaagctactctgacattatgggagcttcaaaattgtatcatgatacagaaacactccttagcactttaagaaagtgagatggaactgccagatttctggaaggagaaaaagtgtaggtatttgggttcattaatctgctcacttgaggactttgttttgaaaaagtaccttctgtggacaaggtattgtgctaccagctatacaaccctgacttcagagtttgcaaccttgccctgagtgaatcatgttaaagctgtctgagtctaaagcaccgtatcttggtgcagaacagataattatacagagatggaatgggacaaccgcagttttactacattctggtgtttggcctatatgagaaaccatcttctcacagattaagggctaagggcaaaaggggtgggaggtgtggaactagccttaatgagtttcccattcctgaaccaaaattcaaagtgagtgagatgtaaatcctgtgattttggtgaagaaaaaaacgggtatcttcatagcagcctaggaaaccttaaccatatctctaacaccacacagaaagaggctggaggagccactggacaaagcttctgtctctgtgtgtacatttataatgttctaaccaagtctcaaaccttgatgaaaaacacaaaatttttccataaacttatcagaagactcacttttctttctttcttggatagagaaaccattttctgacactaggtttacaatctcagtgtccttacaagttaagtcctaagctcacaggatcctccgagcatgtccatcacctgctctttggctaaggtggcagtgtacctctagatcaacctgggaacagtcacaagggagtgtgacttcttggccataataaactcactcgatagtgtttatgttattaatctgaatgcaacagaagacaaaagcacaggcatgcacacacacacaaccccaaaccactaaaaactacctaaacactgacttagtaaatagtaaaaaggtaatgttgggacttttaaaccttgaatccattagccaggcttgggatgaaaggaccatctaaaatcatgctagtctaaaccatgctcttccacacagctgtttaaaaaccactgggtatgaggaatatgctagaaagaaatgttaaaaatagattgttggctcacacttatttttctaataaataggaccattattactaccaggaaagtcttatttattttgcctgaaattggcttaaagaaagtctcatgacgggatgggatgggctgcgcttctcaatgaactctgaggcagaaatatttgccttggattctgtggattctttaaacctgtgtgctaataattcaaacaatgttgcattaattgtataagggtttttgtatagttttcaaacatctgtggtgtaatgatctttgttaaacatatattctgtaaagtgccatagtctttttttatgtgtagcatatttaaaaatatatatgtatattatacatacacaagtttgtgtgaaagatgtgcaataacaaaggtgtatgtatgttttgttgttttgttttggaaactggacaggagtcaaaacagggatgtttgtttctgttttggcaaaggagagttccacatttttgccttcatggcttattcagtaacccataattttaatgctacacaaatcttatgtgaagaaaagactggtatgaaatcattttttcctgggtctaaaataatcgctagtgttatgtcaaagttaagcccgcacgccaggcccagttaatgctagtctttcatgtgaaatgtgaagctgccatgttgccttttctcttagtaggataactagtagctggtacataatcactgaggagctatttcttaacatgcttttatagaccatgctaatgctagaccagtatttaagggctaatctcacacctccttagctgtaagagtctggcttagaacagacctctctgtgcaataacttgtggccactggaaatccctgggccggcatttgtattggggttgcaatgactcccaagggccaaaagagttaaaggcacgactgggatttcttctgagactgtggtgaaactccttccaaggctgagggggtcagtaggtgctctggagggactcggcaccacttgatattcaacagccacttgagccaaatataaaattgtatttacagctgatggactcaatttgagccttcaaacttgtagttatcctattatattgtaaactaatacattgtctagcattgatttggttcctgtgcatatgtattttcactatgtgctcccctccccagatcttaattaaaccagattttgcaattcattcttattctttcaaaaaaaaaaaaaatt

SEQ ID NO: 72 - DNA, 791 bp, Homo sapiens
  misc_feature: Incyte ID No 7506032CB1
cggtggccatgactgcggccgtgttcttcggctgcgccttcattgccttcgggcctgcgctcgccctttatgtcttcaccatcgccaccgagccgttgcgtatcatcttcctcatcgccggagctttcttctggttggtgtctctactgatttcgtcccttgtttggttcatggcaagagtcattattgacaacaaagatggaccaacacagaaatatctgctgatctttggagcgtttgtctctgtctatatccaagaaatgttccgatttgcatattataaactcttaaaaaaagccagtgaaggtttgaagagtataaacccaggtgagacagcaccctctatgcgactgctggcctatgctttcatgacgctggtcattatcttgctgcatgtattctggggcattgtattttttgatggctgtgagaagaaaaagtggggcatcctccttatcgttctcctgacccacctgctggtgtcagcccagaccttcataagttcttattatggaataaacctggcgtcagcatttataatcctggtgctcatgggcacctgggcattcttagctgcgggaggcagctgccgaagcctgaaactctgcctgctctgccaagacaagaactttcttctttacaaccagcgctccagataacctcagggaaccagcacttcccaaaccgcagactacatctttagaggaagcacaactgtgcctttttctgaaaatccctttttctggtggaattgagaaagaaataaaactatgcagatatgaaaaaaaaaa

SEQ ID NO: 73 - DNA, 2036 bp, Homo sapiens
  misc_feature: Incyte ID No 7506034CB1
ctgcgcaggggcctgagcgggagagtcctggcgagggcgctggccgagaggtgctcggcttgtagcaggtcccgcactccagcctctcgctgccagggtttgctctctgcttgtcctgggctgaggtgtccatgacggagtcatccaaggaggaaaaaatctgttccgggtgagcccaggccgccccggatatgcgatggctgaggagcagacaccagggaccacactgaggttgggtttcagaccaagatactggattctcctagttaagataaagagctttgggtgcctgacagtgaaaatggtgtaatctgcgttaacagttcacagcttgaaggcatgacaattaaagaacacacatggacttgtggcacatggaaatgtgcgcacagaaaaaggaaatctataattcttttaaagtaggaaggcattcttccttgccaaaatgggtacattctgttcggttatcaagtttgaaaatctacaagaattaaagagactgtgtcactggggtcccatcatagcccttggtgttatagcaatatgttctaccatggccatgattgactctgtgttgtggtattggcccttacatacaactggaggaagtgtgaatttcatcatgttgataaattggactgtcatgattctttataattacttcaatgccatgtttgtcggtccgggctttgtccctctggggtggaaaccggaaatttctcaggataccatgtatctccagtattgtaaagtctgccaagcatacaaggcaccacgttcacatcactgcagaaagtgtaacagatgtgtgatgaagatggaccatcactgtccttggatcaacaactgttgtggttaccaaaatcatgcttcgttcacactgtttctccttttagcaccactgggttgtatccatgctgctttcatttttgtgatgactatgtacacacagctttatcatcggatgaaaataattctcagaaacaaaacttctattgagtcatggattgaagagaaggctaaagatcgaattcagtattatcaactagatgaagtctttgtttttccatatgatatgggaagtagatggaggaactttaaacaggtatttacgtggtcaggggtccctgaaggagatggacttgagtggccagtaagagaaggctgtcaccaatacagcttaacaatagaacagttgaaacaaaaagcagataagagagtcagaagtgttcgctataaagtaatagaagattatagtggtgcctgctgccctctgaataaaggaatcaaaaccttcttcacaagtccctgcaccgaagagcctcgaatacagctgcaaaaaggggaattcattttagccacaagaggtttacgatactggttatatggagacaaaattcttgatgattcctttatagaaggtgtttcaagaataaggggttggttccctagaaaatgtgtggaaaagtgtccctgtgatgctgaaacagatcaagccccagagggggagaagaaaaatagatagctgctgttaaaacaaaattatcctttaagtctgcttaattacttgaaaattgtacatattactaaagaattatgcaatgagcctactctggttaagatgttcttttcctcaaaggtgccctagtgccatgatttaaatatttttattaccattttgaaatggagaagccattctgcatatgcctttgaattcctgcccctctttaccacctcttcctccccctcaaaggaaaaacatttcatccaagtaagttaacggcattttctgtaggattttcttatgcactgcacactctggacctcacctgcagatacagttccccccttgccaggagcatctgcatgtggtacttctcttttccctcagttgatatttcttatatgatattctagatactatagaactcaatttgtcagattcagtataacctcagattttgttacctgtcttttaaaaatgcagattttgtcaaatcaaataaagatcaatggatgttgggtataaaaaaaaa

SEQ ID NO: 74 - DNA, 1100 bp, Homo sapiens
  misc_feature: Incyte ID No 7506100CB1
ggagcaccttggcgcgcggagctggcaccttggcgctgttggtggcggcggagacagctgtgaagtgtgaggttctttgtctgctggcagctaggggcgacgaggcgggacgtcatggaagtgaaggatgccaattctgcgcttctcagtaactatgaaacgttaaaatacatatcaaaaacaccatgcaggcaccagagtcctgaaattgtcagagaatttctcacagcattgaaaagccacaagttgaccaaagctgagaagctccagctgctgaaccaccggcctgtgactgctgtggagatccagctgatggtggaagagagtgaagagcggctcacggaggagcagattgaagctcttctccacaccgtcaccagcattctgcctgcagagccagaggctgagcagaagaagaatacaaacagcaatgtggcaatggacgaagaggacccagcatagaagagcacagctggccccggcgtttcatgaagtcagaaggcctggcagccatttcctggacgttgagaggattgtttatttgatttttatcctcatcccagcaggcctggctttgtggttagttgggtacatcacaaaaataagttaaaaagaaatatttgtgccttggggagaagaaacatggtgaaaacaggctgaggttgtcagggcagagagctgaaggtggggacagtgaccgcggacccctctgtgcttgaaagatttcctccacggcctttgccccagttgtggggaggtctctgtgcacagcggggaaaatgcttgtgtcgcctttggtgggccatgtcctaattagtttcatctgcttccctgggaacttactaaggggcccagagcactgttggaagtctggttagagtccccagagagttactctaagttaaaatgagccactgaccttggctcaccttagaggaatttcctcgagaacaacagagataagaaaagaaccggcctggccaatccttcaacagctctagagccccttttctctgctggcaggggctttgtttaccagctcactgtttaggctaaatgttagggaccagatcactgcagttgaaaacgggcactccagg

SEQ ID NO: 75 - DNA, 2043 bp, Homo sapiens
  misc_feature: Incyte ID No 1743113CB1
ctttggagtgggaagtgaggtgtgggaagtaggtcgctttctgatgaattcagtggcagtgaattgagaccggagggaatctggcccctagaggctggtacttgggcccgaaacccccatctccggcggagagaccgtccgaggtaattgtctgccacgagtgcacattctgaaaacaggagattttagttcctaaaaatgggaagaacctacattgtagaagagactgttggccagtatctttcaaacataaatctccaaggaaaggcttttgtctctggccttttaataggacagtgttcgtcacaaaaggattatgtgattcttgccactagaacgccacccaaagaggagcaaagtgagaacctcaaacatcccaaagctaagttggataacttggatgaagaatgggccacagaacatgcctgccaggtatccagaatgctaccagggggacttttagttcttggagtatttattattactactttagaactggcaaatgattttcaaaatgccctgcgtagactaatgtttgctgtggaaaagtctataaatagaaagagattgtggaatttcacagaggaggaagtctcagaacgagtgacacttcacatttgtgcttctacaaaaaaaatattttgtcgaacttatgatatccatgatccaaagagttcagcaagaccagcagattggaagtatcaaagtggattatcatcctcatggctttctttagagtgtacagttcacattaatattcacatcccactttctgctacttctgtcagctatactctggagaaaaatacaaagaatggacttacacgctgggccaaggaaatagaaaatggtgtttatttgattaatggacaagttaaagatgaagattgtgacctattagaaggacagaaaaaatcttctagaggaaatactcaagcaactagtcattcttttgatgtcagagtgctaacgcagttgctcctgaattcagaccacagatccacagccacagtccagatatgtagcggttctgtaaaccttaagggtgctgtgaaatgcagagcttatatccacagcagtaaacccaaagttaaagatgctgtgcaggcagtaaagagggatatattgaacacagttgctgatcgttgtgaaatgctatttgaggatctgcttttgaatgaaattccagaaaaaaaagattctgaaaaagagttccacgtcctcccttatcgagtctttgttccccttcctggatccactgtaatgttgtgtgattataaatttgacgatgagtcagctgaagaaatcagggaccattttatggagatgttggatcacacaattcaaatagaagatttggaaattgcagaggaaacaaacacagcttgtatgagttcttctatgaatagtcaagcttcattggacaacacagatgatgaacaaccaaaacaaccaattaaaactacaatgttattgaaaattcagcaaaacataggtgtgattgcagcatttacagttgcagtccttgctgcgggtatctcctttcattacttcagtgattagggtgaggcacaaagagtttcttgatcatccagagaacattgacagacaattatgaataataaagatgttaacaatccatctgtatttaaaacactagcagccagatctgctgccatgatgcctatttggtgtgtttctgattaaaatgaaatcacaagctgccttgtttagcctgctttacattgtaggtggcccgcatttccagaaataacgttatgcatctagatggaagctgcatgtaacaaatcattattatctatttttaaaagcttcaaaatgatgggatatgatcatagattttagtcttactaatctgaatcacatattaatcaggacattaaaaactttaacagaggcatgatggctcacacgtataatcctaatgctttgagaggctgaggtaggagcatcacctggggctgggagggagttggagaccagcctggatgacattatgagattctgtctctactggaaaaa

SEQ ID NO: 76 - DNA, 1128 bp, Homo sapiens
  misc_feature: Incyte ID No 7505144CB1
cccacgcgtccgcggacggtggcgacgtggcctcagtgcttaccagagcgcgttgtctaccctgtaccgaagacagaggctgtggggacagcctaggggcctggatctattgcctacttagagagaggccaactcagacacagccgtgtatgctcccagcagcaacggaggttcagctccgcctgcagggacagaaagacatggtctggaaatggatgccacttctgctgcttctggtctgtgtagccaccatgtgcagtgcccaggacaggactgatctcctcaatgtctgtatggatgccaagcaccacaagacaaagccaggtcctgaggacaagctgcatgaccaatgcagtccctggaagaagaatgcctgctgcacagccagcaccagccaggagctgcacaaggacacctcccgcctgtacaactttaactgggaccactgcggcaagatggagcccgcctgcaagcgccacttcatccaggacacctgtctctatgagtgctcacccaacctggggccctggatccagcaggtgaatcagagctggcgcaaagaacgcttcctggatgtgcccttatgcaaagaggactgtcagcgctggtgggaggattgtcacacctcccacacgtgcaagagcaactggcacagaggatgggactggacctcagctgccctttgtgaaggcctctggagtcactcatacaaggtcagcaactacagccgagggagcggccgctgcatccagatgtggtttgattcagcccagggcaaccccaacgaggaagtggcgaggttctatgctgcagccatgcatgtgaatgctggtgagatgcttcatgggactgggggtctcctgctcagtctggccctgatgctgcaactctggctccttggctgagttcagtcctcccagactacctgccctcagcttggataaccaggctgggctcagctcagctcccacaaatgacagccccttaagcatgcttctattagtcacctaaccctctgtcacccagtctgttgctgctccatggtggggccaagagtcacttctaataaacagactgttttctaataattccaaaaaaaaaaaaaaaaaatcctcgtgccgaattctt

SEQ ID NO: 77 - DNA, 871 bp, Homo sapiens
  misc_feature: Incyte ID No 7506132CB1
ggagcctgccgctccccgcgctcgtagcgcgggcctggggactggggatcccgccgccgggccgcagcatggggcgcttccgcgggggcctgcggtgcatcaagtacctgctgcttggcttcaacctgctcttctggctggctggatcggccgtcattgcttttggactatggtttcggttcggaggtgccataaaggagttatcatcagaggacaagtccccagagtatttctatgtggggctgtatgttctggttggagccggggccctgatgatggccgtggggttcttcgggtgctgcggagccatgcgggagtcgcaatgtgtgcttggatcattttttacctgcctcctggtgatatttgctgctgaagtaaccactggagtatttgcttttataggcaagggggtagctatccgacatgttcagaccatgtatgaagaggcttacaatgattaccttaaagacaggggaaaaggcaatgggacactcatcaccttccactcaacatttcagtgctgtggaaaagaaagctccgaacaggtccaacctacatgcccaaaggagcttctaggacacaagatctttggcatgatattcagcatggtcctctgctgtgcgatacgaaactcacgagatgtgatatgaagctacttctacatgaaaattgcaatctaaagctttcataccaaatgtcacaggagctgtctcccagctcatttttaacactgaaatgacattaggatctaaaataatttgctgtcaattgtacatttgcatgagtacgtatgtttggctcattactggtttaccccttgagtgaatgcctgtttatgatgactgagagcatattcatgtgtgatctgcgt

SEQ ID NO: 78 - DNA, 2300 bp, Homo sapiens
  misc_feature: Incyte ID No 8142016CB1
tatttatattcggctcgttattgtgtggaagaattcggctcgagtgtaaaactgccaaggaaagtaattacctgtaggagtttgctgagcttgaagagtgaaaactgttgtgaatgagcctgatcataaaacggaccaggccattcattattcctcaagtgttaatatactgacttatgcagtattcaaacaaaaacattgcactagatggtgcaagaacagctaaaatgaaagccatcattcatcttactcttcttgctctcctttctgtaaacacagccaccaaccaaggcaactcagctgatgctgtaacaaccacagaaactgcgactagtggtcctacagtagctgcagctgataccactgaaactaatttccctgaaactgctagcaccacagcaaatacaccttctttcccaacagctacttcacctgctccccccataattagtacacatagttcctccacaattcctacacctgctccccccataattagtacacatagttcctccacaattcctatacctactgctgcagacagtgagtcaaccacaaatgtaaattcattagctacctctgacataatcaccgcttcatctccaaatgatggattaatcacaatggttccttctgaaacacaaagtaacaatgaaatgtcccccaccacagaagacaatcaatcatcagggcctcccactggcaccgctttattggagaccagcaccctaaacagcacaggtcccagcaatccttgccaagatgatccctgtgcagataattcgttatgtgttaagctgcataatacaagtttttgcctgtgtttagaagggtattactacaactcttctacatgtaagaaaggaaaggtattccctgggaagatttcagtgacagtatcagaaacatttgacccagaagagaaacattccatggcctatcaagacttgcatagtgaaattactagcttgtttaaagatgtatttggcacatctgtttatggacagactgtaattcttactgtaagcacatctctgtcaccaagatctgaaatgcgtgctgatgacaagtttgttaatgtaacaatagtaacaattttggcagaaaccacaagtgacaatgagaagactgtgactgagaaaattaataaagcaattagaagtagctcaagcaactttctaaactatgatttgacccttcggtgtgattattatggctgtaaccagactgcggatgactgcctcaatggtttagcatgcgattgcaaatctgacctgcaaaggcctaacccacagagccctttctgcgttgcttccagtctcaagtgtcctgatgcctgcaacgcacagcacaagcaatgcttaataaagaagagtggtggggcccctgagtgtgcgtgcgtgcccggctaccaggaagatgctaatgggaactgccaaaagtgtgcatttggctacagtggactcgactgtaaggacaaatttcagctgatcctcactattgtgggcaccatcgctggcattgtcattctcagcatgataattgcattgattgtcacagcaagatcaaataacaaaacgaagcatattgaagaagagaacttgattgacgaagactttcaaaatctaaaactgcggtcgacaggcttcaccaatcttggagcagaagggagcgtctttcctaaggtcaggataacggcctccagagacagccagatgcaaaatccctattcaagccacagcagcatgccccgccctgactattagaatcataagaatgtggaacccgccatggcccccaaccaatgtacaagctattatttagagtgtttagaaagactgatggagaagtgagcaccagtaaagatctggcctccggggtttttcttccatctgacatctgccagcctctctgaatggaagttgtgaatgtttgcaacgaatccagctcacttgctaaataagaatctatgacattaaatgtagtagatgctattagcgcttgtcagagaggtggttttcttcaatcagtacaaagtactgagacaatggttagggttgttttcttaattcttttcctggtagggcaacaagaaccatttccaatctagaggaaagctccccagcattgcttgctcctgggcaaacattgctcttgagttaagtgacctaattccctgggagacatacgcatcaactgtggaggtccgaggggatgagaaggatacccaccacctttcaagggtcacaagctcactctctgaccagtcagaatagggacaacctttccagcactggacc

SEQ ID NO: 79 - DNA, 2903 bp, Homo sapiens
  misc_feature: Incyte ID No 7506135CB1
cccttctctagtccccgacctgcggcagccggagctcggggagcggagcgtggtggggaggggagcgggacaggcgacacaggagacagcggcgccgcggcctctccccaccaggcggccccggatcctactggacgccctgagggcacaccgaccgcgcctctagagtcaccccacgccgacccctcccctcttctctagacttatttccatccttcccgcttttaccctccccacccgtccctgggctccaggccgccgccccctcctcactcctggaccggcccttctcggtgcccctcttccctagggagatgcgatgagccggtgcccccgcgtcctcatcgtcgccccgggcacggtgcccgtccagtgcccgtggtggggagggagcactccgcggtccctccgtgacgcccctcgcttggccccccccacagctggcgtccctcggccatgccccaggggacccagccagggggtgggctctagagcgagtggggtggagaggagaaaggacggggccttgggcgcctctgagatgctcccaagtgccagggagggccgagcgaggcgcaggcaaccgggcagcaggcatgatgccctcgcctagtgactccagccgctcgctgaccagccggcccagcaccaggggccttacccacctccgcctccaccgaccctggctgcaggccctgcttacgctggggctggtccaagtgctcctgggcatcctggtggtcaccttcagcatggtggcctcttccgtcaccaccaccgagagcatcaagaggtcctgcccgtcttgggctgggttctcgaacctgctcttcagcgtctgtgggctcaccatttgtgccgctataatctgtacactctctgctattgtctgctgcatccaaatcttctccctggacctcgtgcatacgcagctggcccctgagcggtcagtctcaggcccactgggacctctgggctgcacgtccccgcccccagcccctctcctacacaccatgctggacctggaggaatttgtcccgcctgtgcccccaccgccctactatcccccagagtatacctgcagctcagaaacagatgcacagagcatcacgtacaatggctccatggacagcccagtgcccttgtaccctaccgattgccccccttcttatgaggcagtcatgggactacgaggagacagccaggccactctctttgaccctcagcttcacgatggctcgtgcatctgtgaacgagtggcctccattgtagacgtgtccatggacagcgggtctctggtgctgtcagccattggtgacctccctgggggctctagcccgtcggaggactcgtgcctgctggagctgcagggctccgtgcgctccgtggactacgttctctttcgctccatccagcgcagccgtgccggctactgcctcagcctggactgtggcctgcggggccccttcgaggaaagccccctgccacggcgccccccacgggctgcccgctcctattcctgctctgcccctgaagctccacccccactgggtgcccccacagctgcccgcagctgccaccggttggagggctggccgccctgggtgggaccctgcttccccgagctgaggcggcgggtcccccggggagggggccgcccagccgcagccccgcccacccgagccccgactcgtcgcttcagcgatagctcaggttccctcaccccaccggggcaccggcctcctcatccggcatccccaccaccgctgctgctgccacggtcccacagcgacccaggcatcacgacctccagtgacactgctgacttcagggacctttataccaaagtgcttgaggaagaagctgcttctgtttcctctgcagatacagggctctgctctgaagcctgcctcttccgcctagcccgctgcccttcccccaagttgctacgtgcccggtcagccgagaaacggcgccctgtgcccaccttccaaaaagttcccctgccctcgggccctgcacctgcccactccctgggggacctaaagggcagctggccaggtcggggcctggtcactcgtttcctccagatatccaggaaagccccagaccccagtgggactggagctcatggacataagcaggtgccccggagcctgtggggccggcctggccgagagagcctccaccttcgcagctgcggagatctgagctctagctcttccctgcggcgtctcctgtctggccgcaggctggagcgtggtacccgcccccacagcctcagcctcaacgggggcagccgggagactgggctctgacctaggcttcttgtcacactgaacacatccagccacaggcaccagctggttgggaccagcagcccccagcatcctcttgcactggctggcacaaaaagaaacctgctgtataccccccaaagtgtccctttcccaattacctctggggtctcttgctgcttgcctctgctgctctggactgggagagcttctgtcctgtgctgcatgggtatttagactgtgggggagatgccccttcttatagcactggaggaggaaaacaaattcttgtccccctcagaatgagagtggctctttctgatttgcaagggcactatggtcagggcaaaggcatggcccaggtgtttaagtacagggtgacgtgtgcctatgcaatggggtggtaaggcaggcacgaagagtccaaaaaatctaggtggcctctcagctctgccacctctagctgcatgaccttgggcaagctatgtaaccccaattgcctgctccattaaagactgtgaaggtagaatgtttgtaaagctcttaacagtatgtaagccttcaataaatttcagttttccccttgttttcttgaaaaaaaaaaaa

SEQ ID NO: 80 - DNA, 1480 bp, Homo sapiens
  misc_feature: Incyte ID No 90086301CB1
agcccggcatagatcttatcttcatcttcactcggttgcaaaatcaatagttaagaaatagcatctaagggaacttttaggtgggaaaaaaaatctagagatggctctaaatgactgtttccttctgaacttggaggtggaccatttcatgcactgcaacatctccagtcacagtgcggatctccccgtgaacgatgactggtcccacccggggatcctctatgtcatccctgcagtttatggggttatcattctgataggcctcattggcaacatcactttgatcaagatcttctgtacagtcaagtccatgcgaaacgttccaaacctgttcatttccagtctggctttgggagacctgctcctcctaataacgtgtgctccagtggatgccagcaggtacctggctgacagatggctatttggcaggattggctgcaaactgatcccctttatacagcttacctctgttggggtgtctgtcttcacactcacggcgctctcggcagacagggagaaaaacagaaaggaaagtgagggcatgaactgtaaagatgcagctgcggggaactcttctctggagtttcaaagcagaccaagttcaccatcaacagaaagggaaagctctttcaaaagccaggttgcactccttcgcggctccagtgtgttcccaatacaaagccattgtccggccaatggatatccaggcctctcatgccctgatgaagatctgcctcaaagccgcctttatctggatcatctccatgctgctggccattccagaggccgtgttttctgacctccatcccttccatgaggaaagcaccaaccagaccttcattagctgtgccccatacccacactctaatgagcttcaccccaaaatccattctatggcttcctttctggtcttctacgtcattccactgtcgatcatctctgtttactactacttcattgctaaaaatctgatccagagtgcttacaatcttcccgtggaagggaatatacatgtcaagaagcagattgaatcccggaagcgacttgccaagacagtgctggtgtttgtgggcctgttcgccttctgctggctccccaatcatgtcatctacctgtaccgctcctaccactactctgaggtggacacctccatgctccactttgtcaccagcatctgtgcccgcctcctggccttcaccaactcctgcgtgaacccctttgccctctacctgctgagcaagagtttcaggaaacagttcaacactcagctgctctgttgccagcctggcctgatcatccggtctcacagcactggaaggagtacaacctgcatgacctccctcaagagtaccaacccctccgtggccacctttagcctcatcaatggaaacatctgtcacgagcggtatgtctagattgacccttgattttgccccctgagggacggttttgctttatggctagacaggaacccttgcat

SEQ ID NO: 81 - DNA, 1498 bp, Homo sapiens
  misc_feature: Incyte ID No 7487373CB1
tattggaatttgctcatatatttcttactttgtgtatttggtttttccatataatcgttatatctgcttttccaatagtgcttagaaaccattttgtctacctgatcttttaggtctcacatagcctgggaaacctttacatccaccctataataccaccagtcaggatgaagagcaccatgaaaagtgacctataagtttcaggccattacagcactattcaaatatagtgatttcttctttcattttgtttttattttctgggttatatgatttacaatgaattcaacaatgtttattctgcatgaatagctgtatccccaacgtagtatccaataatcttacatagagtaagccctaaataatatcttctttatacatcctattttcccatacctaggatctcccaggaatgactaaattatcatccttgctgtcactgaaagtcaggactggtgaacatcttatgaacaggtagaattctcaaggagcaacttttatacaactgattactattgctttacctggagtaaatattcgcctctttgcaaagctggcaatggggctcaataagtctgcttccaccttccagcttactggcttcccaggcatggagaaggcacatcactggatattcatcccattattggcagcctacatctccatacttcttggcaatggcactcttctctttctcatcaggaatgatcataacctccatgagcccatgtactatttcttagctatgttggcagctacagacctcggagtgacattgaccacaatgcccacagtgctaggtgttctgtggttagatcacagggagactggccatggagcctgcttctctcaggcctattttatccatactctttctgtcatggagtcaggtgtcttgcttgccatggcttatgactgtttcattgccatccacaaccccttaagatatatctctatcctgaccaacacccaggtaatgaagattggtgtgggggtattgacaagggctggtctgtccattatgccaatagttgttcgcctacactggtttccctactgtcgagcccatgtattctcccatgctttctgtctacaccaagatgtcatcaagctagcctgtgctgacatcaccctcaaccgtctctatccagttgtggttttatttgcaatggtcttgttggactttctcatcatctttttctcctacattttgattctcaagactgtcatgggcattggttctggaggagaaagggccaaggccctcaacacatgtgtctctcatatctgctgcatcctggtcttctatgtcactgtagtttgtctgacatttattcataggtttggaaagcatgttcctcatgtcgttcacatcacaatgagatacatccacttccttttcccaccttttatgaacccatttatctatagcattaaaactaagcagattcagagtggcatacttcgcttattctctctgcctcactctagagcatga

SEQ ID NO: 82 - DNA, 1788 bp, Homo sapiens
  misc_feature: Incyte ID No 7506228CB1
catcgaccactatagggaattagagcctcgaggcaagaggttcggcacgaggccagcaacggggtgcggcagggtggggaacgcgggagcggggccagctcccaggaaagctggtctgcgagcggcccctgcccggctcccaggtccctgcgcgaccccgcccttcccgagaccccagccgggctgccgcccgcgtcccggaagctccagcctgaaccatgtttttcacttgtggcccaaatgaggccatggtggtctccgggttctgccgaagccccccagtcatggtggctggagggcgtgtctttgtcctgccctgcatccaacagatccagaggatctctctcaacacactgaccctcaatgtcaagagtgaaaaggtttacactcgccatggggtccccatctcagtcactggcattgcccaggagatctataaggacaggcagaaattctcagaacaggttttcaaagtggcctcctcagacctggtcaacatgggcatcagtgtggttagctacactctgaaggacattcacgatgaccaggactatttgcactctttggggaaggctcgaacagctcaagtccaaaaagatgcacggattggagaagcagaggccaagagagatgctgggatccgggaagctaaagccaagcaggaaaaggtgtctgctcagtacctgagtgagatcgagatggccaaggcacagagagattacgaactgaagaaggccgcctatgacatcgaggtcaacacccgccgagcacaggctgacctggcctatcagcttcaggtggccaagactaagcagcagattgaggagcagcgggtgcaggtgcaggtggtggagcgggcccagcaggtggcagtgcaggagcaggagatcgcccggcgggagaaggagctggaggcccgggtgcggaagccagcggaagcggagcgctacaagctggagcgcctagccgaggcagagaagtcccaactaattatgcaggcggaggcagaagccgcgtctgtgcggatgcgtggggaagctgaggcctttgccataggggcccgagcccgagccgaggctgagcagatggccaagaaggcagaagccttccagctgtaccaagaggctgctcagctggacatgctgctagagaagctgccccaggtggcagaggagatcagtggtcccttgacttcagccaataagatcacactggtgtccagcggcagtgggaccatgggggcagccaaagtgactggggaagtactggacattctaactcgcctgccagagagtgtggaaagactcacaggcgtgagcatctcccaggtgaatcacaagcctttgagaacagcctgagccttcagccctcacagatgcccagcctcatagctgaagttgcctgaatgatcctcctgttgcatgtaacccactggcctccctgagcatgtccattgacagtgaggtcccacccctcatctctccttgccaaatagtttgtgccttgtcttgaagggggttgctccccttgccaacctcacactgctatgattgccaactccagcggtcccatgtcagccttctgatgatcccactccaccccacctcaacttatttaacttcctaattaaatcagactgtttgagcctgttgtctagaatattttcctgaccaagactgagggatgggctggaggttttcaactttgctacccaaataaattgctgtaagtaagtactaataaaacagaagcaactggaaaaaaaaaaaaaaaaaaaaaaaaaaaaag

SEQ ID NO: 83 - DNA, 1386 bp, Homo sapiens
  misc_feature: Incyte ID No 7506194CB1
gcgcggagccgggccggcagcaacatgtcatggtttagtggcctcctggtccctaaagtggatgaacggaaaacagcctggggtgaacgcaatgggcagaagcgttcgcggcgccgtggcactcgggcaggtggcttctgcacgccccgctatatgagctgcctccgggatgcagagccacccagccccacccctgcgggcccccctcggtgcccctggcaggatgacgccttcatccggaggggcggcccaggcaagggcaaggagctggggctgcgggcagtggccctgggcttcgaggataccgaggtgacaacgacagcgggcgggacggctgaggtggcgcccgacgcggtgcccaggagtgggcgatcctgctggcgccgtctggtgcaggtgttccagtcgaagcagttccgttcggccaagctggagcgcctgtaccagcggtacttcttccagatgaaccagagcagcctgacgctgctgatggcggtgctggtgctgctcacagcggtgctgctggctttccacgccgcacccgcccgccctcagcctgcctatgtggcactgttggcctgtgccgccgccctgttcgtggggctcatggtggtgtgtaaccggcatagcttccgccaggactccatgtgggtggtgagctacgtggtgctgggcatcctggcggcagtgcaggtcgggggcgctctcgcagcagacccgcgcagcccctctgcgggcctctggtgccctgtgttctttgtctacatcgcctacacgctcctccccatccgcatgcgggctgccgtcctcagcggcctgggcctctccaccttgcatttgatcttggcctggcaacttaaccgtggtgatgccttcctctggaagcagctcggtgccaatgtgctgctgttcctctgcaccaacgtcattggcatctgcacacactatccagcagaggtgtctcagcgccaggcctttcaggagacccgcggttacatccaggcccggctccacctgcagcatgagaatcggcagcaggagcggctgctgctgtcggtattgccccagcacgttgccatggagatgaaagaagacatcaatgagcactccttcaacaatttccagatgaagattgggctgaacatgggcccagtcgtggcaggtgtcatcggggctcggaagccacagtatgacatctgggggaacacagtgaatgtctctagtcgtatggacagcacgggggtccccgaccgaatccaggtgaccacggacctgtaccaggttctagctgccaagggctaccagctggagtgtcgaggggtggtcaaggtgaagggcaagggggagatgaccacctacttcctcaatgggggccccagcagttaa

SEQ ID NO: 84 - DNA, 1885 bp, Homo sapiens
  misc_feature: Incyte ID No 7506434CB1
gatgtggggccttgcgggaggaaggcttttcggcatcttctcggccccggtgctggtggctgtggtgtgctgcgcccagagtgtgaacgatccccggtctgcgtggggatgaacatcgacatcgccagcatcgacatggtttccgaagtcaacatggattataccttaaccatgtattttcaacaatattggagagataaaaggctcgcctattctgggatccctctcaacctcacgcttgacaatcgagtggctgaccagctatgggtgcccgacacatatttcttaaatgacaaaaagtcatttgtgcatggagtgacagtgaaaaaccgcatgatccgtcttcaccctgatgggacagtgctgtatgggctcagaatcaccacgacagcagcatgcatgatggacctcaggagataccccctggacgagcagaactgcactctggaaattgaaagctatggctacaccacggatgacattgagttttactggcgaggcggggacaaggctgttaccggagtggaaaggattgagctcccgcagttctccatcgtggagcaccgtctggtctcgaggaatgttgtcttcgccacaggtgcctatcctcgactgtcactgagctttcggttgaagaggaacattggatacttcattcttcagacttatatgccctctatactgataacgattctgtcgtgggtgtccttctggatcaattatgatgcatctgctgctagagttgccctcgggatcacaactgtgctgacaatgacaaccatcaacacccaccttcgggagaccttgcccaaaatcccctatgtcaaagccattgacatgtaccttatgggctgcttcgtctttgtgttcctggcccttctggagtatgcctttgtcaactacattttctttggaagaggccctcaaaggcagaagaagcttgcagaaaagacagccaaggcaaagaatgaccgttcaaagagcgaaagcaaccgggtggatgctcatggaaatattctgttgacatcgctggaagttcacaatgaaatgaatgaggtctcaggcggcattggcgataccaggaattcagcaatatcctttgacaactcaggaatccagtacaggaaacagagcatgcctcgagaagggcatgggcgattcctgggggacagaagcctcccgcacaagaagacccatctacggaggaggtcttcacagctcaaaattaaaatacctgatctaaccgatgtgaatgccatagacagatggtccaggatcgtgtttccattcactttttctcttttcaacttagtttactggctgtactatgttaactgagtgactgtacttgatttttcaaagacttcatttaacactgagtgaaatattactctgcctgtcaagtttttatacctgtacacacacagacacacaagcagacacacacatatatacatacgcaattgtatatatatgtgaactttctcagcatatatataaaatacacgtgtatatgaggatgtatgtgtatatgtttatacacacaggagtcagtgcccatgtgtatggaagacaaatacacatacatatatacattttgcagctatggacaatttaccacaggatgcatattaaagaaagtcatagtttttttcttttttaattgaaagggacaagtatcatctaaatattatgccttgagaatgagggcgtgaaacacaatatcatccccaaatgtgtcttgtattatcataagttagatgttttagttttaaaatccggaaggccttcttaggttatactttggaaaactcctaccgttgggtttggctaggtttataatgggttcacttacttcatatcctccggttcgttttggtaggtg

SEQ ID NO: 85 - DNA, 1788 bp, Homo sapiens
  misc_feature: Incyte ID No 7490974CB1
tgattgatcttttcatccagcacaatggacagaagtctaaggaacgtccttgtggtttcctttgggttcctgcttctctttacagcctatggaggtctgcagagcctgcagcttcagaggaggctcccagggcagcaagcctgtggctggcggttcctgggagctgtggcggcctccagcctgggggacactgaggacctgctggtgcctcatcccgctgtgtccatcctgtgctctgacagggctggctttgttcccacagagcagcctgtacagcgaggagggcctgggtgtcacagcgctcagcaccctctatggaggcatgctcctgtcctccatgttcctcccaccgctcctcatcgagaggctgggctgcaaggggaccatcatcctctccatgtgtggctacgtggccttctccgtgggcaacttcttcgccagctggtctacaccctcctgggcatctacactgggagtggtgtcctggctgtcctgatgatagctgcgttcctccaacccatacgagatgttcagcgggaaagtgaaggagagaagaaatcagtacctttctggtccactttactgtcgactttcaagctatatagagataaacgtctgtgcctcttaattctgctgccgctgtacagtggattgcagcaaggattcctctccagcgaatacacaaggtcctatgtcacctgcaccctgggcatccagttcgtcggctacgtgatgatctgcttctcggccactgacgcgctgtgctccgtgttgtatggaaaggtctcgcagtacacgggcagggctgtgctgtacgtgctgggcgcggtgacccacgtgtcctgcatgattgccctactgctgtggagacctcgtgctgaccatctggcagtgttcttcgtattctctggcctgtggggcgtggcagatgccgtctggcagacacaaaacaatgctctctacggcgttctgtttgagaagagcaaggaagctgccttcgccaattaccgcctgtgggaggccctgggcttcgtcattgccttcgggtacagcacgtttttgtgcgtgcacgtcaagctctacattctgctgggggtcctgagcctgaccatggtggcgtatgggcttgtggagtgcgtggagtccaagaacccgatcagaccccacgctccaggacaggtcaaccaggcagaggatgaagaaatacaaacaaaaatgtgagagcagtgaggtccgaggaggatgaactcagaaagcaccagccagagaattttcttagaagatgcctcaggacatagagcggctcctcatcaccatctcagcacaatttggccattctgaagagatcatgttatttcactctttatgtattttttttctattctaacaaatttttcgtccaccatcttaacagagatcaagtgtatacatgaaggtatcagttcatttaattttagatgcaaaagaaaaaggtctaacgtacaatcagccaattagaatttgcctgaaatcatagactcaccctagttttattgctgtagttgtttttaagaattggaagcctgcttaaaaaatgtagttgagccccataattttacaaatgggcgaacttttaaacttctaactctacttggatcaaaacctcatacattttacaaaggggtcctgacaagtcagctgactcaacctcacagagtcagggggtgacaaagccagactggggctcaggattcctgaaacgtgtggggtctgcgtttctaaataaagacggttatttaacggaaaaaa

SEQ ID NO: 86 - DNA, 2786 bp, Homo sapiens
  misc_feature: Incyte ID No 7506224CB1
ccgtgctgaggcgggtggcatggcggagaaggatgacaccggagtttgacgaagaggtggtttttgagaattctccactttaccaatacttacaggatctgggacacacagactttgaaatatgttcttctttgtcaccaaaaacagaaaaatgcacaacagagggacaacaaaagcctcctacaagagtcctaccaaaagatattggattccgactcgactcattacataccatcctgcaacaggaagtcctgttacaagaggatgtggagctgattgagctacttgatcccagtatcctgtctgcagggcaatctcaacaacaggaaaatggacaccttccaacactttgctccctggcaacccctaatatttgggatctctcaatgctatttgccttcattagcttgctcgttatgcttcccacttggtggattgtgtcttcctggctggtatggggagtgattctatttgtgtatctggtcataagagctttgagattatggaggacagccaaactacaagtgaccctaaaaaaatacagcgttcatttggaagatatggccacaaacagccgagcttttactaacctcgtgagaaaagctttacgtctcattcaagaaaccgaagtgatttccagaggatttacactggtcagtgctgcttgcccatttaataaagctggacagcatccaagtcagcatctcatcggtcttcggaaagctgtctaccgaactctaagagccaacttccaagcagcaaggctagctaccctatatatgctgaaaaactaccccctgaactctgagagtgacaatgtaaccaactacatctgtgtggtgccttttaaagagctgggccttggacttagtgaagagcagatttcagaagaggaagcacataactttacagatggcttcagcctgcctgcattgaaggttttgttccaactctgggtggcacagagttcagagttcttcagacggttagccctattactttctacagccaattcacctcctgggcccttacttactccagcacttctgcctcatcgtatcttatctgatgtgactcaaggtctacctcatgctcattctgcctgtttggaagagcttaagcgcagctatgagttctatcggtactttgaaactcagcaccagtcagtaccgcagtgtttatccaaaactcaacagaagtcaagagaactgaataatgttcacacagcagtgcgtagcttgcagctccatctgaaagcattactgaatgaggtaataattcttgaagatgaacttgaaaagcttgtttgtactaaagaaacacaagaactagtgtcagaggcttatcccatcctagaacagaaattaaagttgattcagccccacgttcaagcaagcaacaattgctgggaagaggccatttctcaggtcgacaaactgctacgaagaaatacagataaaaaaggcaagcctgaaatagcatgtgaaaacccacattgtacagtagtacctttgaagcagcctactctacacattgcagacaaagatccaatcccagaggagcaggaattagaagcttatgtagatgatatagatattgatagtgatttcagaaaggatgatttttattacttgtctcaagaagacaaagagagacagaagcgtgagcatgaagaatccaagagggtgctccaagaattaaaatctgtgctgggatttaaagcttcagaggcagaaaggcagaagtggaagcaacttctatttagtgatcatgaagccgtgttgaaatccttgtctcctgtagacccagtggaacccataagtaattcagaaccatcaatgaattcagatatgggaaaagtcagtaaaaatgatactgaagaggaaagtaataaatccgccacaacagacaatgaaataagtaggactgagtatttatgtgaaaacgctctagaaggtaaaaataaagataattcttcaaatgaagtcttcccccaaggagcagaagaaagaatgtgttaccaatgtgagagtgaagatgaaccacaagcagatggaagtggtctgaccactgcccctccaactcccagggactcattacagccctccattaagcagaggctggcacggctacagctgtcaccagattttaccttcactgctggccttgctgcagaagtggctgctagatctctctcctttaccaccatgcaggaacagacttttggtgatgaggaggaagaacaaataatagaagaaaataaaaatgagatagaagaaaagtaagaaccaagattcatatgaagtgatattagattgttccttttacaaaagtgtttagcttcaagactggaaagggaatatgagtgtaagtttactatatataaagctaagatgtggatttacaggaagaaccctggtttgaataactgatctgaaattagtagttacctgtaaatggcagatcttttaggaaaataagagaaaggtaagggctcttttgaataaactgctgttttatttgtggcacaactgatcaatcttggaaattctttaagtatttttaataagaaatgaattatcatttcttgccagaatttgctaccttaaggtgattgggaaaattctgttgcaagaacattaacatttagtatgactcctttttactgtattcttgcagttaataactgcagctattatgttaataacaagttgtttgtattttatttttgtttataccagtcttaaagatccaggttctgaataaaaaaattaattgcccaaaaa

SEQ ID NO: 87 - DNA, 2020 bp, Homo sapiens
  misc_feature: Incyte ID No 7506280CB1
gagacggagccgctgtcaactctccaactcagctcagctgatcggttgccgccgccgccgccgccagattctggaggcgaagaacgcaaagctgagaacatggacgttaatatcgccccactccgcgcctgggacgatttcttcccgggttccgatcgctttgcccggccggacttcagggacatttccattgtggggtttctgagtcccttcaacatgatcctgggaggaatcgtggtggtgctggtgttcacagggtttgtgtgggcagcccacaataaagacgtccttcgccggatgaagaagcgctaccccacgacgttcgttatggtggtcatgttggcgagctatttccttatctccatgtttggaggagtcatggtctttgtgtttggcattacttttcctttgctgttgatgtttatccatgcatcgttgagacttcggaacctcaagaacaaactggagaataaaatggaaggaataggtttgaagaggacaccgatgggcattgtcctggatgccctagaacagcaggaagaaggcatcaacagactcactgactatatcagcaaagtgaaggaataaacataacttacctgagctagggttgcagcagaaattgagttgcagcttgcccttgtccagacctatgttctgcttgcgtttttgaaacaggaggtgcacgtaccacccaattatctatggcagcatgcatgtataggccgaactattatcagctctgatgtttcagagagaagacctcagaaaccgaaagaaaaccaccaccctcctattgtgtctgaagtttcacgtgtgtttatgaaatctaatgggaaatggatcacacgatttctttaagggaattaaaaaaaataaaagaattacggcttttacagcaacaatacgattatcttataggaaaaaaaaaatcattgtaaagtatcaagacaatacgagtaaatgaaaaggctgttaaagtagatgacatcatgtgttagcctgttcctaatcccctagaattgtaatgtgtgggatataaattagtttttattattctcttaaaaatcaaagatgatctctatcactttgccacctgtttgatgtgcagtggaaactggttaagccagttgttcatacttcctttacaaatataaagatagctgtttaggatattttgttacatttttgtaaatttttgaaatgctagtaatgtgttttcaccagcaagtatttgttgcaaacttaatgtcattttccttaagatggttacagctatgtaacctgtattattctggacggacttattaaaatacaaacagacaaaaaataaaacaaaacttgagttctatttaccttgcacattttttgttgttacagtgaaaaaaatggtccaagaaaatgtttgccatttttgcattgtttcgtttttaactggaacatttagaaagaaggaaatgaatgtgcattttattaattccttaggggcacaaggaggacaataatagctgatcttttgaaatttgaaaaacgtctttagatgaccaagcaaaaagactttaaaaaatggtaatgaaaatggaatgcagctactgcagctaataaaaaattttagatagcaattgttacaaccatatgcctttatagctagacattagaattatgatagcatgagtttatacattctattatttttcctccctttctcatgtttttataaataggtaataaaaaatgttttgcctgccaattgaatgatttcgtagctgaagtagaaacatttaggtttctgtagcattaaattgtgaagacaactggagtggtacttactgaagaaactctctgtatgtcctagaataagaagcaatgatgtgctgcttctgatttttcttgcattttaaattctcagccaacctacagccatgatctttagcacagtgatatcaccatgacttcacagacatggtctagaatctgtacccttacccacatatgaagaataaaattgattaaaggttaa

SEQ ID NO: 88 - DNA, 1487 bp, Homo sapiens
  misc_feature: Incyte ID No 7508326CB1
cccagggaccctccattttccatatccaggaaaatgtgatgcgccacaggtatcagcgtctggatcgccacttcacgttttagccacaagtgactcagtggaagatccagagtcaacagaggctcgtcaggaagatgtctacagaaaaggtagaccaaaaggaggaagctggggaaaaagaggtgtgcggagaccagatcaaaggaccggacaaagaggaggaaccaccagctgctgcatcccatggccaggggtggcgtccaggtggcagagcagctaggaacgcaaggcctgaacctggggccagacaccctgctctcccggccatggtcaacgaccctccagtacctgccttactgtgggcccaggaggtgggccaagtcttggcaggccgtgcccgcaggctgctgctgcagtttggggtgctcttctgcaccatcctccttttgctctgggtgtctgtcttcctctatggctccttctactattcctatatgccgacagtcagccacctcagccctgtgcatttctactacaggaccgactgtgattcctccaccacctcactctgctccttccctgttgccaatgtctcgctgactaagggtggacgtgatcgggtgctgatgtatggacagccgtatcgtgttaccttagagcttgagctgccagagtcccctgtgaatcaagatttgggcatgttcttggtcaccatttcctgctacaccagaggtggccgaatcatctccacttcttcgcgttcgtacgtgccgaccactggagcgatcattgagatccacagcaagcgcatccagctgtatggagcctacctccgcatccacgcgcacttcactgggctcagatacctgctatacaacttcccgatgacctgcgccttcataggtgttgccagcaacttcaccttcctcagcgtcatcgtgctcttcagctacatgcagtgggtgtgggggggcatctggccccgacaccgcttctctttgcaggttaacatccgaaaaagagacaattcccggaaggaagtccaacgaaggatctctgctcatcagccagggcctgaaggccaggaggagtcaactccgcaatcagatgttacagaggatggtgagagccctgaggatccctcagggacagagggtcagctgtccgaggaggagaaaccagatcagcagcccctgagcggagaagaggagctagagcctgaggccagtgatggttcaggctcctgggaagatgcagctttgctgacggaggccaacctgcctgctcctgctcctgcttctgcttctgcccctgtcctagagactctgggcagctctgaacctgctgggggtgctctccgacagcgccccacctgctctagttcctgaagaaaaggggcagactcctcacattccagcactttcccacctgactcctctcccctcgtttttccttcaataaactattttgtttgaccggtcttcatc

SEQ ID NO: 89 - DNA, 1120 bp, Homo sapiens
  misc_feature: Incyte ID No 7506370CB1
actggcatcgccgtccgcgccggccccggccatgaacgggctgccctcggcagaggcgccgggcggggcgggctgcgctttggccgggctcccaccgctgccgcgcggcctcagcggcctccttaatgcgagcgggggctcgtggcgggagctggagcgcgtctacagccagcgcagccgcatccacgacgagctgagccgcgccgcccgcgccccggacgggccccgccacgccgccggcgccgccaacgcgggacccgcagccggcccgcgtcgtcctgtcaacctcgactcagcgctggccgcgctgcgcaaggagatgctgtggggcctgtacgagtcaatccaggactacaaacacctgtgccaagacctgagcttctgccaggacctgtcatcctccctccattcggacagctcctacccaccggatgcgggcctgtctgacgacgaggagcctcccgatgccagcctgcctcctgacccgccaccccttactgtgccccagacgcacaatgcccgtgaccagtggctgcaggatgccttccacatcagcctctgaagggctggggggcagggggcatgcacccatgcaaaaggctcagaaactccccctccggcaagccctcagacttcggagcctgcgccttcccccctaccgcctcacctcacaggagggccaggcatgtattcctcagaggcgaaactgccaaactctttctcctgtcttgggttggctggcactggggcgggcatctagggtacagcctctgctcatggcactgggcctccagttcttccacatgtgtgcacccccagcttggccaaccctcagccttgcggtggggcccgaagcatcttcccttccgcttggcgtctctgggattgggatgagtgcctggctcccatctcctcctcaccttttgttgctatcggcagctgctggctcaggggcatcccacctccgggctctgggttcctctgccctggaagggctccaggacccgtcccaataaccacccacggccaggagggccaaggccccgtgctggatatttaaatttaggggccggtctccagggcgcgtagataaataaatacactcagcgtcaaaaaaaaa

SEQ ID NO: 90 - DNA, 2846 bp, Homo sapiens
  misc_feature: Incyte ID No 6312989CB1
cccttctctagtccccgacctgcggcagccggagctcggggagcggagcgtggtggggaggggagcgggacaggcgacacaggagacagcggcgccgcggcctctccccaccaggcggccccggatcctactggacgccctgagggcacaccgaccgcgcctctagagtcaccccacgccgacccctcccctcttctctagacttatttccatccttcccgcttttaccctccccacccctccctgggctccaggccgccgccccctcctcactcctggaccggcccttctcggtgcccctcttccctagggagatgcgatgagccggtgcccccgcgtctcatcgtcgccccgggcacggtgcccgtccagtgcccgtggtggggagggagcactccgcggtccctccgtgacgcccctcgcttggccccccccacagctggcgtccctcggccatgccccaggggacccagccagggggtgggctctagagcgagtggggtggagaggagaaaggacggggccttgggcgcctctgagatgctcccaagtgccagggagggccgagcgaggcgcaggcaaccgggcagcaggcatgatgccctcgcctagtgactccagccgctcgctgaccagccggcccagcaccaggggccttacccacctccgcctccaccgaccctggctgcaggccctgcttacgctggggctggtccaagtgctcctgggcatcctggtggtcaccttcagcatggtggcctcttccgtcaccaccaccgagagcatcaagaggtcctgcccgtcttgggctgggttctcgctggcgttctccggggtggttggcattgtgtcctggaagcggccattcactctagtgatctccttcttctccttgctttcggtgctctgtgtcatgcttagcatggctggctctgttctctcctgtaagaatgctcaactggcccgagacttccaacagtgctctctggaaggaaaggtctgtgtgtgctgtccctctgttcccctcctccggccctgtccagagtcggggcaggaactgaaagttgcccctaactccacctgtgatgaagcccgaggggccctcaagaacctgctcttcagcgtctgtgggctcaccatttgtgccgctataatctgtacactctctgctattgtctgctgcatccaaatcttctccctggacctcgtgcatacgcagctggcccctgagcggtcagtctcaggcccactgggacctctgggctgcacgtccccgcccccagcccctctcctacacaccatgctggacctggaggaatttgtcccgcctgtgcccccaccgccctactatcccccagagtatacctgcagctcagaaacagatgcacagagcatcacgtacaatggctccatggacagcccagtgcccttgtaccctaccgattgccccccttcttatgaggcagtcatgggactacgaggagacagccaggccactctctttgaccctcagcttcacgatggctcgtgcatctgtgaacgagtggcctccattgtagacgctgacttcagggacctttataccaaagtgcttgaggaagaagctgcttctgtttcctctgcagatacagggctctgctctgaagcctgcctcttccgcctagcccgctgcccttcccccaagttgctacgtgcccggtcagccgagaaacggcgccctgtgcccaccttccaaaaagttcccctgccctcgggccctgcacctgcccactccctgggggacctaaagggcagctggccaggtcggggcctggtcactcgtttcctccagatatccaggaaagccccagaccccagtgggactggagctcatggacataagcaggtgccccggagcctgtggggccggcctggccgagagagcctccaccttcgcagctgcggagatctgagctctagctcttccctgcggcgtctcctgtctggccgcaggctggagcgtggtacccgcccccacagcctcagcctcaacgggggcagccgggagactgggctctgacctaggcttcttgtcacactgaacacatccagccacaggcaccagctggttgggaccagcagcccccagcatcctcttgcactggctggcacaaaaagaaacctgctgtataccccccaaagtgtccctttccctcctacctctggggtctcttgctgcttgcctctgctgctctggactgggagagcttctgtcctgtgctgcatgggtatttagactgtgggggagatgccccttcttatagcactggaggaggaaaacaaattcttgtccccctcagaatgagagtggctctttctgatttgcaagggcactatggtcagggcaaaggcatggcccaggtgtttaagtacagggtgacgtgtgcctatgcaatggggtggtaaggcaggcacgaagagtccaaaaaatctaggtggcctctcagctctgccacctctagctgcatgaccttgggcaagctatgtaaccccaattgcctgctccattaaagactgtgaaggtagaatgtttgtaaagctcttaacagtatgtaagccttcaataaatttcagttttccccttgttttcttgatcaaaaaaaaaaaaaaaaaaaaaaaaaaaaagggggggggcggccggatataggagagttcggtgagcccggggaaattaaattcgggagacggggaaccgggagggggttttccaaggaaaattggggaggttaagggggcccggaggtttattggggggccccaaaccggtttggtgtttggaacacaaaagggttggtttaaatgggacg

SEQ ID NO: 91 - DNA, 1451 bp, Homo sapiens
  misc_feature: Incyte ID No 7501108CB1
cctcttccgtcggctgaattgcggccgtatgcgcggctctgtggagtgcacctggggttgggggcactgtgcccccagccccctgctcctttggactctacttctgtttgcagccccatttggcctgctgggggagaagacccgccaggtgtctctggaggtcatccctaactggctgggccccctgcagaacctgcttcatatacgggcagtgggcaccaattccacactgcactatgtgtggagcagcctggggcctctggcagtggtaatggtggccaccaacaccccccacagcaccctgagcgtcaactggagcctcctgctatcccctgagcccgatgggggcctgatggtgctccctaaggacagcattcagttttcttctgcccttgtttttaccaggctgcttgagtttgacagcaccaacgtgtccgatacggcagcaaagcctttgggaagaccatatcctccatactccttggccgatttctcttggaacaacatcactgattcattggatcctgccaccctgagtgccacatttcaaggccaccccatgaacgaccctaccaggacttttgccaatggcagcctggccttcagggtccaggccttttccaggtccagccgaccagcccaaccccctcgcctcctgcacacagcagacacctgtcagctagaggtggccctgattggagcctctccccggggaaaccgttccctgtttgggctggaggtagccacattgggcccgggccctgactgcccctcaatgcaggagcagcactccatcgacgatgaatatgcaccggccgtcttccagtcacccattgtccgagccttctttgggtcccagaataacttctgtgccttcaatctgacgttcggggcttccacaggccctggctattgggaccaacactacctcagctggtcgatgctcctgggtgtgggcttccctccagtggacggcttgtccccactagtcctgggcatcatggcagtggccctgggtgccccagggctcatgctgctagggggcggcttggttctgctgctgcaccacaagaagtactcagagtaccagtccataaattaaggcccgctctctggagggaaggacattactgaacctgtcttgctgtgcctcgaaactctggaggttggagcatcaagttccagccggccccttcactcccccatcttgcttttctgtggaacctcagaggccagcctcgacttcctggagacccccaggtggggcttccttcatactttgttgggggactttggaggcgggcaggggacagggctattgataaggtccccttggtgttgccttcttgcatctccacacatttcccttggatgggacttgcaggcctaaatgagaggcattctgactggttggctgccctggaaggcaagaaaatagatttattttttta

SEQ ID NO: 92 - DNA, 1488 bp, Homo sapiens
  misc_feature: Incyte ID No 7507581CB1
gccagcggcgccgcgggcctgcgtgctgggtgcagcgggcacttcttcgacctcgtcctcctcgtcctgtgcggccggccgggtgaggccgggcccgcgtagggggcagtcggcggctgcctccggcggaggtgcctcgcggcgcccgggccggcccgcgcctcggcggcgtgctccatgcatccggagcccgccccgcccccgagccgcagcagtcccgagcttcccccaagcggcggcagcaccaccagcggcagccgccggagccgccgccgcagcggggacggggagcccccgggggccccgccaccgccgccgtccgccgtcacctacccggactggatcggccagagttactccgaggtgatgagcctcaacgagcactccatgcaggcgctgtcctggcgcaagctctacttgagccgcgccaagcttaaagcctccagccggacctcggctctgctctccggcttcgccatggtggcaatggtggaggtgcagctggacgctgaccacgactacccaccggggctgctcatcgccttcagtgcctgcaccacagtgctggtggctgtgcacctgtttgcgctcatgatcagcacctgcatcctgcccaacatcgaggcggtgagcaacgtgcacaatctcaactcggtcaaggagtccccccatgagcgcatgcaccgccacatcgagctggcctgggccttctccaccgtcatcggcacgctgctcttcctagctgaggtggtgctgctctgctgggtcaagttcttgcccctcaagaagcagccaggccagccaaggcccaccagcaagccccccgccagtggcgcagcagccaacgtcagcaccagcggcatcaccccgggccaggcagctgccatcgcctcgaccaccatcatggtgcccttcggcctgatctttatcgtcttcgccgtccacttctaccgctcactggttagccataagactgaccgacagttccaggagctcaacgagctggcggagtttgcccgcttacaggaccagctggaccacagaggggaccaccccctgacgcccggcagccactatgcctaggcccatgtggtctgggcccttccagtgctttggccttacgcccttccccttgaccttgtcctgccccagcctcacggacagcctgcgcagggggctgggcttcagcaaggggcagagcatggagggaagaggatttttataagagaaatttctgcactttgaaactgtcctctaagagaataagcatttcctgttcttccagctccaggtccacctcctgttgggaggcggtggggggccaaagtggggccacacactcgctgtgtcccctctcctcccctgtgccagtgccacctgggtgcctcctcctgtcctgtccgtctcaacctccctcccgtccagcattgagtgtgtacatgtgtgtgtgacacataaatatactcataaggacaaaaaaaaaaaaaa

SEQ ID NO: 93 - DNA, 1875 bp, Homo sapiens
  misc_feature: Incyte ID No 7506361CB1
gggccccgctggaattcggagggcccctgggtaatggggcagagagatgggacctggggcaaaggctaagcgaaggagagctggagcgggtgaactaagagcgggggcgagatctgaggatggaaggctttgggggtgtcggaggcagagggacccgggggtttgcagcgaagggtgtctggagagggagagctgaggaggggccggttctgggggctgcagaacggggatttatggtgtcgactgggagcaggaggagggtcttcgaggggcctgggggcgggggactaagatggacgcctgggaagggaactgggaggcagcggggtgcctgggggccgagggctgaggacggggtgcggaggcgcactctgggaatgccgagagggtcccgcagagacgtcagggcgccgtgcgggccggcggggagctggggggctaggggcggacgccgacgtgatggcccttcccgcaggcgccgcggctctgctactgctgctgcccgccaccatgttccacctgctcctggcggcccgttcgggccccgcgcgcctgctgggtccacccgcgtccctgcccgggctggaggtgctgtggagcccacgggcgctgctgctgtggctcgcctggctcggcctgcaggcggcgctctacctactgccggcgcgcaaggtgcgggccccgctcgcggacgctcgggggagggaagcgaatgggctcggcgagggaaaggacgccccgggccttatcagagcccccttggacccgcagtggccgaggggcaggaattgaaggacaagagtcgcctgcgctatcctattaacggcttccaggccctggtgctgacagccctgttggtggggctggggatgtcagcggggctgcctctgggggcgctcccggaaatgctcctgcccttggcgtttgtcgccaccctcaccgctttcatcttcagcctctttctctacatgaaggcgcaggtagccccagtttcggccctggcacctggggggaactcaggcaatccgatttacgacttttttctgggacgagagctcaaccctcgtatctgtttcttcgacttcaaatatttctgtgaactgcgacccggcctcatcggctgggtcctcatcaacctggccctgttgatgaaggaggcagagcttcgaggcagtccctcactggccatgtggctggtcaatggcttccagttgctctacgtgggtgatgccctctggcacgaggaggccgtcctcaccaccatggatatcacacatgacgggtttggcttcatgctggcgtttggggacatggcctgggtgcccttcacctacagcctgcaggcccagttcctgctgcaccacccgcagcccctggggttgcccatggcctctgtcatctgcctcatcaatgggcttgagaccatctctacagccacagggcggaaactgctggtgtctgggtggtggggtatggtccgccatcccaactatcttggagacctcatcatggctctggcttggtccttgccctgcggggtgtcacacctgctgccctacttctacctcctctacttcaccgcgctgctggtgcaccgtgaggcccgggatgagcggagtgcctgcagaagtacggcctggcctggcaggagtactgccggcgtgtgccttaccgcatcatgccctacatctactgaagcggctccaccaccccaggtggggcatgtgcccactcatccaccagcacacccaggaccaggagcctcgacacacttgggactcaagggcttgcaccccacccagccctgaggatgaacaacctcagagaagaggtggtttagagcaaggaaaaaaatgaaaccagtgaccacaaaaa

SEQ ID NO: 94 - DNA, 3153 bp, Homo sapiens
  misc_feature: Incyte ID No 7509211CB1
cccttctctagtccccgacctgcggcagccggagctcggggagcggagcgtggtggggaggggagcgggacaggcgacacaggagacagcggcgccgcggcctctccccaccaggcggccccggatcctactggacgccctgagggcacaccgaccgcgcctctagagtcaccccacgccgacccctcccctcttctctagacttatttccatccttcccgcttttaccctccccacccctccctgggctccaggccgccgccccctcctcactcctggaccggcccttctcggtgcccctcttccctagggagatgcgatgagccggtgcccccgcgtctcatcgtcgccccgggcacggtgcccgtccagtgcccgtggtggggagggagcactccgcggtccctccgtgacgcccctcgcttggccccccccacagctggcgtccctcggccatgccccaggggacccagccagggggtgggctctagagcgagtggggtggagaggagaaaggacggggccttgggcgcctctgagatgctcccaagtgccagggagggccgagcgaggcgcaggcaaccgggcagcaggcatgatgccctcgcctagtgactccagccgctcgctgaccagccggcccagcaccaggggccttacccacctccgcctccaccgaccctggctgcaggccctgcttacgctggggctggtccaagtgctcctgggcatcctggtggtcaccttcagcatggtggcctcttccgtcaccaccaccgagagcatcaagaggtcctgcccgtcttgggctgggttctcgatctccttcttctccttgctttcggtgctctgtgtcatgcttagcatggctggctctgttctctcctgtaagaatgctcaactggcccgagacttccaacagtgctctctggaaggaaaggtctgtgtgtgctgtccctctgttcccctcctccggccctgtccagagtcggggcaggaactgaaagttgcccctaactccacctgtgatgaagcccgaggggccctcaagaacctgctcttcagcgtctgtgggctcaccatttgtgccgctataatctgtacactctctgctattgtctgctgcatccaaatcttctccctggacctcgtgcatacgcagctggcccctgagcggtcagtctcaggcccactgggacctctgggctgcacgtccccgcccccagcccctctcctacacaccatgctggacctggaggaatttgtcccgcctgtgcccccaccgccctactatcccccagagtatacctgcagctcagaaacagatgcacagagcatcacgtacaatggctccatggacagcccagtgcccttgtaccctaccgattgccccccttcttatgaggcagtcatgggactacgaggagacagccaggccactctctttgaccctcagcttcacgatggctcgtgcatctgtgaacgagtggcctccattgtagacgtgtccatggacagcgggtctctggtgctgtcagccattggtgacctccctgggggctctagcccgtcggaggactcgtgcctgctggagctgcagggctccgtgcgctccgtggactacgttctctttcgctccatccagcgcagccgtgccggctactgcctcagcctggactgtggcctgcggggccccttcgaggaaagccccctgccacggcgccccccacgggctgcccgctcctattcctgctctgcccctgaagctccacccccactgggtgcccccacagctgcccgcagctgccaccggttggagggctggccgccctgggtgggaccctgcttccccgagctgaggcggcgggtcccccggggagggggccgcccagccgcagccccgcccacccgagccccgactcgtcgcttcagcgatagctcaggttccctcaccccaccggggcaccggcctcctcatccggcatccccaccaccgctgctgctgccacggtcccacagcgacccaggcatcacgacctccagtgacactgctgacttcagggacctttataccaaagtgcttgaggaagaagctgcttctgtttcctctgcagatacagggctctgctctgaagcctgcctcttccgcctagcccgctgcccttcccccaagttgctacgtgcccggtcagccgagaaacggcgccctgtgcccaccttccaaaaagttcccctgccctcgggccctgcacctgcccactccctgggggacctaaagggcagctggcccaaggtcggggccctggtcactcgtttcctccagatatccaggaaagccccagaccccagtgggactggagctcatggacataagcaggtgccccggagcctgtggggccggcctggccgagagagcctccaccttcgcagctgcggagatctgagctctagctcttccctgcggcgtctcctgtctggccgcaggctggagcgtggtacccgcccccacagcctcagcctcaacgggggcagccgggagactgggctctgacctaggcttcttgtcacactgaacacatccagccacaggcaccagctggttgggaccagcagcccccagcatcctcttgcactggctggcacaaaaagaaacctgctgtataccccccaaagtgtccctttccctcctacctctggggtctcttgctgcttgcctctgctgctctggactgggagagcttctgtcctgtgctgcatgggtatttagactgtgggggagatgccccttcttatagcactggaggaggaaaacaaattcttgtccccctcagaatgagagtggctctttctgatttgcaagggcactatggtcagggcaaaggcatggcccaggtgtttaagtacagggtgacgtgtgcctatgcaatggggtggtaaggcaggcacgaagagtccaaaaaatctaggtggcctctcagctctgccacctctagctgcatgaccttgggcaagctatgtaaccccaattgcctgctccattaaagactgtgaaggtagaatgtttgtaaagctcttaacagtatgtaagccttcaataaatttcagttttccccttgttttcttgatcaaaaaaaaaaaaaaaaaaaagatcgg

(19) United States
(12) Patent Application Publication
(10) Pub. No.: US 2005/0107588 A1
(43) Pub. Date: May 19, 2005

(54) Receptors and membrane-associated proteins

(76) Inventors: Brendan M. Duggan, Sunnyvale, CA (US); Junming Yang, San Jose, CA (US); Kimberly J. Gietzen, San Jose, CA (US); Soo Yeun Lee, Mountain View, CA (US); Y. Tom Tang, San Jose, CA (US); Yalda Azimzai, Oakland, CA (US); Narinder K. Chawla, Union City, CA (US); Bridget A. Warren, San Marcos, CA (US); Ines Barroso, Cambridge (GB); Shanya D. Becha, San Francisco, CA (US); Henry Yue, Sunnyvale, CA (US); Patricia M. Lehr-Mason, Morgan Hill, CA (US); Kavitha Thangavelu, Sunnyvale, CA (US); Sally Lee, San Jose, CA (US); Brooke M. Emerling, Chicago, IL (US); Amy E. Kable, Silver Spring, MD (US); Reena Khare, Saratoga, CA (US); Mariah R. Baughn, Los Angeles, CA (US); Ameena R. Gandhi, San Francisco, CA (US); Uyen K. Tran, San Jose, CA (US); Thomas W. Richardson, Redwood City, CA (US); Joseph P. Marquis, San Jose, CA (US); Preeti G. Lal, Santa Clara, CA (US); Ian J. Forsythe, Edmonton (CA); Ernestine A. Lee, Kensington, CA (US); Anita Swarnakar, San Francisco, CA (US); Deborah A. Kallick, Galveston, TX (US); Jennifer A. Griffin, Fremont, CA (US); Vicki S. Elliott, San Jose, CA (US); Ann E. Gorvad, Bellingham, WA (US); April J.A. Hafalia, Daly City, CA (US); Craig H. Ison, San Jose, CA (US); Pei Jin, Palo Alto, CA (US); Xin Jiang, Saratoga, CA (US); Alan A. Jackson, Los Gatos, CA (US); Umesh G. Bhatia, San Jose, CA (US); John D. Burrill, Redwood City, CA (US); Julie J. Blake, San Francisco, CA (US); Anne Ho, Sunnyvale, CA (US); Wenjin Zheng, San Diego, CA (US); Jing Gao, Santa Clara, CA (US)

Correspondence Address: INCYTE CORPORATION;EXPERIMENTAL STATION, ROUTE 141 & HENRY CLAY ROAD, BLDG. E336, WILMINGTON, DE, 19880, US

(21) Appl. No.: 10/495,148

(22) PCT Filed: Nov. 13, 2002
(86) PCT No.: PCT/US02/36759

Related U.S. Application Data
(60) Provisional application No. 60/333,097, filed on Nov. 13, 2001.
(60) Provisional application No. 60/335,274, filed on Nov. 15, 2001.
(60) Provisional application No. 60/340,542, filed on Dec. 14, 2001.
(60) Provisional application No. 60/342,166, filed on Dec. 18, 2001.
(60) Provisional application No. 60/347,580, filed on Jan. 11, 2002.
(60) Provisional application No. 60/348,687, filed on Jan. 14, 2002.

(51) Int. Cl. C07K 14/705; C07H 21/04

(52) U.S. Cl. 530/350; 435/69.1; 435/320.1; 435/325; 536/23.5

(57) ABSTRACT

Various embodiments of the invention provide human receptors and membrane-associated proteins (REMAP) and polynucleotides which identify and encode REMAP. Embodiments of the invention also provide expression vectors, host cells, anti-bodies, agonists, and antagonists. Other embodiments provide methods for diagnosing, treating, or preventing disorders associated with aberrant expression of REMAP.

### TECHNICAL FIELD

The invention relates to novel nucleic acids, receptors and membrane-associated proteins encoded by these nucleic acids, and to the use of these nucleic acids and proteins in the diagnosis, treatment, and prevention of cell proliferative, autoimmune/inflammatory, neurological, metabolic, developmental, and endocrine disorders. The invention also relates to the assessment of the effects of exogenous compounds on the expression of nucleic acids and receptors and membrane-associated proteins.

### BACKGROUND OF THE INVENTION

Signal transduction is the general process by which cells respond to extracellular signals. Signal transduction across the plasma membrane begins with the binding of a signal molecule, e.g., a hormone, neurotransmitter, or growth factor, to a cell membrane receptor. The receptor, thus activated, triggers an intracellular biochemical cascade that ends with the activation of an intracellular target molecule, such as a transcription factor. This process of signal transduction regulates all types of cell functions including cell proliferation, differentiation, and gene transcription.

Biological membranes surround organelles, vesicles, and the cell itself. Membranes are highly selective permeability barriers made up of lipid bilayer sheets composed of phosphoglycerides, fatty acids, cholesterol, phospholipids, glycolipids, proteoglycans, and proteins. Membranes contain ion pumps, ion channels, and specific receptors for external stimuli which transmit biochemical signals across the membranes. These membranes also contain second messenger proteins which interact with these pumps, channels, and receptors to amplify and regulate transmission of these signals.

Plasma Membrane Proteins

Plasma membrane proteins (MPs) are divided into two groups based upon methods of protein extraction from the membrane. Extrinsic or peripheral membrane proteins can be released using extremes of ionic strength or pH, urea, or other disruptors of protein interactions. Intrinsic or integral membrane proteins are released only when the lipid bilayer of the membrane is dissolved by detergent.

The majority of known integral membrane proteins are transmembrane proteins (TM) which are characterized by an extracellular, a transmembrane, and an intracellular domain. TM domains are typically comprised of 15 to 25 hydrophobic amino acids which are predicted to adopt an α-helical conformation. TM proteins are classified as bitopic (Types I and II) and polytopic (Types III and IV) (Singer, S. J. (1990) Annu. Rev. Cell Biol. 6:247-296). Bitopic proteins span the membrane once while polytopic proteins contain multiple membrane-spanning segments. TM proteins carry out a variety of important cellular functions, including acting as cell-surface receptor proteins involved in signal transduction. These functions are represented by growth and differentiation factor receptors, and receptor-interacting proteins such as Drosophila pecanex and frizzled proteins, LIV-1 protein, NF2 protein, and GNS1/SUR4 eukaryotic integral membrane proteins. TM proteins also act as transporters of ions or metabolites, such as gap junction channels (connexins), and ion channels, and as cell anchoring proteins, such as lectins, integrins, and fibronectins. TM proteins may be vesicle organelle-forming molecules, such as caveolins, or cell recognition molecules, such as cluster of differentiation (CD) antigens, glycoproteins, and mucins.

Cell surface markets include cell surface antigens identified on leukocytic cells of the immune system. These antigens have been identified using systematic, monoclonal antibody (mAb)-based “shot gun” techniques. These techniques have resulted in the production of hundreds of mAbs directed against unknown cell surface leukocytic antigens. These antigens have been grouped into “clusters of differentiation” based on common immunocytochemical localization patterns in various differentiated and undifferentiated leukocytic cell types. Antigens in a given cluster are presumed to identify a single cell surface protein and are assigned a “cluster of differentiation” or “CD” designation. Some of tee genes encoding proteins identified by CD antigens have been cloned and verified by standard molecular biology techniques. CD antigens have been characterized as both transmembrane proteins and cell surface proteins anchored to the plasma membrane via covalent attachment to fatty acid-containing glycolipids such as glycosylphosphatidylinositol (GPI). (Reviewed in Barclay, A. N. et al. (1995) The Leucocyte Antigen Facts Book, Academic Press, San Diego, Calif. pp. 17-20.) The human cDNA sequence of myeloid Ag CD33 predicts a 40-kDa polypeptide with features of a glycosylated integral membrane protein. The extracellular part of CD33 contains two Ig-like domains which are highly related to the first two domains of the neural cell myelin-associated glycoprotein and the B cell Ag CD22 (Simmons, D. and Seed, B. (1988) J. Immunol. 141:2797-2800).

Many MPs contain amino acid sequence motifs that serve to localize proteins to specific subcellular sites. Examples of these motifs include PDZ domains, KDEL, RGD, NGR, and GSL sequence motifs, von Willebrand factor A (vWFA) domains, and EGF-like domains. RGD, NGR, and GSL motif-containing peptides have been used as drug delivery agents in targeted cancer treatment of tumor vasculature (Arap, W. et al. (1998) Science, 279:377-380). Furthermore, MPs may also contain amino acid sequence motifs that serve to interact with extracellular or intracellular molecules, such as carbohydrate recognition domains (CRD).

GMP-140 is an integral membrane glycoprotein found in secretory granules of platelets and endothelial cells. After cellular activation, it is rapidly redistributed to the plasma membrane. The cDNA-derived primary structure of GMP-140 predicts a cysteine-rich protein with multiple domains, including a lectin region, an EGF domain, nine tandem consensus repeats related to those in complement-binding proteins, a transmembrane domain, and a short cytoplasmic tail. Some cDNAs also predict a soluble protein with a deleted transmembrane segment. The domain organization of GMP-140 is similar to that of ELAM-1, a cytokine-inducible endothelial cell receptor that binds neutrophils. This similarity suggests that GMP-140 belongs to a new family of inducible receptors with related structure and function on vascular cell (Johnston, G. I. et al. (1989) Cell 56:1033-1044).

Chemical modification of amino acid residue side chains alters the manner in which MPs interact with other molecules, for example, phospholipid membranes. Examples of such chemical modifications to amino acid residue side chains are covalent bond formation with glycosaminoglycans, oligosaccharides, phospholipids, acetyl and palmitoyl moieties, ADP-ribose, phosphate, and sulphate groups.

RNA encoding membrane proteins may have alternative splice sites which give rise to proteins encoded by the same gene but with different messenger RNA and amino acid sequences. Splice variant membrane proteins may interact with other ligand and protein isoforms.

Receptors

The term receptor describes proteins that specifically recognize other molecules. The category is broad and includes proteins with a variety of functions. The bulk of receptors are cell surface proteins which bind extracellular ligands and produce cellular responses in the areas of growth, differentiation, endocytosis, and immune response. Other receptors facilitate the selective transport of proteins out of the endoplasmic reticulum and localize enzymes to particular locations in the cell. The term may also be applied to proteins which act as receptors for ligands with known or unknown chemical composition and which interact with other cellular components. For example, the steroid hormone receptors bind to and regulate transcription of DNA.

Cell surface receptors are typically integral plasma membrane proteins. These receptors recognize hormones such as catecholamines; peptide hormones; growth and differentiation factors; small peptide factors such as thyrotropin-releasing hormone; galanin, somatostatin, and tachykinins; and circulatory system-borne signaling molecules. Cell surface receptors on immune system cells recognize antigens, antibodies, and major histocompatibility complex (MHC)-bound peptides. Other cell surface receptors bind ligands to be internalized by the cell. This receptor-mediated endocytosis functions in the uptake of low density lipoproteins (LDL), transferrin, glucose- or mannose-terminal glycoproteins, galactose-terminal glycoproteins, immunoglobulins, phosphovitellogenins, fibrin, proteinase-inhibitor complexes, plasminogen activators, and thrombospondin (Lodish, H. et al. (1995) Molecular Cell Biology, Scientific American Books, New York N.Y., p. 723; Mikhailenko, I. et al. (1997) J. Biol. Chem. 272:6784-6791).

Receptor Protein Kinases

Many growth factor receptors, including receptors for epidermal growth factor, platelet-derived growth factor, fibroblast growth factor, as well as the growth modulator α-thrombin, contain intrinsic protein kinase activities. When growth factor binds to the receptor, it triggers the autophosphorylation of a serine, threonine, or tyrosine residue on the receptor. These phosphorylated sites are recognition sites for the binding of other cytoplasmic signaling proteins. These proteins participate in signaling pathways that eventually link the initial receptor activation at the cell surface to the activation of a specific intracellular target molecule. In the case of tyrosine residue autophosphorylation, these signaling proteins contain a common domain referred to as a Src homology (SH) domain. SH2 domains and SH3 domains are found in phospholipase C-γ, PI-3-K p85 regulatory subunit, Ras-GTPase activating protein, and pp600^{c-src} (Lowenstein, E. J. et al. (1992) Cell 70:431-442). The cytokine family of receptors share a different common binding domain and include transmembrane receptors for growth hormone (GH), interleukins, erythropoietin, and prolactin.

Other receptors and second messenger-binding proteins have intrinsic serine/threonine protein kinase activity. These include activin/TGF-β/BMP-superfamily receptors, calcium- and diacylglycerol-activated/phospholipid-dependant protein kinase (PK-C), and RNA-dependant protein kinase (PK-R). In addition, other serine/threonine protein kinases, including nematode Twitchin, have fibronectin-like, immunoglobulin C2-like domains.

G-Protein Coupled Receptors

The G-protein coupled receptors (GPCRs), encoded by one of the largest families of genes yet identified, play a central role in the transduction of extracellular signals across the plasma membrane. GPCRs have a proven history of being successful therapeutic targets.

GPCRs are integral membrane proteins characterized by the presence of seven hydrophobic transmembrane domains which together form a bundle of antiparallel alpha (α) helices. GPCRs range in size from under 400 to over 1000 amino acids (Strosberg, A. D. (1991) Eur. J. Biochem. 196:1-10; Coughlin, S. R. (1994) Curr. Opin. Cell Biol. 6:191-197). The amino-terminus of a GPCR is extracellular, is of variable length, and is often glycosylated. The carboxy-terminus is cytoplasmic and generally phosphorylated. Extracellular loops alternate with intracellular loops and link the transmembrane domains. Cysteine disulfide bridges linking the second and third extracellular loops may interact with agonists and antagonists. The most conserved domains of GPCRs are the part, for structural and functional features of the receptor. In most cases, the bundle of a helices forms a ligand-binding pocket. The extracellular N-terminal segment, or one or more of the three extracellular loops, may also participate in ligand-binding. Ligand binding activates the receptor by inducing a conformational change in intracellular portions of the receptor. In turn, the large, third intracellular loop of the activated receptor interacts with a heterotrimeric guanine nucleotide binding (G) protein complex which mediates further intracellular signaling activities, including the activation of second messengers such as cyclic AMP (cAMP), phospholipase C, and inositol triphosphate, and the interaction of the activated GPCR with ion channel proteins. (See, e.g., Watson, S. and S. Arkinstall (1994) The G-protein Linked Receptor Facts Book, Academic Press, San Diego Calif., pp. 2-6; Bolander, F. F. (1994) Molecular Endocrinology, Academic Press, San Diego Calif., pp. 162-176; Baldwin, J. M. (1994) Curr. Opin. Cell Biol. 6:180-190.)

GPCRs include receptors for sensory signal mediators (e.g., light and olfactory stimulatory molecules); adenosine, γ-aminobutyric acid (GABA), hepatocyte growth factor, melanocortins, neuropeptide Y, opioid peptides, opsins, somatostatin, tachykinins, vasoactive intestinal polypeptide family, and vasdpressin; biogenic amines (e.g., dopamine, epinephrine and norepinephrine, histamine, glutamate (metabotropic effect), acetylcholine (muscarinic effect), and serotonin); chemokines; lipid mediators of inflammation (e.g., prostaglandins and prostanoids, platelet activating factor, and leukotrienes); and peptide hormones (e.g., bombesin, bradykinin, calcitonin, C5a anaphylatoxin, endothelin, follicle-stimulating hormone (FSH), gonadotropic-releasing hormone (GnRH), neurokinin, and thyrotropin-releasing hormone (TRH), and oxytocin). GPCRs which act as receptors for stimuli that have yet to be identified are known as orphan receptors.

GPCR mutations, which may cause loss of function or constitutive activation, have been associated with numerous human diseases (Coughlin, supra). For instance, retinitis pigmentosa may arise from mutations in the rhodopsin gene. Furthermore, somatic activating mutations in the thyrotropin receptor have been reported to cause hyperfunctioning thyroid adenomas, suggesting that certain GPCRs susceptible to constitutive activation may behave as protooncogenes (Parma, J. et al. (1993) Nature 365:649-651). GPCR receptors for the following ligands also contain mutations associated with human disease: luteinizing hormone (precocious puberty); vasopressin V₂ (X-linked nephrogenic diabetes); glucagon (diabetes and hypertension); calcium (hyperparathyroidism, hypocalcuria, hypercalcemia); parathyroid hormone (short limbed dwarfism); β₃-adrenoceptor (obesity, non-insulin-dependent diabetes mellitus); growth hormone releasing hormone (dwarfism); and adrenocorticotropin (glucocorticoid deficiency) (Wilson, S. et al. (1998) Br. J. Pharmocol. 125:1387-1392; Stadel, J. M. et al. (1997) Trends Pharmacol. Sci. 18:430-437). GPCRs are also involved in depression, schizophrenia, sleeplessness, hypertension, anxiety, stress, renal failure, and several cardiovascular disorders (Horn, F. and G. Vriend (1998) J. Mol. Med. 76:464-468).

In addition, within the past 20 years several hundred new drugs have been recognized that are directed towards activating or inhibiting GPCRs. The therapeutic targets of these drugs span a wide range of diseases and disorders, including cardiovascular, gastrointestinal, and central nervous system disorders as well as cancer, osteoporosis and endometriosis (Wilson et al., supra; Stadel et al., supra). For example, the dopamine agonist L-dopa is used to treat Parkinson's disease, while a dopamine antagonist is used to treat schizophrenia and the early stages of Huntington's disease. Agonists and antagonists of adrenoceptors have been used for the treatment of asthma, high blood pressure, other cardiovascular disorders, and anxiety; muscarinic agonists are used in the treatment of glaucoma and tachycardia; serotonin 5HT1D antagonists are used against migraine; and histamine H1 antagonists are used against allergic and anaphylactic reactions, hay fever, itching, and motion sickness (Horn et al., supra).

Nuclear Receptors

Nuclear receptors bind small molecules such as hormones or second messengers, leading to increased receptor-binding affinity to specific chromosomal DNA elements. In addition the affinity for other nuclear proteins may also be altered. Such binding and protein-protein interactions may regulate and modulate gene expression. Examples of such receptors include the steroid hormone receptors family, the retinoic acid receptors family, and the thyroid hormone receptors family.

Ligand-Gated Receptor Ion Channels

Ligand-gated receptor ion channels fall into two categories. The first category, extracellular ligand-gated receptor ion channels (ELGs), rapidly transduce neurotransmitter-binding events into electrical signals, such as fast synaptic neurotransmission. ELG function is regulated by post-translational modification. The second category, intracellular ligand-gated receptor ion channels (ILGs), are activated by many intracellular second messengers and do not require post-translational modification(s) to effect a channel-opening response.

ELGs depolarize excitable cells to the threshold of action potential generation. In non-excitable cells, ELGs permit a limited calcium ion-influx during the presence of agonist. ELGs include channels directly gated by neurotransmitters such as acetylcholine, L-glutamate, glycine, ATP, serotonin, GABA, and histamine. ELG genes encode proteins having strong structural and functional similarities. ILGs are encoded by distinct and unrelated gene families and include receptors for cAMP, cGMP, calcium ions, ATP, and metabolites of arachidonic acid.

Macrophage Scavenger Receptors

Macrophage scavenger receptors with broad ligand specificity may participate in the binding of low density lipoproteins (LDL) and foreign antigens. Scavenger receptors types I and II are trimeric membrane proteins with each subunit containing a small N-terminal intracellular domain, a transmembrane domain, a large extracellular domain, and a C-terminal cysteine-rich domain. The extracellular domain contains a short spacer domain, an c-helical coiled-coil domain, and a triple helical collagenous domain. These receptors have been shown to bind a spectrum of ligands, including chemically modified lipoproteins and albumin, polyribonucleotides, polysaccharides, phospholipids, and asbestos (Matsumoto, A. et al. (1990) Proc. Natl. Acad. Sci. USA 87:9133-9137; Elomaa, O. et al. (1995) Cell 80:603-609). The scavenger receptors are thought to play a key role in atherogenesis by mediating uptake of modified LDL in arterial walls, and in host defense by binding bacterial endotoxins, bacteria, and protozoa.

T-Cell Receptors

T cells play a dual role in the immune system as effectors and regulators, coupling antigen recognition with the transmission of signals that induce cell death in infected cells and stimulate proliferation of other immune cells. Although a population of T cells can recognize a wide range of different antigens, an individual T cell can only recognize a single antigen and only when it is presented to the T cell receptor (TCR) as a peptide complexed with a major histocompatibility molecule (MHC) on the surface of an antigen presenting cell. The TCR on most T cells consists of immunoglobulin-like integral membrane glycoproteins containing two polypeptide subunits, a and A, of similar molecular weight. Both TCR subunits have an extracellular domain containing both variable and constant regions, a transmembrane domain that traverses the membrane once, and a short intracellular domain (Saito, H. et al. (1984) Nature 309:757-762). The genes for the TCR subunits are constructed through somatic rearrangement of different gene segments. Interaction of antigen in the proper MHC context with the TCR initiates signaling cascades that induce the proliferation, maturation, and function of cellular components of the immune system (Weiss, A. (1991) Annu. Rev. Genet. 25:487-510). Rearrangements in TCR genes and alterations in TCR expression have been noted in lymphomas, leukemias, autoimmune disorders, and immunodeficiency disorders (Aisenberg, A. C. et al. (1985) N. Engl. J. Med. 313:529-533; Weiss, supra).

Netrin Receptors:

The netrins are a family of molecules that function as diffusible attractants and repellants to guide migrating cells and axons to their targets within the developing nervous system. The netrin receptors include the C. elegans protein UNC-5, as well as homologues recently identified in vertebrates (Leonardo, E. D. et al. (1997) Nature 386:833-838). These receptors are members of the immunoglobulin superfamily, and also contain a characteristic domain called the ZU5 domain. Mutations in the mouse member of the netrin receptor family, Rcm (rostral cerebellar malformation) result in cerebellar and midbrain defects as an apparent result of abnormal neuronal migration (Ackerman, S. L. et al. (1997) Nature 386:838-842).

VPS10 Domain Containing Receptors

The members of the VPS10 domain containing receptor family all contain a domain with homology to the yeast vacuolar sorting protein 10 (VPS10) receptor. This family includes the mosaic receptor SorLA, the neurotensin receptor sortilin, and SorCS, which is expressed during mouse embryonal and early postnatal nervous system development (Hermey, G. et al. (1999) Biochem. Biophys. Res. Commun. 266:347-351; Hermey, G. et al. (2001) Neuroreport 12:29-32). A recently identified member of this family, SorCS2, is highly expressed in the developing and mature mouse central nervous system. Its main site of expression is the floor plate, and high levels are also detected transiently in brain regions including the dopaminergic brain nuclei and the dorsal thalamus (Rezgaoui, M. (2001) Mech. Dev. 100:335-338).

Membrane-Associated Proteins

Tetraspan Family Proteins

The transmembrane 4 superfamily (TM4SF) or tetraspan family is a multigene family encoding type III integral membrane proteins (Wright, M. D. and M. G. Tomlinson (1994) Immunol. Today 15:588-594). The TM4SF is comprised of membrane proteins which traverse the cell membrane four times. Members of the TM4SF include platelet and endothelial cell membrane proteins, melanoma-associated antigens, leukocyte surface glycoproteins, colonal carcinoma antigens, tumor-associated antigens, and surface proteins of the schistosome parasites (Jankowski, S. A. (1994) Oncogene 9:1205-1211). Members of the TM4SF share about 25-30% amino acid sequence identity with one another. A number of TM4SF members have been implicated in signal transduction, control of cell adhesion, regulation of cell growth and proliferation, including development and oncogenesis, and cell motility, including tumor cell metastasis. Expression of TM4SF proteins is associated with a variety of tumors and the level of expression may be altered when cells are growing or activated.

Tetraspan Family Proteins

The transmembrane 4 superfamily (TM4SF; tetraspanin) proteins encode type III integral membrane proteins and traverse the cell membrane four times (Wright, M. D. and Tomlinson, M. G. (1994) Immunol. Today 15:588-594). They are found predominantly in cells of hematopoietic origin and in tumors and include a number of platelet and endothelial cell membrane proteins; CD9 (the lung adenocarcinoma antigen MRP-1); CD53, CD37 (the human melanoma associated antigen; Classon, B. J. et al. (1989) J. Exp. Med. 169:1497-1502), CD63, and R2 (leukocyte surface glycoproteins); CD81 (the tumor associated antigen, TAPA-1); CO-029 (the colonal carcinoma antigen); the tumor-associated SAS gene (amplified in human sarcomas) (Wright and Tomlinson, supra; Jankowski et al. (1994) Oncogene 9:1205-1211; and TI-1 (the mink lung epithelial protein) (Kallin et al. (1991) Mol. Cell. Biol. 11:5338-5345). These proteins all share about 25-30% amino acid sequence identity.

The tetraspanin proteins reveal a topology where the N- and C-termini are intracellular and the major hydrophilic domain, located between transmembrane domains 3 and 4, is extracellular. Tetraspanin proteins are most conserved in their transmembrane and cytoplasmic domains and most divergent in their hydrophilic extracellular domains which contain N-linked glycosylation sites. The high level of conservation in the transmembrane and cytoplasmic domains suggests an effector/signaling function. The divergence of the extracellular domains suggests that these hydrophilic domains provide functions specific to each protein such as ligand binding or protein-protein interaction (Wright and Tomlinson, supra).

Tetraspanin proteins have been implicated in signal transduction, control of cell adhesion, cell motility, and regulation of cell growth and proliferation (Wright and Tomlinson, supra; Jankowski supra). In particular, TM4SF expression has been found to be negatively associated with cell motility and, consequently, tetraspanin proteins appear to function in tumor cells as metastasis suppressors by acting as brakes on the motility of tumor cells (Mollinedo et al. (1998) J. Leukoc. Biol. 63:699-706). This is further substantiated by the finding that low levels of these proteins correlate with increased metastatic potential of various tumors, and thus poor prognosis (Mollinedo, supra). It has been proposed that these effects on cell motility result from the association of various TM4SF proteins with integrins, a class of cell surface receptors long known to be associated with the growth and metastasis of tumors (Hemler et al. (1996) Biochem. Biophys. Acta 1287:67-71).

TM4-B is a typical tetraspanin protein with significant homology to other superfamily members. It is most similar to Tspan-1. TM4-B is expressed in most human tissues and cell lines including neural- and bone marrow-derived tissues. TM4-B has been mapped to the q34 on human chromosome 9 (Puls, K. L. et al. (1999) Biochim. Biophys. Acta 1447:93-99). Further, seven new members of the tetraspanin superfamily have been isolated. They are called NET (new EST tetraspan)-1 through NET-7. They each contain four transmembrane domains delimiting two extracellular regions as well as conserved amino acid residues. They are differentially expressed in human cell lines (Serru, V. et al. (2000) Biochim. Biophys. Acta 12478:159-163).

Tumor Antigens

Tumor antigens are surface molecules that are differentially expressed in tumor cells relative to normal cells. Tumor antigens distinguish tumor cells immunologically from normal cells and provide diagnostic and therapeutic targets for human cancers (Takagi, S. et al. (1995) Int. J. Cancer 61:706-715; Liu, E. et al. (1992) Oncogene 7:1027-1032).

Ion Channels

Ion channels are found in the plasma membranes of virtually every cell in the body. For example, chloride channels mediate a variety of cellular functions including regulation of membrane potentials and absorption and secretion of ions across epithelial membranes. When present in intracellular membranes of the Golgi apparatus and endocytic vesicles, chloride channels also regulate organelle pH. (See, e.g., Greger, R. (1988) Annu. Rev. Physiol. 50:111-122.) Electrophysiological and pharmacological properties of chloride channels, including ion conductance, current-voltage relationships, and sensitivity to modulators, suggest that different chloride channels exist in muscles, neurons, fibroblasts, epithelial cells, and lymphocytes. Many channels have sites for phosphorylation by one or more protein kinases including protein kinase A, protein kinase C, tyrosine kinase, and casein kinase II, all of which regulate ion channel activity in cells. Inappropriate phosphorylation of proteins in cells has been linked to changes in cell cycle progression and cell differentiation. Changes in the cell cycle have been linked to induction of apoptosis or cancer. Changes in cell differentiation have been linked to diseases and disorders of the reproductive system, immune system, and skeletal muscle.

Cerebellar granule neurons possess a non-inactivating potassium current which modulates firing frequency upon receptor stimulation by neurotransmitters and controls the resting membrane potential. Potassium channels that exhibit non-inactivating currents include the ether a go-go (EAG) channel. A membrane protein designated KCR1 specifically binds to rat EAG by means of its C-terminal region and regulates the cerebellar non-inactivating potassium current. KCR1 is predicted to contain 12 transmembrane domains, with intracellular amino and carboxyl termini. Structural characteristics of these transmembrane regions appear to be similar to those of the transporter superfamily, but no homology between KCR1 and known transporters was found, suggesting that KCR1 belongs to a novel class of transporters. KCR1 appears to be the regulatory component of non-inactivating potassium channels (Hoshi, N. et al. (1998) J. Biol. Chem. 273:23080-23085).

ABC Transporters

ATP-binding cassette (ABC) transporters, also called the “traffic ATPases”, are a superfamily of membrane proteins that mediate transport and channel functions in prokaryotes and eukaryotes (Higgins, C. F. (1992) Annu. Rev. Cell Biol. 8:67-113). ABC proteins share a similar overall structure and significant sequence homology. All ABC proteins contain a conserved domain of domains. Mutations in ABC transporter genes are associated with various disorders, such as hyperbilirubinemia II/Dubin-Johnson syndrome, recessive Stargardt's disease, X-linked adrenoleukodystrophy, multidrug resistance, celiac disease, and cystic fibrosis.

Cell Adhesion Proteins

The surface of a cell is rich in transmembrane proteoglycans, glycoproteins, glycolipids, and receptors. These macromolecules mediate adhesion with other cells and with components of the ECM. The interaction of the cell with its surroundings profoundly influences cell shape, strength, flexibility, motility, and adhesion. These dynamic properties are intimately associated with signal transduction pathways controlling cell proliferation and differentiation, tissue construction, and embryonic development. Families of cell adhesion molecules include the cadherins, integrins, lectins, neural cell adhesion proteins, and some members of the proline-rich proteins.

Vezatin is a ubiquitous protein of adherens cell-cell junctions, where it interacts with both myosin VIIA and the cadherin-catenins complex (Kussel-Andermann, P. et al. (2000) EMBO J. 19:6020-6029).

Semaphorins and Neuropilins

Semaphorins are a large group of axonal guidance molecules consisting of at least 30 different members and are found in vertebrates, invertebrates, and even certain viruses. All semaphorins contain the sema domain which is approximately 500 amino acids in length. Neuropilin, a semaphorin receptor, has been shown to promote neurite outgrowth in vitro. The extracellular region of neuropilins consists of three different domains: CUB, discoidin, and MAM domains. The CUB and the MAM motifs of neuropilin have been suggested to have roles in protein-protein interactions and are thought to be involved in the binding of semaphorins through the sema and the C-terminal domains (reviewed in Raper, J. A. (2000) Curr. Opin. Neurobiol. 10:88-94).

Membrane Proteins Associated with Intercellular Communication

Intercellular communication is essential for the development and survival of multicellular organisms. Cells communicate with one another through the secretion and uptake of protein signaling molecules. The uptake of proteins into the cell is achieved by endocytosis, in which the interaction of signaling molecules with the plasma membrane surface, often via binding to specific receptors, results in the formation of plasma membrane-derived vesicles that enclose and transport the molecules into the cytosol. The secretion of proteins from the cell is achieved by exocytosis, in which molecules inside of the cell are packaged into membrane-bound transport vesicles derived from the trans Golgi network. These vesicles fuse with the plasma membrane and release their contents into the surrounding extracellular space. Endocytosis and exocytosis result in the removal and addition of plasma membrane components, and the recycling of these components is essential to maintain the integrity, identity, and functionality of both the plasma membrane and internal membrane-bound compartments.

Lipid rafts are microdomains of the plasma membrane enriched in cholesterol and sphingolipids. These regions concentrate certain signaling molecules, including heterotrimeric and small G proteins, Src-family tyrosine kinases, endothelial nitric oxide synthase, G-protein-coupled receptors, and certain tyrosine kinase receptors. This concentration of signaling molecules suggests that these microdomains might function as a site for compartmentalization of signaling events. Lipid rafts may also represent sites for the sequestered localization of certain membrane proteins. Among these are proteins wide lipid modifications, such as glycosylphosphatidylinositol-anchored cell surface proteins and cytoplasmically oriented proteins with closely spaced myristoylation and palmitoylation, as well as other hydrophobic integral membrane proteins such as caveolin and flotillin (Baumann, C. A. et al. (2000) Nature (London) 407:202-207).

An essential role in intracellular signaling pathways is filled by second messenger molecules, intermediates that are activated upon binding of ligands to surface receptors and serve as activators of downstream effector molecules. The cyclic nucleotides, adenosine 3′,5′-cyclic monophosphate (cAMP) and guanosine 3′5′-cyclic monophosphate (cGMP) are critical second messengers in a wide variety of signaling pathways. cAMP and cGMP are generated by the enzymes adenylyl (adenylate) cyclase (AC) and guanylyl (guanylate) cyclase (GC) from ATP and GTP. Thus a key step in regulating intracellular cAMP and cGMP levels is modulation of AC and GC activity.

Nogo has been identified as a component of the central nervous system myelin that prevents axonal regeneration in adult vertebrates. Cleavage of the Nogo-66 receptor and other glycophosphatidylinositol-linked proteins from axonal surfaces renders neurons insensitive to Nogo-66, facilitating potential recovery from CNS damage (Fournier, A. E. et al. (2001) Nature 409:341-346).

The slit proteins are extracellular matrix proteins expressed by cells at the ventral midline of the nervous system. Slit proteins are ligands for the repulsive guidance receptor Roundabout (Robo) and thus play a role in repulsive axon guidance (Brose, K. et al. (1999) Cell 96:795-806).

Lysosomes are the site of degradation of intracellular material during autophagy and of extracellular molecules following endocytosis. Lysosomal enzymes are packaged into vesicles which bud from the trans-Golgi network. These vesicles fuse with endosomes to form the mature lysosome in which hydrolytic digestion of endocytosed material occurs. Lysosomes can fuse with autophagosomes to form a unique compartment in which the degradation of organelles and other intracellular components occurs.

Protein sorting by transport vesicles, such as the endosome, has important consequences for a variety of physiological processes including cell surface growth, the biogenesis of distinct intracellular organelles, endocytosis, and the controlled secretion of hormones and neurotransmitters (Rothman, J. E. and F. T. Wieland (1996) Science 272:227-234). In particular, neurodegenerative disorders and other neuronal pathologies are associated with biochemical flaws during endosomal protein sorting or endosomal biogenesis (Mayer, R. J. et al. (1996) Adv. Exp. Med. Biol. 389:261-269).

Peroxisomes are organelles independent from the secretory pathway. They are the site of many peroxide-generating oxidative reactions in the cell. Peroxisomes are unique among eukaryotic organelles in that their size, number, and enzyme content vary depending upon organism, cell type, and metabolic needs (Waterham, H. R. and J. M. Cregg (1997) BioEssays 19:57-66). Genetic defects in peroxisome proteins which result in peroxisomal deficiencies have been linked to a number of human pathologies, including Zellweger syndrome, rhizomelic chonrodysplasia punctata, X-linked adrenoleukodystrophy, acyl-CoA oxidase deficiency, bifunctional enzyme deficiency, classical Refsum's disease, DHAP alkyl transferase deficiency, and acatalasemia (Moser, H. W. and A. B. Moser (1996) Ann. NY Acad. Sci. 804:427-441). In addition, Gartner, J. et al. (1991; Pediatr. Res. 29:141-146) found a 22 kDa integral membrane protein associated with lower density peroxisome-like subcellular fractions in patients with Zellweger syndrome.

Normal embryonic development and control of germ cell maturation is modulated by a number of secretory proteins which interact with their respective membrane-bound receptors. Cell fate during embryonic development is determined by members of the activin/TGF-β superfamily, cadherins, IGF-2, and other morphogens. In addition, proliferation, maturation, and redifferentiation of germ cell and reproductive tissues are regulated, for example, by IGF-2, inhibins, activins, and follistatins (Petraglia, F. (1997) Placenta 18:3-8; Mather, J. P. et al. (1997) Proc. Soc. Exp. Biol. Med. 215:209-222). Transforming growth factor beta (TGFbeta) signal transduction is mediated by two receptor Ser/Thr kinases acting in series, type II TGFbeta receptor and (TbetaR-II) phosphorylating type I TGFbeta receptor (ThetaR-I). ThetaR-1-associated protein-1 (TRECAP-1), which distinguishes between quiescent and activated forms of the type I transforming growth factor beta receptor, has been associated with TGFbeta signaling (Charng, M. J. et al. (1998) J. Biol. Chem. 273:9365-9368).

Retinoic acid receptor alpha (RAR alpha) mediates retinoic-acid induced maturation and has been implicated in myeloid development. Genes induced by retinoic acid during granulocytic differentiation include E3, a hematopoietic-specific gene that is an immediate target for the activated RAR alpha during myelopoiesis (Scott, L. M. et al. (1996) Blood 88:2517-2530).

The μ-opioid receptor (MOR) mediates the actions of analgesic agents including morphine, codeine, methadone, and fentanyl as well as heroin. MOR is functionally coupled to a G-protein-activated potassium channel (Mestek A. et al. (1995) J. Neurosci. 15:2396-2406). A variety of MOR subtypes exist. Alternative splicing has been observed with MOR-1 as with a number of G protein-coupled receptors including somatostatin 2, dopamine D2, prostaglandin EP3, and serotonin receptor subtypes 5-hydroxytryptamine4 and 5-hydroxytryptamine7 (Pan, Y. X. et al. (1999) Mol. Pharm. 56:396-403).

Peripheral and Anchored Membrane Proteins

Some membrane proteins are not membrane-spanning but are attached to the plasma membrane via membrane anchors or interactions with integral membrane proteins. Membrane anchors are covalently joined to a protein post-translationally and include such moieties as prenyl, myristyl, and glycosylphosphatidyl inositol groups. Membrane localization of peripheral and anchored proteins is important for their function in processes such as receptor-mediated signal transduction. For example, prenylation of Ras is required for its localization to the plasma membrane and for its normal and oncogenic functions in signal transduction.

Glycosylphosphatidylinositol (GPI)-anchored proteins are cell surface-localized proteins that serve many important cellular functions. The pathway mediating synthesis and attachment of the GPI anchor to these proteins in eukaryotic cells is complex, highly conserved, and plays a critical role in the proper targeting, transport, and function of all GPI-anchored protein family members. MCD4 is an essential gene initially identified in Saccharomyces cerevisiae mutants defective for bud emergence. MCD4 encodes a conserved component of the GPI anchor synthesis pathway. Mcd4p is a multimembrane-spanning protein that localizes to the endoplasmic reticulum (ER) and contains a large NH2-terminal ER lumenal domain. Mcd4p is both highly conserved throughout eukaryotes and has two yeast homologues. Mcd4p's lumenal domain contains three conserved motifs found in mammalian phosphodiesterases and nucleotide pyrophosphases. Mcd4p functions in GPI anchoring, bud emergence, cell wall function, and feedback mechanisms likely to be involved in regulating each of these essential processes (Gaynor, E. C. et al. (1999) Mol. Biol. Cell 10:627-648).

Expression Profiling

Microarrays are analytical tools used in bioanalysis. A microarray has a plurality of molecules spatially distributed over, and stably associated with, the surface of a solid support. Microarrays of polypeptides, polynucleotides, and/or antibodies have been developed and find use in a variety of applications, such as gene sequencing, monitoring gene expression, gene mapping, bacterial identification, drug discovery, and combinatorial chemistry.

One area in particular in which micro arrays find use is in gene expression analysis. Array technology can provide a simple way to explore the expression of a single polymorphic gene or the expression profile of a large number of related or unrelated genes. When the expression of a single gene is examined, arrays are employed to detect the expression of a specific gene or its variants. When an expression profile is examined, arrays provide a platform for identifying genes that are tissue specific, are affected by a substance being tested in a toxicology assay, are part of a signaling cascade, carry out housekeeping functions, or are specifically related to a particular genetic predisposition, condition, disease, or disorder.

Osteosarcoma is the most common malignant bone tumor in children. With currently available treatment regimens, approximately 30-40% of patients with non-metastatic disease relapse after therapy. Currently, there is no prognostic factor that can be used at the time of initial diagnosis to predict which patients will have a high risk of relapse. The only significant prognostic factor predicting the outcome in a patient with non-metastatic osteosarcoma is the histopathologic response of the primary tumor resected at the time of definitive surgery.

The potential application of gene expression profiling is also relevant to measuring the toxic response to potential therapeutic compounds and of the metabolic response to therapeutic agents. For instance, diseases treated with steroids and disorders caused by the metabolic response to treatment with steroids include adenomatosis, cholestasis, cirrhosis, hemangioma, Henoch-Schonlein purpura, hepatitis, hepatocellular and metastatic carcinomas, idiopathic thrombocytopenic purpura, porphyria, sarcoidosis, and Wilson disease. It is desirable to measure the toxic response to potential therapeutic compounds and of the metabolic response to therapeutic agents.

Ovarian Cancer

Ovarian cancer is the leading cause of death from a gynecologic cancer. The majority of ovarian cancers are derived from epithelial cells, and 70% of patients with epithelial ovarian cancers present with late-stage disease. As a result, the long-term survival rates for this disease is very low. Identification of early-stage markers for ovarian cancer would significantly increase the survival rate. The molecular events that lead to ovarian cancer are poorly understood. Some of the known aberrations include mutation of p53 and microsatellite instability. Since gene expression patterns are likely to vary when normal ovary is compared to ovarian tumors, examination of gene expression in these tissues to identify possible markers for ovarian cancer is particularly relevant to improving diagnosis, prognosis, and treatment of this disease.

Lung Cancer

Lung cancer is the leading cause of cancer death in the United States, affecting more than 100,000 men and 50,000 women each year. Nearly 90% of the patients diagnosed with lung cancer are cigarette smokers. Tobacco smoke contains thousands of noxious substances that induce carcinogen metabolizing enzymes and covalent DNA adduct formation in the exposed bronchial epithelium. In nearly 80% of patients diagnosed with lung cancer, metastasis has already occurred. Most commonly lung cancers metastasize to pleura, brain, bone, pericardium, and liver. The decision to treat with surgery, radiation therapy, or chemotherapy is made on the basis of tumor histology, response to growth factors or hormones, and sensitivity to inhibitors or drugs. With current treatments, most patients die within one year of diagnosis. Earlier diagnosis and a systematic approach to identification, staging, and treatment of lung cancer could positively affect patient outcome.

Lung cancers progress through a series of morphologically distinct stages from hyperplasia to invasive carcinoma. Malignant lung cancers are divided into two groups comprising four histopathological classes. The Non Small Cell Lung Carcinoma (NSCLC) group includes squamous cell carcinomas, adenocarcinomas, and large cell carcinomas and accounts for about 70% of all lung cancer cases. Adenocarcinomas typically arise in the peripheral airways and often form mucin secreting glands. Squamous cell carcinomas typically arise in proximal airways. The histogenesis of squamous cell carcinomas may be related to chronic inflammation and injury to the bronchial epithelium, leading to squamous metaplasia. The Small Cell Lung Carcinoma (SCLC) group accounts for about 20% of lung cancer cases. SCLCs typically arise in proximal airways and exhibit a number of paraneoplastic syndromes including inappropriate production of adrenocorticotropin and anti-diuretic hormone.

Lung cancer cells accumulate numerous genetic lesions, many of which are associated with cytologically visible chromosomal aberrations. The high frequency of chromosomal deletions associated with lung cancer may reflect the role of multiple tumor suppressor loci in the etiology of this disease. Deletion of the short arm of chromosome 3 is found in over 90% of cases and represents one of the earliest genetic lesions leading to lung cancer. Deletions at chromosome arms 9p and 17p are also common. Other frequently observed genetic lesions include overexpression of telomerase, activation of oncogenes such as K-ras and c-myc, and inactivation of tumor suppressor genes such as RB, p53 and CDKN2.

Genes differentially regulated in lung cancer have been identified by a variety of methods. Using mRNA differential display technology, Manda et al. (1999; Genomics 51:5-14) identified five genes differentially expressed in lung cancer cell lines compared to normal bronchial epithelial cells. Among the known genes, pulmonary surfactant apoprotein A and alpha 2 macroglobulin were down regulated whereas nm23H1 was upregulated. Petersen et al. (2000; Int J. Cancer, 86:512-517) used suppression subtractive hybridization to identify 552 clones differentially expressed in lung tumor derived cell lines, 205 of which represented known genes. Among the known genes, thrombospondin-1, fibronectin, intercellular adhesion molecule 1, and cytokeratins 6 and 18 were previously observed to be differentially expressed in lung cancers. Wang et al. (2000; Oncogene 19:1519-1528) used a combination of microarray analysis and subtractive hybridization to identify 17 genes differentially overexpresssed in squamous cell carcinoma compared with normal lung epithelium. Among the known genes they identified were keratin isoform 6, KOC, SPRC, IGFb2, connexin 26, plakofillin 1 and cytokeratin 13.

Array technology can provide a simple way to explore the expression profile of a large number of related or unrelated genes. When an expression profile is examined, arrays provide a platform for examining which genes are tissue specific, carrying out housekeeping functions, parts of a signaling cascade, or specifically related to a particular genetic predisposition, condition, disease, or disorder. The potential application of gene expression profiling is particularly relevant to improving diagnosis, prognosis, and treatment of disease. For example, both the levels and sequences expressed in tissues from subjects with lung cancer may be compared with the levels and sequences expressed in normal tissue.

Colon Cancer

Colorectal cancer is the second leading cause of cancer deaths in the United States. Colon cancer is associated with aging, since 90% of the total cases occur in individuals over the age of 55. A widely accepted hypothesis is that several contributing genetic mutations must accumulate over time in an individual who develops the disease. To understand the nature of genetic alterations in colorectal cancer, a number of studies have focused on the inherited syndromes. The first known inherited syndrome, Familial Adenomatous Polyposis (FAP), is caused by mutations in the Adenomatous Polyposis Coli gene (APC), resulting in truncated or inactive forms of the protein. This tumor suppressor gene has been mapped to chromosome 5q. The second known inherited syndrome is hereditary nonpolyposis colorectal cancer (HNPCC), which is caused by mutations in mismatch repair genes.

Although hereditary colon cancer syndromes occur in a small percentage of the population and most colorectal cancers are considered sporadic, knowledge from studies of the hereditary syndromes can be generally applied. For instance, somatic mutations in APC occur in at least 80% of indiscriminate colon tumors. APC mutations are thought to be the initiating event in the disease. Other mutations occur subsequently. Approximately 50% of colorectal cancers contain activating mutations in ras, while 85% contain inactivating mutations in p53. Changes in these genes lead to gene expression changes in colon cancer. Less is understood about downstream targets of these mutations and the role they may play in cancer development and progression.

Steroids

The potential application of gene expression profiling is particularly relevant to measuring the toxic response to potential therapeutic compounds and of the metabolic response to therapeutic agents. Diseases treated with steroids and disorders caused by the metabolic response to treatment with steroids include adenomatosis, cholestasis, cirrhosis, hemangioma, Henoch-Schonlein purpura, hepatitis, hepatocellular and metastatic carcinomas, idiopathic thrombocytopenic purpura, porphyria, sarcoidosis, and Wilson disease. Response may be measured by comparing both the levels and sequences expressed in tissues from subjects exposed to or treated with steroid compounds such as mifepristone, progesterone, beclomethasone, medroxyprogesterone, budesonide, prednisone, dexamethasone, betamethasone, or danazol with the levels and sequences expressed in normal untreated tissue.

Steroids are a class of lipid-soluble molecules, including cholesterol, bile acids, vitamin D, and hormones, that share a common four-ring structure based on cyclopentanoperhydrophenanthrene and that carrry out a wide variety of functions. Cholesterol, for example, is a component of cell membranes that controls membrane fluidity. It is also a precursor for bile acids which solubilize lipids and facilitate absorption in the small intestine during digestion. Vitamin D regulates the absorption of calcium in the small intestine and controls the concentration of calcium in plasma. Steroid hormones, produced by the adrenal cortex, ovaries, and testes, include glucocorticoids, mineralocorticoids, androgens, and estrogens. They control various biological processes by binding to intracellular receptors that regulate transcription of specific genes in the nucleus. Glucocorticoids, for example, increase blood glucose concentrations by regulation of gluconeogenesis in the liver, increase blood concentrations of fatty acids by promoting lipolysis in adipose tissues, modulate sensitivity to catcholarrines in the central nervous system, and reduce inflammation. The principal mineralocorticoid, aldosterone, is produced by the adrenal cortex and acts on cells of the distal tubules of the kidney to enhance sodium ion reabsorption. Androgens, produced by the interstitial cells of Leydig in the testis, include the male sex hormone testosterone, which triggers changes at puberty, the production of sperm and maintenance of secondary sexual characteristics. Female sex hormones, estrogen and progesterone, are produced by the ovaries and also by the placenta and adrenal cortex of the fetus during pregnancy. Estrogen regulates female reproductive processes and secondary sexual characteristics. Progesterone regulates changes in the endometrium during the menstrual cycle and pregnancy.

Steroid hormones are widely used for fertility control and in anti-inflammatory treatments for physical injuries and diseases such as arthritis, asthma, and auto-immune disorders. Progesterone, a naturally occurring progestin, is primarily used to treat amenorrhea, abnormal uterine bleeding, or as a contraceptive. Endogenous progesterone is responsible for inducing secretory activity in the endometrium of the estrogen-primed uterus in preparation for the implantation of a fertilized egg and for the maintenance of pregnancy. It is secreted from the corpus luteum in response to luteinizing hormone (LH). The primary contraceptive effect of exogenous progestins involves the suppression of the midcycle surge of LH. At the cellular level, progestins diffuse freely into target cells and bind to the progesterone receptor. Target cells include the female reproductive tract, the mammary gland, the hypothalamus, and the pituitary. Once bound to the receptor, progestins slow the frequency of release of gonadotropin releasing hormone from the hypothalamus and blunt the pre-ovulatory LH surge, thereby preventing follicular maturation and ovulation. Progesterone has minimal estrogenic and androgenic activity. Progesterone is metabolized hepatically to pregnanediol and conjugated with glucuronic acid.

Medroxyprogesterone (MAH), also known as 6α-methyl-17-hydroxyprogesterone, is a synthetic progestin with a pharmacological activity about 15 times greater than progesterone. MAH is used for the treatment of renal and endometrial carcinomas, amenorrhea, abnormal uterine bleeding, and endometriosis associated with hormonal imbalance. MAH has a stimulatory effect on respiratory centers and has been used in cases of low blood oxygenation caused by sleep apnea, chronic obstructive pulmonary disease, or hypercapnia.

Mifepristone, also known as RU-486, is an antiprogesterone drug that blocks receptors of progesterone. It counteracts the effects of progesterone, which is needed to sustain pregnancy. Mifepristone induces spontaneous abortion when administered in early pregnancy followed by treatment with the prostaglandin, misoprostol. Further, studies show that mifepristone at a substantially lower dose can be highly effective as a postcoital contraceptive when administered within five days after unprotected intercourse, thus providing women with a “morning-after pill” in case of contraceptive failure or sexual assault. Mifepristone also has potential uses in the treatment of breast and ovarian cancers in cases in which tumors are progesterone-dependent. It interferes with steroid-dependent growth of brain meningiomas, and may be useful in treatment of endometriosis where it blocks the estrogen-dependent growth of endometrial tissues. It may also be useful in treatment of uterine fibroid tumors and Cushing's Syndrome. Mifepristone binds to glucocorticoid receptors and interferes with cortisol binding. Mifepristone also may act as an anti-glucocorticoid and be effective for treating conditions where cortisol levels are elevated such as AIDS, anorexia nervosa, ulcers, diabetes, Parkinson's disease, multiple sclerosis, and Alzheirner's disease.

Danazol is a synthetic steroid derived from ethinyl testosterone. Danazol indirectly reduces estrogen production by lowering pituitary synthesis of follicle-stimulating hormone and LH. Danazol also binds to sex hormone receptors in target tissues, thereby exhibiting anabolic, antiestrognic, and weakly androgenic activity. Danazol does not possess any progestogenic activity, and does not suppress normal pituitary release of corticotropin or release of cortisol by the adrenal glands. Danazol is used in the treatment of endometriosis to relieve pain and inhibit endometrial cell growth. It is also used to treat fibrocystic breast disease and hereditary angioedema.

Corticosteroids are used to relieve inflammation and to suppress the immune response. They inhibit eosinophil, basophil, and airway epithelial cell function by regulation of cytokines that mediate the inflammatory response. They inhibit leukocyte infiltration at the site of inflammation, interfere in the function of mediators of the inflammatory response, and suppress the humoral immune response. Corticosteroids are used to treat allergies, asthma, arthritis, and skin conditions. Beclomethasone is a synthetic glucocorticoid that is used to treat steroid-dependent asthma, to relieve symptoms associated with allergic or nonallergic (vasomotor) rhinitis, or to prevent recurrent nasal polyps following surgical removal. The anti-inflammatory and vasoconstrictive effects of intranasal beclomethasone are 5000 times greater than those produced by hydrocortisone. Budesonide is a corticosteroid used to control symptoms associated with allergic rhinitis or asthma. Budesonide has high topical anti-inflammatory activity but low systemic activity. Dexamethasone is a synthetic glucocorticoid used in anti-inflammatory or immunosuppressive compositions. It is also used in inhalants to prevent symptoms of asthma. Due to its greater ability to reach the central nervous system, dexamethasone is usually the treatment of choice to control cerebral edema. Dexamethasone is approximately 20-30 times more potent than hydrocortisone and 5-7 times more potent than prednisone. Prednisone is metabolized in the liver to its active form, prednisolone, a glucocorticoid with anti-inflammatory properties. Prednisone is approximately 4 times more potent than hydrocortisone and the duration of action of prednisone is intermediate between hydrocortisone and dexamethasone. Prednisone is used to treat allograft rejection, asthma, systemic lupus erythematosus, arthritis, ulcerative colitis, and other inflammatory conditions. Betamethasone is a synthetic glucocorticoid with antiinflammatory and immunosuppressive activity and is used to treat psoriasis and fungal infections, such as athlete's foot and ringworm.

The anti-inflammatory actions of corticosteroids are thought to involve phospholipase A₂ inhibitory proteins, collectively called lipocortins. Lipocortins, in turn, control the biosynthesis of potent mediators of inflammation such as prostaglandins and leukotrienes by inhibiting the release of the precursor molecule arachidonic acid. Proposed mechanisms of action include decreased IgE synthesis, increased number of β-adrenergic receptors on leukocytes, and decreased arachidonic acid metabolism. During an immediate allergic reaction, such as in chronic bronchial asthma, allergens bridge the IgE antibodies on the surface of mast cells, which triggers these cells to release chemotactic substances. Mast cell influx and activation, therefore, is partially responsible for the inflammation and hyperirritability of the oral mucosa in asthmatic patients. This inflammation can be retarded by administration of corticosteroids.

The effects upon liver metabolism and hormone clearance mechanisms are important to understand the pharmacodynamics of a drug. The human C3A cell line is a clonal derivative of HepG2/C3 (hepatoma cell line, isolated from a 15-year-old male with liver tumor), which was selected for strong contact inhibition of growth. The use of a clonal population enhances the reproducibility of the cells. C3A cells have many characteristics of primary human hepatocytes in culture: i) expression of insulin receptor and insulin-like growth factor II receptor; ii) secretion of a high ratio of serum albumin compared with α-fetoprotein iii) conversion of ammonia to urea and glutamine; iv) metabolize aromatic amino acids; and v) proliferate in glucose-free and insulin-free medium. The C3A cell line is now well established as an in vitro model of the mature human liver (Mickelson et al. (1995) Hepatology 22:866-875; Nagendra et al. (1997) Am J Physiol 272:G408-G416).

Breast Cancer

There are more than 180,000 new cases of breast cancer diagnosed each year, and the mortality rate for breast cancer approaches 10% of all deaths in females between the ages of 45-54 (K. Gish (1999) A WIS Magazine 28:7-10). However the survival rate based on early diagnosis of localized breast cancer is extremely high (97%), compared with the advanced stage of the disease in which the tumor has spread beyond the breast (22%). Current procedures for clinical breast examination are lacking in sensitivity and specificity, and efforts are underway to develop comprehensive gene expression profiles for breast cancer that may be used in conjunction with conventional screening methods to improve diagnosis and prognosis of this disease (Perou C M et al. (2000) Nature 406:747-752).

Breast cancer is a genetic disease commonly caused by mutations in cellular disease. Mutations in two genes, BRCA1 and BRCA2, are known to greatly predispose a woman to breast cancer and may be passed on from parents to children (Gish, supra). However, this type of hereditary breast cancer accounts for only about 5% to 9% of breast cancers, while the vast majority of breast cancer is due to noninherited mutations that occur in breast epithelial cells.

A good deal is already known about the expression of specific genes associated with breast cancer. For example, the relationship between expression of epidermal growth factor (EGF) and its receptor, EGFR, to human mammary carcinoma has been particularly well studied. (See Khazaie et al., supra, and references cited therein for a review of this area.) Overexpression of EGFR, particularly coupled with down-regulation of the estrogen receptor, is a marker of poor prognosis in breast cancer patients. In addition, EGFR expression in breast tumor metastases is frequently elevated relative to the primary tumor, suggesting that EGFR is involved in tumor progression and metastasis. This is supported by accumulating evidence that EGF has effects on cell functions related to metastatic potential, such as cell motility, chemotaxis, secretion and differentiation. Changes in expression of other members of the erbB receptor family, of which EGFR is one, have also been implicated in breast cancer. The abundance of erbB receptors, such as HER-2/neu, HER-3, and HER-4, and their ligands in breast cancer points to their functional importance in the pathogenesis of the disease, and may therefore provide targets for therapy of the disease (Bacus, SS et al. (1994) Am J Clin Pathol 102:S 13-S24). Other known markers of breast cancer include a human secreted frizzled protein mRNA that is downregulated in breast tumors; the matrix G1a protein which is overexpressed is human breast carcinoma cells; Drg1 or RTP, a gene whose expression is diminished in colon, breast, and prostate tumors; maspin, a tumor suppressor gene downregulated in invasive breast carcinomas; and CaN19, a member of the S100 protein family, all of which are down regulated in mammary carcinoma cells relative to normal mammary epithelial cells (Zhou Z et al. (1998) Int J Cancer 78:95-99; Chen, L et al. (1990) Oncogene 5:1391-1395; Ulrix W et al (1999) FEBS Lett 455:23-26; Sager, R et al. (1996) Curr Top Microbiol Immunol 213:51-64; and Lee, SW et al. (1992) Proc Natl Acad Sci USA 89:2504-2508).

Cell lines derived from human mammary epithelial cells at various stages of breast cancer provide a useful model to study the process of malignant transformation and tumor progression as it has been shown that these cell lines retain many of the properties of their parental tumors for lengthy culture periods (Wistuba II et al. (1998) Clin Cancer Res 4:2931-2938). Such a model is particularly useful for comparing phenotypic and molecular characteristics of human mammary epithelial cells at various stages of malignant transformation.

Prostate Cancer

Prostate cancer is a common malignancy in men over the age of 50, and the incidence increases with age. In the US, there are approximately 132,000 newly diagnosed cases of prostate cancer and more than 33,000 deaths from the disorder each year.

Once cancer cells arise in the prostate, they are stimulated by testosterone to a more rapid growth. Thus, removal of the testes can indirectly reduce both rapid growth and metastasis of the cancer. Over 95 percent of prostatic cancers are adenocarcinomas which originate in the prostatic acini. The remaining 5 percent are divided between squamous cell and transitional cell carcinomas, both of which arise in the prostatic ducts or other parts of the prostate gland.

As with most cancers, prostate cancer develops through a multistage progression ultimately resulting in an aggressive, metastatic phenotype. The initial step in tumor progression involves the hyperproliferation of normal luminal and/or basal epithelial cells that become hyperplastic and evolve into early-stage tumors. The early-stage tumors are localized in the prostate but eventually may metastasize, particularly to the bone, brain or lung. About 80% of these tumors remain responsive to androgen treatment, an important hormone controlling the growth of prostate epithelial cells. However, in its most advanced state, cancer growth becomes androgen-independent and there is currently no known treatment for this condition.

A primary diagnostic marker for prostate cancer is prostate specific antigen (PSA). PSA is a tissue-specific serine protease almost exclusively produced by prostatic epithelial cells. The quantity of PSA correlates with the number and volume of the prostatic epithelial cells, and consequently, the levels of PSA are an excellent indicator of abnormal prostate growth. Men with prostate cancer exhibit an early linear increase in PSA levels followed by an exponential increase prior to diagnosis. However, since PSA levels are also influenced by factors such as inflammation, androgen and other growth factors, some scientists maintain that changes in PSA levels are not useful in detecting individual cases of prostate cancer.

Current areas of cancer research provide additional prospects for markers as well as potential therapeutic targets for prostate cancer. Several growth factors have been shown to play a critical role in tumor development, growth, and progression. The growth factors Epidermal Growth Factor (EGF), Fibroblast Growth Factor (FGF), and Tumor Growth Factor alpha (TGFα) are important in the growth of normal as well as hyperproliferative prostate epithelial cells, particularly at early stages of tumor development and progression, and affect signaling pathways in these cells in various ways (Lin J et al. (1999) Cancer Res. 59:2891-2897; Putz T et al. (1999) Cancer Res 59:227-233). The TGF-β family of growth factors are generally expressed at increased levels in human cancers and the high expression levels in many cases correlates with advanced stages of malignancy and poor survival (Gold L1 (1999) Crit Rev Oncog 10:303-360). Finally, there are human cell lines representing both the androgen-dependent stage of prostate cancer (LNCap) as well as the androgen-independent, hormone refractory stage of the disease (PC3 and DU-145) that have proved useful in studying gene expression patterns associated with the progression of prostate cancer, and the effects of cell treatments on these expressed genes (Chung T D (1999) Prostate 15:199-207).

Tangier Disease

Tangier disease (TD) is a rare genetic disorder characterized by near absence of circulating high density lipoprotein (HDL) and the accumulation of cholesterol esters in many tissues, including tonsils, lymph nodes, liver, spleen, thymus, and intestine. Low levels of HDL represent a clear predictor of premature coronary artery disease and homozygous TD correlates with a four- to six-fold increase in cardiovascular disease compared to controls. The major cardio-protective activity of HDL is ascribed to its role in reverse cholesterol transport, the flux of cholesterol from peripheral cells such as tissue macrophages, through plasma lipoproteins to the liver. The HDL protein, apolipoprotein A-I, plays a major role in this process, interacting with the cell surface to remove excess cholesterol and phospholipids. Recent studies have shown that this pathway is severely impaired in TD and the defect lies in a specific gene, the ABC1 transporter. This gene is a member of the family of ATP-binding cassette transporters, which utilize ATP hydrolysis to transport a variety of substrates across membranes.

Adipocyte Differentiation

The primary function of adipose tissue is the ability to store and release fat during periods of feeding and fasting. White adipose tissue is the major energy reserve in periods of fasting, and its reserve is mobilized during energy deprivation. Adipose tissue is one of the primary target tissues for insulin, and adipogenesis and insulin resistance are linked in type II diabetes, non-insulin dependent diabetes mellitus (NIDDM). Cytologically the conversion of a preadipocytes into mature adipocytes is characterized by deposition of fat droplets around the nuclei. The conversion process in vivo can be induced by thiazolidinediones (TZDs) and other PPARγ agonists (Adams et al. (1997) J. Clin. Invest. 100:3149-3153) which also lead to increased sensitivity to insulin and reduced plasma glucose and blood pressure.

Thiazolidinediones (TZDs) act as agonists for the peroxisome-proliferator-activated receptor gamma (PPARγ), a member of the nuclear hormone receptor superfamily. TZDs reduce hyperglycemia, hyperinsulinemia, and hypertension, in part by promoting glucose metabolism and inhibiting gluconeogenesis. Roles for PPARγ and its agonists have been demonstrated in a wide range of pathological conditions including diabetes, obesity, hypertension, atherosclerosis, polycystic ovarian syndrome, and cancers such as breast, prostate, liposarcoma, and colon cancer.

The mechanism by which TZDs and other PPARγ agonists enhance insulin sensitivity is not fully understood, but may involve the ability of PPARγ to promote adipogenesis. When ectopically expressed in cultured preadipocytes, PPARγ is a potent inducer of adipocyte differentiation. TZDs, in combination with insulin and other factors, can also enhance differentiation of human preadipocytes in culture (Adams et al. (1997) J. Clin. Invest. 100:3149-3153). The relative potency of different TZDs in promoting adipogenesis in vitro is proportional to both their insulin sensitizing effects in vivo, and their ability to bind and activate PPARγ in vitro. Interestingly, adipocytes derived from omental adipose depots are refractory to the effects of TZDs. It has therefore been suggested that the insulin sensitizing effects of TZDs may result from their ability to promote adipogenesis in subcutaneous adipose depots (Adams et al., ibid). Further, dominant negative mutations in the PPARγ gene have been identified in two non-obese subjects with severe insulin resistance, hypertension, and overt non-insulin dependent diabetes mellitus (NIDDM) (Barroso et al. (1998) Nature 402:880-883).

NIDDM is the most common form of diabetes mellitus, a chronic metabolic disease that affects 143 million people worldwide. NIDDM is characterized by abnormal glucose and lipid metabolism that result from a combination of peripheral insulin resistance and defective insulin secretion. NIDDM has a complex, progressive etiology and a high degree of heritability. Numerous complications of diabetes including heart disease, stroke, renal failure, retinopathy, and peripheral neuropathy contribute to the high rate of morbidity and mortality.

At the molecular level, PPARγ functions as a ligand activated transcription factor. In the presence of ligand, PPARγ forms a heterodimer with the retinoid X receptor (RXR) which then activates transcription of target genes containing one or more copies of a PPARγ response element (PPRE). Many genes important in lipid storage and metabolism contain PPREs and have been identified as PPARγ targets, including PEPCK, aP2, LPL, ACS, and FAT-P (Auwerx, J. (1999) Diabetologia 42:1033-1049). Multiple ligands for PPARγ have been identified. These include a variety of fatty acid metabolites; synthetic drugs belonging to the TZD class, such as Pioglitazone and Rosiglitazone (BRL49653); and certain non-glitazone tyrosine analogs such as GI262570 and GW1929. The prostaglandin derivative 15-dPGJ2 is a potent endogenous ligand for PPARγ.

Expression of PPARγ is very high in adipose but barely detectable in skeletal muscle, the primary site for insulin stimulated glucose disposal in the body. PPARγ is also moderately expressed in large intestine, kidney, liver, vascular smooth muscle, hematopoietic cells, and macrophages. The high expression of PPARγ in adipose suggests that the insulin sensitizing effects of TZDs may result from alterations in the expression of one or more PPARγ regulated genes in adipose tissue. Identification of PPARγ target genes will contribute to better drug design and the development of novel therapeutic strategies for diabetes, obesity, and other conditions.

Systematic attempts to identify PPARγ target genes have been made in several rodent models of obesity and diabetes (Suzuki et al. (2000) Jpn. J. Pharmacol. 84:113-123; Way et al. (2001) Endocrinology 142:1269-1277). However, a serious drawback of the rodent gene expression studies is that significant differences exist between human and rodent models of adipogenesis, diabetes, and obesity (Taylor (1999) Cell 97:9-12; Gregoire et al. (1998) Physiol. Reviews 78:783-809). Therefore, an unbiased approach to identifying TZD regulated genes in primary cultures of human tissues is necessary to fully elucidate the molecular basis for diseases associated with PPARγ activity.

The majority of research in adipocyte biology to date has been done using transformed mouse preadipocyte cell lines. The culture condition, which stimulates mouse preadipocyte differentiation is different from that for inducing human primary preadipocyte differentiation. In addition, primary cells are diploid and may therefore reflect the in vivo context better than aneuploid cell lines. Understanding the gene expression profile during adipogenesis in human will lead to understanding the fundamental mechanism of adiposity regulation. Furthermore, through comparing the gene expression profiles of adipogenesis between donor with normal weight and donor with obesity, identification of crucial genes, potential drug targets for obesity and type II diabetes, will be possible.

There is a need in the art for new compositions, including nucleic acids and proteins, for the diagnosis, prevention, and treatment of cell proliferative, autoimmune/inflammatory, neurological, metabolic, developmental, and endocrine disorders.

### SUMMARY OF THE INVENTION

Various embodiments of the invention provide purified polypeptides, receptors and membrane-associated proteins, referred to collectively as ‘REMAP’ and individually as ‘REMAP-1,’ ‘REMAP-2,’ ‘REMAP-3,’ ‘REMAP-4,’ ‘REMAP-5,’ ‘REMAP-6,’ ‘REMAP-7,’ ‘REMAP-8,’ ‘REMAP-9,’ ‘REMAP-10,’ ‘REMAP-11,’ ‘REMAP-12,’ ‘REMAP-13,’ ‘REMAP-14,’ ‘REMAP-15,’ ‘REMAP-16,’ ‘REMAP-17,’ ‘REMAP-18,’ ‘REMAP-19,’ ‘REMAP-20,’ ‘REMAP-21,’ ‘REMAP-22,’ ‘REMAP-23,’ ‘REMAP-24,’ ‘REMAP-25,’ ‘REMAP-26,’ ‘REMAP-27,’ ‘REMAP-28,’ ‘REMAP-29,’ ‘REMAP-30,’ ‘REMAP-31,’ ‘REMAP-32,’ ‘REMAP-33,’ ‘REMAP-34,’ ‘REMAP-35,’ ‘REMAP-36,’ ‘REMAP-37,’ ‘REMAP-38,’ ‘REMAP-39,’ ‘REMAP-40,’ ‘REMAP-41,’ ‘REMAP-42,’ ‘REMAP-43,’ ‘REMAP-44,’ ‘REMAP-45,’ ‘REMAP-46,’ and ‘REMAP-47’ and methods for using these proteins and their encoding polynucleotides for the detection, diagnosis, and treatment of diseases and medical conditions. Embodiments also provide methods for utilizing the purified receptors and membrane-associated proteins and/or their encoding polynucleotides for facilitating the drug discovery process, including determination of efficacy, dosage, toxicity, and pharmacology. Related embodiments provide methods for utilizing the purified receptors and membrane-associated proteins and/or their encoding polynucleotides for investigating the pathogenesis of diseases and medical conditions.

An embodiment provides an isolated polypeptide selected from the group consisting of a) a polypeptide comprising an amino acid sequence selected from the group consisting of SEQ ID NO:1-47, b) a polypeptide comprising a naturally occurring amino acid sequence at least 90% identical or at least about 90% identical to an amino acid sequence selected from the group consisting of SEQ ID NO:1-47, c) a biologically active fragment of a polypeptide having an amino acid sequence selected from the group consisting of SEQ ID NO:1-47, and d) an immunogenic fragment of a polypeptide having an amino acid sequence selected from the group consisting of SEQ ID NO:1-47. Another embodiment provides an isolated polypeptide comprising an amino acid sequence of SEQ ID NO:1-47.

Still another embodiment provides an isolated polynucleotide encoding a polypeptide selected from the group consisting of a) a polypeptide comprising an amino acid sequence selected from the group consisting of SEQ ID NO:1-47, b) a polypeptide comprising a naturally occurring amino acid sequence at least 90% identical or at least about 90% identical to an amino acid sequence selected from the group consisting of SEQ ID NO:1-47, c) a biologically active fragment of a polypeptide having an amino acid sequence selected from the group consisting of SEQ ID NO:1-47, and d) an immunogenic fragment of a polypeptide having an amino acid sequence selected from the group consisting of SEQ ID NO:1-47. In another embodiment, the polynucleotide encodes a polypeptide selected from the group consisting of SEQ ID NO:1-47. In an alternative embodiment, the polynucleotide is selected from the group consisting of SEQ ID NO:48-94.

Still another embodiment provides a recombinant polynucleotide comprising a promoter sequence operably linked to a polynucleotide encoding a polypeptide selected from the group consisting of a) a polypeptide comprising an amino acid sequence selected from the group consisting of SEQ ID NO:1-47, b) a polypeptide comprising a naturally occurring amino acid sequence at least 90% identical or at least about 90% identical to an amino acid sequence selected from the group consisting of SEQ ID NO:1-47, c) a biologically active fragment of a polypeptide having an amino acid sequence selected from the group consisting of SEQ ID NO:1-47, and d) an immunogenic fragment of a polypeptide having an amino acid sequence selected from the group consisting of SEQ ID NO:1-47. Another embodiment provides a cell transformed with the recombinant polynucleotide. Yet another embodiment provides a transgenic organism comprising the recombinant polynucleotide.

Another embodiment provides a method for producing a polypeptide selected from the group consisting of a) a polypeptide comprising an amino acid sequence selected from the group consisting of SEQ ID NO:1-47, b) a polypeptide comprising a naturally occurring amino acid sequence at least 90% identical or at least about 90% identical to an amino acid sequence selected from the group consisting of SEQ ID NO:1-47, c) a biologically active fragment of a polypeptide having an amino acid sequence selected from the group consisting of SEQ ID NO:1-47, and d) an immunogenic fragment of a polypeptide having an amino acid sequence selected from the group consisting of SEQ ID NO:1-47. The method comprises a) culturing a cell under conditions suitable for expression of the polypeptide, wherein said cell is transformed with a recombinant polynucleotide comprising a promoter sequence operably linked to a polynucleotide encoding the polypeptide, and b) recovering the polypeptide so expressed.

Yet another embodiment provides an isolated antibody which specifically binds to a polypeptide selected from the group consisting of a) a polypeptide comprising an amino acid sequence selected from the group consisting of SEQ ID NO:1-47, b) a polypeptide comprising a naturally occurring amino acid sequence at least 90% identical or at least about 90% identical to an amino acid sequence selected from the group consisting of SEQ ID NO:1-47, c) a biologically active fragment of a polypeptide having an amino acid sequence selected from the group consisting of SEQ ID NO:1-47, and d) an immunogenic fragment of a polypeptide having an amino acid sequence selected from the group consisting of SEQ ID NO:1-47.

Still yet another embodiment provides an isolated polynucleotide selected from the group consisting of a) a polynucleotide comprising a polynucleotide sequence selected from the group consisting of SEQ ID NO:48-94, b) a polynucleotide comprising a naturally occurring polynucleotide sequence at least 90% identical or at least about 90% identical to a polynucleotide sequence selected from the group consisting of SEQ ID NO:48-94, c) a polynucleotide complementary to the polynucleotide of a), d) a polynucleotide complementary to the polynucleotide of b), and e) an RNA equivalent of a)-d). In other embodiments, the polynucleotide can comprise at least about 20, 30, 40, 60, 80, or 100 contiguous nucleotides.

Yet another embodiment provides a method for detecting a target polynucleotide in a sample, said target polynucleotide being selected from the group consisting of a) a polynucleotide comprising a polynucleotide sequence selected from the group consisting of SEQ ID NO:48-94, b) a polynucleotide comprising a naturally occurring polynucleotide sequence at least 90% identical or at least about 90% identical to a polynucleotide sequence selected from the group consisting of SEQ ID NO:48-94, c) a polynucleotide complementary to the polynucleotide of a), d) a polynucleotide complementary to the polynucleotide of b), and e) an RNA equivalent of a)-d). The method comprises a) hybridizing the sample with a probe comprising at least 20 contiguous nucleotides comprising a sequence complementary to said target polynucleotide in the sample, and which probe specifically hybridizes to said target polynucleotide, under conditions whereby a hybridization complex is formed between said probe and said target polynucleotide or fragments thereof, and b) detecting the presence or absence of said hybridization complex. In a related embodiment, the method can include detecting the amount of the hybridization complex. In still other embodiments, the probe can comprise at least about 20, 30, 40, 60, 80, or 100 contiguous nucleotides.

Still yet another embodiment provides a method for detecting a target polynucleotide in a sample, said target polynucleotide being selected from the group consisting of a) a polynucleotide comprising a polynucleotide sequence selected from the group consisting of SEQ ID NO:48-94, b) a polynucleotide comprising a naturally occurring polynucleotide sequence at least 90% identical or at least about 90% identical to a polynucleotide sequence selected from the group consisting of SEQ ID NO:48-94, c) a polynucleotide complementary to the polynucleotide of a), d) a polynucleotide complementary to the polynucleotide of b), and e) an RNA equivalent of a)-d). The method comprises a) amplifying said target polynucleotide or fragment thereof using polymerase chain reaction amplification, and b) detecting the presence or absence of said amplified target polynucleotide or fragment thereof. In a related embodiment, the method can include detecting the amount of the amplified target polynucleotide or fragment thereof.

Another embodiment provides a composition comprising an effective amount of a polypeptide selected from the group consisting of a) a polypeptide comprising an amino acid sequence selected from the group consisting of SEQ ID NO:1-47, b) a polypeptide comprising a naturally occurring amino acid sequence at least 90% identical or at least about 90% identical to an amino acid sequence selected from the group consisting of SEQ ID NO:147, c) a biologically active fragment of a polypeptide having an amino acid sequence selected from the group consisting of SEQ ID NO:1-47, and d) an immunogenic fragment of a polypeptide having an amino acid sequence selected from the group consisting of SEQ ID NO:1-47, and a pharmaceutically acceptable excipient. In one embodiment, the composition can comprise an amino acid sequence selected from the group consisting of SEQ ID NO:1-47. Other embodiments provide a method of treating a disease or condition associated with decreased or abnormal expression of functional REMAP, comprising administering to a patient in need of such treatment the composition.

Yet another embodiment provides a method for screening a compound for effectiveness as an agonist of a polypeptide selected from the group consisting of a) a polypeptide comprising an amino acid sequence selected from the group consisting of SEQ ID NO:1-47, b) a polypeptide comprising a naturally occurring amino acid sequence at least 90% identical or at least about 90% identical to an amino acid sequence selected from the group consisting of SEQ ID NO:1-47, c) a biologically active fragment of a polypeptide having an amino acid sequence selected from the group consisting of SEQ ID NO:1-47, and d) an immunogenic fragment of a polypeptide having an amino acid sequence selected from the group consisting of SEQ ID NO:1-47. The method comprises a) exposing a sample comprising the polypeptide to a compound, and b) detecting agonist activity in the sample. Another embodiment provides a composition comprising an agonist compound identified by the method and a pharmaceutically acceptable excipient. Yet another embodiment provides a method of treating a disease or condition associated with decreased expression of functional REMAP, comprising administering to a patient in need of such treatment the composition.

Still yet another embodiment provides a method for screening a compound for effectiveness as an antagonist of a polypeptide selected from the group consisting of a) a polypeptide comprising an amino acid sequence selected from the group consisting of SEQ ID NO:1-47, b) a polypeptide comprising a naturally occurring amino acid sequence at least 90% identical or at least about 90% identical to an amino acid sequence selected from the group consisting of SEQ ID NO:1-47, c) a biologically active fragment of a polypeptide having an amino acid sequence selected from the group consisting of SEQ ID NO:1-47, and d) an immunogenic fragment of a polypeptide having an amino acid sequence selected from the group consisting of SEQ ID NO:1-47. The method comprises a) exposing a sample comprising the polypeptide to a compound, and b) detecting antagonist activity in the sample. Another embodiment provides a composition comprising an antagonist compound identified by the method and a pharmaceutically acceptable excipient. Yet another embodiment provides a method of treating a disease or condition associated with overexpression of functional REMAP, comprising administering to a patient in need of such treatment the composition.

Another embodiment provides a method of screening for a compound that specifically binds to a polypeptide selected from the group consisting of a) a polypeptide comprising an amino acid sequence selected from the group consisting of SEQ ID NO:1-47, b) a polypeptide comprising a naturally occurring amino acid sequence at least 90% identical or at least about 90% identical to an amino acid sequence selected from the group consisting of SEQ ID NO:147, c) a biologically active fragment of a polypeptide having an amino acid sequence selected from the group consisting of SEQ ID NO:1-47, and d) an immunogenic fragment of a polypeptide having an amino acid sequence selected from the group consisting of SEQ ID NO:1-47. The method comprises a) combining the polypeptide with at least one test compound under suitable conditions, and b) detecting binding of the polypeptide to the test compound, thereby identifying a compound that specifically binds to the polypeptide.

Yet another embodiment provides a method of screening for a compound that modulates the activity of a polypeptide selected from the group consisting of a) a polypeptide comprising an amino acid sequence selected from the group consisting of SEQ ID NO:1-47, b) a polypeptide comprising a naturally occurring amino acid sequence at least 90% identical or at least about 90% identical to an amino acid sequence selected from the group consisting of SEQ ID NO:1-47, c) a biologically active fragment of a polypeptide having an amino acid sequence selected from the group consisting of SEQ ID NO:1-47, and d) an immunogenic fragment of a polypeptide having an amino acid sequence selected from the group consisting of SEQ ID NO:1-47. The method comprises a) combining the polypeptide with at least one test compound under conditions permissive for the activity of the polypeptide, b) assessing the activity of the polypeptide in the presence of the test compound, and c) comparing the activity of the polypeptide in the presence of the test compound with the activity of the polypeptide in the absence of the test compound, wherein a change in the activity of the polypeptide in the presence of the test compound is indicative of a compound that modulates the activity of the polypeptide.

Still yet another embodiment provides a method for screening a compound for effectiveness in altering expression of a target polynucleotide, wherein said target polynucleotide comprises a polynucleotide sequence selected from the group consisting of SEQ ID NO:48-94, the method comprising a) exposing a sample comprising the target polynucleotide to a compound, b) detecting altered expression of the target polynucleotide, and c) comparing the expression of the target polynucleotide in the presence of varying amounts of the compound and in the absence of the compound.

Another embodiment provides a method for assessing toxicity of a test compound, said method comprising a) treating a biological sample containing nucleic acids with the test compound; b) hybridizing the nucleic acids of the treated biological sample with a probe comprising at least 20 contiguous nucleotides of a polynucleotide selected from the group consisting of i) a polynucleotide comprising a polynucleotide sequence selected from the group consisting of SEQ ID NO:48-94, ii) a polynucleotide comprising a naturally occurring polynucleotide sequence at least 90% identical or at least about 90% identical to a polynucleotide sequence selected from the group consisting of SEQ ID NO:48-94, iii) a polynucleotide having a sequence complementary to i), iv) a polynucleotide complementary to the polynucleotide of ii), and v) an RNA equivalent of i)-iv). Hybridization occurs under conditions whereby a specific hybridization complex is formed between said probe and a target polynucleotide in the biological sample, said target polynucleotide selected from the group consisting of i) a polynucleotide comprising a polynucleotide sequence selected from the group consisting of SEQ ID NO:48-94, ii) a polynucleotide comprising a naturally occurring polynucleotide sequence at least 90% identical or at least about 90% identical to a polynucleotide sequence selected from the group consisting of SEQ ID NO:48-94, iii) a polynucleotide complementary to the polynucleotide of i), iv) a polynucleotide complementary to the polynucleotide of ii), and v) an RNA equivalent of i)-iv). Alternatively, the target polynucleotide can comprise a fragment of a polynucleotide selected from the group consisting of i)-v) above; c) quantifying the amount of hybridization complex; and d) comparing the amount of hybridization complex in the treated biological sample with the amount of hybridization complex in an untreated biological sample, wherein a difference in the amount of hybridization complex in the treated biological sample is indicative of toxicity of the test compound.

### BRIEF DESCRIPTION OF THE TABLES

Table 1 summarizes the nomenclature for full length polynucleotide and polypeptide embodiments of the invention.

Table 2 shows the GenBank identification number and annotation of the nearest GenBank homolog, and the PROTEOME database identification numbers and annotations of PROTEOME database homologs, for polypeptide embodiments of the invention. The probability scores for the matches between each polypeptide and its homolog(s) are also shown.

Table 3 shows structural features of polypeptide embodiments, including predicted motifs and domains, along with the methods, algorithms, and searchable databases used for analysis of the polypeptides.

Table 4 lists the cDNA and/or genomic DNA fragments which were used to assemble polynucleotide embodiments, along with selected fragments of the polynucleotides.

Table 5 shows representative cDNA libraries for polynucleotide embodiments.

Table 6 provides an appendix which describes the tissues and vectors used for construction of the cDNA libraries shown in Table 5.

Table 7 shows the tools, programs, and algorithms used to analyze polynucleotides and polypeptides, along with applicable descriptions, references, and threshold parameters.

Table 8 shows single nucleotide polymorphisms found in polynucleotide sequences of the invention, along with allele frequencies in different human populations.

### DESCRIPTION OF THE INVENTION

Before the present proteins, nucleic acids, and methods are described, it is understood that embodiments of the invention are not limited to the particular machines, instruments, materials, and methods described, as these may vary. It is also to be understood that the terminology used herein is for the purpose of describing particular embodiments only, and is not intended to limit the scope of the invention.

As used herein and in the appended claims, the singular forms “a,” “an,” and “the” include plural reference unless the context clearly dictates otherwise. Thus, for example, a reference to “a host cell” includes a plurality of such host cells, and a reference to “an antibody” is a reference to one or more antibodies and equivalents thereof known to those skilled in the art, and so forth.

Unless defined otherwise, all technical and scientific terms used herein have the same meanings as commonly understood by one of ordinary skill in the art to which this invention belongs. Although any machines, materials, and methods similar or equivalent to those described herein can be used to practice or test the present invention, the preferred machines, materials and methods are now described. All publications mentioned herein are cited for the purpose of describing and disclosing the cell lines, protocols, reagents and vectors which are reported in the publications and which might be used in connection with various embodiments of the invention. Nothing herein is to be construed as an admission that the invention is not entitled to antedate such disclosure by virtue of prior invention.

DEFINITIONS

“REMAP” refers to the amino acid sequences of substantially purified REMAP obtained from any species, particularly a mammalian species, including bovine, ovine, porcine, murine, equine, and human, and from any source, whether natural, synthetic, semi-synthetic, or recombinant.

The term “agonist” refers to a molecule which intensifies or mimics the biological activity of REMAP. Agonists may include proteins, nucleic acids, carbohydrates, small molecules, or any other compound or composition which modulates the activity of REMAP either by directly interacting with REMAP or by acting on components of the biological pathway in which REMAP participates.

An “allelic variant” is an alternative form of the gene encoding REMAP. Allelic variants may result from at least one mutation in the nucleic acid sequence and may result in altered mRNAs or in polypeptides whose structure or function may or may not be altered. A gene may have none, one, or many allelic variants of its naturally occurring form. Common mutational changes which give rise to allelic variants are generally ascribed to natural deletions, additions, or substitutions of nucleotides. Each of these types of changes may occur alone, or in combination with the others, one or more times in a given sequence.

“Altered” nucleic acid sequences encoding REMAP include those sequences with deletions, insertions, or substitutions of different nucleotides, resulting in a polypeptide the same as REMAP or a polypeptide with at least one functional characteristic of REMAP. Included within this definition are polymorphisms which may or may not be readily detectable using a particular oligonucleotide probe of the polynucleotide encoding REMAP, and improper or unexpected hybridization to allelic variants, with a locus other than the normal chromosomal locus for the polynucleotide encoding REMAP. The encoded protein may also be “altered,” and may contain deletions, insertions, or substitutions of amino acid residues which produce a silent change and result in a functionally equivalent REMAP. Deliberate amino acid substitutions may be made on the basis of one or more similarities in polarity, charge, solubility, hydrophobicity, hydrophilicity, and/or the amphipathic nature of the residues, as long as the biological or immunological activity of REMAP is retained. For example, negatively charged amino acids may include aspartic acid and glutamic acid, and positively charged amino acids may include lysine and arginine. Amino acids with uncharged polar side chains having similar hydrophilicity values may include: asparagine and glutamine; and serine and threonine. Amino acids with uncharged side chains having similar hydrophilicity values may include: leucine, isoleucine, and valine; glycine and alanine; and phenylalanine and tyrosine.

The terms “ammo acid” and “amino acid sequence” can refer to an oligopeptide, a peptide, a polypeptide, or a protein sequence, or a fragment of any of these, and to naturally occurring or synthetic molecules. Where “amino acid sequence” is recited to refer to a sequence of a naturally occurring protein molecule, “amino acid sequence” and like terms are not meant to limit the amino acid sequence to the complete native amino acid sequence associated with the recited protein molecule.

“Amplification” relates to the production of additional copies of a nucleic acid. Amplification may be carried out using polymerase chain reaction (PCR) technologies or other nucleic acid amplification technologies well known in the art.

The term “antagonist” refers to a molecule which inhibits or attenuates the biological activity of REMAP. Antagonists may include proteins such as antibodies, anticalins, nucleic acids, carbohydrates, small molecules, or any other compound or composition which modulates the activity of REMAP either by directly interacting with REMAP or by acting on components of the biological pathway in which REMAP participates.

The term “antibody” refers to intact immunoglobulin molecules as well as to fragments thereof, such as Fab, F(ab′)₂, and Fv fragments, which are capable of binding an epitopic determinant. Antibodies that bind REMAP polypeptides can be prepared using intact polypeptides or using fragments containing small peptides of interest as the immunizing antigen. The polypeptide or oligopeptide used to immunize an animal (e.g., a mouse, a rat, or a rabbit) can be derived from the translation of RNA, or synthesized chemically, and can be conjugated to a carrier protein if desired. Commonly used carriers that are chemically coupled to peptides include bovine serum albumin, thyroglobulin, and keyhole limpet hemocyanin (KLH). The coupled peptide is then used to immunize the animal.

The term “antigenic determinant” refers to that region of a molecule (i.e., an epitope) that makes contact with a particular antibody. When a protein or a fragment of a protein is used to immunize a host animal, numerous regions of the protein may induce the production of antibodies which bind specifically to antigenic determinants (particular regions or three-dimensional structures on the protein). An antigenic determinant may compete with the intact antigen (i.e., the immunogen used to elicit the immune response) for binding to an antibody.

The term “aptamer” refers to a nucleic acid or oligonucleotide molecule that binds to a specific molecular target. Aptamers are derived from an in vitro evolutionary process (e.g., SELEX (Systematic Evolution of Ligands by EXponential Enrichment), described in U.S. Pat. No. 5,270,163), which selects for target-specific aptamer sequences from large combinatorial libraries. Aptamer compositions may be double-stranded or single-stranded, and may include deoxyribonucleotides, ribonucleotides, nucleotide derivatives, or other nucleotide-like molecules. The nucleotide components of an aptamer may have modified sugar groups (e.g., the 2′-OH group of a ribonucleotide may be replaced by 2′-F or 2′-NH₂), which may improve a desired property, e.g., resistance to nucleases or longer lifetime in blood. Aptamers may be conjugated to other molecules, e.g., a high molecular weight carrier to slow clearance of the aptamer from the circulatory system. Aptamers may be specifically cross-linked to their cognate ligands, e.g., by photo-activation of a cross-linker (Brody, E. N. and L. Gold (2000) J. Biotechnol. 74:5-13).

The term “intramer” refers to an aptamer which is expressed in vivo. For example, a vaccinia virus-based RNA expression system has been used to express specific RNA aptamers at high levels in the cytoplasm of leukocytes (Blind, M. et al. (1999) Proc. Natl. Acad. Sci. USA 96:3606-3610).

The term “spiegelmer” refers to an aptamer which includes L-DNA, L-RNA, or other left-handed nucleotide derivatives or nucleotide-like molecules. Aptamers containing left-handed nucleotides are resistant to degradation by naturally occurring enzymes, which normally act on substrates containing right-handed nucleotides.

The term “antisense” refers to any composition capable of base-pairing with the “sense” (coding) strand of a polynucleotide having a specific nucleic acid sequence. Antisense compositions may include DNA; RNA; peptide nucleic acid (PNA); oligonucleotides having modified backbone linkages such as phosphorothioates, methylphosphonates, or benzylphosphonates; oligonucleotides having modified sugar groups such as 2′-methoxyethyl sugars or 2′-methoxyethoxy sugars; or oligonucleotides having modified bases such as 5-methyl cytosine, 2′-deoxyuracil, or 7-deaza-2′-deoxyguanosine. Antisense molecules may be produced by any method including chemical synthesis or transcription. Once introduced into a cell, the complementary antisense molecule base-pairs with a naturally occurring nucleic acid sequence produced by the cell to form duplexes which block either transcription or translation. The designation “negative” or “minus” can refer to the antisense strand, and the designation “positive” or “plus” can refer to the sense strand of a reference DNA molecule.

The term “biologically active” refers to a protein having structural, regulatory, or biochemical functions of a naturally occurring molecule. Likewise, “immunologically active” or “immunogenic” refers to the capability of the natural, recombinant, or synthetic REMAP, or of any oligopeptide thereof, to induce a specific immune response in appropriate animals or cells and to bind with specific antibodies.

“Complementary” describes the relationship between two single-stranded nucleic acid sequences that anneal by base-pairing. For example, 5′-AGT-3′ pairs with its complement, 3′-TCA-5′.

A “composition comprising a given polynucleotide” and a “composition comprising a given polypeptide” can refer to any composition containing the given polynucleotide or polypeptide. The composition may comprise a dry formulation or an aqueous solution. Compositions comprising polynucleotides encoding REMAP or fragments of REMAP may be employed as hybridization probes. The probes may be stored in freeze dried form and may be associated with a stabilizing agent such as a carbohydrate. In hybridizations, the probe may be deployed in an aqueous solution containing salts (e.g., NaCl), detergents (e.g., sodium dodecyl sulfate; SDS), and other components (e.g., Denhardt's solution, dry milk, salmon sperm DNA, etc.).

“Consensus sequence” refers to a nucleic acid sequence which has been subjected to repeated DNA sequence analysis to resolve uncalled bases, extended using the XL-PCR kit (Applied Biosystems, Foster City Calif.) in the 5′ and/or the 3′ direction, and resequenced, or which has been assembled from one or more overlapping cDNA, EST, or genomic DNA fragments using a computer program for fragment assembly, such as the GELVIEW fragment assembly system (Accelrys, Burlington Mass.) or Phrap (University of Washington, Seattle Wash.). Some sequences have been both extended and assembled to produce the consensus sequence.

“Conservative amino acid substitutions” are those substitutions that are predicted to least interfere with the properties of the original protein, i.e., the structure and especially the function of the protein is conserved and not significantly changed by such substitutions. The table below shows amino acids which may be substituted for an original amino acid in a protein and which are regarded as conservative amino acid substitutions.

| Original Residue | Conservative Substitution |
|---|---|
| Ala | Gly, Ser |
| Arg | His, Lys |
| Asn | Asp, Gln, His |
| Asp | Asn, Glu |
| Cys | Ala, Ser |
| Gln | Asn, Glu, His |
| Glu | Asp, Gln, His |
| Gly | Ala |
| His | Asn, Arg, Gln, Glu |
| Ile | Leu, Val |
| Leu | Ile, Val |
| Lys | Arg, Gln, Glu |
| Met | Leu, Ile |
| Phe | His, Met, Leu, Trp, Tyr |
| Ser | Cys, Thr |
| Thr | Ser, Val |
| Trp | Phe, Tyr |
| Tyr | His, Phe, Trp |
| Val | Ile, Leu, Thr |

Conservative amino acid substitutions generally maintain (a) the structure of the polypeptide backbone in the area of the substitution, for example, as a beta sheet or alpha helical conformation, (b) the charge or hydrophobicity of the molecule at the site of the substitution, and/or (c) the bulk of the side chain.

A “deletion” refers to a change in the amino acid or nucleotide sequence that results in the absence of one or more amino acid residues or nucleotides.

The term “derivative” refers to a chemically modified polynucleotide or polypeptide. Chemical modifications of a polynucleotide can include, for example, replacement of hydrogen by an alkyl, acyl, hydroxyl, or amino group. A derivative polynucleotide encodes a polypeptide which retains at least one biological or immunological function of the natural molecule. A derivative polypeptide is one modified by glycosylation, pegylation, or any similar process that retains at least one biological or immunological function of the polypeptide from which it was derived.

A “detectable label” refers to a reporter molecule or enzyme that is capable of generating a measurable signal and is covalently or noncovalently joined to a polynucleotide or polypeptide.

“Differential expression” refers to increased or upregulated; or decreased, downregulated, or absent gene or protein expression, determined by comparing at least two different samples. Such comparisons may be carried out between, for example, a treated and an untreated sample, or a diseased and a normal sample.

“Exon shuffling” refers to the recombination of different coding regions (exons). Since an exon may represent a structural or functional domain of the encoded protein, new proteins may be assembled through the novel reassortment of stable substructures, thus allowing acceleration of the evolution of new protein functions.

A “fragment” is a unique portion of REMAP or a polynucleotide encoding REMAP which can be identical in sequence to, but shorter in length than, the parent sequence. A fragment may comprise up to the entire length of the defined sequence, minus one nucleotide/amino acid residue. For example, a fragment may comprise from about 5 to about 1000 contiguous nucleotides or amino acid residues. A fragment used as a probe, primer, antigen, therapeutic molecule, or for other purposes, may be at least 5, 10, 15, 16, 20, 25, 30, 40, 50, 60, 75, 100, 150, 250 or at least 500 contiguous nucleotides or amino acid residues in length. Fragments may be preferentially selected from certain regions of a molecule. For example, a polypeptide fragment may comprise a certain length of contiguous amino acids selected from the first 250 or 500 amino acids (or first 25% or 50%) of a polypeptide as shown in a certain defined sequence. Clearly these lengths are exemplary, and any length that is supported by the specification, including the Sequence Listing, tables, and figures, may be encompassed by the present embodiments.

A fragment of SEQ ID NO:48-94 can comprise a region of unique polynucleotide sequence that specifically identifies SEQ ID NO:48-94, for example, as distinct from any other sequence in the genome from which the fragment was obtained. A fragment of SEQ ID NO:48-94 can be employed in one or more embodiments of methods of the invention, for example, in hybridization and amplification technologies and in analogous methods that distinguish SEQ ID NO:49-94 from related polynucleotides. The precise length of a fragment of SEQ ID NO:48-94 and the region of SEQ ID NO:48-94 to which the fragment corresponds are routinely determinable by one of ordinary skill in the art based on the intended purpose for the fragment.

A fragment of SEQ ID NO:1-47 is encoded by a fragment of SEQ ID NO:48-94. A fragment of SEQ ID NO:1-47 can comprise a region of unique amino acid sequence that specifically identifies SEQ ID NO:1-47. For example, a fragment of SEQ ID NO:1-47 can be used as an immunogenic peptide for the development of antibodies that specifically recognize SEQ ID NO:1-47. The precise length of a fragment of SEQ ID NO:1-47 and the region of SEQ ID NO:1-47 to which the fragment corresponds can be determined based on the intended purpose for the fragment using one or more analytical methods described herein or otherwise known in the art.

A “full length” polynucleotide is one containing at least a translation initiation codon (e.g., methionine) followed by an open reading frame and a translation termination codon. A “full length” polynucleotide sequence encodes a “full length” polypeptide sequence.

“Homology” refers to sequence similarity or, alternatively, sequence identity, between two or more polynucleotide sequences or two or more polypeptide sequences.

The terms “percent identity” and “% identity,” as applied to polynucleotide sequences, refer to the percentage of identical residue matches between at least two polynucleotide sequences aligned using a standardized algorithm. Such an algorithm may insert, in a standardized and reproducible way, gaps in the sequences being compared in order to optimize alignment between two sequences, and therefore achieve a more meaningful comparison of the two sequences.

Percent identity between polynucleotide sequences may be determined using one or more computer algorithms or programs known in the art or described herein. For example, percent identity can be determined using the default parameters of the CLUSTAL V algorithm as incorporated into the MEGALIGN version 3.12e sequence alignment program. This program is part of the LASERGENE software package, a suite of molecular biological analysis programs (DNASTAR, Madison Wis.). CLUSTAL V is described in Higgins, D. G. and P. M. Sharp (1989; CABIOS 5:151-153) and in Higgins, D. G. et al. (1992; CABIOS 8:189-191). For pairwise alignments of polynucleotide sequences, the default parameters are set as follows: Ktuple=2, gap penalty=5, window=4, and “diagonals saved”=4. The “weighted” residue weight table is selected as the default.

Alternatively, a suite of commonly used and freely available sequence comparison algorithms which can be used is provided by the National Center for Biotechnology Information (NCBI) Basic Local Alignment Search Tool (BLAST) (Altschul, S. F. et al. (1990) J. Mol. Biol. 215:403-410), which is available from several sources, including the NCBI, Bethesda, Md., and on the Internet at http://www.ncbi.nlm.nih.gov/BLAST/. The BLAST software suite includes various sequence analysis programs including “blastn,” that is used to align a known polynucleotide sequence with other polynucleotide sequences from a variety of databases. Also available is a tool called “BLAST 2 Sequences” that is used for direct pairwise comparison of two nucleotide sequences. “BLAST 2 Sequences” can be accessed and used interactively at http://www.ncbi.nlm.nih.gov/gorf/b12.html. The “BLAST 2 Sequences” tool can be used for both blastn and blastp (discussed below). BLAST programs are commonly used with gap and other parameters set to default settings. For example, to compare two nucleotide sequences, one may use blastn with the “BLAST 2 Sequences” tool Version 2.0.12 (April-21-2000) set at default parameters. Such default parameters may be, for example:
Matrix: BLOSUM62
Reward for match: 1
Penalty for mismatch: −2
Open Gap: 5 and Extension Gap: 2 penalties
Gap x drop-off: 50
Expect: 10
Word Size: 11
Filter: on

Percent identity may be measured over the length of an entire defined sequence, for example, as defined by a particular SEQ ID number, or may be measured over a shorter length, for example, over the length of a fragment taken from a larger, defined sequence, for instance, a fragment of at least 20, at least 30, at least 40, at least 50, at least 70, at least 100, or at least 200 contiguous nucleotides. Such lengths are exemplary only, and it is understood that any fragment length supported by the sequences shown herein, in the tables, figures, or Sequence Listing, may be used to describe a length over which percentage identity may be measured.

Nucleic acid sequences that do not show a high degree of identity may nevertheless encode similar amino acid sequences due to the degeneracy of the genetic code. It is understood that changes in a nucleic acid sequence can be made using this degeneracy to produce multiple nucleic acid sequences that all encode substantially the same protein.

The phrases “percent identity” and “% identity,” as applied to polypeptide sequences, refer to the percentage of identical residue matches between at least two polypeptide sequences aligned using a standardized algorithm. Methods of polypeptide sequence alignment are well-known. Some alignment methods take into account conservative amino acid substitutions. Such conservative substitutions, explained in more detail above, generally preserve the charge and hydrophobicity at the site of substitution, thus preserving the structure (and therefore function) of the polypeptide. The phrases “percent similarity” and “% similarity,” as applied to polypeptide sequences, refer to the percentage of residue matches, including identical residue matches and conservative substitutions, between at least two polypeptide sequences aligned using a standardized algorithm. In contrast, conservative substitutions are not included in the calculation of percent identity between polypeptide sequences.

Percent identity between polypeptide sequences may be determined using the default parameters of the CLUSTAL V algorithm as incorporated into the MEGALIGN version 3.12e sequence alignment program (described and referenced above). For pairwise alignments of polypeptide sequences using CLUSTAL V, the default parameters are set as follows: Ktuple=1, gap penalty-3, window=5, and “diagonals saved”=5. The PAM250 matrix is selected as the default residue weight table.

Alternatively the NCBI BLAST software suite may be used. For example, for a pairwise comparison of two polypeptide sequences, one may use the “BLAST 2 Sequences” tool Version 2.0.12 (April-21-2000) with blastp set at default parameters. Such default parameters may be, for example:
Matrix: BLOSUM62
Open Gap: 11 and Extension Gap: 1 penalties
Gap x drop-off: 50
Expect: 10
Word Size: 3
Filter: on

Percent identity may be measured over the length of an entire defined polypeptide sequence, for example, as defined by a particular SEQ ID number, or may be measured over a shorter length, for example, over the length of a fragment taken from a larger, defined polypeptide sequence, for instance, a fragment of at least 15, at least 20, at least 30, at least 40, at least 50, at least 70 or at least 150 contiguous residues. Such lengths are exemplary only, and it is understood that any fragment length supported by the sequences shown herein, in the tables, figures or Sequence Listing, may be used to describe a length over which percentage identity may be measured.

“Human artificial chromosomes” (HACs) are linear microchromosomes which may contain DNA sequences of about 6 kb to 10 Mb in size and which contain all of the elements required for chromosome replication, segregation and maintenance.

The term “humanized antibody” refers to an antibody molecule in which the amino acid sequence in the non-antigen binding regions has been altered so that the antibody more closely resembles a human antibody, and still retains its original binding ability.

“Hybridization” refers to the process by which a polynucleotide strand anneals with a complementary strand through base pairing under defined hybridization conditions. Specific hybridization is an indication that two nucleic acid sequences share a high degree of complementarity. Specific hybridization complexes form under permissive annealing conditions and remain hybridized after the “washing” step(s). The washing step(s) is particularly important in determining the stringency of the hybridization process, with more stringent conditions allowing less non-specific binding, i.e., binding between pairs of nucleic acid strands that are not perfectly matched. Permissive conditions for annealing of nucleic acid sequences are routinely determinable by one of ordinary skill in the art and may be consistent among hybridization experiments, whereas wash conditions may be varied among experiments to achieve the desired stringency, and therefore hybridization specificity. Permissive annealing conditions occur, for example, at 68° C. in the presence of about 6×SSC, about 1% (w/v) SDS, and about 100 μg/ml sheared, denatured salmon sperm DNA.

Generally, stringency of hybridization is expressed, in part, with reference to the temperature under which the wash step is carried out. Such wash temperatures are typically selected to be about 5° C. to 20° C. lower than the thermal melting point (Tₘ) for the specific sequence at a defined ionic strength and pH. The Tₘ is the temperature (under defined ionic strength and pH) at which 50% of the target sequence hybridizes to a perfectly matched probe. An equation for calculating Tₘ and conditions for nucleic acid hybridization are well known and can be found in Sambrook, J. and D. W. Russell (2001; Molecular Cloning: A Laboratory Manual, 3rd ed., vol. 1-3, Cold Spring Harbor Press, Cold Spring Harbor N.Y., ch. 9).

High stringency conditions for hybridization between polynucleotides of the present invention include wash conditions of 68° C. in the presence of about 0.2×SSC and about 0.1% SDS, for 1 hour. Alternatively, temperatures of about 65° C., 60° C., 55° C., or 42° C. may be used. SSC concentration may be varied from about 0.1 to 2×SSC, with SDS being present at about 0.1%. Typically, blocking reagents are used to block non-specific hybridization. Such blocking reagents include, for instance, sheared and denatured salmon sperm DNA at about 100-200 μg/1 ml. Organic solvent, such as formamide at a concentration of about 35-50% v/v, may also be used under particular circumstances, such as for RNA:DNA hybridizations. Useful variations on these wash conditions will be readily apparent to those of ordinary skill in the art. Hybridization, particularly under high stringency conditions, may be suggestive of evolutionary similarity between the nucleotides. Such similarity is strongly indicative of a similar role for the nucleotides and their encoded polypeptides.

The term “hybridization complex” refers to a complex formed between two nucleic acids by virtue of the formation of hydrogen bonds between complementary bases. A hybridization complex may be formed in solution (e.g., C₀t or R₀t analysis) or formed between one nucleic acid present in solution and another nucleic acid immobilized on a solid support (e.g., paper, membranes, filters, chips, pins or glass slides, or any other appropriate substrate to which cells or their nucleic acids have been fixed).

The words “insertion” and “addition” refer to changes in an amino acid or polynucleotide sequence resulting in the addition of one or more amino acid residues or nucleotides, respectively.

“Immune response” can refer to conditions associated with inflammation, trauma, immune disorders, or infectious or genetic disease, etc. These conditions can be characterized by expression of various factors, e.g., cytokines, chemokines, and other signaling molecules, which may affect cellular and systemic defense systems.

An “immunogenic fragment” is a polypeptide or oligopeptide fragment of REMAP which is capable of eliciting an immune response when introduced into a living organism, for example, a mammal. The term “immunogenic fragment” also includes any polypeptide or oligopeptide fragment of REMAP which is useful in any of the antibody production methods disclosed herein or known in the art.

The term “mnicroarray” refers to an arrangement of a plurality of polynucleotides, polypeptides, antibodies, or other chemical compounds on a substrate.

The terms “element” and “array element” refer to a polynucleotide, polypeptide, antibody, or other chemical compound having a unique and defined position on a microarray.

The term “modulate” refers to a change in the activity of REMAP. For example, modulation may cause an increase or a decrease in protein activity, binding characteristics, or any other biological, functional, or immunological properties of REMAP.

The phrases “nucleic acid” and “nucleic acid sequence” refer to a nucleotide, oligonucleotide, polynucleotide, or any fragment thereof. These phrases also refer to DNA or RNA of genomic or synthetic origin which may be single-stranded or double-stranded and may represent the sense or the antisense strand, to peptide nucleic acid (PNA), or to any DNA-like or RNA-like material.

“Operably linked” refers to the situation in which a first nucleic acid sequence is placed in a functional relationship with a second nucleic acid sequence. For instance, a promoter is operably linked to a coding sequence if the promoter affects the transcription or expression of the coding sequence. Operably linked DNA sequences may be in close proximity or contiguous and, where necessary to join two protein coding regions, in the same reading frame.

“Peptide nucleic acid” (PNA) refers to an antisense molecule or anti-gene agent which comprises an oligonucleotide of at least about 5 nucleotides in length linked to a peptide backbone of amino acid residues ending in lysine. The terminal lysine confers solubility to the composition. PNAs preferentially bind complementary single stranded DNA or RNA and stop transcript elongation, and may be pegylated to extend their lifespan in the cell.

“Post-translational modification” of an REMAP may involve lipidation, glycosylation, phosphorylation, acetylation, racemization, proteolytic cleavage, and other modifications known in the art. These processes may occur synthetically or biochemically. Biochemical modifications will vary by cell type depending on the enzymatic milieu of REMAP.

“Probe” refers to nucleic acids encoding REMAP, their complements, or fragments thereof, which are used to detect identical, allelic or related nucleic acids. Probes are isolated oligonucleotides or polynucleotides attached to a detectable label or reporter molecule. Typical labels include radioactive isotopes, ligands, chemiluminescent agents, and enzymes. “Primers” are short nucleic acids, usually DNA oligonucleotides, which may be annealed to a target polynucleotide by complementary base-pairing. The primer may then be extended along the target DNA strand by a DNA polymerase enzyme. Primer pairs can be used for amplification (and identification) of a nucleic acid, e.g., by the polymerase chain reaction (PCR).

Probes and primers as used in the present invention typically comprise at least 15 contiguous nucleotides of a known sequence. In order to enhance specificity, longer probes and primers may also be employed, such as probes and primers that comprise at least 20, 25, 30, 40, 50, 60, 70, 80, 90, 100, or at least 150 consecutive nucleotides of the disclosed nucleic acid sequences. Probes and primers may be considerably longer than these examples, and it is understood that any length supported by the specification, including the tables, figures, and Sequence Listing, may be used.

Methods for preparing and using probes and primers are described in, for example, Sambrook, J. and D. W. Russell (2001; Molecular Cloning: A Laboratory Manual, 3rd ed., vol. 1-3, Cold Spring Harbor Press, Cold Spring Harbor N.Y.), Ausubel, F. M. et al. (1999; Short Protocols in Molecular Biology, 4′ ed., John Wiley & Sons, New York N.Y.), and Innis, M. et al. (1990; PCR Protocols, A Guide to Methods and Applications, Academic Press, San Diego Calif.). PCR primer pairs can be derived from a known sequence, for example, by using computer programs intended for that purpose such as Primer (Version 0.5, 1991, Whitehead Institute for Biomedical Research, Cambridge Mass.).

Oligonucleotides for use as primers are selected using software known in the art for such purpose. For example, OLIGO 4.06 software is useful for the selection of PCR primer pairs of up to 100 nucleotides each, and for the analysis of oligonucleotides and larger polynucleotides of up to 5,000 nucleotides from an input polynucleotide sequence of up to 32 kilobases. Similar primer selection programs have incorporated additional features for expanded capabilities. For example, the PrimOU primer selection program (available to the public from the Genome Center at University of Texas South West Medical Center, Dallas Tex.) is capable of choosing specific primers from megabase sequences and is thus useful for designing primers on a genome-wide scope. The Primer3 primer selection program (available to the public from the Whitehead Institute/MIT Center for Genome Research, Cambridge Mass.) allows the user to input a “mispriming library,” in which sequences to avoid as primer binding sites are user-specified. Primer3 is useful, in particular, for the selection of oligonucleotides for microarrays. (The source code for the latter two primer selection programs may also be obtained from their respective sources and modified to meet the user's specific needs.) The PrimeGen program (available to the public from the UK Human Genome Mapping Project Resource Centre, Cambridge UK) designs primers based on multiple sequence alignments, thereby allowing selection of primers that hybridize to either the most conserved or least conserved regions of aligned nucleic acid sequences. Hence, this program is useful for identification of both unique and conserved oligonucleotides and polynucleotide fragments. The oligonucleotides and polynucleotide fragments identified by any of the above selection methods are useful in hybridization technologies, for example, as PCR or sequencing primers, microarray elements, or specific probes to identify fully or partially complementary polynucleotides in a sample of nucleic acids. Methods of oligonucleotide selection are not limited to those described above.

A “recombinant nucleic acid” is a nucleic acid that is not naturally occurring or has a sequence that is made by an artificial combination of two or more otherwise separated segments of sequence. This artificial combination is often accomplished by chemical synthesis or, more commonly, by the artificial manipulation of isolated segments of nucleic acids, e.g., by genetic engineering techniques such as those described in Sambrook and Russell (supra). The term recombinant includes nucleic acids that have been altered solely by addition, substitution, or deletion of a portion of the nucleic acid. Frequently, a recombinant nucleic acid may include a nucleic acid sequence operably linked to a promoter sequence. Such a recombinant nucleic acid may be part of a vector that is used, for example, to transform a cell.

Alternatively, such recombinant nucleic acids may be part of a viral vector, e.g., based on a vaccinia virus, that could be use to vaccinate a mammal wherein the recombinant nucleic acid is expressed, inducing a protective immunological response in the mammal.

A “regulatory element” refers to a nucleic acid sequence usually derived from untranslated regions of a gene and includes enhancers, promoters, introns, and 5′ and 3′ untranslated regions (UTRs). Regulatory elements interact with host or viral proteins which control transcription, translation, or RNA stability.

“Reporter molecules” are chemical or biochemical moieties used for labeling a nucleic acid, amino acid, or antibody. Reporter molecules include radionuclides; enzymes; fluorescent, chemiluminescent, or chromogenic agents; substrates; cofactors; inhibitors; magnetic particles; and other moieties known in the art.

An “RNA equivalent,” in reference to a DNA molecule, is composed of the same linear sequence of nucleotides as the reference DNA molecule with the exception that all occurrences of the nitrogenous base thymine are replaced with uracil, and the sugar backbone is composed of ribose instead of deoxyribose.

The term “sample” is used in its broadest sense. A sample suspected of containing REMAP, nucleic acids encoding REMAP, or fragments thereof may comprise a bodily fluid; an extract from a cell, chromosome, organelle, or membrane isolated from a cell; a cell; genomic DNA, RNA, or cDNA, in solution or bound to a substrate; a tissue; a tissue print; etc.

The terms “specific binding” and “specifically binding” refer to that interaction between a protein or peptide and an agonist, an antibody, an antagonist, a small molecule, or any natural or synthetic binding composition. The interaction is dependent upon the presence of a particular structure of the protein, e.g., the antigenic determinant or epitope, recognized by the binding molecule. For example, if an antibody is specific for epitope “A,” the presence of a polypeptide comprising the epitope A, or the presence of free unlabeled A, in a reaction containing free labeled A and the antibody will reduce the amount of labeled A that binds to the antibody.

The term “substantially purified” refers to nucleic acid or amino acid sequences that are removed from their natural environment and are isolated or separated, and are at least about 60% free, preferably at least about 75% free, and most preferably at least about 90% free from other components with which they are naturally associated.

A “substitution” refers to the replacement of one or more amino acid residues or nucleotides by different amino acid residues or nucleotides, respectively.

“Substrate” refers to any suitable rigid or semi-rigid support including membranes, filters, chips, slides, wafers, fibers, magnetic or nonmagnetic beads, gels, tubing, plates, polymers, microparticles and capillaries. The substrate can have a variety of surface forms, such as wells, trenches, pins, channels and pores, to which polynucleotides or polypeptides are bound.

A “transcript image” or “expression profile” refers to the collective pattern of gene expression by a particular cell type or tissue under given conditions at a given time.

“Transformation” describes a process by which exogenous DNA is introduced into a recipient cell. Transformation may occur under natural or artificial conditions according to various methods well known in the art, and may rely on any known method for the insertion of foreign nucleic acid sequences into a prokaryotic or eukaryotic host cell. The method for transformation is selected based on the type of host cell being transformed and may include, but is not limited to, bacteriophage or viral infection, electroporation, heat shock, lipofection, and particle bombardment. The term “transformed cells” includes stably transformed cells in which the inserted DNA is capable of replication either as an autonomously replicating plasmid or as part of the host chromosome, as well as transiently transformed cells which express the inserted DNA or RNA for limited periods of time.

A “transgenic organism,” as used herein, is any organism, including but not limited to animals and plants, in which one or more of the cells of the organism contains heterologous nucleic acid introduced by way of human intervention, such as by transgenic techniques well known in the art. The nucleic acid is introduced into the cell, directly or indirectly by introduction into a precursor of the cell, by way of deliberate genetic manipulation, such as by microinjection or by infection with a recombinant virus. In another embodiment, the nucleic acid can be introduced by infection with a recombinant viral vector, such as a lentiviral vector (Lois, C. et al. (2002) Science 295:868-872). The term genetic manipulation does not include classical cross-breeding, or in vitro fertilization, but rather is directed to the introduction of a recombinant DNA molecule. The transgenic organisms contemplated in accordance with the present invention include bacteria, cyanobacteria, fungi, plants and animals. The isolated DNA of the present invention can be introduced into the host by methods known in the art, for example infection, transfection, transformation or transconjugation. Techniques for transferring the DNA of the present invention into such organisms are widely known and provided in references such as Sambrook and Russell (supra).

A “variant” of a particular nucleic acid sequence is defined as a nucleic acid sequence having at least 40% sequence identity to the particular nucleic acid sequence over a certain length of one of the nucleic acid sequences using blastn with the “BLAST 2 Sequences” tool Version 2.0.9 (May-07-1999) set at default parameters. Such a pair of nucleic acids may show, for example, at least 50%, at least 60%, at least 70%, at least 80%, at least 85%, at least 90%, at least 91%, at least 92%, at least 93%, at least 94%, at least 95%, at least 96%, at least 97%, at least 98%, or at least 99% or greater sequence identity over a certain defined length. A variant may be described as, for example, an “allelic” (as defined above), “splice,” “species,” or “polymorphic” variant. A splice variant may have significant identity to a reference molecule, but will generally have a greater or lesser number of polynucleotides due to alternate splicing of exons during mRNA processing. The corresponding polypeptide may possess additional functional domains or lack domains that are present in the reference molecule. Species variants are polynucleotides that vary from one species to another. The resulting polypeptides will generally have significant amino acid identity relative to each other. A polymorphic variant is a variation in the polynucleotide sequence of a particular gene between individuals of a given species. Polymorphic variants also may encompass “single nucleotide polymorphisms” (SNPs) in which the polynucleotide sequence varies by one nucleotide base. The presence of SNPs may be indicative of, for example, a certain population, a disease state, or a propensity for a disease state.

A “variant” of a particular polypeptide sequence is defined as a polypeptide sequence having at least 40% sequence identity or sequence similarity to the particular polypeptide sequence over a certain length of one of the polypeptide sequences using blastp with the “BLAST 2 Sequences” tool Version 2.0.9 (May-07-1999) set at default parameters. Such a pair of polypeptides may show, for example, at least 50%, at least 60%, at least 70%, at least 80%, at least 85%, at least 90%, at least 91%, at least 92%, at least 93%, at least 94%, at least 95%, at least 96%, at least 97%, at least 98%, or at least 99% or greater sequence identity or sequence similarity over a certain defined length of one of the polypeptides.

### THE INVENTION

Various embodiments of the invention include new human receptors and membrane-associated proteins (REMAP), the polynucleotides encoding REMAP, and the use of these compositions for the diagnosis, treatment, or prevention of cell proliferative, autoimmune/inflammatory, neurological, metabolic, developmental, and endocrine disorders.

Table 1 summarizes the nomenclature for the full length polynucleotide and polypeptide embodiments of the invention. Each polynucleotide and its corresponding polypeptide are correlated to a single Incyte project identification number (Incyte Project ID). Each polypeptide sequence is denoted by both a polypeptide sequence identification number (Polypeptide SEQ ID NO:) and an Incyte polypeptide sequence number (Incyte Polypeptide ID) as shown. Each polynucleotide sequence is denoted by both a polynucleotide sequence identification number (Polynucleotide SEQ ID NO:) and an Incyte polynucleotide consensus sequence number (Incyte Polynucleotide ID) as shown. Column 6 shows the Incyte ID numbers of physical, full length clones corresponding to the polypeptide and polynucleotide sequences of the invention. The full length clones encode polypeptides which have at least 95% sequence identity to the polypeptide sequences shown in column 3.

Table 2 shows sequences with homology to polypeptide embodiments of the invention as identified by BLAST analysis against the GenBank protein (genpept) database and the PROTEOME database. Columns 1 and 2 show the polypeptide sequence identification number (Polypeptide SEQ ID NO:) and the corresponding Incyte polypeptide sequence number (Incyte Polypeptide ID) for polypeptides of the invention. Column 3 shows the GenBank identification number (GenBank ID NO:) of the nearest GenBank homolog and the PROTEOME database identification numbers (PROTEOME ID NO:) of the nearest PROTEOME database homologs. Column 4 shows the probability scores for the matches between each polypeptide and its homolog(s). Column 5 shows the annotation of the GenBank and PROTEOME database homolog(s) along with relevant citations where applicable, all of which are expressly incorporated by reference herein.

Table 3 shows various structural features of the polypeptides of the invention. Columns 1 and 2 show the polypeptide sequence identification number (SEQ ID NO:) and the corresponding Incyte polypeptide sequence number (Incyte Polypeptide ID) for each polypeptide of the invention. Column 3 shows the number of amino acid residues in each polypeptide. Column 4 shows potential phosphorylation sites, and column 5 shows potential glycosylation sites, as determined by the MOTIFS program of the GCG sequence analysis software package (Accelrys, Burlington Mass.). Column 6 shows amino acid residues comprising signature sequences, domains, and motifs. Column 7 shows analytical methods for protein structure/function analysis and in some cases, searchable databases to which the analytical methods were applied.

Together, Tables 2 and 3 summarize the properties of polypeptides of the invention, and these properties establish that the claimed polypeptides are receptors and membrane-associated proteins.

For example, SEQ ID NO:4 is 97% identical, from residue MI to residue P145, to human LOX1 (GenBank ID g4468344) as determined by the Basic Local Alignment Search Tool (BLAST). (See Table 2.) The BLAST probability score is 3.8e-70, which indicates the probability of obtaining the observed polypeptide sequence alignment by chance. SEQ ID NO:4 is localized to the plasma membrane and is a lectin-like oxidized low density lipoprotein receptor, as determined by BLAST analysis using the PROTEOME database. Data from BLIMPS, BLAST, TMHMMER and MOTIFS analyses provide further corroborative evidence that SEQ ID NO:4 is a lectin-like oxidized low density lipoprotein receptor.

In a further example, SEQ ID NO:6 is 94% identical, from residue MI to residue C180, to human dlk (GenBank ID g562106) as determined by BLAST. (See Table 2.) The BLAST probability score is 1.8e-102. SEQ ID NO:6 also has homology to proteins that are localized to the extracellular space, and are members of the EGF-like superfamily, as determined by BLAST analysis using the PROTEOME database. SEQ ID NO:6 also contains an EGF-like domain as determined by searching for statistically significant matches in the hidden Markov model (HMM)-based PFAM database of conserved protein family domains. (See Table 3.) Data from TMHMMR, BLIMPS, MOTIFS, and further BLAST analyses provide further corroborative evidence that SEQ ID NO:6 is an EGF-like glycoprotein.

In another example, SEQ ID NO:16 is 100% identical, from residue MI to residue Q229, to human tetraspanin, TM4-B (GenBank ID g6434902) as determined by BLAST. (See Table 2.) The BLAST probability score is 2.5e-121. SEQ ID NO:16 also has homology to proteins that function in cell proliferation, differentiation, adhesion, and migration, and are tetraspanins, as determined by BLAST analysis using the PROTEOME database. SEQ ID NO:16 also contains a tetraspanin family domain as determined by searching for statistically significant matches in the hidden Markov model (HMM)-based PFAM database of conserved protein family domains. (See Table 3.) Data from BLIMPS, TMHMMER, PROFILESCAN, and other BLAST analyses provide further corroborative evidence that SEQ ID NO:16 is a TM4-B tetraspanin.

In yet another example, SEQ ID NO:31 is 99% identical, from residue M18 to residue Y529, to human transmembrane mucin MUC13 (GenBank ID g14209832) as determined by BLAST. (See Table 2.) The BLAST probability score is 1.1e-276. SEQ ID NO:31 also has homology to proteins that are highly expressed in myeloid progenitor cells, may have a role in regulation of cellular responses to IL-3, and have a high similarity to lymphocyte antigen 64, as determined by BLAST analysis using the PROTEOME database. SEQ ID NO:31 also contains an SEA domain as determined by searching for statistically significant matches in the hidden Markov model (HMM)-based PFAM database of conserved protein family domains. (See Table 3.) Data from MOTIFS and additional BLAST analyses provide further corroborative evidence that SEQ ID NO:31 is a cell surface antigen.

In a further example, SEQ ID NO:46 is 100% identical, from residue M1 to residue N431, and is 98% identical, from residue 1430 to residue P562 to human lamin B receptor homolog TM7SF2; ANG1 (GenBank ID g3211722) as determined by BLAST. (See Table 2.) The BLAST probability scores are 0.0 and 0.0 respectively. As determined by BLAST analysis using the PROTEOME database, SEQ ID NO:46 also has homology to transmembrane 7 superfamily member 2, a member of the lamin B receptor-sterol reductase family of proteins which is localized exclusively to the endoplasmic reticulum and contains seven putative C-terminal transmembrane domains (PROTEOME ID 338558|TM7SF2). SEQ ID NO:46 also contains an ergosterol biosynthesis ERG4/ERG24 family domain as determined by searching for statistically significant matches in the hidden Markov model (HMM)-based PFAM database of conserved protein family domains. (See Table 3.) Data from BLIMPS, MOTIFS, and additional BLAST analyses provide further corroborative evidence that SEQ ID NO:46 is a member of the lamin B receptor family of proteins. SEQ ID NO:1-3, SEQ ID NO:5, SEQ ID NO:7-15, SEQ ID NO:17-30, SEQ ID NO:32-45, and SEQ ID NO:47 were analyzed and annotated in a similar manner. The algorithms and parameters for the analysis of SEQ ID NO:1-47 are described in Table 7.

As shown in Table 4, the full length polynucleotide embodiments were assembled using cDNA sequences or coding (exon) sequences derived from genomic DNA, or any combination of these two types of sequences. Column 1 lists the polynucleotide sequence identification number (Polynucleotide SEQ ID NO:), the corresponding Incyte polynucleotide consensus sequence number (Incyte ID) for each polynucleotide of the invention, and the length of each polynucleotide sequence in basepairs. Column 2 shows the nucleotide start (5′) and stop (3′) positions of the cDNA and/or genomic sequences used to assemble the full length polynucleotide embodiments, and of fragments of the polynucleotides which are useful, for example, in hybridization or amplification technologies that identify SEQ ID NO:48-94 or that distinguish between SEQ ID NO:48-94 and related polynucleotides.

The polynucleotide fragments described in Column 2 of Table 4 may refer specifically, for example, to Incyte cDNAs derived from tissue-specific cDNA libraries or from pooled cDNA libraries. Alternatively, the polynucleotide fragments described in column 2 may refer to GenBank cDNAs or ESTs which contributed to the assembly of the full length polynucleotides. In addition, the polynucleotide fragments described in column 2 may identify sequences derived from the ENSEMBL (The Sanger Centre, Cambridge, UK) database (i.e., those sequences including the designation “ENST”). Alternatively, the polynucleotide fragments described in column 2 may be derived from the NCBI RefSeq Nucleotide Sequence Records Database (i.e., those sequences including the designation “NM” or “NT”) or the NCBI RefSeq Protein Sequence Records (i.e., those sequences including the designation “NP”). Alternatively, the polynucleotide fragments described in column 2 may refer to assemblages of both cDNA and Genscan-predicted exons brought together by an “exon stitching” algorithm. For example, a polynucleotide sequence identified as FL_XXXXXX_N_{1—}N_{2—}YYYYY_N_{3—}N₄ represents a “stitched” sequence in which XXXXXX is He identification number of the cluster of sequences to which the algorithm was applied, and YYYYY is the number of the prediction generated by the algorithm, and N_{1,2,3} . . . , if present, represent specific exons that may have been manually edited during analysis (See Example V). Alternatively, the polynucleotide fragments in column 2 may refer to assemblages of exons brought together by an “exon-stretching” algorithm. For example, a polynucleotide sequence identified as FLXXXXXX_gAAAAA_gBBBBB_{—}1_N is a “stretched” sequence, with XXXXXX being the Incyte project identification number, gAAAAA being the GenBank identification number of the human genomic sequence to which the “exon-stretching” algorithm was applied, gBBBBB being the GenBank identification number or NCBI RefSeq identification number of the nearest GenBank protein homolog, and N referring to specific exons (See Example V). In instances where a RefSeq sequence was used as a protein homolog for the “exon-stretching” algorithm, a RefSeq identifier (denoted by “NM,” “NP,” or “NT”) may be used in place of the GenBank identifier (i.e., gBBBBB).

Alternatively, a prefix identifies component sequences that were hand-edited, predicted from genomic DNA sequences, or derived from a combination of sequence analysis methods. The following Table lists examples of component sequence prefixes and corresponding sequence analysis methods associated with the prefixes (see Example IV and Example V).

| Prefix | Type of analysis and/or examples of programs |
|---|---|
| GNN, GFG, ENST | Exon prediction from genomic sequences using, |
| | for example, GENSCAN (Stanford University, CA, |
| | USA) or FGENES (Computer Genomics Group, The |
| | Sanger Centre, Cambridge, UK). |
| GBI | Hand-edited analysis of genomic sequences. |
| FL | Stitched or stretched genomic sequences |
| | (see Example V). |
| INCY | Full length transcript and exon prediction |
| | from mapping of EST sequences to the genome. |
| | Genomic location and EST composition data are |
| | combined to predict the exons and resulting |
| | transcript. |

In some cases, Incyte cDNA coverage redundant with the sequence coverage shown in Table 4 was obtained to confirm the final consensus polynucleotide sequence, but the relevant Incyte cDNA identification numbers are not shown.

Table 5 shows the representative cDNA libraries for those full length polynucleotides which were assembled using Incyte cDNA sequences. The representative cDNA library is the Incyte cDNA library which is most frequently represented by the Incyte cDNA sequences which were used to assemble and confirm the above polynucleotides. The tissues and vectors which were used to construct the cDNA libraries shown in Table 5 are described in Table 6.

Table 8 shows single nucleotide polymorphisms (SNPs) found in polynucleotide sequences of the invention, along with allele frequencies in different human populations. Columns 1 and 2 show the polynucleotide sequence identification number (SEQ ID NO:) and the corresponding Incyte project identification number (PID) for polynucleotides of the invention. Column 3 shows the Incyte identification number for the EST in which the SNP was detected (EST ID), and column 4 shows the identification number for the SNP (SNP ID). Column 5 shows the position within the EST sequence at which the SNP is located (EST SNP), and column 6 shows the position of the SNP within the full-length polynucleotide sequence (CB1 SNP). Column 7 shows the allele found in the EST sequence. Columns 8 and 9 show the two alleles found at the SNP site. Column 10 shows the amino acid encoded by the codon including the SNP site, based upon the allele found in the EST. Columns 11-14 show the frequency of allele 1 in four different human populations. An entry of n/d (not detected) indicates that the frequency of allele 1 in the population was too low to be detected, while n/a (not available) indicates that the allele frequency was not determined for the population.

The invention also encompasses REMAP variants. Various embodiments of REMAP variants can have at least about 80%, at least about 90%, or at least about 95% amino acid sequence identity to the REMAP amino acid sequence, and can contain at least one functional or structural characteristic of REMAP.

Various embodiments also encompass polynucleotides which encode REMAP. In a particular embodiment, the invention encompasses a polynucleotide sequence comprising a sequence selected from the group consisting of SEQ ID NO:48-94, which encodes REMAP. The polynucleotide sequences of SEQ ID NO:48-94, as presented in the Sequence Listing, embrace the equivalent RNA sequences, wherein occurrences of the nitrogenous base thymine are replaced with uracil, and the sugar backbone is composed of ribose instead of deoxyribose.

The invention also encompasses variants of a polynucleotide encoding REMAP. In particular, such a variant polynucleotide will have at least about 70%, or alternatively at least about 85%, or even at least about 95% polynucleotide sequence identity to a polynucleotide encoding REMAP. A particular aspect of the invention encompasses a variant of a polynucleotide comprising a sequence selected from the group consisting of SEQ ID NO:48-94 which has at least about 70%, or alternatively at least about 85%, or even at least about 95% polynucleotide sequence identity to a nucleic acid sequence selected from the group consisting of SEQ ID NO:48-94. Any one of the polynucleotide variants described above can encode a polypeptide which contains at least one functional or structural characteristic of REMAP.

In addition, or in the alternative, a polynucleotide variant of the invention is a splice variant of a polynucleotide encoding REMAP. A splice variant may have portions which have significant sequence identity to a polynucleotide encoding REMAP, but will generally have a greater or lesser number of polynucleotides due to additions or deletions of blocks of sequence arising from alternate splicing of exons during mRNA processing. A splice variant may have less than about 70%, or alternatively less than about 60%, or alternatively less than about 50% polynucleotide sequence identity to a polynucleotide encoding REMAP over its entire length; however, portions of the splice variant will have at least about 70%, or alternatively at least about 85%, or alternatively at least about 95%, or alternatively 100% polynucleotide sequence identity to portions of the polynucleotide encoding REMAP. For example, a polynucleotide comprising a sequence of SEQ ID NO:52 and a polynucleotide comprising a sequence of SEQ ID NO:53 are splice variants of each other. Any one of the splice variants described above can encode a polypeptide which contains at least one functional or structural characteristic of REMAP.

It will be appreciated by those skilled in the art that as a result of the degeneracy of the genetic code, a multitude of polynucleotide sequences encoding REMAP, some bearing minimal similarity to the polynucleotide sequences of any known and naturally occurring gene, may be produced. Thus, the invention contemplates each and every possible variation of polynucleotide sequence that could be made by selecting combinations based on possible codon choices. These combinations are made in accordance with the standard triplet genetic code as applied to the polynucleotide sequence of naturally occurring REMAP, and all such variations are to be considered as being specifically disclosed.

Although polynucleotides which encode REMAP and its variants are generally capable of hybridizing to polynucleotides encoding naturally occurring REMAP under appropriately selected conditions of stringency, it may be advantageous to produce polynucleotides encoding REMAP or its derivatives possessing a substantially different codon usage, e.g., inclusion of non-naturally occurring codons. Codons may be selected to increase the rate at which expression of the peptide occurs in a particular prokaryotic or eukaryotic host in accordance with the frequency with which particular codons are utilized by the host. Other reasons for substantially altering the nucleotide sequence encoding REMAP and its derivatives without altering the encoded amino acid sequences include the production of RNA transcripts having more desirable properties, such as a greater half-life, than transcripts produced from the naturally occurring sequence.

The invention also encompasses production of polynucleotides which encode REMAP and REMAP derivatives, or fragments thereof, entirely by synthetic chemistry. After production, the synthetic polynucleotide may be inserted into any of the many available expression vectors and cell systems using reagents well known in the art. Moreover, synthetic chemistry may be used to introduce mutations into a polynucleotide encoding REMAP or any fragment thereof.

Embodiments of the invention can also include polynucleotides that are capable of hybridizing to the claimed polynucleotides, and, in particular, to those having the sequences shown in SEQ ID NO:48-94 and fragments thereof, under various conditions of stringency (Wahl, G. M. and S. L. Berger (1987) Methods Enzymol. 152:399-407; Kimmel, A. R. (1987) Methods Enzymol. 152:507-511). Hybridization conditions, including annealing and wash conditions, are described in “Definitions.”

Methods for DNA sequencing are well known in the art and may be used to practice any of the embodiments of the invention. The methods may employ such enzymes as the Klenow fragment of DNA polymerase I, SEQUENASE (US Biochemical, Cleveland Ohio), Taq polymerase (Applied Biosystems), thermostable T7 polymerase (Amersham Biosciences, Piscataway N.J.), or combinations of polymerases and proofreading exonucleases such as those found in the ELONGASE amplification system (Invitrogen, Carlsbad Calif.). Preferably, sequence preparation is automated with machines such as the MICROLAB 2200 liquid transfer system (Hamilton, Reno Nev.), PTC200 thermal cycler (MJ Research, Watertown Mass.) and ABI CATALYST 800 thermal cycler (Applied Biosystems). Sequencing is then carried out using either the ABI 373 or 377 DNA sequencing system (Applied Biosystems), the MEGABACE 1000 DNA sequencing system (Amersham Biosciences), or other systems known in the art. The resulting sequences are analyzed using a variety of algorithms which are well known in the art (Ausubel et al., supra, ch. 7; Meyers, R. A. (1995) Molecular Biology and Biotechnology, Wiley VCH, New York N.Y., pp. 856-853).

The nucleic acids encoding REMAP may be extended utilizing a partial nucleotide sequence and employing various PCR-based methods known in the art to detect upstream sequences, such as promoters and regulatory elements. For example, one method which maybe employed, restriction-site PCR, uses universal and nested primers to amplify unknown sequence from genomic DNA within a cloning vector (Sarkar, G. (1993) PCR Methods Applic. 2:318-322). Another method, inverse PCR, uses primers that extend in divergent directions to amplify unknown sequence from a circularized template. The template is derived from restriction fragments comprising a known genomic locus and surrounding sequences (Triglia, T. et al. (1988) Nucleic Acids Res. 16:8186). A third method, capture PCR, involves PCR amplification of DNA fragments adjacent to known sequences in human and yeast artificial chromosome DNA (Lagerstrom, M. et al. (1991) PCR Methods Applic. 1:111-119). In this method, multiple restriction enzyme digestions and ligations may be used to insert an engineered double-stranded sequence into a region of unknown sequence before performing PCR. Other methods which may be used to retrieve unknown sequences are known in the art (Parker, J. D. et al. (1991) Nucleic Acids Res. 19:3055-3060). Additionally, one may use PCR, nested primers, and PROMOTERFINDER libraries (Clontech, Palo Alto Calif.) to walk genomic DNA. This procedure avoids the need to screen libraries and is useful in finding intron/exon junctions. For all PCR-based methods, primers may be designed using commercially available software, such as OLIGO 4.06 primer analysis software (National Biosciences, Plymouth Minn.) or another appropriate program, to be about 22 to 30 nucleotides in length, to have a GC content of about 50% or more, and to anneal to the template at temperatures of about 68° C. to 72° C.

When screening for full length cDNAs, it is preferable to use libraries that have been size-selected to include larger cDNAs. In addition, random-primed libraries, which often include sequences containing the 5′ regions of genes, are preferable for situations in which an oligo d(T) library does not yield a full-length cDNA. Genomic libraries may be useful for extension of sequence into 5′ non-transcribed regulatory regions.

Capillary electrophoresis systems which are commercially available may be used to analyze the size or confirm the nucleotide sequence of sequencing or PCR products. In particular, capillary sequencing may employ flowable polymers for electrophoretic separation, four different nucleotide-specific, laser-stimulated fluorescent dyes, and a charge coupled device camera for detection of the emitted wavelengths. Output/light intensity may be converted to electrical signal using appropriate software (e.g., GENOTYPER and SEQUENCE NAVIGATOR, Applied Biosystems), and the entire process from loading of samples to computer analysis and electronic data display may be computer controlled. Capillary electrophoresis is especially preferable for sequencing small DNA fragments which may be present in limited amounts in a particular sample.

In another embodiment of the invention, polynucleotides or fragments thereof which encode REMAP may be cloned in recombinant DNA molecules that direct expression of REMAP, or fragments or functional equivalents thereof, in appropriate host cells. Due to the inherent degeneracy of the genetic code, other polynucleotides which encode substantially the same or a functionally equivalent polypeptides may be produced and used to express REMAP.

The polynucleotides of the invention can be engineered using methods generally known in the art in order to alter REMAP-encoding sequences for a variety of purposes including, but not limited to, modification of the cloning, processing, and/or expression of the gene product. DNA shuffling by random fragmentation and PCR reassembly of gene fragments and synthetic oligonucleotides may be used to engineer the nucleotide sequences. For example, oligonucleotide-mediated site-directed mutagenesis may be used to introduce mutations that create new restriction sites, alter glycosylation patterns, change codon preference, produce splice variants, and so forth.

The nucleotides of the present invention may be subjected to DNA shuffling techniques such as MOLECULARBREEDING (Maxygen Inc., Santa Clara Calif.; described in U.S. Pat. No. 5,837,458; Chang, C. -C. et al. (1999) Nat. Biotechnol. 17:793-797; Christians, F. C. et al. (1999) Nat. Biotechnol. 17:259-264; and Crameri, A. et al. (1996) Nat. Biotechnol. 14:315-319) to alter or improve the biological properties of REMAP, such as its biological or enzymatic activity or its ability to bind to other molecules or compounds. DNA shuffling is a process by which a library of gene variants is produced using PCR-mediated recombination of gene fragments. The library is then subjected to selection or screening procedures that identify those gene variants with the desired properties. These preferred variants may then be pooled and further subjected to recursive rounds of DNA shuffling and selection/screening. Thus, genetic diversity is created through “artificial” breeding and rapid molecular evolution. For example, fragments of a single gene containing random point mutations may be recombined, screened, and then reshuffled until the desired properties are optimized. Alternatively, fragments of a given gene may be recombined with fragments of homologous genes in the same gene family, either from the same or different species, thereby maximizing the genetic diversity of multiple naturally occurring genes in a directed and controllable manner.

In another embodiment, polynucleotides encoding REMAP may be synthesized, in whole or in part, using one or more chemical methods well known in the art (Caruthers, M. H. et al. (1980) Nucleic Acids Symp. Ser. 7:215-223; Horn, T. et al. (1980) Nucleic Acids Symp. Ser. 7:225-232). Alternatively, REMAP itself or a fragment thereof may be synthesized using chemical methods known in the art. For example, peptide synthesis can be performed using various solution-phase or solid-phase techniques (Creighton, T. (1984) Proteins, Structures and Molecular Properties, W H Freeman, New York N.Y., pp. 55-60; Roberge, J. Y. et al. (1995) Science 269:202-204). Automated synthesis may be achieved using the ABI 431A peptide synthesizer (Applied Biosystems). Additionally, the amino acid sequence of REMAP, or any part thereof, may be altered during direct synthesis and/or combined with sequences from other proteins, or any part thereof, to produce a variant polypeptide or a polypeptide having a sequence of a naturally occurring polypeptide.

The peptide may be substantially purified by preparative high performance liquid chromatography (Chiez, R. M. and F. Z. Regnier (1990) Methods Enzymol. 182:392-421). The composition of the synthetic peptides may be confirmed by amino acid analysis or by sequencing (Creighton, supra, pp. 28-53).

In order to express a biologically active REMAP, the polynucleotides encoding REMAP or derivatives thereof may be inserted into an appropriate expression vector, i.e., a vector which contains the necessary elements for transcriptional and translational control of the inserted coding sequence in a suitable host. These elements include regulatory sequences, such as enhancers, constitutive and inducible promoters, and 5′ and 3′ untranslated regions in the vector and in polynucleotides encoding REMAP. Such elements may vary in their strength and specificity. Specific initiation signals may also be used to achieve more efficient translation of polynucleotides encoding REMAP. Such signals include the ATG initiation codon and adjacent sequences, e.g. the Kozak sequence. In cases where a polynucleotide sequence encoding REMAP and its initiation codon and upstream regulatory sequences are inserted into the appropriate expression vector, no additional transcriptional or translational control signals may be needed. However, in cases where only coding sequence, or a fragment thereof, is inserted, exogenous translational control signals including an in-frame ATG initiation codon should be provided by the vector. Exogenous translational elements and initiation codons may be of various origins, both natural and synthetic. The efficiency of expression may be enhanced by the inclusion of enhancers appropriate for the particular host cell system used (Scharf, D. et al (1994) Results Probl. Cell Differ. 20:125-162).

Methods which are well known to those skilled in the art may be used to construct expression vectors containing polynucleotides encoding REMAP and appropriate transcriptional and translational control elements. These methods include in vitro recombinant DNA techniques, synthetic techniques, and in vivo genetic recombination (Sambrook and Russell, supra, ch. 1-4, and 8; Ausubel et al., supra, ch. 1, 3, and 15).

A variety of expression vector/host systems may be utilized to contain and express polynucleotides encoding REMAP. These include, but are not limited to, microorganisms such as bacteria transformed with recombinant bacteriophage, plasmid, or cosmid DNA expression vectors; yeast transformed with yeast expression vectors; insect cell systems infected with viral expression vectors (e.g., baculovirus); plant cell systems transformed with viral expression vectors (e.g., cauliflower mosaic virus, CaMV, or tobacco mosaic virus, TMV) or with bacterial expression vectors (e.g., Ti or pBR322 plasmids); or animal cell systems (Sambrook and Russell, supra; Ausubel et al., supra; Van Heeke, G. and S. M. Schuster (1989) J. Biol. Chem. 264:5503-5509; Engelhard, E. K. et al. (1994) Proc. Natl. Acad. Sci. USA 91:3224-3227; Sandig, V. et al. (1996) Hum. Gene Ther. 7:1937-1945; Takamatsu, N. (1987) EMBO J. 6:307-311; The McGraw Hill Yearbook of Science and Technology (1992) McGraw Hill, New York N.Y., pp. 191-196; Logan, J. and T. Shenk (1984) Proc. Natl. Acad. Sci. USA 81:3655-3659; Harrington, J. J. et al. (1997) Nat. Genet. 15:345-355). Expression vectors derived from retroviruses, adenoviruses, or herpes or vaccinia viruses, or from various bacterial plasmids, may be used for delivery of polynucleotides to the targeted organ, tissue, or cell population (Di Nicola, M. et al. (1998) Cancer Gen. Ther. 5:350-356; Yu, M. et al. (1993) Proc. Natl. Acad. Sci. USA 90:6340-6344; Buller, R. M. et al. (1985) Nature 317:813-815; McGregor, D. P. et al. (1994) Mol. Immunol. 31:219-226; Verma, I. M. and N. Somia (1997) Nature 389:239-242). The invention is not limited by the host cell employed.

In bacterial systems, a number of cloning and expression vectors may be selected depending upon the use intended for polynucleotides encoding REMAP. For example, routine cloning, subcloning, and propagation of polynucleotides encoding REMAP can be achieved using a multifunctional E. coli vector such as PBLUESCRIPT (Stratagene, La Jolla Calif.) or PSPORT1 plasmid (Invitrogen). Ligation of polynucleotides encoding REMAP into the vector's multiple cloning site disrupts the lacZ gene, allowing a colorimetric screening procedure for identification of transformed bacteria containing recombinant molecules. In addition, these vectors may be useful for in vitro transcription, dideoxy sequencing, single strand rescue with helper phage, and creation of nested deletions in the cloned sequence (Van Heeke, G. and S. M. Schuster (1989) J. Biol. Chem. 264:5503-5509). When large quantities of REMAP are needed, e.g. for the production of antibodies, vectors which direct high level expression of REMAP may be used. For example, vectors containing the strong, inducible SP6 or T7 bacteriophage promoter may be used.

Yeast expression systems may be used for production of REMAP. A number of vectors containing constitutive or inducible promoters, such as alpha factor, alcohol oxidase, and PGH promoters, may be used in the yeast Saccharomyces cerevisiae or Pichia pastoris. In addition, such vectors direct either the secretion or intracellular retention of expressed proteins and enable integration of foreign polynucleotide sequences into the host genome for stable propagation (Ausubel et al., supra; Bitter, G. A. et al. (1987) Methods Enzymol. 153:516-544; Scorer, C. A. et al. (1994) Bio/Technology 12:181-184).

Plant systems may also be used for expression of REMAP. Transcription of polynucleotides encoding REMAP may be driven by viral promoters, e.g., the 35S and 19S promoters of CaMV used alone or in combination with the omega leader sequence from TMV (Takamatsu, N. (1987) EMBO J. 6:307-311). Alternatively, plant promoters such as the small subunit of RUBISCO or heat shock promoters may be used (Coruzzi, G. et al. (1984) EMBO J. 3:1671-1680; Broglie, R. et al. (1984) Science 224:838-843; Winter, J. et al. (1991) Results Probl. Cell Differ. 17:85-105). These constructs can be introduced into plant cells by direct DNA transformation or pathogen-mediated transfection (The McGraw Hill Yearbook of Science and Technology (1992) McGraw Hill, New York N.Y., pp. 191-196).

In mammalian cells, a number of viral-based expression systems may be utilized. In cases where an adenovirus is used as an expression vector, polynucleotides encoding REMAP may be ligated into an adenovirus transcription/translation complex consisting of the late promoter and tripartite leader sequence. Insertion in a non-essential E1 or E3 region of the viral genome may be used to obtain infective virus which expresses REMAP in host cells (Logan, J. and T. Shenk (1984) Proc. Natl. Acad. Sci. USA 81:3655-3659). In addition, transcription enhancers, such as the Rous sarcoma virus (RSV) enhancer, may be used to increase expression in mammalian host cells. SV40 or EBV-based vectors may also be used for high-level protein expression.

Human artificial chromosomes (HACs) may also be employed to deliver larger fragments of DNA than can be contained in and expressed from a plasmid. HACs of about 6 kb to 10 Mb are constructed and delivered via conventional delivery methods (liposomes, polycationic amino polymers, or vesicles) for therapeutic purposes (Harrington, J. J. et al. (1997) Nat. Genet 15:345-355).

For long term production of recombinant proteins in mammalian systems, stable expression of REMAP in cell lines is preferred. For example, polynucleotides encoding REMAP can be transformed into cell lines using expression vectors which may contain viral origins of replication and/or endogenous expression elements and a selectable marker gene on the same or on a separate vector. Following the introduction of the vector, cells may be allowed to grow for about 1 to 2 days in enriched media before being switched to selective media. The purpose of the selectable marker is to confer resistance to a selective agent, and its presence allows growth and recovery of cells which successfully express the introduced sequences. Resistant clones of stably transformed cells may be propagated using tissue culture techniques appropriate to the cell type.

Any number of selection systems may be used to recover transformed cell lines. These include, but are not limited to, the herpes simplex virus thymidine kinase and adenine phosphoribosyltransferase genes, for use in tk and apr cells, respectively (Wigler, M. et al. (1977) Cell 11:223-232; Lowy, I. et al. (1980) Cell 22:817-823). Also, antimetabolite, antibiotic, or herbicide resistance can be used as the basis for selection. For example, dhfr confers resistance to methotrexate; neo confers resistance to the aminoglycosides neomycin and G-418; and als and pat confer resistance to chlorsulfuron and phosphinotricin acetyltransferase, respectively (Wigler, M. et al. (1980) Proc. Natl. Acad. Sci. USA 77:3567-3570; Colbere-Garapin, F. et al. (1981) J. Mol. Biol. 150:1-14). Additional selectable genes have been described, e.g., IrpB and hisD, which alter cellular requirements for metabolites (Hartman, S. C. and R. C. Mulligan (1988) Proc. Natl. Acad. Sci. USA 85:8047-8051). Visible markers, e.g., anthocyanins, green fluorescent proteins (GFP; Clontech), O-glucuronidase and its substrate β-glucuronide, or luciferase and its substrate luciferin may be used. These markers can be used not only to identify transformants, but also to quantify the amount of transient or stable protein expression attributable to a specific vector system (Rhodes, C. A. (1995) Methods Mol. Biol. 55:121-131).

Although the presence/absence of marker gene expression suggests that the gene of interest is also present, the presence and expression of the gene may need to be confirmed. For example, if the sequence encoding REMAP is inserted within a marker gene sequence, transformed cells containing polynucleotides encoding REMAP can be identified by the absence of marker gene function. Alternatively, a marker gene can be placed in tandem with a sequence encoding REMAP under the control of a single promoter. Expression of the marker gene in response to induction or selection usually indicates expression of the tandem gene as well.

In general, host cells that contain the polynucleotide encoding REMAP and that express REMAP may be identified by a variety of procedures known to those of skill in the art. These procedures include, but are not limited to, DNA-DNA or DNA-RNA hybridizations, PCR amplification, and protein bioassay or immunoassay techniques which include membrane, solution, or chip based technologies for the detection and/or quantification of nucleic acid or protein sequences.

Immunological methods for detecting and measuring the expression of REMAP using either specific polyclonal or monoclonal antibodies are known in the art. Examples of such techniques include enzyme-linked immunosorbent assays (ELISAs), radioimmunoassays (RIAs), and fluorescence activated cell sorting (FACS). A two-site, monoclonal-based immunoassay utilizing monoclonal antibodies reactive to two non-interfering epitopes on REMAP is preferred, but a competitive binding assay may be employed. These and other assays are well known in the art (Hampton, R. et al. (1990) Serological Methods, a Laboratory Manual, APS Press, St. Paul Minn., Sect. IV; Coligan, J. E. et al. (1997) Current Protocols in Immunology, Greene Pub. Associates and Wiley-Interscience, New York N.Y.; Pound, J. D. (1998) Immunochemical Protocols, Humana Press, Totowa N.J.).

A wide variety of labels and conjugation techniques are known by those skilled in the art and may be used in various nucleic acid and amino acid assays. Means for producing labeled hybridization or PCR probes for detecting sequences related to polynucleotides encoding REMAP include oligolabeling, nick translation, end-labeling, or PCR amplification using a labeled nucleotide. Alternatively, polynucleotides encoding REMAP, or any fragments thereof, may be cloned into a vector for the production of an mRNA probe. Such vectors are known in the art, are commercially available, and may be used to synthesize RNA probes in vitro by addition of an appropriate RNA polymerase such as T7, T3, or SP6 and labeled nucleotides. These procedures may be conducted using a variety of commercially available kits, such as those provided by Amersham Biosciences, Promega (Madison Wis.), and US Biochemical. Suitable reporter molecules or labels which may be used for ease of detection include radionuclides, enzymes, fluorescent, chemiluminescent, or chromogenic agents, as well as substrates, cofactors, inhibitors, magnetic particles, and the like.

Host cells transformed with polynucleotides encoding REMAP may be cultured under conditions suitable for the expression and recovery of the protein from cell culture. The protein produced by a transformed cell may be secreted or retained intracellularly depending on the sequence and/or the vector used. As will be understood by those of skill in the art, expression vectors containing polynucleotides which encode REMAP may be designed to contain signal sequences which direct secretion of REMAP through a prokaryotic or eukaryotic cell membrane.

In addition, a host cell strain may be chosen for its ability to modulate expression of the inserted polynucleotides or to process the expressed protein in the desired fashion. Such modifications of the polypeptide include, but are not limited to, acetylation, carboxylation, glycosylation, phosphorylation, lipidation, and acylation. Post-translational processing which cleaves a “prepro” or “pro” form of the protein may also be used to specify protein targeting, folding, and/or activity. Different host cells which have specific cellular machinery and characteristic mechanisms for post-translational activities (e.g., CHO, HeLa, MDCK, HEK293, and W138) are available from the American Type Culture Collection (ATCC, Manassas Va.) and may be chosen to ensure the correct modification and processing of the foreign protein.

In another embodiment of the invention, natural, modified, or recombinant polynucleotides encoding REMAP may be ligated to a heterologous sequence resulting in translation of a fusion protein in any of the aforementioned host systems. For example, a chimeric REMAP protein containing a heterologous moiety that can be recognized by a commercially available antibody may facilitate the screening of peptide libraries for inhibitors of REMAP activity. Heterologous protein and peptide moieties may also facilitate purification of fusion proteins using commercially available affinity matrices. Such moieties include, but are not limited to, glutathione S-transferase (GST), maltose binding protein (MBP), thioredoxin (Trx), calmodulin binding peptide (CBP), 6-His, FLAG, c-myc, and hemagglutinin (HA). GST, MBP, Trx, CBP, and 6-His enable purification of their cognate fusion proteins on immobilized glutathione, maltose, phenylarsine oxide, calmodulin, and metal-chelate resins, respectively. FLAG, c-myc, and hemagglutinin (HA) enable immunoaffinity purification of fusion proteins using commercially available monoclonal and polyclonal antibodies that specifically recognize these epitope tags. A fusion protein may also be engineered to contain a proteolytic cleavage site located between the REMAP encoding sequence and the heterologous protein sequence, so that REMAP may be cleaved away from the heterologous moiety following purification. Methods for fusion protein expression and purification are discussed in Ausubel et al. (supra, ch. 10 and 16). A variety of commercially available kits may also be used to facilitate expression and purification of fusion proteins.

In another embodiment, synthesis of radiolabeled REMAP may be achieved in vitro using the TNT rabbit reticulocyte lysate or wheat germ extract system (Promega). These systems couple transcription and translation of protein-coding sequences operably associated with the T7, T3, or SP6 promoters. Translation takes place in the presence of a radiolabeled amino acid precursor, for example, ³⁵S-methionine.

REMAP, fragments of REMAP, or variants of REMAP may be used to screen for compounds that specifically bind to REMAP. One or more test compounds may be screened for specific binding to REMAP. In various embodiments, 1, 2, 3, 4, 5, 10, 20, 50, 100, or 200 test compounds can be screened for specific binding to REMAP. Examples of test compounds can include antibodies, anticalins, oligonucleotides, proteins (e.g., ligands or receptors), or small molecules.

In related embodiments, variants of REMAP can be used to screen for binding of test compounds, such as antibodies, to REMAP, a variant of REMAP, or a combination of REMAP and/or one or more variants REMAP. In an embodiment, a variant of REMAP can be used to screen for compounds that bind to a variant of REMAP, but not to REMAP having the exact sequence of a sequence of SEQ ID NO:1-47. REMAP variants used to perform such screening can have a range of about 50% to about 99% sequence identity to REMAP, with various embodiments having 60%, 70%, 75%, 80%, 85%, 90%, and 95% sequence identity.

In an embodiment, a compound identified in a screen for specific binding to REMAP can be closely related to the natural ligand of REMAP, e.g., a ligand or fragment thereof, a natural substrate, a structural or functional mimetic, or a natural binding partner (Coligan, J. E. et al. (1991) Current Protocols in Immunology 1(2):Chapter 5). In another embodiment, the compound thus identified can be a natural ligand of a receptor REMAP (Howard, A. D. et al. (2001) Trends Pharmacol. Sci. 22:132-140; Wise, A. et al. (2002) Drug Discovery Today 7:235-246).

In other embodiments, a compound identified in a screen for specific binding to REMAP can be closely related to the natural receptor to which REMAP binds, at least a fragment of the receptor, or a fragment of the receptor including all or a portion of the ligand binding site or binding pocket. For example, the compound may be a receptor for REMAP which is capable of propagating a signal, or a decoy receptor for REMAP which is not capable of propagating a signal (Ashkenazi, A. and V. M. Divit (1999) Curr. Opin. Cell Biol. 11:255-260; Mantovani, A. et al. (2001) Trends Immunol. 22:328-336). The compound can be rationally designed using known techniques. Examples of such techniques include those used to construct the compound etanercept (ENBREL; Amgen Inc., Thousand Oaks Calif.), which is efficacious for treating rheumatoid arthritis in humans. Etanercept is an engineered p75 tumor necrosis factor (TNF) receptor dimer linked to the Fc portion of human IgG, (Taylor, P. C. et al. (2001) Curr. Opin. Immunol. 13:611-616).

In one embodiment, two or more antibodies having similar or, alternatively, different specificities can be screened for specific binding to REMAP, fragments of REMAP, or variants of REMAP. The binding specificity of the antibodies thus screened can thereby be selected to identify particular fragments or variants of REMAP. In one embodiment, an antibody can be selected such that its binding specificity allows for preferential identification of specific fragments or variants of REMAP. In another embodiment, an antibody can be selected such that its binding specificity allows for preferential diagnosis of a specific disease or condition having increased, decreased, or otherwise abnormal production of REMAP.

In an embodiment, anticalins can be screened for specific binding to REMAP, fragments of REMAP, or variants of REMAP. Anticalins are ligand-binding proteins that have been constructed based on a lipocalin scaffold (Weiss, G. A. and H. B. Lowman (2000) Chem. Biol. 7:R177-R184; Skerra, A. (2001) J. Biotechnol. 74:257-275). The protein architecture of lipocalins can include a beta-barrel having eight antiparallel beta-strands, which supports four loops at its open end. These loops form the natural ligand-binding site of the lipocalins, a site which can be re-engineered in vitro by amino acid substitutions to impart novel binding specificities. The amino acid substitutions can be made using methods known in the art or described herein, and can include conservative substitutions (e.g., substitutions that do not alter binding specificity) or substitutions that modestly, moderately, or significantly alter binding specificity.

In one embodiment, screening for compounds which specifically bind to, stimulate, or inhibit REMAP involves producing appropriate cells which express REMAP, either as a secreted protein or on the cell membrane. Preferred cells can include cells from mammals, yeast, Drosophila, or E. coli. Cells expressing REMAP or cell membrane fractions which contain REMAP are then contacted with a test compound and binding, stimulation, or inhibition of activity of either REMAP or the compound is analyzed.

An assay may simply test binding of a test compound to the polypeptide, wherein binding is detected by a fluorophore, radioisotope, enzyme conjugate, or other detectable label. For example, the assay may comprise the steps of combining at least one test compound with REMAP, either in solution or affixed to a solid support, and detecting the binding of REMAP to the compound. Alternatively, the assay may detect or measure binding of a test compound in the presence of a labeled competitor. Additionally, the assay may be carried out using cell-free preparations, chemical libraries, or natural product mixtures, and the test compound(s) may be free in solution or affixed to a solid support.

An assay can be used to assess the ability of a compound to bind to its natural ligand and/or to inhibit the binding of its natural ligand to its natural receptors. Examples of such assays include radio-labeling assays such as those described in U.S. Pat. No. 5,914,236 and U.S. Pat. No. 6,372,724. In a related embodiment, one or more amino acid substitutions can be introduced into a polypeptide compound (such as a receptor) to improve or alter its ability to bind to its natural ligands (Matthews, D. J. and J. A. Wells. (1994) Chem. Biol. 1:25-30). In another related embodiment, one or more amino acid substitutions can be introduced into a polypeptide compound (such as a ligand) to improve or alter its ability to bind to its natural receptors (Cunningham, B. C. and J. A. Wells (1991) Proc. Natl. Acad. Sci. USA 88:3407-3411; Lowman, H. B. et al. (1991) J. Biol. Chem. 266:10982-10988).

REMAP, fragments of REMAP, or variants of REMAP may be used to screen for compounds that modulate the activity of REMAP. Such compounds may include agonists, antagonists, or partial or inverse agonists. In one embodiment, an assay is performed under conditions permissive for REMAP activity, wherein REMAP is combined with at least one test compound, and the activity of REMAP in the presence of a test compound is compared with the activity of REMAP in the absence of the test compound. A change in the activity of REMAP in the presence of the test compound is indicative of a compound that modulates the activity of REMAP. Alternatively, a test compound is combined with an in vitro or cell-free system comprising REMAP under conditions suitable for REMAP activity, and the assay is performed. In either of these assays, a test compound which modulates the activity of REMAP may do so indirectly and need not come in direct contact with the test compound. At least one and up to a plurality of test compounds may be screened.

In another embodiment, polynucleotides encoding REMAP or their mammalian homologs may be “knocked out” in an animal model system using homologous recombination in embryonic stem (ES) cells. Such techniques are well known in the art and are useful for the generation of animal models of human disease (see, e.g., U.S. Pat. No. 5,175,383 and U.S. Pat. No. 5,767,337). For example, mouse ES cells, such as the mouse 129/SvJ cell line, are derived from the early mouse embryo and grown in culture. The ES cells are transformed with a vector containing the gene of interest disrupted by a marker gene, e.g., the neomycin phosphotransferase gene (neo; Capecchi, M. R. (1989) Science 244:1288-1292). The vector integrates into the corresponding region of the host genome by homologous recombination. Alternatively, homologous recombination takes place using the Cre-loxP system to knockout a gene of interest in a tissue- or developmental stage-specific manner (Marth, J. D. (1996) Clin. Invest. 97:1999-2002; Wagner, K. U. et al. (1997) Nucleic Acids Res. 25:4323-4330). Transformed ES cells are identified and microinjected into mouse cell blastocysts such as those from the C57BL/6 mouse strain. The blastocysts are surgically transferred to pseudopregnant dams, and the resulting chimeric progeny are genotyped and bred to produce heterozygous or homozygous strains. Transgenic animals thus generated may be tested with potential therapeutic or toxic agents.

Polynucleotides encoding REMAP may also be manipulated in vitro in ES cells derived from human blastocysts. Human ES cells have the potential to differentiate into at least eight separate cell lineages including endoderm, mesoderm, and ectodermal cell types. These cell lineages differentiate into, for example, neural cells, hematopoietic lineages, and cardiomyocytes (Thomson, J. A. et al. (1998) Science 282:1145-1147).

Polynucleotides encoding REMAP can also be used to create “knockin” humanized animals (pigs) or transgenic animals (mice or rats) to model human disease. With knockin technology, a region of a polynucleotide encoding REMAP is injected into animal ES cells, and the injected sequence integrates into the animal cell genome. Transformed cells are injected into blastulae, and the blastulae are implanted as described above. Transgenic progeny or inbred lines are studied and treated with potential pharmaceutical agents to obtain information on treatment of a human disease. Alternatively, a mammal inbred to overexpress REMAP, e.g., by secreting REMAP in its milk, may also serve as a convenient source of that protein (Janne, J. et al. (1998) Biotechnol. Annu. Rev. 4:55-74).

Therapeutics

Chemical and structural similarity, e.g., in the context of sequences and motifs, exists between regions of REMAP and receptors and membrane-associated proteins. In addition, examples of tissues expressing REMAP can be found in Table 6 and can also be found in Example XI. Therefore, REMAP appears to play a role in cell proliferative, autoimmune/inflammatory, neurological, metabolic, developmental, and endocrine disorders. In the treatment of disorders associated with increased REMAP expression or activity, it is desirable to decrease the expression or activity of REMAP. In the treatment of disorders associated with decreased REMAP expression or activity, it is desirable to increase the expression or activity of REMAP.

Therefore, in one embodiment, REMAP or a fragment or derivative thereof may be administered to a subject to treat or prevent a disorder associated with decreased expression or activity of REMAP. Examples of such disorders include, but are not limited to, a cell proliferative disorder such as actinic keratosis, arteriosclerosis, atherosclerosis, bursitis, cirrhosis, hepatitis, mixed connective tissue disease (MCTD), myelofibrosis, paroxysmal nocturnal hemoglobinuria, polycythemia vera, psoriasis, primary thrombocythemia, and cancers including adenocarcinoma, leukemia, lymphoma, melanoma, myeloma, sarcoma, teratocarcinoma, and, in particular, cancers of the adrenal gland, bladder, bone, bone marrow, brain, breast, cervix, colon, gall bladder, ganglia, gastrointestinal tract, heart, kidney, liver, lung, muscle, ovary, pancreas, parathyroid, penis, prostate, salivary glands, skin, spleen, testis, thymus, thyroid, and uterus; an autoimmune/inflammatory disorder such as acquired immunodeficiency syndrome (AIDS), Addison's disease, adult respiratory distress syndrome, allergies, ankylosing spondylitis, amyloidosis, anemia, asthma, atherosclerosis, autoimmune hemolytic anemia, autoimmune thyroiditis, autoimmune polyendocrinopathy-candidiasis-ectodermal dystrophy (APECED), bronchitis, cholecystitis, contact dermatitis, Crohn's disease, atopic dermatitis, dermatomyositis, diabetes mellitus, emphysema, episodic lymphopenia with lymphocytotoxins, erythroblastosis fetalis, erythema nodosum, atrophic gastritis, glomerulonephritis, Goodpasture's syndrome, gout, Graves' disease, Hashimoto's thyroiditis, hypereosinophilia, irritable bowel syndrome, multiple sclerosis, myasthenia gravis, myocardial or pericardial inflammation, osteoarthritis, osteoporosis, pancreatitis, polymyositis, psoriasis, Reiter's syndrome, rheumatoid arthritis, scleroderma, Sjögren's syndrome, systemic anaphylaxis, systemic lupus erythematosus, systemic sclerosis, thrombocytopenic purpura, ulcerative colitis, uveitis, Werner syndrome, complications of cancer, hemodialysis, and extracorporeal circulation, viral, bacterial, fungal, parasitic, protozoal, and helminthic infections, and trauma; a neurological disorder such as epilepsy, ischemic cerebrovascular disease, stroke, cerebral neoplasms, Alzheimer's disease, Pick's disease, Huntington's disease, dementia, Parkinson's disease and other extrapyramidal disorders, amyotrophic lateral sclerosis and other motor neuron disorders, progressive neural muscular atrophy, retinitis pigmentosa, hereditary ataxias, multiple sclerosis and other demyelinating diseases, bacterial and viral meningitis, brain abscess, subdural empyema, epidural abscess, suppurative intracranial thrombophlebitis, myelitis and radiculitis, viral central nervous system disease, prion diseases including kuru, Creutzfeldt-Jakob disease, and Gerstmann-Straussler-Scheinker syndrome, fatal familial insomnia, nutritional and metabolic diseases of the nervous system, neurofibromatosis, tuberous sclerosis, cerebelloretinal hemangioblastomatosis, encephalotrigerninal syndrome, mental retardation and other developmental disorders of the central nervous system, cerebral palsy, neuroskeletal disorders, autonomic nervous system disorders, cranial nerve disorders, spinal cord diseases, muscular dystrophy and other neuromuscular disorders, peripheral nervous system disorders, dermatomyositis and polymyositis, inherited, metabolic, endocrine, and toxic myopathies, myasthenia gravis, periodic paralysis, mental disorders including mood, anxiety, and schizophrenic disorders, seasonal affective disorder (SAD), akathesia, amnesia, catatonia, diabetic neuropathy, tardive dyskinesia, dystonias, paranoid psychoses, postherpetic neuralgia, Tourette's disorder, progressive supranuclear palsy, corticobasal degeneration, and familial frontotemporal dementia; a metabolic disorder such as Addison's disease, cerebrotendinous xanthomatosis, congenital adrenal hyperplasia, coumarin resistance, cystic fibrosis, fatty hepatocirrhosis, fructose-1,6-diphosphatase deficiency, galactosemia, goiter, glucagonoma, glycogen storage diseases, hereditary fructose intolerance, hyperadrenalism, hypoadrenalism, hyperparathyroidism, hypoparathyroidism, hypercholesterolemia, hyperthyroidism, hypoglycemia, hypothyroidism, hyperlipidemia, hyperlipemia, lipid myopathies, lipodystrophies, lysosomal storage diseases, mannosidosis, neuraminidase deficiency, obesity, osteoporosis, phenylketonuria, pseudovitamin D-deficiency rickets, disorders of carbohydrate metabolism such as congenital type II dyserythropoietic anemia, diabetes, insulin-dependent diabetes mellitus, non-insulin-dependent diabetes mellitus, galactose epimerase deficiency, glycogen storage diseases, lysosomal storage diseases, fructosuria, pentosuria, and inherited abnormalities of pyruvate metabolism, disorders of lipid metabolism such as fatty liver, cholestasis, primary biliary cirrhosis, carnitine deficiency, carnitine palmitoyltransferase deficiency, myoadenylate deaminase deficiency, hypertriglyceridemia, lipid storage disorders such Fabry's disease, Gaucher's disease, Niemann-Pick's disease, metachromatic leukodystrophy, adrenoleukodystrophy, GM₂ gangliosidosis, and ceroid lipofuscinosis, abetalipoproteinemia, Tangier disease, hyperlipoproteinemia, lipodystrophy, lipomatoses, acute panniculitis, disseminated fat necrosis, adiposis dolorosa, lipoid adrenal hyperplasia, minimal change disease, lipomas, atherosclerosis, hypercholesterolemia, hypercholesterolemia with hypertriglyceridemia, primary hypoalphalipoproteinemia, hypothyroidism, renal disease, liver disease, lecithin:cholesterol acyltransferase deficiency, cerebrotendinous xanthomatosis, sitosterolemia, hypocholesterolemia, Tay-Sachs disease, Sandhoff's disease, hyperlipidemia, hyperlipemia, and lipid myopathies, and disorders of copper metabolism such as Menke's disease, Wilson's disease, and Ehlers-Danlos syndrome type DX diabetes; a developmental disorder such as renal tubular acidosis, anemia, Cushing's syndrome, achondroplastic dwarfism, Duchenne and Becker muscular dystrophy, epilepsy, gonadal dysgenesis, WAGR syndrome (Wilms' tumor, aniridia, genitourinary abnormalities, and mental retardation), Smith-Magenis syndrome, myelodysplastic syndrome, hereditary mucoepithelial dysplasia, hereditary keratodermas, hereditary neuropathies such as Charcot-Marie-Tooth disease and neurofibromatosis, hypothyroidism, hydrocephalus, a seizure disorder such as Syndenham's chorea and cerebral palsy, spina bifida, anencephaly, craniorachischisis, congenital glaucoma, cataract, and sensorineural hearing loss; and an endocrine disorder such as a disorder of the hypothalamus and/or pituitary resulting from lesions such as a primary brain tumor, adenoma, infarction associated with pregnancy, hypophysectomy, aneurysm, vascular malformation, thrombosis, infection, immunological disorder, and complication due to head trauma, a disorder associated with hypopituitarism including hypogonadism, Sheehan syndrome, diabetes insipidus, Kallman's disease, Hand-Schuller-Christian disease, Letterer-Siwe disease, sarcoidosis, empty sella syndrome, and dwarfism, a disorder associated with hyperpituitarism including acromegaly, giantism, and syndrome of inappropriate antidiuretic hormone (ADH) secretion (SLIDH) often caused by benign adenoma, a disorder associated with hypothyroidism including goiter, myxedema, acute thyroiditis associated with bacterial infection, subacute thyroiditis associated with viral infection, autoimmune thyroiditis (Hashinoto's disease), and cretinism, a disorder associated with hyperthyroidism including thyrotoxicosis and its various forms, Grave's disease, pretibial myxedema, toxic multinodular goiter, thyroid carcinoma, and Plummer's disease, a disorder associated with hyperparathyroidism including Conn disease (chronic hypercalemia), a pancreatic disorder such as Type I or Type II diabetes mellitus and associated complications, a disorder associated with the adrenals such as hyperplasia, carcinoma, or adenoma of the adrenal cortex, hypertension associated with alkalosis, amyloidosis, hypokalemia, Cushing's disease, Liddle's syndrome, and Arnold-Healy-Gordon syndrome, pheochromocytoma tumors, and Addison's disease, a disorder associated with gonadal steroid hormones such as: in women, abnormal prolactin production, infertility, endometriosis, perturbation of the menstrual cycle, polycystic ovarian disease, hyperprolactinemia, isolated gonadotropin deficiency, amenorrhea, galactorrhea, hermaphroditism, hirsutism and virilization, breast cancer, and, in post-menopausal women, osteoporosis, and, in men, Leydig cell deficiency, male climacteric phase, and germinal cell aplasia, a hypergonadal disorder associated with Leydig cell tumors, androgen resistance associated with absence of androgen receptors, syndrome of 5 α-reductase, and gynecomastia.

In another embodiment, a vector capable of expressing REMAP or a fragment or derivative thereof may be administered to a subject to treat or prevent a disorder associated with decreased expression or activity of REMAP including, but not limited to, those described above.

In a further embodiment, a composition comprising a substantially purified REMAP in conjunction with a suitable pharmaceutical carrier may be administered to a subject to treat or prevent a disorder associated with decreased expression or activity of REMAP including, but not limited to, those provided above.

In still another embodiment, an agonist which modulates the activity of REMAP may be administered to a subject to treat or prevent a disorder associated with decreased expression or activity of REMAP including, but not limited to, those listed above.

In a further embodiment, an antagonist of REMAP may be administered to a subject to treat or prevent a disorder associated with increased expression or activity of REMAP. Examples of such disorders include, but are not limited to, those cell proliferative, autoimmune/inflammatory, neurological, metabolic, developmental, and endocrine disorders described above. In one aspect, an antibody which specifically binds REMAP may be used directly as an antagonist or indirectly as a targeting or delivery mechanism for bringing a pharmaceutical agent to cells or tissues which express REMAP.

In an additional embodiment, a vector expressing the complement of the polynucleotide encoding REMAP may be administered to a subject to treat or prevent a disorder associated with increased expression or activity of REMAP including, but not limited to, those described above.

In other embodiments, any protein, agonist, antagonist, antibody, complementary sequence, or vector embodiments may be administered in combination with other appropriate therapeutic agents. Selection of the appropriate agents for use in combination therapy may be made by one of ordinary skill in the art, according to conventional pharmaceutical principles. The combination of therapeutic agents may act synergistically to effect the treatment or prevention of the various disorders described above. Using this approach, one may be able to achieve therapeutic efficacy with lower dosages of each agent, thus reducing the potential for adverse side effects.

An antagonist of REMAP may be produced using methods which are generally known in the art. In particular, purified REMAP may be used to produce antibodies or to screen libraries of pharmaceutical agents to identify those which specifically bind REMAP. Antibodies to REMAP may also be generated using methods that are well known in the art. Such antibodies may include, but are not limited to, polyclonal, monoclonal, chimeric, and single chain antibodies, Fab fragments, and fragments produced by a Fab expression library. In an embodiment, neutralizing antibodies (i.e., those which inhibit dimer formation) can be used therapeutically. Single chain antibodies (e.g., from camels or llamas) may be potent enzyme inhibitors and may have application in the design of peptide mimetics, and in the development of immuno-adsorbents and biosensors (Muyldermans, S. (2001) J. Biotechnol. 74:277-302).

For the production of antibodies, various hosts including goats, rabbits, rats, mice, camels, dromedaries, llamas, humans, and others may be immunized by injection with REMAP or with any fragment or oligopeptide thereof which has immunogenic properties. Depending on the host species, various adjuvants may be used to increase immunological response. Such adjuvants include, but are not limited to, Freund's, mineral gels such as aluminum hydroxide, and surface active substances such as lysolecithin, pluronic polyols, polyanions, peptides, oil emulsions, KLH, and dinitrophenol. Among adjuvants used in humans, BCG (bacilli Calmette-Guerin) and Corynebacterium parvum are especially preferable.

It is preferred that the oligopeptides, peptides, or fragments used to induce antibodies to REMAP have an amino acid sequence consisting of at least about 5 amino acids, and generally will consist of at least about 10 amino acids. It is also preferable that these oligopeptides, peptides, or fragments are substantially identical to a portion of the amino acid sequence of the natural protein. Short stretches of REMAP amino acids may be fused with those of another protein, such as KLH, and antibodies to the chimeric molecule may be produced.

Monoclonal antibodies to REMAP may be prepared using any technique which provides for the production of antibody molecules by continuous cell lines in culture. These include, but are not limited to, the hybridoma technique, the human B-cell hybridoma technique, and the EBV-hybridoma technique (Kohler, G. et al. (1975) Nature 256:495-497; Kozbor, D. et al. (1985) J. Immunol. Methods 81:31-42; Cote, R. J. et al. (1983) Proc. Natl. Acad. Sci. USA 80:2026-2030; Cole, S. P. et al. (1984) Mol. Cell Biol. 62:109-120).

In addition, techniques developed for the production of “chimeric antibodies,” such as the splicing of mouse antibody genes to human antibody genes to obtain a molecule with appropriate antigen specificity and biological activity, can be used (Morrison, S. L. et al. (1984) Proc. Natl. Acad. Sci. USA 81:6851-6855; Neuberger, M. S. et al. (1984) Nature 312:604-608; Takeda, S. et al. (1985) Nature 314:452-454). Alternatively, techniques described for the production of single chain antibodies may be adapted, using methods known in the art, to produce REMAP-specific single chain antibodies. Antibodies with related specificity, but of distinct idiotypic composition, may be generated by chain shuffling from random combinatorial immunoglobulin libraries (Burton, D. R. (1991) Proc. Natl. Acad. Sci. USA 88:10134-10137).

Antibodies may also be produced by inducing in vivo production in the lymphocyte population or by screening immunoglobulin libraries or panels of highly specific binding reagents as disclosed in the literature (Orlandi, R. et al. (1989) Proc. Natl. Acad. Sci. USA 86:3833-3837; Winter, G. et al. (1991) Nature 349:293-299).

Antibody fragments which contain specific binding sites for REMAP may also be generated. For example, such fragments include, but are not limited to, F(ab′)₂ fragments produced by pepsin digestion of the antibody molecule and Fab fragments generated by reducing the disulfide bridges of the F(ab′)₂ fragments. Alternatively, Fab expression libraries may be constructed to allow rapid and easy identification of monoclonal Fab fragments with the desired specificity (Huse, W. D. et al. (1989) Science 246:1275-1281).

Various immunoassays may be used for screening to identify antibodies having the desired specificity. Numerous protocols for competitive binding or immunoradiometric assays using either polyclonal or monoclonal antibodies with established specificities are well known in the art. Such immunoassays typically involve the measurement of complex formation between REMAP and its specific antibody. A two-site, monoclonal-based immunoassay utilizing monoclonal antibodies reactive employed (Pound, supra).

Various methods such as Scatchard analysis in conjunction with radioimmunoassay techniques may be used to assess the affinity of antibodies for REMAP. Affinity is expressed as an association constant, Kₐ, which is defined as the molar concentration of REMAP-antibody complex divided by the molar concentrations of free antigen and free antibody under equilibrium conditions. The Kₐ determined for a preparation of polyclonal antibodies, which are heterogeneous in their affinities for multiple REMAP epitopes, represents the average affinity, or avidity, of the antibodies for REMAP. The Kₐ determined for a preparation of monoclonal antibodies, which are monospecific for a particular REMAP epitope, represents a true measure of affinity. High-affinity antibody preparations with Kₐ ranging from about 10⁹ to 10¹² L/mole are preferred for use in immunoassays in which the REMAP-antibody complex must withstand rigorous manipulations. Low-affinity antibody preparations with Kₐ ranging from about 10⁶ to 10⁷ L/mole are preferred for use in immunopurification and similar procedures which ultimately require dissociation of REMAP, preferably in active form, from the antibody (Catty, D. (1988) Antibodies, Volume I: A Practical Approach, IRL Press, Washington D.C.; Liddell, J. E. and A. Cryer (1991) A Practical Guide to Monoclonal Antibodies, John Wiley & Sons, New York N.Y.).

The titer and avidity of polyclonal antibody preparations may be further evaluated to determine the quality and suitability of such preparations for certain downstream applications. For example, a polyclonal antibody preparation containing at least 1-2 mg specific antibody/ml, preferably 5-10 mg specific antibody/ml, is generally employed in procedures requiring precipitation of REMAP-antibody complexes. Procedures for evaluating antibody specificity, titer, and avidity, and guidelines for antibody quality and usage in various applications, are generally available (Catty, supra; Coligan et al., supra).

In another embodiment of the invention, polynucleotides encoding REMAP, or any fragment or complement thereof, may be used for therapeutic purposes. In one aspect, modifications of gene expression can be achieved by designing complementary sequences or antisense molecules (DNA, RNA, PNA, or modified oligonucleotides) to the coding or regulatory regions of the gene encoding REMAP. Such technology is well known in the art, and antisense oligonucleotides or larger fragments can be designed from various locations along the coding or control regions of sequences encoding REMAP (Agrawal, S., ed. (1996) Antisense Therapeutics, Humana Press, Totawa N.J.).

In therapeutic use, any gene delivery system suitable for introduction of the antisense sequences into appropriate target cells can be used. Antisense sequences can be delivered intracellularly in the form of an expression plasmid which, upon transcription, produces a sequence complementary to at least a portion of the cellular sequence encoding the target protein (Slater, J. E. et al. (1998) J. Allergy Clin. Immunol. 102:469-475; Scanlon, K. J. et al. (1995) 9:1288-1296). Antisense sequences can also be introduced intracellularly through the use of viral vectors, such as retrovirus and adeno-associated virus vectors (Miller, A. D. (1990) Blood 76:271; Ausubel et al., supra; Uckert, W. and W. Walther (1994) Pharmacol. Ther. 63:323-347). Other gene delivery mechanisms include liposome-derived systems, artificial viral envelopes, and other systems known in the art (Rossi, J. J. (1995) Br. Med. Bull. 51:217-225; Boado, R. J. et al. (1998) J. Pharm. Sci. 87:1308-1315; Morris, M. C. et al. (1997) Nucleic Acids Res. 25:2730-2736).

In another embodiment of the invention, polynucleotides encoding REMAP may be used for somatic or germline gene therapy. Gene therapy may be performed to (i) correct a genetic deficiency (e.g., in the cases of severe combined immunodeficiency (SCID)-X1 disease characterized by X-linked inheritance (Cavazzana-Calvo, M. et al. (2000) Science 288:669-672), severe combined immunodeficiency syndrome associated with an inherited adenosine deaminase (ADA) deficiency (Blaese, R. M. et al. (1995) Science 270:475-480; Bordignon, C. et al. (1995) Science 270:470-475), cystic fibrosis (Zabner, J. et al. (1993) Cell 75:207-216; Crystal, R. G. et al. (1995) Hum. Gene Therapy 6:643-666; Crystal, R. G. et al. (1995) Hum. Gene Therapy 6:667-703), thalassamias, familial hypercholesterolemia, and hemophilia resulting from Factor VIII or Factor IX deficiencies (Crystal, R. G. (1995) Science 270:404-410; Verma, I. M. and N. Somia (1997) Nature 389:239-242)), (ii) express a conditionally lethal gene product (e.g., in the case of cancers which result from unregulated cell proliferation), or (iii) express a protein which affords protection against intracellular parasites (e.g., against human retroviruses, such as human immunodeficiency virus (HIV) (Baltimore, D. (1988) Nature 335:395-396; Poeschla, E. et al. (1996) Proc. Natl. Acad. Sci. USA 93:11395-11399), hepatitis B or C virus (HBV, HCV); fungal parasites, such as Candida albicans and Paracoccidioides brasiliensis; and protozoan parasites such as Plasmodium falciparum and Trypanosoma cruzi). In the case where a genetic deficiency in REMAP expression or regulation causes disease, the expression of REMAP from an appropriate population of transduced cells may alleviate the clinical manifestations caused by the genetic deficiency.

In a further embodiment of the invention, diseases or disorders caused by deficiencies in REMAP are treated by constructing mammalian expression vectors encoding REMAP and introducing these vectors by mechanical means into REMAP-deficient cells. Mechanical transfer technologies for use with cells in vivo or ex vitro include (i) direct DNA microinjection into individual cells, (ii) ballistic gold particle delivery, (iii) liposome-mediated transfection, (iv) receptor-mediated gene transfer, and (v) the use of DNA transposons (Morgan, R. A. and W. F. Anderson (1993) Annu. Rev. Biochem. 62:191-217; Ivics, Z. (1997) Cell 91:501-510; Boulay, J. -L. and H. Récipon (1998) Curr. Opin. Biotechnol. 9:445-450).

Expression vectors that may be effective for the expression of REMAP include, but are not limited to, the PCDNA 3.1, EPITAG, PRCCMV2, PREP, PVAX, PCR2-TOPOTA vectors (Invitrogen, Carlsbad Calif.), PCMV-SCRIPT, PCMV-TAG, PEGSH/PERV (Stratagene, La Jolla Calif.), and PTET-OFF, PTET-ON, PTRE2, PTRE2-LUC, PTK-HYG (Clontech, Palo Alto Calif.). REMAP may be expressed using (i) a constitutively active promoter, (e.g., from cytomegalovirus (CMV), Rous sarcoma virus (RSV), SV40 virus, thymidine kinase (TK), or β-actin genes), (ii) an inducible promoter (e.g., the tetracycline-regulated promoter (Gossen, M. and H. Bujard (1992) Proc. Natl. Acad. Sci. USA 89:5547-5551; Gossen, M. et al. (1995) Science 268:1766-1769; Rossi, F. M. V. and H. M. Blau (1998) Curr. Opin. Biotechnol. 9:451-456), commercially available in the T-REX plasmid (Invitrogen)); the ecdysone-inducible promoter (available in the plasmids PVGRXR and PIND; Invitrogen); the FK506/rapamycin inducible promoter; or the RU486/mifepristone inducible promoter (Rossi, F. M. V. and H. M. Blau, supra)), or (iii) a tissue-specific promoter or the native promoter of the endogenous gene encoding REMAP from a normal individual.

Commercially available liposome transformation kits (e.g., the PERFECT LIPID TRANSFECTION KIT, available from Invitrogen) allow one with ordinary skill in the art to deliver polynucleotides to target cells in culture and require minimal effort to optimize experimental parameters. In the alternative, transformation is performed using the calcium phosphate method (Graham, F. L. and A. J. Eb (1973) Virology 52:456-467), or by electroporation (Neumann, E. et al. (1982) EMBO J. 1:841-845). The introduction of DNA to primary cells requires modification of these standardized mammalian transfection protocols.

In another embodiment of the invention, diseases or disorders caused by genetic defects with respect to REMAP expression are treated by constructing a retrovirus vector consisting of (i) the polynucleotide encoding REMAP under the control of an independent promoter or the retrovirus long terminal repeat (LTR) promoter, (bi) appropriate RNA packaging signals, and (iii) a Rev-responsive element (RRE) along with additional retrovirus cis-acting RNA sequences and coding sequences required for efficient vector propagation. Retrovirus vectors (e.g., PFB and PFBNEO) are commercially available (Stratagene) and are based on published data (Riviere, I. et al. (1995) Proc. Natl. Acad. Sci. USA 92:6733-6737), incorporated by reference herein. The vector is propagated in an appropriate vector producing cell line (VPCL) that expresses an envelope gene with a tropism for receptors on the target cells or a promiscuous envelope protein such as VSVg (Armentano, D. et al. (1987) J. Virol. 61:1647-1650; Bender, M. A. et al. (1987) J. Virol. 61:1639-1646; Adam, M. A. and A. D. Miller (1988) J. Virol. 62:3802-3806; Dull, T. et al. (1998) J. Virol. 72:8463-8471; Zufferey, R. et al. (1998) J. Virol. 72:9873-9880). U.S. Pat. No. 5,910,434 to Rigg (“Method for obtaining retrovirus packaging cell lines producing high transducing efficiency retroviral supernatant”) discloses a method for obtaining retrovirus packaging cell lines and is hereby incorporated by reference. Propagation of retrovirus vectors, transduction of a population of cells (e.g., CD4⁺ T-cells), and the return of transduced cells to a patient are procedures well known to persons skilled in the art of gene therapy and have been well documented (Ranga, U. et al. (1997) J. Virol. 71:7020-7029; Bauer, G. et al. (1997) Blood 89:2259-2267; Bonyhadi, M. L. (1997) J. Virol. 71:4707-4716; Ranga, U. et al. (1998) Proc. Natl. Acad. Sci. USA 95:1201-1206; Su, L. (1997) Blood 89:2283-2290).

In an embodiment, an adenovirus-based gene therapy delivery system is used to deliver polynucleotides encoding REMAP to cells which have one or more genetic abnormalities with respect to the expression of REMAP. The construction and packaging of adenovirus-based vectors are well known to those with ordinary skill in the art. Replication defective adenovirus vectors have proven to be versatile for importing genes encoding immunoregulatory proteins into intact islets in the pancreas (Csete, M. E. et al. (1995) Transplantation 27:263-268). Potentially useful adenoviral vectors are described in U.S. Pat. No. 5,707,618 to Armentano (“Adenovirus vectors for gene therapy”), hereby incorporated by reference. For adenoviral vectors, see also Antinozzi, P. A. et al. (1999; Annu. Rev. Nutr. 19:511-544) and Verma, I. M. and N. Somia (1997; Nature 18:389:239-242).

In another embodiment, a herpes-based, gene therapy delivery system is used to deliver polynucleotides encoding REMAP to target cells which have one or more genetic abnormalities with respect to the expression of REMAP. The use of herpes simplex virus (HSV)-based vectors may be especially valuable for introducing REMAP to cells of the central nervous system, for which HSV has a tropism. The construction and packaging of herpes-based vectors are well known to those with ordinary skill in the art. A replication-competent herpes simplex virus (HSV) type 1-based vector has been used to deliver a reporter gene to the eyes of primates (Liu, X. et al. (1999) Exp. Eye Res. 169:385-395). The construction of a HSV-1 virus vector has also been disclosed in detail in U.S. Pat. No. 5,804,413 to DeLuca (“Herpes simplex virus strains for gene transfer”), which is hereby incorporated by reference. U.S. Pat. No. 5,804,413 teaches the use of recombinant HSV d92 which consists of a genome containing at least one exogenous gene to be transferred to a cell under the control of the appropriate promoter for purposes including human gene therapy. Also taught by this patent are the construction and use of recombinant HSV strains deleted for ICP4, ICP27 and ICP22. For HSV vectors, see also Goins, W. F. et al. (1999; J. Virol. 73:519-532) and Xu, H. et al. (1994; Dev. Biol. 163:152-161). The manipulation of cloned herpesvirus sequences, the generation of recombinant virus following the transfection of multiple plasmids containing different segments of the large herpesvirus genomes, the growth and propagation of herpesvirus, and the infection of cells with herpesvirus are techniques well known to those of ordinary skill in the art.

In another embodiment, an alphavirus (positive, single-stranded RNA virus) vector is used to deliver polynucleotides encoding REMAP to target cells. The biology of the prototypic alphavirus, Semliki Forest Virus (SFV), has been studied extensively and gene transfer vectors have been based on the SFV genome (Garoff, H. and K. -J. Li (1998) Curr. Opin. Biotechnol. 9:464-469). During alphavinus RNA replication, a subgenomic RNA is generated that normally encodes the viral capsid proteins. This subgenomic RNA replicates to higher levels than the full length genomic RNA, resulting in the overproduction of capsid proteins relative to the viral proteins with enzymatic activity (e.g., protease and polymerase). Similarly, inserting the coding sequence for REMAP into the alphavirus genome in place of the capsid-coding region results in the production of a large number of REMAP-coding RNAs and the synthesis of high levels of REMAP in vector transduced cells. While alphavirus infection is typically associated with cell lysis within a few days, the ability to establish a persistent infection in hamster normal kidney cells (BHK-21) with a variant of Sindbis virus (SIN) indicates that the lytic replication of alphaviruses can be altered to suit the needs of the gene therapy application (Dryga, S. A. et al. (1997) Virology 228:74-83). The wide host range of alphaviruses will allow the introduction of REMAP into a variety of cell types. The specific transduction of a subset of cells in a population may require the sorting of cells prior to transduction. The methods of manipulating infectious cDNA clones of alphaviruses, performing alphavirus cDNA and RNA transfections, and performing alphavirus infections, are well known to those with ordinary skill in the art.

Oligonucleotides derived from the transcription initiation site, e.g., between about positions −10 and +10 from the start site, may also be employed to inhibit gene expression. Similarly, inhibition can be achieved using triple helix base-pairing methodology. Triple helix pairing is useful because it causes inhibition of the ability of the double helix to open sufficiently for the binding of polymerases, transcription factors, or regulatory molecules. Recent therapeutic advances using triplex DNA have been described in the literature (Gee, J. E. et al. (1994) in Huber, B. E. and B. I. Carr, Molecular and Immunologic Approaches, Futura Publishing, Mt. Kisco N.Y., pp. 163-177). A complementary sequence or antisense molecule may also be designed to block translation of mRNA by preventing the transcript from binding to ribosomes.

Ribozymes, enzymatic RNA molecules, may also be used to catalyze the specific cleavage of RNA. The mechanism of ribozyme action involves sequence-specific hybridization of the ribozyme molecule to complementary target RNA, followed by endonucleolytic cleavage. For example, engineered hammerhead motif ribozyme molecules may specifically and efficiently catalyze endonucleolytic cleavage of RNA molecules encoding REMAP.

Specific ribozyme cleavage sites within any potential RNA target are initially identified by scanning the target molecule for ribozyme cleavage sites, including the following sequences: GUA, GUU, and GUC. Once identified, short RNA sequences of between 15 and 20 ribonucleotides, corresponding to the region of the target gene containing the cleavage site, may be evaluated for secondary structural features which may render the oligonucleotide inoperable. The suitability of candidate targets may also be evaluated by testing accessibility to hybridization with complementary oligonucleotides using ribonuclease protection assays.

Complementary ribonucleic acid molecules and ribozymes may be prepared by any method known in the art for the synthesis of nucleic acid molecules. These include techniques for chemically synthesizing oligonucleotides such as solid phase phosphoramidite chemical synthesis. Alternatively, RNA molecules may be generated by in vitro and in vivo transcription of DNA molecules encoding REMAP. Such DNA sequences may be incorporated into a wide variety of vectors with suitable RNA polymerase promoters such as 17 or SP6. Alternatively, these cDNA constructs that synthesize complementary RNA, constitutively or inducibly, can be introduced into cell lines, cells, or tissues.

RNA molecules may be modified to increase intracellular stability and half-life. Possible modifications include, but are not limited to, the addition of flanking sequences at the 5′ and/or 3′ ends of the molecule, or the use of phosphorothioate or 2′ O-methyl rather than phosphodiesterase linkages within the backbone of the molecule. This concept is inherent in the production of PNAs and can be extended in all of these molecules by the inclusion of nontraditional bases such as inosine, queosine, and wybutosine, as well as acetyl-, methyl-, thio-, and similarly modified forms of adenine, cytidine, guanine, thymine, and uridine which are not as easily recognized by endogenous endonucleases.

In other embodiments of the invention, the expression of one or more selected polynucleotides of the present invention can be altered, inhibited, decreased, or silenced using RNA interference (RNAi) or post-transcriptional gene silencing (PTGS) methods known in the art. RNAi is a post-transcriptional mode of gene silencing in which double-stranded RNA (dsRNA) introduced into a targeted cell specifically suppresses the expression of the homologous gene (i.e., the gene bearing the sequence complementary to the dsRNA). This effectively knocks out or substantially reduces the expression of the targeted gene. PTGS can also be accomplished by use of DNA or DNA fragments as well. RNAi methods are described by Fire, A. et al. (1998; Nature 391:806-811) and Gura, T. (2000; Nature 404:804-808). PTGS can also be initiated by introduction of a complementary segment of DNA into the selected tissue using gene delivery and/or viral vector delivery methods described herein or known in the art.

RNAi can be induced in mammalian cells by the use of small interfering RNA also known as siRNA. SiRNA are shorter segments of dsRNA (typically about 21 to 23 nucleotides in length) that result in vivo from cleavage of introduced dsRNA by the action of an endogenous ribonuclease. SiRNA appear to be the mediators of the RNAi effect in mammals. The most effective siRNAs appear to be 21 nucleotide dsRNAs with 2 nucleotide 3′ overhangs. The use of siRNA for inducing RNAi in mammalian cells is described by Elbashir, S. M. et al. (2001; Nature 411:494-498).

SiRNA can either be generated indirectly by introduction of dsRNA into the targeted cell, or directly by mammalian transfection methods and agents described herein or known in the art (such as liposome-mediated transfection, viral vector methods, or other polynucleotide delivery/introductory methods). Suitable SiRNAs can be selected by examining a transcript of the target polynucleotide (e.g., mRNA) for nucleotide sequences downstream from the AUG start codon and recording the occurrence of each nucleotide and the 3′ adjacent 19 to 23 nucleotides as potential siRNA target sites, with sequences having a 21 nucleotide length being preferred. Regions to be avoided for target siRNA sites include the 5′ and 3′ untranslated regions (UTRs) and regions near the start codon (within 75 bases), as these may be richer in regulatory protein binding sites. UTR-binding proteins and/or translation initiation complexes may interfere with binding of the siRNP endonuclease complex. The selected target sites for siRNA can then be compared to the appropriate genome database (e.g., human, etc.) using BLAST or other sequence comparison algorithms known in the art. Target sequences with significant homology to other coding sequences can be eliminated from consideration. The selected SiRNAs can be produced by chemical synthesis methods known in the art or by in vitro transcription using commercially available methods and kits such as the SILENCER siRNA construction kit (Ambion, Austin Tex.).

In alternative embodiments, long-term gene silencing and/or RNAi effects can be induced in selected tissue using expression vectors that continuously express siRNA. This can be accomplished using expression vectors that are engineered to express hairpin RNAs (shRNAs) using methods known in the art (see, e.g., Brummelkamp, T. R. et al. (2002) Science 296:550-553; and Paddison, P. J. et al. (2002) Genes Dev. 16:948-958). In these and related embodiments, shRNAs can be delivered to target cells using expression vectors known in the art. An example of a suitable expression vector for delivery of siRNA is the PSELENCER1.0-U6 (circular) plasmid (Ambion). Once delivered to the target tissue, shRNAs are processed in vivo into siRNA-like molecules capable of carrying out gene-specific silencing.

In various embodiments, the expression levels of genes targeted by RNAi or PTGS methods can be determined by assays for mRNA and/or protein analysis. Expression levels of the mRNA of a targeted gene, can be determined by northern analysis methods using, for example, the NORTHERNMAX-GLY kit (Ambion); by microarray methods; by PCR methods; by real time PCR methods; and by other RNA/polynucleotide assays known in the art or described herein. Expression levels of the protein encoded by the targeted gene can be determined by Western analysis using standard techniques known in the art.

An additional embodiment of the invention encompasses a method for screening for a compound which is effective in altering expression of a polynucleotide encoding REMAP. Compounds which may be effective in altering expression of a specific polynucleotide may include, but are not limited to, oligonucleotides, antisense oligonucleotides, triple helix-forming oligonucleotides, transcription factors and other polypeptide transcriptional regulators, and non-macromolecular chemical entities which are capable of interacting with specific polynucleotide sequences. Effective compounds may alter polynucleotide expression by acting as either inhibitors or promoters of polynucleotide expression. Thus, in the treatment of disorders associated with increased REMAP expression or activity, a compound which specifically inhibits expression of the polynucleotide encoding REMAP may be therapeutically useful, and in the treatment of disorders associated with decreased REMAP expression or activity, a compound which specifically promotes expression of the polynucleotide encoding REMAP may be therapeutically useful.

In various embodiments, one or more test compounds may be screened for effectiveness in altering expression of a specific polynucleotide. A test compound may be obtained by any method commonly known in the art, including chemical modification of a compound known to be effective in altering polynucleotide expression; selection from an existing, commercially-available or proprietary library of naturally-occurring or non-natural chemical compounds; rational design of a compound based on chemical and/or structural properties of the target polynucleotide; and selection from a library of chemical compounds created combinatorially or randomly. A sample comprising a polynucleotide encoding REMAP is exposed to at least one test compound thus obtained. The sample may comprise, for example, an intact or permeabilized cell, or an in vitro cell-free or reconstituted biochemical system. Alterations in the expression of a polynucleotide encoding REMAP are assayed by any method commonly known in the art. Typically, the expression of a specific nucleotide is detected by hybridization with a probe having a nucleotide sequence complementary to the sequence of the polynucleotide encoding REMAP. The amount of hybridization may be quantified, thus forming the basis for a comparison of the expression of the polynucleotide both with and without exposure to one or more test compounds. Detection of a change in the expression of a polynucleotide exposed to a test compound indicates that the test compound is effective in altering the expression of the polynucleotide. A screen for a compound effective in altering expression of a specific polynucleotide can be carried out, for example, using a Schizosaccharomyces pombe gene expression system (Atkins, D. et al. (1999) U.S. Pat. No. 5,932,435; Arndt, G. M. et al. (2000) Nucleic Acids Res. 28:E15) or a human cell line such as HeLa cell (Clarke, M. L. et al. (2000) Biochem. Biophys. Res. Commun. 268:8-13). A particular embodiment of the present invention involves screening a combinatorial library of oligonucleotides (such as deoxyribonucleotides, ribonucleotides, peptide nucleic acids, and modified oligonucleotides) for antisense activity against a specific polynucleotide sequence (Bruice, T. W. et al. (1997) U.S. Pat. No. 5,686,242; Bruice, T. W. et al. (2000) U.S. Pat. No. 6,022,691).

Many methods for introducing vectors into cells or tissues are available and equally suitable for use in vivo, in vitro, and ex vivo. For ex vivo therapy, vectors may be introduced into stem cells taken from the patient and clonally propagated for autologous transplant back into that same patient. Delivery by transfection, by liposome injections, or by polycationic amino polymers may be achieved using methods which are well known in the art (Goldman, C. K. et al. (1997) Nat. Biotechnol. 15:462-466).

Any of the therapeutic methods described above may be applied to any subject in need of such therapy, including, for example, mammals such as humans, dogs, cats, cows, horses, rabbits, and monkeys.

An additional embodiment of the invention relates to the administration of a composition which generally comprises an active ingredient formulated with a pharmaceutically acceptable excipient. Excipients may include, for example, sugars, starches, celluloses, gums, and proteins. Various formulations are commonly known and are thoroughly discussed in the latest edition of Remington's Pharmaceutical Sciences (Maack Publishing, Easton Pa.). Such compositions may consist of REMAP, antibodies to REMAP, and mimetics, agonists, antagonists, or inhibitors of REMAP.

In various embodiments, the compositions described herein, such as pharmaceutical compositions, may be administered by any number of routes including, but not limited to, oral, intravenous, intramuscular, intra-arterial, intramedullary, intrathecal, intraventricular, pulmonary, transdermal, subcutaneous, intraperitoneal, intranasal, enteral, topical, sublingual, or rectal means.

Compositions for pulmonary administration may be prepared in liquid or dry powder form. These compositions are generally aerosolized immediately prior to inhalation by the patient. In the case of small molecules (e.g. traditional low molecular weight organic drugs), aerosol delivery of fast-acting formulations is well-known in the art. In the case of macromolecules (e.g. larger peptides and proteins), recent developments in the field of pulmonary delivery via the alveolar region of the lung have enabled the practical delivery of drugs such as insulin to blood circulation (see, e.g., Patton, J. S. et al., U.S. Pat. No. 5,997,848). Pulmonary delivery allows administration without needle injection, and obviates the need for potentially toxic penetration enhancers.

Compositions suitable for use in the invention include compositions wherein the active ingredients are contained in an effective amount to achieve the intended purpose. The determination of an effective dose is well within the capability of those skilled in the art.

Specialized forms of compositions may be prepared for direct intracellular delivery of macromolecules comprising REMAP or fragments thereof. For example, liposome preparations containing a cell-impermeable macromolecule may promote cell fusion and intracellular delivery of the macromolecule. Alternatively, REMAP or a fragment thereof may be joined to a short cationic N-terminal portion from the HIV Tat-1 protein. Fusion proteins thus generated have been found to transduce into the cells of all tissues, including the brain, in a mouse model system (Schwarze, S. R. et al. (1999) Science 285:1569-1572).

For any compound, the therapeutically effective dose can be estimated initially either in cell culture assays, e.g., of neoplastic cells, or in animal models such as mice, rats, rabbits, dogs, monkeys, or pigs. An animal model may also be used to determine the appropriate concentration range and route of administration. Such information can then be used to determine useful doses and routes for administration in humans.

A therapeutically effective dose refers to that amount of active ingredient, for example REMAP or fragments thereof, antibodies of REMAP, and agonists, antagonists or inhibitors of REMAP, which ameliorates the symptoms or condition. Therapeutic efficacy and toxicity may be determined by standard pharmaceutical procedures in cell cultures or with experimental animals, such as by calculating the ED₅₀ (the dose therapeutically effective in 50% of the population) or LD₅₀ (the dose lethal to 50% of the population) statistics. The dose ratio of toxic to therapeutic effects is the therapeutic index, which can be expressed as the LD₅₀/ED₅₀ ratio. Compositions which exhibit large therapeutic indices are preferred. The data obtained from cell culture assays and animal studies are used to formulate a range of dosage for human use. The dosage contained in such compositions is preferably within a range of circulating concentrations that includes the ED₅₀ with little or no toxicity. The dosage varies within this range depending upon the dosage form employed, the sensitivity of the patient, and the route of administration.

The exact dosage will be determined by the practitioner, in light of factors related to the subject requiring treatment. Dosage and administration are adjusted to provide sufficient levels of the active moiety or to maintain the desired effect. Factors which may be taken into account include the severity of the disease state, the general health of the subject, the age, weight, and gender of the subject, time and frequency of administration, drug combination(s), reaction sensitivities, and response to therapy. Long-acting compositions may be administered every 3 to 4 days, every week, or biweekly depending on the half-life and clearance rate of the particular formulation.

Normal dosage amounts may vary from about 0.1 μg to 100,000 μg, up to a total dose of about 1 gram, depending upon the route of administration. Guidance as to particular dosages and methods of delivery is provided in the literature and generally available to practitioners in the art. Those skilled in the art will employ different formulations for nucleotides than for proteins or their inhibitors. Similarly, delivery of polynucleotides or polypeptides will be specific to particular cells, conditions, locations, etc.

Diagnostics

In another embodiment, antibodies which specifically bind REMAP may be used for the diagnosis of disorders characterized by expression of REMAP, or in assays to monitor patients being treated with REMAP or agonists, antagonists, or inhibitors of REMAP. Antibodies useful for diagnostic purposes may be prepared in the same manner as described above for therapeutics. Diagnostic assays for REMAP include methods which utilize the antibody and a label to detect REMAP in human body fluids or in extracts of cells or tissues. The antibodies may be used with or without modification, and may be labeled by covalent or non-covalent attachment of a reporter molecule. A wide variety of reporter molecules, several of which are described above, are known in the art and may be used.

A variety of protocols for measuring REMAP, including ELISAs, RIAs, and FACS, are known in the art and provide a basis for diagnosing altered or abnormal levels of REMAP expression. Normal or standard values for REMAP expression are established by combining body fluids or cell extracts taken from normal mammalian subjects, for example, human subjects, with antibodies to REMAP under conditions suitable for complex formation. The amount of standard complex formation may be quantitated by various methods, such as photometric means. Quantities of REMAP expressed in subject, control, and disease samples from biopsied tissues are compared with the standard values. Deviation between standard and subject values establishes the parameters for diagnosing disease.

In another embodiment of the invention, polynucleotides encoding REMAP may be used for diagnostic purposes. The polynucleotides which may be used include oligonucleotides, complementary RNA and DNA molecules, and PNAs. The polynucleotides may be used to detect and quantify gene expression in biopsied tissues in which expression of REMAP may be correlated with disease. The diagnostic assay may be used to determine absence, presence, and excess expression of REMAP, and to monitor regulation of REMAP levels during therapeutic intervention.

In one aspect, hybridization with PCR probes which are capable of detecting polynucleotides, including genomic sequences, encoding REMAP or closely related molecules may be used to identify nucleic acid sequences which encode REMAP. The specificity of the probe, whether it is made from a highly specific region, e.g., the 5′ regulatory region, or from a less specific region, e.g., a conserved motif, and the stringency of the hybridization or amplification will determine whether the probe identifies only naturally occurring sequences encoding REMAP, allelic variants, or related sequences.

Probes may also be used for the detection of related sequences, and may have at least 50% sequence identity to any of the REMAP encoding sequences. The hybridization probes of the subject invention may be DNA or RNA and may be derived from the sequence of SEQ ID NO:48-94 or from genomic sequences including promoters, enhancers, and introns of the REMAP gene.

Means for producing specific hybridization probes for polynucleotides encoding REMAP include the cloning of polynucleotides encoding REMAP or REMAP derivatives into vectors for the production of mRNA probes. Such vectors are known in the art, are commercially available, and may be used to synthesize RNA probes in vitro by means of the addition of the appropriate RNA polymerases and the appropriate labeled nucleotides. Hybridization probes may be labeled by a variety of reporter groups, for example, by radionuclides such as 32 or ³⁵S, or by enzymatic labels, such as alkaline phosphatase coupled to the probe via avidin/biotin coupling systems, and the like.

Polynucleotides encoding REMAP may be used for the diagnosis of disorders associated with expression of REMAP. Examples of such disorders include, but are not limited to, a cell proliferative disorder such as actinic keratosis, arteriosclerosis, atherosclerosis, bursitis, cirrhosis, hepatitis, mixed connective tissue disease (MCTD), myelofibrosis, paroxysmal nocturnal hemoglobinuria, polycythemia vera, psoriasis, primary thrombocythemia, and cancers including adenocarcinoma, leukemia, lymphoma, melanoma, myeloma, sarcoma, teratocarcinoma, and, in particular, cancers of the adrenal gland, bladder, bone, bone marrow, brain, breast, cervix, colon, gall bladder, ganglia, gastrointestinal tract, heart, kidney, liver, lung, muscle, ovary, pancreas, parathyroid, penis, prostate, salivary glands, skin, spleen, testis, thymus, thyroid, and uterus; an autoimmune/inflammatory disorder such as acquired immunodeficiency syndrome (AIDS), Addison's disease, adult respiratory distress syndrome, allergies, ankylosing spondylitis, amyloidosis, anemia, asthma, atherosclerosis, autoimmune hemolytic anemia, autoimmune thyroiditis, autoimmune polyendocrinopathy-candidiasis-ectodermal dystrophy (APECED), bronchitis, cholecystitis, contact dermatitis, Crohn's disease, atopic dermatitis, dermatomyositis, diabetes mellitus, emphysema, episodic lymphopenia with lymphocytotoxins, erythroblastosis fetalis, erythema nodosum, atrophic gastritis, glomerulonephritis, Goodpasture's syndrome, gout, Graves' disease, Hashirnoto's thyroiditis, hypereosinophilia, irritable bowel syndrome, multiple sclerosis, myasthenia gravis, myocardial or pericardial inflammation, osteoarthritis, osteoporosis, pancreatitis, polymyositis, psoriasis, Reiter's syndrome, rheumatoid arthritis, scleroderma, Sjögren's syndrome, systemic anaphylaxis, systemic lupus erythematosus, systemic sclerosis, thrombocytopenic purpura, ulcerative colitis, uveitis, Wemer syndrome, complications of cancer, hemodialysis, and extracorporeal circulation, viral, bacterial, fungal, parasitic, protozoal, and helminthic infections, and trauma; a neurological disorder such as epilepsy, ischemic cerebrovascular disease, stroke, cerebral neoplasms, Alzheimer's disease, Pick's disease, Huntington's disease, dementia, Parkinson's disease and other extrapyramidal disorders, amyotrophic lateral sclerosis and other motor neuron disorders, progressive neural muscular atrophy, retinitis pigmentosa, hereditary ataxias, multiple sclerosis and other demyelinating diseases, bacterial and viral meningitis, brain abscess, subdural empyema, epidural abscess, suppurative intracranial thrombophlebitis, myelitis and radiculitis, viral central nervous system disease, prion diseases including kuru, Creutzfeldt-Jakob disease, and Gerstmann-Straussler-Scheinker syndrome, fatal familial insomnia, nutritional and metabolic diseases of the nervous system, neurofibromatosis, tuberous sclerosis, cerebelloretinal hemangioblastomatosis, encephalotrigeminal syndrome, mental retardation and other developmental disorders of the central nervous system, cerebral palsy, neuroskeletal disorders, autonomic nervous system disorders, cranial nerve disorders, spinal cord diseases, muscular dystrophy and other neuromuscular disorders, peripheral nervous system disorders, dermatomyositis and polymyositis, inherited, metabolic, endocrine, and toxic myopathies, myasthenia gravis, periodic paralysis, mental disorders including mood, anxiety, and schizophrenic disorders, seasonal affective disorder (SAD), akathesia, amnesia, catatonia, diabetic neuropathy, tardive dyskinesia, dystonias, paranoid psychoses, postherpetic neuralgia, Tourette's disorder, progressive supranuclear palsy, corticobasal degeneration, and familial frontotemporal dementia; a metabolic disorder such as Addison's disease, cerebrotendinous xanthomatosis, congenital adrenal hyperplasia, coumarin resistance, cystic fibrosis, fatty hepatocirrhosis, fructose-1,6-diphosphatase deficiency, galactosemia, goiter, glucagonoma, glycogen storage diseases, hereditary fructose intolerance, hyperadrenalism, hypoadrenalism, hyperparathyroidism, hypoparathyroidism, hypercholesterolemia, hyperthyroidism, hypoglycemia, hypothyroidism, hyperlipidemia, hyperlipemia, lipid myopathies, lipodystrophies, lysosomal storage diseases, mannosidosis, neuraminidase deficiency, obesity, osteoporosis, phenylketonuria, pseudo vitamin D-deficiency rickets, disorders of carbohydrate metabolism such as congenital type II dyserythropoietic anemia, diabetes, insulin-dependent diabetes mellitus, non-insulin-dependent diabetes mellitus, galactose epimerase deficiency, glycogen storage diseases, lysosomal storage diseases, fructosuria, pentosuria, and inherited abnormalities of pyruvate metabolism, disorders of lipid metabolism such as fatty liver, cholestasis, primary biliary cirrhosis, carnitine deficiency, carnitine palmitoyltransferase deficiency, myoadenylate deaminase deficiency, hypertriglyceridemia, lipid storage disorders such Fabry's disease, Gaucher's disease, Niemann-Pick's disease, metachromatic leukodystrophy, adrenoleukodystrophy, GM₂ gangliosidosis, and ceroid lipofuscinosis, abetalipoproteinemia, Tangier disease, hyperlipoproteinemia, lipodystrophy, lipomatoses, acute panniculitis, disseminated fat necrosis, adiposis dolorosa, lipoid adrenal hyperplasia, minimal change disease, lipomas, atherosclerosis, hypercholesterolemia, hypercholesterolemia with hypertriglyceridemia, primary hypoalphalipoproteinemia, hypothyroidism, renal disease, liver disease, lecithin:cholesterol acyltransferase deficiency, cerebrotendinous xanthomatosis, sitosterolemia, hypocholesterolemia, Tay-Sachs disease, Sandhoff's disease, hyperlipidemia, hyperlipemia, and lipid myopathies, and disorders of copper metabolism such as Menke's disease, Wilson's disease, and Ehlers-Danlos syndrome type IX diabetes; a developmental disorder such as renal tubular acidosis, anemia, Cushing's syndrome, achondroplastic dwarfism, Duchenne and Becker muscular dystrophy, epilepsy, gonadal dysgenesis, WAGR syndrome (Wilms' tumor, aniridia, genitourinary abnormalities, and mental retardation), Smith-Magenis syndrome, myelodysplastic syndrome, hereditary mucoepithelial dysplasia, hereditary keratodermas, hereditary neuropathies such as Charcot-Marie-Tooth disease and neurofibromatosis, hypothyroidism, hydrocephalus, a seizure disorder such as Syndenham's chorea and cerebral palsy, spina bifida, anencephaly, craniorachischisis, congenital glaucoma, cataract, and sensorineural hearing loss; and an endocrine disorder such as a disorder of the hypothalamus and/or pituitary resulting from lesions such as a primary brain tumor, adenoma, infarction associated with pregnancy, hypophysectomy, aneurysm, vascular malformation, thrombosis, infection, immunological disorder, and complication due to head trauma, a disorder associated with hypopituitarism including hypogonadism, Sheehan syndrome, diabetes insipidus, Kallman's disease, Hand-Schuller-Christian disease, Letterer-Siwe disease, sarcoidosis, empty sella syndrome, and dwarfism, a disorder associated with hyperpituitarism including acromegaly, giantism, and syndrome of inappropriate antidiuretic hormone (ADH) secretion (SIADH) often caused by benign adenoma, a disorder associated with hypothyroidism including goiter, myxedema, acute thyroiditis associated with bacterial infection, subacute thyroiditis associated with viral infection, autoimmune thyroiditis (Hashimoto's disease), and cretinism, a disorder associated with hyperthyroidism including thyrotoxicosis and its various forms, Grave's disease, pretibial myxedema, toxic multinodular goiter, thyroid carcinoma, and Plummer's disease, a disorder associated with hyperparathyroidism including Conn disease (chronic hypercalemia), a pancreatic disorder such as Type I or Type II diabetes mellitus and associated complications, a disorder associated with the adrenals such as hyperplasia, carcinoma, or adenoma of the adrenal cortex, hypertension associated with alkalosis, amyloidosis, hypokalemia, Cushing's disease, Liddle's syndrome, and Arnold-Healy-Gordon syndrome, pheochromocytoma tumors, and Addison's disease, a disorder associated with gonadal steroid hormones such as: in women, abnormal prolactin production, infertility, endometriosis, perturbation of the menstrual cycle, polycystic ovarian disease, hyperprolactinemia, isolated gonadotropin deficiency, amenorrhea, galactorrhea, hermaphroditism, hirsutism and virilization, breast cancer, and, in post-menopausal women, osteoporosis, and, in men, Leydig cell deficiency, male climacteric phase, and germinal cell aplasia, a hypergonadal disorder associated with Leydig cell tumors, androgen resistance associated with absence of androgen receptors, syndrome of 5 α-reductase, and gynecomastia. Polynucleotides encoding REMAP may be used in Southern or northern analysis, dot blot, or other membrane-based technologies; in PCR technologies; in dipstick, pin, and multiformat ELISA-like assays; and in microarrays utilizing fluids or tissues from patients to detect altered REMAP expression. Such qualitative or quantitative methods are well known in the art.

In a particular embodiment, polynucleotides encoding REMAP may be used in assays that detect the presence of associated disorders, particularly those mentioned above. Polynucleotides complementary to sequences encoding REMAP may be labeled by standard methods and added to a fluid or tissue sample from a patient under conditions suitable for the formation of hybridization complexes. After a suitable incubation period, the sample is washed and the signal is quantified and compared with a standard value. If the amount of signal in the patient sample is significantly altered in comparison to a control sample then the presence of altered levels of polynucleotides encoding REMAP in the sample indicates the presence of the associated disorder. Such assays may also be used to evaluate the efficacy of a particular therapeutic treatment regimen in animal studies, in clinical trials, or to monitor the treatment of an individual patient.

In order to provide a basis for the diagnosis of a disorder associated with expression of REMAP, a normal or standard profile for expression is established. This may be accomplished by combining body fluids or cell extracts taken from normal subjects, either animal or human, with a sequence, or a fragment thereof, encoding REMAP, under conditions suitable for hybridization or amplification. Standard hybridization may be quantified by comparing the values obtained from normal subjects with values from an experiment in which a known amount of a substantially purified polynucleotide is used. Standard values obtained in this manner may be compared with values obtained from samples from patients who are symptomatic for a disorder. Deviation from standard values is used to establish the presence of a disorder.

Once the presence of a disorder is established and a treatment protocol is initiated, hybridization assays may be repeated on a regular basis to determine if the level of expression in the patient begins to approximate that which is observed in the normal subject. The results obtained from successive assays may be used to show the efficacy of treatment over a period ranging from several days to months.

With respect to cancer, the presence of an abnormal amount of transcript (either under- or overexpressed) in biopsied tissue from an individual may indicate a predisposition for the development of the disease, or may provide a means for detecting the disease prior to the appearance of actual clinical symptoms. A more definitive diagnosis of this type may allow health professionals to employ preventative measures or aggressive treatment earlier, thereby preventing the development or further progression of the cancer.

Additional diagnostic uses for oligonucleotides designed from the sequences encoding REMAP may involve the use of PCR. These oligomers may be chemically synthesized, generated enzymatically, or produced in vitro. Oligomers will preferably contain a fragment of a polynucleotide encoding REMAP, or a fragment of a polynucleotide complementary to the polynucleotide encoding REMAP, and will be employed under optimized conditions for identification of a specific gene or condition. Oligomers may also be employed under less stringent conditions for detection or quantification of closely related DNA or RNA sequences.

In a particular aspect, oligonucleotide primers derived from polynucleotides encoding REMAP may be used to detect single nucleotide polymorphisms (SNPs). SNPs are substitutions, insertions and deletions that are a frequent cause of inherited or acquired genetic disease in humans. Methods of SNP detection include, but are not limited to, single-stranded conformation polymorphism (SSCP) and fluorescent SSCP (fSSCP) methods. In SSCP, oligonucleotide primers derived from polynucleotides encoding REMAP are used to amplify DNA using the polymerase chain reaction (PCR). The DNA may be derived, for example, from diseased or normal tissue, biopsy samples, bodily fluids, and the like. SNPs in the DNA cause differences in the secondary and tertiary structures of PCR products in single-stranded form, and these differences are detectable using gel electrophoresis in non-denaturing gels. In fSCCP, the oligonucleotide primers are fluorescently labeled, which allows detection of the amplimers in high-throughput equipment such as DNA sequencing machines. Additionally, sequence database analysis methods, termed in silico SNP (is SNP), are capable of identifying polymorphisms by comparing the sequence of individual overlapping DNA fragments which assemble into a common consensus sequence. These computer-based methods filter out sequence variations due to laboratory preparation of DNA and sequencing errors using statistical models and automated analyses of DNA sequence chromatograms. In the alternative, SNPs may be detected and characterized by mass spectrometry using, for example, the high throughput MASSARRAY system (Sequenom, Inc., San Diego Calif.).

SNPs may be used to study the genetic basis of human disease. For example, at least 16 common SNPs have been associated with non-insulin-dependent diabetes mellitus. SNPs are also useful for examining differences in disease outcomes in monogenic disorders, such as cystic fibrosis, sickle cell anemia, or chronic granulomatous disease. For example, variants in the mannose-binding lectin, MBL2, have been shown to be correlated with deleterious pulmonary outcomes in cystic fibrosis. SNPs also have utility in pharmacogenomics, the identification of genetic variants that influence a patient's response to a drug, such as life-threatening toxicity. For example, a variation in N-acetyl transferase is associated with a high incidence of peripheral neuropathy in response to the anti-tuberculosis drug isoniazid, while a variation in the core promoter of the ALOX5 gene results in diminished clinical response to treatment with an anti-asthma drug that targets the 5-lipoxygenase pathway. Analysis of the distribution of SNPs in different populations is useful for investigating genetic drift, mutation, recombination, and selection, as well as for tracing the origins of populations and their migrations (Taylor, J. G. et al. (2001) Trends Mol. Med. 7:507-512; Kwok, P. -Y. and Z. Gu (1999) Mol. Med. Today 5:538-543; Nowotny, P. et al. (2001) Curr. Opin. Neurobiol. 11:637-641).

Methods which may also be used to quantify the expression of REMAP include radiolabeling or biotinylating nucleotides, coamplification of a control nucleic acid, and interpolating results from standard curves (Melby, P. C. et al. (1993) J. Immunol. Methods 159:235-244; Duplaa, C. et al. (1993) Anal. Biochem. 212:229-236). The speed of quantitation of multiple samples may be accelerated by running the assay in a high-throughput format where the oligomer or polynucleotide of interest is presented in various dilutions and a spectrophotometric or colorimetric response gives rapid quantitation.

In further embodiments, oligonucleotides or longer fragments derived from any of the polynucleotides described herein may be used as elements on a microarray. The microarray can be used in transcript imaging techniques which monitor the relative expression levels of large numbers of genes simultaneously as described below. The microarray may also be used to identify genetic variants, mutations, and polymorphisms. This information may be used to determine gene function, to understand the genetic basis of a disorder, to diagnose a disorder, to monitor progression/regression of disease as a function of gene expression, and to develop and monitor the activities of therapeutic agents in the treatment of disease. In particular, this information may be used to develop a pharmacogenomic profile of a patient in order to select the most appropriate and effective treatment regimen for that patient. For example, therapeutic agents which are highly effective and display the fewest side effects may be selected for a patient based on his/her pharmacogenomic profile.

In another embodiment, REMAP, fragments of REMAP, or antibodies specific for REMAP may be used as elements on a microarray. The microarray may be used to monitor or measure protein-protein interactions, drug-target interactions, and gene expression profiles, as described above.

A particular embodiment relates to the use of the polynucleotides of the present invention to generate a transcript image of a tissue or cell type. A transcript image represents the global pattern of gene expression by a particular tissue or cell type. Global gene expression patterns are analyzed by quantifying the number of expressed genes and their relative abundance under given conditions and at a given time (Seilhamer et al., “Comparative Gene Transcript Analysis,” U.S. Pat. No. 5,840,484; hereby expressly incorporated by reference herein). Thus a transcript image may be generated by hybridizing the polynucleotides of the present invention or their complements to the totality of transcripts or reverse transcripts of a particular tissue or cell type. In one embodiment, the hybridization takes place in high-throughput format, wherein the polynucleotides of the present invention or their complements comprise a subset of a plurality of elements on a microarray. The resultant transcript image would provide a profile of gene activity.

Transcript images may be generated using transcripts isolated from tissues, cell lines, biopsies, or other biological samples. The transcript image may thus reflect gene expression in vivo, as in the case of a tissue or biopsy sample, or in vitro, as in the case of a cell line.

Transcript images which profile the expression of the polynucleotides of the present invention may also be used in conjunction with in vitro model systems and preclinical evaluation of pharmaceuticals, as well as toxicological testing of industrial and naturally-occurring environmental compounds. All compounds induce characteristic gene expression patterns, frequently termed molecular fingerprints or toxicant signatures, which are indicative of mechanisms of action and toxicity (Nuwaysir, E. F. et al. (1999) Mol. Carcinog. 24:153-159; Steiner, S. and N. L. Anderson (2000) Toxicol. Lett. 112-113:467-471). If a test compound has a signature similar to that of a compound with known toxicity, it is likely to share those toxic properties. These fingerprints or signatures are most useful and refined when they contain expression information from a large number of genes and gene families. Ideally, a genome-wide measurement of expression provides the highest quality signature. Even genes whose expression is not altered by any tested compounds are important as well, as the levels of expression of these genes are used to normalize the rest of the expression data. The normalization procedure is useful for comparison of expression data after treatment with different compounds. While the assignment of gene function to elements of a toxicant signature aids in interpretation of toxicity mechanisms, knowledge of gene function is not necessary for the statistical matching of signatures which leads to prediction of toxicity (see, for example, Press Release 00-02 from the National Institute of Environmental Health Sciences, released Feb. 29, 2000, available at http://www.niehs.nih.gov/oc/news/toxchip.htm). Therefore, it is important and desirable in toxicological screening using toxicant signatures to include all expressed gene sequences.

In an embodiment, the toxicity of a test compound can be assessed by treating a biological sample containing nucleic acids with the test compound. Nucleic acids that are expressed in the treated biological sample are hybridized with one or more probes specific to the polynucleotides of the present invention, so that transcript levels corresponding to the polynucleotides of the present invention may be quantified. The transcript levels in the treated biological sample are compared with levels in an untreated biological sample. Differences in the transcript levels between the two samples are indicative of a toxic response caused by the test compound in the treated sample.

Another embodiment relates to the use of the polypeptides disclosed herein to analyze the proteome of a tissue or cell type. The term proteome refers to the global pattern of protein expression in a particular tissue or cell type. Each protein component of a proteome can be subjected individually to further analysis. Proteome expression patterns, or profiles, are analyzed by quantifying the number of expressed proteins and their relative abundance under given conditions and at a given time. A profile of a cell's proteome may thus be generated by separating and analyzing the polypeptides of a particular tissue or cell type. In one embodiment, the separation is achieved using two-dimensional gel electrophoresis, in which proteins from a sample are separated by isoelectric focusing in the first dimension, and then according to molecular weight by sodium dodecyl sulfate slab gel electrophoresis in the second dimension (Steiner and Anderson, supra). The proteins are visualized in the gel as discrete and uniquely positioned spots, typically by staining the gel with an agent such as Coomassie Blue or silver or fluorescent stains. The optical density of each protein spot is generally proportional to the level of the protein in the sample. The optical densities of equivalently positioned protein spots from different samples, for example, from biological samples either treated or untreated with a test compound or therapeutic agent, are compared to identify any changes in protein spot density related to the treatment. The proteins in the spots are partially sequenced using, for example, standard methods employing chemical or enzymatic cleavage followed by mass spectrometry. The identity of the protein in a spot may be determined by comparing its partial sequence, preferably of at least 5 contiguous amino acid residues, to the polypeptide sequences of interest. In some cases, further sequence data may be obtained for definitive protein identification.

A proteomic profile may also be generated using antibodies specific for REMAP to quantify the levels of REMAP expression. In one embodiment, the antibodies are used as elements on a microarray, and protein expression levels are quantified by exposing the microarray to the sample and detecting the levels of protein bound to each array element (Lueking, A. et al. (1999) Anal. Biochem. 270:103-111; Mendoze, L. G. et al. (1999) Biotechniques 27:778-788). Detection may be performed by a variety of methods known in the art, for example, by reacting the proteins in the sample with a thiol- or amino-reactive fluorescent compound and detecting the amount of fluorescence bound at each array element.

Toxicant signatures at the proteome level are also useful for toxicological screening, and should be analyzed in parallel with toxicant signatures at the transcript level. There is a poor correlation between transcript and protein abundances for some proteins in some tissues (Anderson, N. L. and J. Seilhamer (1997) Electrophoresis 18:533-537), so proteome toxicant signatures may be useful in the analysis of compounds which do not significantly affect the transcript image, but which alter the proteomic profile. In addition, the analysis of transcripts in body fluids is difficult, due to rapid degradation of mRNA, so proteomic profiling may be more reliable and informative in such cases.

In another embodiment, the toxicity of a test compound is assessed by treating a biological sample containing proteins with the test compound. Proteins that are expressed in the treated biological sample are separated so that the amount of each protein can be quantified. The amount of each protein is compared to the amount of the corresponding protein in an untreated biological sample. A difference in the amount of protein between the two samples is indicative of a toxic response to the test compound in the treated sample. Individual proteins are identified by sequencing the amino acid residues of the individual proteins and comparing these partial sequences to the polypeptides of the present invention.

In another embodiment, the toxicity of a test compound is assessed by treating a biological sample containing proteins with the test compound. Proteins from the biological sample are incubated with antibodies specific to the polypeptides of the present invention. The amount of protein recognized by the antibodies is quantified. The amount of protein in the treated biological sample is compared with the amount in an untreated biological sample. A difference in the amount of protein between the two samples is indicative of a toxic response to the test compound in the treated sample.

Microarrays may be prepared, used, and analyzed using methods known in the art (Brennan, T. M. et al. (1995) U.S. Pat. No. 5,474,796; Schena, M. et al. (1996) Proc. Natl. Acad. Sci. USA 93:10614-10619; Baldeschweiler et al. (1995) PCT application WO95/251116; Shalon, D. et al. (1995) PCT application WO95/35505; Heller, R. A. et al. (1997) Proc. Natl. Acad. Sci. USA 94:2150-2155; Heller, M. J. et al. (1997) U.S. Pat. No. 5,605,662). Various types of microarrays are well known and thoroughly described in Schena, M., ed. (1999; DNA Microarrays: A Practical Approach, Oxford University Press, London).

In another embodiment of the invention, nucleic acid sequences encoding REMAP may be used to generate hybridization probes useful in mapping the naturally occurring genomic sequence. Either coding or noncoding sequences may be used, and in some instances, noncoding sequences may be preferable over coding sequences. For example, conservation of a coding sequence among members of a multi-gene family may potentially cause undesired cross hybridization during chromosomal mapping. The sequences may be mapped to a particular chromosome, to a specific region of a chromosome, or to artificial chromosome constructions, e.g., human artificial chromosomes (HACs), yeast artificial chromosomes (YACs), bacterial artificial chromosomes (BACs), bacterial P1 constructions, or single chromosome cDNA libraries (Harrington, J. J. et al. (1997) Nat. Genet. 15:345-355; Price, C. M. (1993) Blood Rev. 7:127-134; Trask, B. J. (1991) Trends Genet. 7:149-154). Once mapped, the nucleic acid sequences may be used to develop genetic linkage maps, for example, which correlate the inheritance of a disease state with the inheritance of a particular chromosome region or restriction fragment length polymorphism (RFLP) (Lander, E. S. and D. Botstein (1986) Proc. Natl. Acad. Sci. USA 83:7353-7357).

Fluorescent in situ hybridization (FISH) may be correlated with other physical and genetic map data (Heinz-Ulrich, et al. (1995) in Meyers, supra, pp. 965-968). Examples of genetic map data can be found in various scientific journals or at the Online Mendelian Inheritance in Man (OMIM) World Wide Web site. Correlation between the location of the gene encoding REMAP on a physical map and a specific disorder, or a predisposition to a specific disorder, may help define the region of DNA associated with that disorder and thus may further positional cloning efforts.

In situ hybridization of chromosomal preparations and physical mapping techniques, such as linkage analysis-using established chromosomal markers, may be used for extending genetic maps. Often the placement of a gene on the chromosome of another mammalian species, such as mouse, may reveal associated markers even if the exact chromosomal locus is not known. This information is valuable to investigators searching for disease genes using positional cloning or other gene discovery techniques. Once the gene or genes responsible for a disease or syndrome have been crudely localized by genetic linkage to a particular genomic region, e.g., ataxia-telangiectasia to 11q22-23, any sequences mapping to that area may represent associated or regulatory genes for further investigation (Gatti, R. A. et al. (1988) Nature 336:577-580). The nucleotide sequence of the instant invention may also be used to detect differences in the chromosomal location due to translocation, inversion, etc., among normal, carrier, or affected individuals.

In another embodiment of the invention, REMAP, its catalytic or immunogenic fragments, or oligopeptides thereof can be used for screening libraries of compounds in any of a variety of drug screening techniques. The fragment employed in such screening may be free in solution, affixed to a solid support, borne on a cell surface, or located intracellularly. The formation of binding complexes between REMAP and the agent being tested may be measured.

Another technique for drug screening provides for high throughput screening of compounds having suitable binding affinity to the protein of interest (Geysen, et al. (1984) PCT application WO84/03564). In this method, large numbers of different small test compounds are synthesized on a solid substrate. The test compounds are reacted with REMAP, or fragments thereof, and washed. Bound REMAP is then detected by methods well known in the art. Purified REMAP can also be coated directly onto plates for use in the aforementioned drug screening techniques. Alternatively, non-neutralizing antibodies can be used to capture the peptide and immobilize it on a solid support.

In another embodiment, one may use competitive drug screening assays in which neutralizing antibodies capable of binding REMAP specifically compete with a test compound for binding REMAP. In this manner, antibodies can be used to detect the presence of any peptide which shares one or more antigenic determinants with REMAP.

In additional embodiments, the nucleotide sequences which encode REMAP may be used in any molecular biology techniques that have yet to be developed, provided the new techniques rely on properties of nucleotide sequences that are currently known, including, but not limited to, such properties as the triplet genetic code and specific base pair interactions.

Without further elaboration, it is believed that one skilled in the art can, using the preceding description, utilize the present invention to its fullest extent. The following embodiments are, therefore, to be construed as merely illustrative, and not limitative of the remainder of the disclosure in any way whatsoever.

The disclosures of all patents, applications, and publications mentioned above and below, including U.S. Ser. No. 60/333,097, U.S. Ser. No. 60/335,274, U.S. Ser. No. 60/340,542, U.S. Ser. No. 60/342,166, U.S. Ser. No. 60/348,687, and U.S. Ser. No. 60/347,580, are hereby expressly incorporated by reference.

### EXAMPLES

I. Construction of cDNA Libraries

Incyte cDNAs were derived from cDNA libraries described in the LIFESEQ GOLD database (Incyte Genomics, Palo Alto Calif.). Some tissues were homogenized and lysed in guanidinium isothiocyanate, while others were homogenized and lysed in phenol or in a suitable mixture of denaturants, such as TRIZOL (Invitrogen), a monophasic solution of phenol and guanidine isothiocyanate. The resulting lysates were centrifuged over CsCl cushions or extracted with chloroform. RNA was precipitated from the lysates with either isopropanol or sodium acetate and ethanol, or by other routine methods.

Phenol extraction and precipitation of RNA were repeated as necessary to increase RNA purity. In some cases, RNA was treated with DNase. For most libraries, poly(A)+ RNA was isolated using oligo d(T)-coupled paramagnetic particles (Promega), OLIGOTEX latex particles (QIAGEN, Chatsworth Calif.), or an OLIGOTEX mRNA purification kit (QIAGEN). Alternatively, RNA was isolated directly from tissue lysates using other RNA isolation kits, e.g., the POLY(A)PURE mRNA purification kit (Ambion, Austin Tex.).

In some cases, Stratagene was provided with RNA and constructed the corresponding cDNA libraries. Otherwise, cDNA was synthesized and cDNA libraries were constructed with the UNIZAP vector system (Stratagene) or SUPERSCRIPT plasmid system (Invitrogen), using the recommended procedures or similar methods known in the art (Ausubel et al., supra, ch. 5). Reverse transcription was initiated using oligo d(T) or random primers. Synthetic oligonucleotide adapters were ligated to double stranded cDNA, and the cDNA was digested with the appropriate restriction enzyme or enzymes. For most libraries, the cDNA was size-selected (300-1000 bp) using SEPHACRYL S1000, SEPHAROSE CL2B, or SEPHAROSE CLAB column chromatography (Amersham Biosciences) or preparative agarose gel electrophoresis. cDNAs were ligated into compatible restriction enzyme sites of the polylinker of a suitable plasmid, e.g., PBLUESCRIPT plasmid (Stratagene), PSPORT1 plasmid (Invitrogen, Carlsbad Calif.), PCDNA2.1 plasmid (Invitrogen), PBK-CMV plasmid (Stratagene), PCR2-TOPOTA plasmid (Invitrogen), PCMV-ICIS plasmid (Stratagene), pIGEN (Incyte Genomics, Palo Alto Calif.), pRARE (Incyte Genomics), or pINCY (Incyte Genomics), or derivatives thereof. Recombinant plasmids were transformed into competent E. coli cells including XL1-Blue, XL1-BlueMRF, or SOLR from Stratagene or DH5α, DH10B, or ElectroMAX DH10B from Invitrogen.

II. Isolation of cDNA Clones

Plasmids obtained as described in Example I were recovered from host cells by in vivo excision using the UNIZAP vector system (Stratagene) or by cell lysis. Plasmids were purified using at least one of the following: a Magic or WIZARD Minipreps DNA purification system (Promega); an AGTC Miniprep purification kit (Edge Biosystems, Gaithersburg Md.); and QIAWELL 8 Plasmid, QIAWELL 8 Plus Plasmid, QIAWELL 8 Ultra Plasmid purification systems or the R.E.A.L. PREP 96 plasmid purification kit from QIAGEN. Following precipitation, plasmids were resuspended in 0.1 ml of distilled water and stored, with or without lyophilization, at 4° C.

Alternatively, plasmid DNA was amplified from host cell lysates using direct link PCR in a high-throughput format (Rao, V. B. (1994) Anal. Biochem. 216:1-14). Host cell lysis and thermal cycling steps were carried out in a single reaction mixture. Samples were processed and stored in 384-well plates, and the concentration of amplified plasmid DNA was quantified fluorometrically using PICOGREEN dye (Molecular Probes, Eugene Oreg.) and a FLUOROSKAN II fluorescence scanner (Labsystems Oy, Helsinki, Finland).

III. Sequencing and Analysis

Incyte cDNA recovered in plasmids as described in Example II were sequenced as follows. Sequencing reactions were processed using standard methods or high-throughput instrumentation such as the ABI CATALYST 800 (Applied Biosystems) thermal cycler or the PTC-200 thermal cycler (MJ Research) in conjunction with the HYDRA microdispenser (Robbins Scientific) or the MICROLAB 2200 (Hamilton) liquid transfer system. cDNA sequencing reactions were prepared using reagents provided by Amersham Biosciences or supplied in ABI sequencing kits such as the ABI PRISM BIGDYE Terminator cycle sequencing ready reaction kit (Applied Biosystems). Electrophoretic separation of cDNA sequencing reactions and detection of labeled polynucleotides were carried out using the MEGABACE 1000 DNA sequencing system (Amersham Biosciences); the ABI PRISM 373 or 377 sequencing system (Applied Biosystems) in conjunction with standard ABI protocols and base calling software; or other sequence analysis systems known in the art. Reading frames within the cDNA sequences were identified using standard methods (Ausubel et al., supra, ch. 7). Some of the cDNA sequences were selected for extension using the techniques disclosed in Example VIII.

The polynucleotide sequences derived from Incyte cDNAs were validated by removing vector, linker, and poly(A) sequences and by masking ambiguous bases, using algorithms and programs based on BLAST, dynamic programming, and dinucleotide nearest neighbor analysis. The Incyte cDNA sequences or translations thereof were then queried against a selection of public databases such as the GenBank primate, rodent, mammalian, vertebrate, and eukaryote databases, and BLOCKS, PRINTS, DOMO, PRODOM; PROTEOME databases with sequences from Homo sapiens, Ratius norvegicus, Mus musculus, Caenorhabditis elegans, Saccharomyces cerevisiae, Schizosaccharomyces pombe, and Candida albicans (Incyte Genomics, Palo Alto Calif.); hidden Markov model (HMM)-based protein family databases such as PFAM, INCY, and TIGRFAM (Haft, D. H. et al. (2001) Nucleic Acids Res. 29:41-43); and HMM-based protein domain databases such as SMART (Schultz, J. et al. (1998) Proc. Natl. Acad. Sci. USA 95:5857-5864; Letunic, I. et al. (2002) Nucleic Acids Res. 30:242-244). (EM is a probabilistic approach which analyzes consensus primary structures of gene families; see, for example, Eddy, S. R. (1996) Curr. Opin. Struct. Biol. 6:361-365.) The queries were performed using programs based on BLAST, FASTA, BLIMPS, and HMMER. The Incyte cDNA sequences were assembled to produce full length polynucleotide sequences. Alternatively, GenBank cDNAs, GenBank ESTs, stitched sequences, stretched sequences, or Genscan-predicted coding sequences (see Examples IV and V) were used to extend Incyte cDNA assemblages to full length. Assembly was performed using programs based on Phred, Phrap, and Consed, and cDNA assemblages were screened for open reading frames using programs based on GeneMark, BLAST, and FASTA. The full length polynucleotide sequences were translated to derive the corresponding full length polypeptide sequences. Alternatively, a polypeptide may begin at any of the methionine residues of the full length translated polypeptide. Full length polypeptide sequences were subsequently analyzed by querying against databases such as the GenBank protein databases (genpept), SwissProt, the PROTEOME databases, BLOCKS, PRINTS, DOMO, PRODOM, Prosite, hidden Markov model (HMM)-based protein family databases such as PFAM, INCY, and TIGRFAM; and HMM-based protein domain databases such as SMART. Full length polynucleotide sequences are also analyzed using MACDNASIS PRO software (MiraiBio, Alameda Calif.) and LASERGENE software (DNASTAR). Polynucleotide and polypeptide sequence alignments are generated using default parameters specified by the CLUSTAL algorithm as incorporated into the MEGALIGN multisequence alignment program (DNASTAR), which also calculates the percent identity between aligned sequences.

Table 7 summarizes the tools, programs, and algorithms used for the analysis and assembly of Incyte cDNA and full length sequences and provides applicable descriptions, references, and threshold parameters. The first column of Table 7 shows the tools, programs, and algorithms used, the second column provides brief descriptions thereof, the third column presents appropriate references, all of which are incorporated by reference herein in their entirety, and the fourth column presents, where applicable, the scores, probability values, and other parameters used to evaluate the strength of a match between two sequences (the higher the score or the lower the probability value, the greater the identity between two sequences).

The programs described above for the assembly and analysis of full length polynucleotide and polypeptide sequences were also used to identify polynucleotide sequence fragments from SEQ ID NO:48-94. Fragments from about 20 to about 4000 nucleotides which are useful in hybridization and amplification technologies are described in Table 4, column 2.

IV. Identification and Editing of Coding Sequences from Genomic DNA

Putative receptors and membrane-associated proteins were initially identified by running the Genscan gene identification program against public genomic sequence databases (e.g., gbpri and gbhtg). Genscan is a general-purpose gene identification program which analyzes genomic DNA sequences from a variety of organisms (Burge, C. and S. Karlin (1997) J. Mol. Biol. 268:78-94; Burge, C. and S. Karlin (1998) Curr. Opin. Struct. Biol. 8:346-354). The program concatenates predicted exons to form an assembled cDNA sequence extending from a methionine to a stop codon. The output of Genscan is a FASTA database of polynucleotide and polypeptide sequences. The maximum range of sequence for Genscan to analyze at once was set to 30 kb. To determine which of these Genscan predicted cDNA sequences encode receptors and membrane-associated proteins, the encoded polypeptides were analyzed by querying against PFAM models for receptors and membrane-associated proteins. Potential receptors and membrane-associated proteins were also identified by homology to Incyte cDNA sequences that had been annotated as receptors and membrane-associated proteins. These selected Genscan-predicted sequences were then compared by BLAST analysis to the genpept and gbpri public databases. Where necessary, the Genscan-predicted sequences were then edited by comparison to the top BLAST hit from genpept to correct errors in the sequence predicted by Genscan, such as extra or omitted exons. BLAST analysis was also used to find any Incyte cDNA or public cDNA coverage of the Genscan-predicted sequences, thus providing evidence for transcription. When Incyte cDNA coverage was available, this information was used to correct or confirm the Genscan predicted sequence. Full length polynucleotide sequences were obtained by assembling Genscan-predicted coding sequences with Incyte cDNA sequences and/or public cDNA sequences using the assembly process described in Example III. Alternatively, full length polynucleotide sequences were derived entirely from edited or unedited Genscan-predicted coding sequences.

V. Assembly of Genomic Sequence Data with cDNA Sequence Data

“Stitched” Sequences

Partial cDNA sequences were extended with exons predicted by the Genscan gene identification program described in Example IV. Partial cDNAs assembled as described in Example III were mapped to genomic DNA and parsed into clusters containing related cDNAs and Genscan exon predictions from one or more genomic sequences. Each cluster was analyzed using an algorithm based on graph theory and dynamic programming to integrate cDNA and genomic information, generating possible splice variants that were subsequently confirmed, edited, or extended to create a full length sequence. Sequence intervals in which the entire length of the interval was present on more than one sequence in the cluster were identified, and intervals thus identified were considered to be equivalent by transitivity. For example, if an interval was present on a cDNA and two genomic sequences, then all three intervals were considered to be equivalent. This process allows unrelated but consecutive genomic sequences to be brought together, bridged by cDNA sequence. Intervals thus identified were then “stitched” together by the stitching algorithm in the order that they appear along their parent sequences to generate the longest possible sequence, as well as sequence variants. Linkages between intervals which proceed along one type of parent sequence (cDNA to cDNA or genomic sequence to genomic sequence) were given preference over linkages which change parent type (cDNA to genomic sequence). The resultant stitched sequences were translated and compared by BLAST analysis to the genpept and gbpri public databases. Incorrect exons predicted by Genscan were corrected by comparison to the top BLAST hit from genpept. Sequences were further extended with additional cDNA sequences, or by inspection of genomic DNA, when necessary.

“Stretched” Sequences

Partial DNA sequences were extended to full length with an algorithm based on BLAST analysis. First, partial cDNAs assembled as described in Example III were queried against public databases such as the GenBank primate, rodent, mammalian, vertebrate, and eukaryote databases using the BLAST program. The nearest GenBank protein homolog was then compared by BLAST analysis to either Incyte cDNA sequences or GenScan exon predicted sequences described in Example IV. A chimeric protein was generated by using the resultant high-scoring segment pairs (HSPs) to map the translated sequences onto the GenBank protein homolog. Insertions or deletions may occur in the chimeric protein with respect to the original GenBank protein homolog. The GenBank protein homolog, the chimeric protein, or both were used as probes to search for homologous genomic sequences from the public human genome databases. Partial DNA sequences were therefore “stretched” or extended by the addition of homologous genomic sequences. The resultant stretched sequences were examined to determine whether it contained a complete gene.

VI. Chromosomal Mapping of REMAP Encoding Polynucleotides

The sequences which were used to assemble SEQ ID NO:48-94 were compared with sequences from the Incyte LIFESEQ database and public domain databases using BLAST and other implementations of the Smith-Waterman algorithm. Sequences from these databases that matched SEQ ID NO:48-94 were assembled into clusters of contiguous and overlapping sequences using assembly algorithms such as Phrap (Table 7). Radiation hybrid and genetic mapping data available from public resources such as the Stanford Human Genome Center (SHGC), Whitehead Institute for Genome Research (WIGR), and Généthon were used to determine if any of the clustered sequences had been previously mapped. Inclusion of a mapped sequence in a cluster resulted in the assignment of all sequences of that cluster, including its particular SEQ ID NO:, to that map location.

Map locations are represented by ranges, or intervals, of human chromosomes. The map position of an interval, in centiMorgans, is measured relative to the terminus of the chromosome's p-arm. (The centiMorgan (cM) is a unit of measurement based on recombination frequencies between chromosomal markers. On average, 1 cM is roughly equivalent to 1 megabase (Mb) of DNA in humans, although this can vary widely due to hot and cold spots of recombination.) The cM distances are based on genetic markers mapped by Généthon which provide boundaries for radiation hybrid markers whose sequences were included in each of the clusters. Human genome maps and other resources available to the public, such as the NCBI “GeneMap'99” World Wide Web site (http://www.ncbi.nlm.nih.gov/genemap/), can be employed to determine if previously identified disease genes map within or in proximity to the intervals indicated above.

Association of RMAP Polynucleotides with Alzheimer's Disease

RMAP polynucleotides were mapped to NT_Contigs, available from NCBI, using the following procedures. Contigs longer than 1 Mb were broken into subcontigs of 1 Mb in length with overlapping sections of 100 kb. A preliminary step used an algorithm, similar to MEGABLAST, to define the mRNA sequence/masked genomic DNA contig pairings. The cDNA/genomic pairings identified by the first algorithm were run through Sim4 (Florea, L. et al. (1998) Genome Res. 8:967-74, version May 2000) that had been optimized in house for high throughput and strand assignment confidence). The Sim4 output of the mRNA sequence/genomic contig pairs was further processed to determine the correct location of the RMAP polynucleotides on the genomic contig, and also their strand identity.

Loci on chromosomes that map to regions associated with particular diseases can be used as markers for these particular diseases. These markers then can be used to develop diagnostic and therapeutic tools for these diseases. For example, loci on chromosome 10 are associated with or linked to Alzheimer's disease (AD), a progressive neurodegenerative disease that represents the most common form of dementia (Ait-Ghezala, G. et al. (2002) Neurosci Lett. 325:87-90). AD can be inherited as an autosomal dominant trait. Further, genetic studies have focused on identification of genes that are potential targets for new treatments or improved diagnostics. The deposition and aggregation of β-amyloid in specific regions of the brain are key neuropathological hallmarks of AD. Insulin-degrading enzyme (IDE) can degrade O-amyloid Abraham, R. et al. (2001) Hum. Genet. 109:646-652). The IDE gene has been mapped near an AD-associated locus, 10q23-q25 (Espinosa R. 3rd et al. (1991) Cytogenet. Cell Genet. 57:184-186). Linkage analysis using IDE gene markers was performed on 1426 subjects from 435 families in which at least two family members were affected with AD.

A logarithm of the odds ratio for linkage (lod) score of over 3 indicates a probability of 1 in 1000 that a particular marker was found solely by chance in affected individuals. Significant linkage (lod score of 3.3) was reported between the polymorphic marker D10S583, located at 115.3 cM on chromosome 10, and AD with age of onset ≧50 years (Betram, L. et al. (2000) Science 290:2302-2303). D10S583 maps 36 kb upstream of the IDE gene. Further analysis of this region, however, failed to show association of SNPs (single nucleotide polymorphisms) within the IDE gene and flanking regions with late-onset AD (LOAD), in a study of 134 Caucasian LOAD cases and 111 matched controls from the United Kingdom (Abraham, R. et al, supra). Thus, although the activity of IDE may not influence the susceptibility to LOAD, there is substantial linkage in the chromosomal region containing the EDE gene, marker D10S583, and AD. The IDE gene and D10S583 both map to contig NT_{—}008769, which contains a region of chromosome 10 that is 9.16 Mb in size.

SEQ ID NO:58 mapped to a region of contig NT_{—}008804 from the Feb. 2, 2002 release of NCBI., localizing SEQ ID NO:58 to within 9.16 Mb of the Alzheimer's disease locus on chromosome 10q. Thus, SEQ ID NO:58 is in proximity with loci shown to consistently associate with Alzheimer's disease.

II. Analysis of Polynucleotide Expression

Northern analysis is a laboratory technique used to detect the presence of a transcript of a gene and involves the hybridization of a labeled nucleotide sequence to a membrane on which RNAs from a particular cell type or tissue have been bound (Sambrook and Russell, supra, ch. 7; Ausubel et al., supra, ch. 4).

Analogous computer techniques applying BLAST were used to search for identical or related molecules in databases such as GenBank or LIFESEQ (Incyte Genomics). This analysis is much faster than multiple membrane-based hybridizations. In addition, the sensitivity of the computer search can be modified to determine whether any particular match is categorized as exact or similar. The basis of the search is the product score, which is defined as:

$$\frac{BLAST Score \times Percent Identity}{5 \times minimum \{ length ( Seq . 1 ) , length ( Seq . 2 ) \}}$$

The product score takes into account both the degree of similarity between two sequences and the length of the sequence match. The product score is a normalized value between 0 and 100, and is calculated as follows: the BLAST score is multiplied by the percent nucleotide identity and the product is divided by (5 times the length of the shorter of the two sequences). The BLAST score is calculated by assigning a score of +5 for every base that matches in a high-scoring segment pair (HSP), and −4 for every mismatch. Two sequences may share more than one HSP (separated by gaps). If there is more than one HSP, then the pair with the highest BLAST score is used to calculate the product score. The product score represents a balance between fractional overlap and quality in a BLAST alignment. For example, a product score of 100 is produced only for 100% identity over the entire length of the shorter of the two sequences being compared. A product score of 70 is produced either by 100% identity and 70% overlap at one end, or by 88% identity and 100% overlap at the other. A product score of 50 is produced either by 100% identity and 50% overlap at one end, or 79% identity and 100% overlap.

Alternatively, polynucleotides encoding REMAP are analyzed with respect to the tissue sources from which they were derived. For example, some full length sequences are assembled, at least in part, with overlapping Incyte cDNA sequences (see Example E). Each cDNA sequence is derived from a cDNA library constructed from a human tissue. Each human tissue is classified into one of the following organ/tissue categories: cardiovascular system; connective tissue; digestive system; embryonic structures; endocrine system; exocrine glands; genitalia, female; genitalia, male; germ cells; hemic and immune system; liver; musculoskeletal system; nervous system; pancreas; respiratory system; sense organs; skin; stomatognathic system; unclassified/mixed; or urinary tract. The number of libraries in each category is counted and divided by the total number of libraries across all categories. Similarly, each human tissue is classified into one of the following disease/condition categories: cancer, cell line, developmental, inflammation, neurological, trauma, cardiovascular, pooled, and other, and the number of libraries in each category is counted and divided by the total number of libraries across all categories. The resulting percentages reflect the tissue- and disease-specific expression of cDNA encoding REMAP. cDNA sequences and cDNA library/tissue information are found in the LIFESEQ GOLD database (Incyte Genomics, Palo Alto Calif.).

VIII. Extension of REMAP Encoding Polynucleotides

Full length polynucleotides are produced by extension of an appropriate fragment of the full length molecule using oligonucleotide primers designed from this fragment. One primer was synthesized to initiate 5′ extension of the known fragment, and the other primer was synthesized to initiate 3′ extension of the known fragment. The initial primers were designed using OLIGO 4.06 software (National Biosciences), or another appropriate program, to be about 22 to 30 nucleotides in length, to have a GC content of about 50% or more, and to anneal to the target sequence at temperatures of about 68° C. to about 72° C. Any stretch of nucleotides which would result in hairpin structures and primer-primer dimerizations was avoided.

Selected human cDNA libraries were used to extend the sequence. If more than one extension was necessary or desired, additional or nested sets of primers were designed.

High fidelity amplification was obtained by PCR using methods well known in the art. PCR was performed in 96-well plates using the PTC-200 thermal cycler (MJ Research, Inc.). The reaction mix contained DNA template, 200 nmol of each primer, reaction buffer containing Mg²⁺, (NH₄)₂SO₄, and 2-mercaptoethanol, Taq DNA polymerase (Amersham Biosciences), ELONGASE enzyme (Invitrogen), and Pfu DNA polymerase (Stratagene), with the following parameters for primer pair PCI A and PCI B: Step 1: 94° C., 3 min; Step 2: 94° C., 15 sec; Step 3: 60° C., 1 min; Step 4: 68° C., 2 min; Step 5: Steps 2, 3, and 4 repeated 20 times; Step 6: 68° C., 5 min; Step 7: storage at 4° C. In the alternative, the parameters for primer pair T7 and SK+ were as follows: Step 1: 94° C., 3 min; Step 2: 94° C., 15 sec; Step 3: 57° C., 1 min; Step 4: 68° C., 2 min; Step 5: Steps 2, 3, and 4 repeated 20 times; Step 6: 68° C., 5 min; Step 7: storage at 4° C.

The concentration of DNA in each well was determined by dispensing 100 μl PICOGREEN quantitation reagent (0.25% (v/v) PICOGREEN; Molecular Probes, Eugene Oreg.) dissolved in 1×TE and 0.5 μl of undiluted PCR product into each well of an opaque fluorimeter plate (Corning Costar, Acton Mass.), allowing the DNA to bind to the reagent. The plate was scanned in a Fluoroskan II (Labsystems Oy, Helsinki, Finland) to measure the fluorescence of the sample and to quantify the concentration of DNA. A 5 μl to 10 μl aliquot of the reaction mixture was analyzed by electrophoresis on a 1% agarose gel to determine which reactions were successful in extending the sequence.

The extended nucleotides were desalted and concentrated, transferred to 384-well plates, digested with CviJI cholera virus endonuclease (Molecular Biology Research, Madison Wis.), and sonicated or sheared prior to religation into pUC 18 vector (Amersham Biosciences). For shotgun sequencing, the digested nucleotides were separated on low concentration (0.6 to 0.8%) agarose gels, fragments were excised, and agar digested with Agar ACE (Promega). Extended clones were religated using T4 ligase (New England Biolabs, Beverly Mass.) into pUC 18 vector (Amersham Biosciences), treated with Pfu DNA polymerase (Stratagene) to fill-in restriction site overhangs, and transfected into competent E. coli cells. Transformed cells were selected on antibiotic-containing media, and individual colonies were picked and cultured overnight at 37° C. in 384-well plates in LB/2× carb liquid media.

The cells were lysed, and DNA was amplified by PCR using Taq DNA polymerase (Amersham Biosciences) and Pfu DNA polymerase (Stratagene) with the following parameters: Step 1: 94° C., 3 min; Step 2: 94° C., 15 sec; Step 3: 60° C., 1 min; Step 4: 72° C., 2 min; Step 5: steps 2, 3, and 4 repeated 29 times; Step 6: 72° C., 5 min; Step 7: storage at 4° C. DNA was quantified by PICOGREEN reagent (Molecular Probes) as described above. Samples with low DNA recoveries were reamplified using the same conditions as described above. Samples were diluted with 20% dimethysulfoxide (1:2, v/v), and sequenced using DYNAMIC energy transfer sequencing primers and the DYNAMIC DIRECT kit (Amersham Biosciences) or the ABI PRISM BIGDYE Terminator cycle sequencing ready reaction kit (Applied Biosystems).

In like manner, full length polynucleotides are verified using the above procedure or are used to obtain 5′ regulatory sequences using the above procedure along with oligonucleotides designed for such extension, and an appropriate genomic library.

IX. Identification of Single Nucleotide Polymorphisms in REMAP Encoding Polynucleotides

Common DNA sequence variants known as single nucleotide polymorphisms (SNPs) were identified in SEQ ID NO:48-94 using the LIFESEQ database (Incyte Genomics). Sequences from the same gene were clustered together and assembled as described in Example III, allowing the identification of all sequence variants in the gene. An algorithm consisting of a series of filters was used to distinguish SNPs from other sequence variants. Preliminary filters removed the majority of basecall errors by requiring a minimum Phred quality score of 15, and removed sequence alignment errors and errors resulting from improper trimming of vector sequences, chimeras, and splice variants. An automated procedure of advanced chromosome analysis analysed the original chromatogram files in the vicinity of the putative SNP. Clone error filters used statistically generated algorithms to identify errors introduced during laboratory processing, such as those caused by reverse transcriptase, polymerase, or somatic mutation. Clustering error filters used statistically generated algorithms to identify errors resulting from clustering of close homologs or pseudogenes, or due to contamination by non-human sequences. A final set of filters removed duplicates and SNPs found in immunoglobulins or T-cell receptors.

Certain SNPs were selected for further characterization by mass spectrometry using the high throughput MASSARRAY system (Sequenom, Inc.) to analyze allele frequencies at the SNP sites in four different human populations. The Caucasian population comprised 92 individuals (46 male, 46 female), including 83 from Utah, four French, three Venezualan, and two Amish individuals. The African population comprised 194 individuals (97 male, 97 female), all African Americans. The Hispanic population comprised 324 individuals (162 male, 162 female), all Mexican Hispanic. The Asian population comprised 126 individuals (64 male, 62 female) with a reported parental breakdown of 43% Chinese, 31% Japanese, 13% Korean, 5% Vietnamese, and 8% other Asian. Allele frequencies were first analyzed in the Caucasian population; in some cases those SNPs which showed no allelic variance in this population were not further tested in the other three populations.

X. Labeling and Use of Individual Hybridization Probes

Hybridization probes derived from SEQ ID NO:48-94 are employed to screen cDNAs, genomic DNAs, or mRNAs. Although the labeling of oligonucleotides, consisting of about 20 base pairs, is specifically described, essentially the same procedure is used with larger nucleotide fragments. Oligonucleotides are designed using state-of-the-art software such as OLIGO 4.06 software (National Biosciences) and labeled by combining 50 pmol of each oligomer, 250 μCi of [γ-³²P] adenosine triphosphate (Amersham Biosciences), and T4 polynucleotide kinase (DuPont NEN, Boston Mass.). The labeled oligonucleotides are substantially purified using a SEPHADEX G-25 superfine size exclusion dextran bead column (Amersham Biosciences). An aliquot containing 10⁷ counts per minute of the labeled probe is used in a typical membrane-based hybridization analysis of human genomic DNA digested with one of the following endonucleases: Ase I, Bgl II, Eco RI, Pst I, Xba I, or Pvu II (DuPont NEN).

The DNA from each digest is fractionated on a 0.7% agarose gel and transferred to nylon membranes (Nytran Plus, Schleicher & Schuell, Durham N.H.). Hybridization is carried out for 16 hours at 40° C. To remove nonspecific signals, blots are sequentially washed at room temperature under conditions of up to, for example, 0.1× saline sodium citrate and 0.5% sodium dodecyl sulfate. Hybridization patterns are visualized using autoradiography or an alternative imaging means and compared.

XI. Microarrays

The linkage or synthesis of array elements upon a microarray can be achieved utilizing photolithography, piezoelectric printing (ink-jet printing; see, e.g., Baldeschweiler et al., supra), mechanical microspotting technologies, and derivatives thereof. The substrate in each of the aforementioned technologies should be uniform and solid with a non-porous surface (Schena, M., ed. (1999) DNA Microarrays: A Practical Approach, Oxford University Press, London). Suggested substrates include silicon, silica, glass slides, glass chips, and silicon wafers. Alternatively, a procedure analogous to a dot or slot blot may also be used to arrange and link elements to the surface of a substrate using thermal, UV, chemical, or mechanical bonding procedures. A typical array may be produced using available methods and machines well known to those of ordinary skill in the art and may contain any appropriate number of elements (Schena, M. et al. (1995) Science 270:467-470; Shalon, D. et al. (1996) Genome Res. 6:639-645; Marshall, A. and J. Hodgson (1998) Nat. Biotechnol. 16:27-31).

Full length cDNAs, Expressed Sequence Tags (ESTs), or fragments or oligomers thereof may comprise the elements of the microarray. Fragments or oligomers suitable for hybridization can be selected using software well known in the art such as LASERGENE software (DNASTAR). The array elements are hybridized with polynucleotides in a biological sample. The polynucleotides in the biological sample are conjugated to a fluorescent label or other molecular tag for ease of detection. After hybridization, nonhybridized nucleotides from the biological sample are removed, and a fluorescence scanner is used to detect hybridization at each array element. Alternatively, laser desorbtion and mass spectrometry may be used for detection of hybridization. The degree of complementarity and the relative abundance of each polynucleotide which hybridizes to an element on the microarray may be assessed. In one embodiment, microarray preparation and usage is described in detail below.

Tissue or Cell Sample Preparation

Total RNA is isolated from tissue samples using the guanidinium thiocyanate method and poly(A)⁺ RNA is purified using the oligo-(dT) cellulose method. Each poly(A)⁺ RNA sample is reverse transcribed using MMLV reverse-transcriptase, 0.05 μg/μl oligo-(dT) primer (21mer), 1× first strand buffer, 0.03 units/μl RNase inhibitor, 500 μM dATP, 500 μM dGTP, 500 μM dTTP, 40 μM dCTP, 40 μM dCTP-Cy3 (BDS) or dCTP-Cy5 (Amersham Biosciences). The reverse transcription reaction is performed in a 25 ml volume containing 200 ng poly(A)⁺ RNA with GEMBRIGHT kits (Incyte Genomics). Specific control poly(A)⁺ RNAs are synthesized by in vitro transcription from non-coding yeast genomic DNA. After incubation at 37° C. for 2 hr, each reaction sample (one with Cy3 and another with Cy5 labeling) is treated with 2.5 ml of 0.5M sodium hydroxide and incubated for 20 minutes at 85° C. to the stop the reaction and degrade the RNA. Samples are purified using two successive CHROMA SPIN 30 gel filtration spin columns (Clontech, Palo Alto Calif.) and after combining, both reaction samples are ethanol precipitated using 1 ml of glycogen (1 mg/ml), 60 ml sodium acetate, and 300 ml of 100% ethanol. The sample is then dried to completion using a SpeedVAC (Savant Instruments Inc., Holbrook N.Y.) and resuspended in 14 μl 5×SSC/0.2% SDS.

Microarray Preparation

Sequences of the present invention are used to generate array elements. Each array element is amplified from bacterial cells containing vectors with cloned cDNA inserts. PCR amplification uses primers complementary to the vector sequences flanking the cDNA insert. Array elements are amplified in thirty cycles of PCR from an initial quantity of 1-2 ng to a final quantity greater than 5 μg. Amplified array elements are then purified using SEPHACRYL-400 (Amersham Biosciences).

Purified array elements are immobilized on polymer-coated glass slides. Glass microscope slides (Corning) are cleaned by ultrasound in 0.1% SDS and acetone, with extensive distilled water washes between and after treatments. Glass slides are etched in 4% hydrofluoric acid (VWR Scientific Products Corporation (VWR), West Chester Pa.), washed extensively in distilled water, and coated with 0.05% aminopropyl silane (Sigma) in 95% ethanol. Coated slides are cured in a 110° C. oven.

Array elements are applied to the coated glass substrate using a procedure described in U.S. Pat. No. 5,807,522, incorporated herein by reference. 1 μl of the array element DNA, at an average concentration of 100 ng/μl, is loaded into the open capillary printing element by a high-speed robotic apparatus. The apparatus then deposits about 5 nl of array element sample per slide.

Microarrays are UV-crosslinked using a STRATALINKER UV-crosslinker (Stratagene). Microarrays are washed at room temperature once in 0.2% SDS and three times in distilled water. Non-specific binding sites are blocked by incubation of microarrays in 0.2% casein in phosphate buffered saline (PBS) (Tropix, Inc., Bedford Mass.) for 30 minutes at 60° C. followed by washes in 0.2% SDS and distilled water as before.

Hybridization

Hybridization reactions contain 9 μl of sample mixture consisting of 0.2 μg each of Cy3 and Cy5 labeled cDNA synthesis products in 5×SSC, 0.2% SDS hybridization buffer. The sample mixture is heated to 65° C. for 5 minutes and is aliquoted onto the microarray surface and covered with an 1.8 cm² coverslip. The arrays are transferred to a waterproof chamber having a cavity just slightly larger than a microscope slide. The chamber is kept at 100% humidity internally by the addition of 140 μl of 5×SSC in a corner of the chamber. The chamber containing the arrays is incubated for about 6.5 hours at 60° C. The arrays are washed for 10 min at 45° C. in a first wash buffer (1×SSC, 0.1% SDS), three times for 10 minutes each at 45° C. in a second wash buffer (0.1×SSC), and dried.

Detection

Reporter-labeled hybridization complexes are detected with a microscope equipped with an Innova 70 mixed gas 10 W laser (Coherent, Inc., Santa Clara Calif.) capable of generating spectral lines at 488 nm for excitation of Cy3 and at 632 nm for excitation of Cy5. The excitation laser light is focused on the array using a 20× microscope objective (Nikon, Inc., Melville N.Y.). The slide containing the array is placed on a computer-controlled X-Y stage on the microscope and raster-scanned past the objective. The 1.8 cm×1.8 cm array used in the present example is scanned with a resolution of 20 micrometers.

In two separate scans, a mixed gas multiline laser excites the two fluorophores sequentially. Emitted light is split, based on wavelength, into two photomultiplier tube detectors (PMT R1477, Hamamatsu Photonics Systems, Bridgewater N.J.) corresponding to the two fluorophores. Appropriate filters positioned between the array and the photomultiplier tubes are used to filter the signals. The emission maxima of the fluorophores used are 565 nm for Cy3 and 650 nm for Cy5. Each array is typically scanned twice, one scan per fluorophore using the appropriate filters at the laser source, although the apparatus is capable of recording the spectra from both fluorophores simultaneously.

The sensitivity of the scans is typically calibrated using the signal intensity generated by a cDNA control species added to the sample mixture at a known concentration. A specific location on the array contains a complementary DNA sequence, allowing the intensity of the signal at that location to be correlated with a weight ratio of hybridizing species of 1:100,000. When two samples from different sources (e.g., representing test and control cells), each labeled with a different fluorophore, are hybridized to a single array for the purpose of identifying genes that are differentially expressed, the calibration is done by labeling samples of the calibrating cDNA with the two fluorophores and adding identical amounts of each to the hybridization mixture.

The output of the photomultiplier tube is digitized using a 12-bit RTI-835H analog-to-digital (A/D) conversion board (Analog Devices, Inc., Norwood Mass.) installed in an IBM-compatible PC computer. The digitized data are displayed as an image where the signal intensity is mapped using a linear 20-color transformation to a pseudocolor scale ranging from blue (low signal) to red (high signal). The data is also analyzed quantitatively. Where two different fluorophores are excited and measured simultaneously, the data are first corrected for optical crosstalk (due to overlapping emission spectra) between the fluorophores using each fluorophore's emission spectrum.

A grid is superimposed over the fluorescence signal image such that the signal from each spot is centered in each element of the grid. The fluorescence signal within each element is then integrated to obtain a numerical value corresponding to the average intensity of the signal. The software used for signal analysis is the GEMTOOLS gene expression analysis program (Incyte Genomics). Array elements that exhibit at least about a two-fold change in expression, a signal-to-background ratio of at least about 2.5, and an element spot size of at least about 40%, are considered to be differentially expressed.

Expression

SEQ ID NO:51 showed differential expression, as determined by microarray analysis, associated with inflammatory responses. Human peripheral blood mononuclear cells (PBMCs) (52% lymphocytes, 20% NK cells, 25% monocytes, and 3% various cells that include dendritic and progenitor cells) were treated with one of the following: interleukin 5 (IL-5), interleukin 6 (IL-6), interleukin 1beta (IL-1β) or tumor necrosis factor alpha (TNF-α). IL-5 is a T-cell derived factor that promotes the proliferation, differentiation, and activation of eosinophils. IL-5 exerts its activity on target cells by binding to specific cell surface receptors. IL-6 is a multifunctional protein that is important in host defense, acute phase reactions, immune responses and hematopoiesis. The production of IL-6 is upregulated by numerous signals, including mitogenic or antigenic stimulation, lipopolysaccharide, IL-1, IL-2, interferon, TNF and viruses. IL-1β is a cytokine associated with acute inflammatory responses and is generally considered a prototypical pro-inflammatory cytokine. Both cells of the immune system (monocytes, dendritic cells, NK cells, platelets, and neutrophils) and somatic cells (osteoblasts, neurons, oligodendrocytes, Schwann cells and adrenal cortical cells) can produce IL-1β. TNF-α is produced by a variety of cell types, including neutrophils, activated lymphocytes, macrophages, NK cells, LAK cells, astrocytes and some transformed cells. TNF-α plays a critical role in mediation of the inflammatory response and in mediation of resistance to infections and tumor growth. RNA was collected from PBMCs cultured in the presence or absence of IL-5, IL-6, IL1β or TNF-α for 2 hours. The expression of SEQ ID NO:51 was increased by at least two-fold in all treatments, as compared to untreated PBMCs. Therefore, in various embodiments, SEQ ID NO:51 can be used for one or more of the following: i) monitoring treatment of immune disorders and related diseases and conditions, ii) diagnostic assays for immune disorders and related diseases and conditions, and iii) developing therapeutics and/or other treatments for immune disorders and related diseases and conditions.

SEQ ID NO:5 and SEQ ID NO:53 showed differential expression, as determined by microarray analysis, in liver C3A cells treated with the steroid betamethasone. The human C3A cell line is a clonal derivative of HepG2/C3 and has been established as an in vitro model of the mature human liver (Mickelson et al. (1995) Hepatology 22:866-875; Nagendra et al. (1997) Am J Physiol 272:G408-G416). SEQ ID NO:5 and SEQ ID NO:53 showed at least a two-fold increase in expression in early confluent C3A cells treated with betamethasone, for 1, 3, and 6 hours. Therefore, in various embodiments, SEQ ID NO:5 and SEQ ID NO:53 can be used for one or more of the following: i) monitoring treatment of liver disease, ii) diagnostic assays for liver disease, and iii) developing therapeutics and/or other treatments for liver disease.

SEQ ID NO:54 showed differential expression associated with chondroblastic osteosarcoma, as determined by microarray analysis. mRNA from normal human osteoblast (primary culture, NHOst 5488) was compared with mRNA from biopsy specimens and osteosarcoma tissues. As compared with normal osteoblasts, the expression of SEQ ID NO:54 was decreased by at least two-fold in femur bone tumor tissue from a 12-year-old female with chondroblastic osteosarcoma and in femur bone tumor tissue and associated cartilage from a 16-year-old female donor with chondroblastic osteosarcoma. Therefore, in various embodiments, SEQ ID NO:54 can be used for one or more of the following: i) monitoring treatment of osteosarcoma, ii) diagnostic assays for osteosarcoma, and iii) developing therapeutics and/or other treatments for osteosarcoma.

The expression of SEQ ID NO:59 was decreased at least two-fold and the expression of SEQ ID NO:61 was increased at least two-fold in an ovarian adenocarcinoma when matched with normal tissue from the same donor. The tumorous ovary tissue was obtained from ovarian adenocarcinoma from a 79-year-old female. Normal ovary tissue was obtained from ovary from the same donor. Matched normal and tumorigenic ovary tissue samples are provided by the Huntsman Cancer Institute, (Salt Lake City, Utah). Therefore, in various embodiments, SEQ ID NO:59 and SEQ ID NO:61 can be used for one or more of the following: i) monitoring treatment of ovarian adenocarcinoma, ii) diagnostic assays for ovarian adenocarcinoma, and iii) developing therapeutics and/or other treatments for ovarian adenocarcinoma.

The expression of SEQ ID NO:65 was decreased at least three-fold in lung squamous cell carcinoma in one of five donors and the expression of SEQ ID NO:67 was decreased at least 2.8-fold in lung squamous cell carcinoma in two of five donors when matched with normal tissue from the same donor. The tumorous lung tissue was obtained from the lung of a 66-year-old male with lung squamous cell carcinoma for SEQ ID NO:65 and SEQ ID NO:67, and tumorous lung tissue was obtained from the lung a 68-year-old female with lung squamous cell carcinoma for SEQ ID NO:67. Normal lung tissue was obtained from grossly uninvolved tissue from the lung of the same donor, respectively. Matched normal and tumorigenic lung tissue samples are provided by the Roy Castle International Centre for Lung Cancer Research (Liverpool, UK). Therefore, in various embodiments, SEQ ID NO:65 and SEQ ID NO:67 can be used for one or more of the following: i) monitoring treatment of lung squamous cell carcinoma, ii) diagnostic assays for lung squamous cell carcinoma, and iii) developing therapeutics and/or other treatments for lung squamous cell carcinoma.

Further, the expression of SEQ ID NO:67 was decreased at least two-fold in human colon adenocarcinoma tissue when matched with normal tissue from the same donor. The colon tumor tissue was obtained from a 73-year old female with colon cancer. Normal colon tissue was obtained from grossly uninvolved colon tissue from the same donor. Matched normal and tumorigenic colon tissue samples are provided by the Huntsman Cancer Institute, (Salt Lake City, Utah). Therefore, in various embodiments, SEQ ID NO:67 can be used for one or more of the following: i) monitoring treatment of colon cancer, ii) diagnostic assays for colon cancer, and iii) developing therapeutics and/or other treatments for colon cancer.

SEQ ID NO:71 was differentially expressed in human breast tumor cell lines when compared to primary breast epithelial cells (HMECs) isolated from a normal donor. Breast carcinoma lines at different stages of tumor progression were evaluated: breast adenocarcinoma (MCF-7, SK-BR-3, BT-20, and MDA-mb-231), and breast ductal carcinoma (T-47D, and MDA-mb-435S). The expression of SEQ ID NO:71 in these breast carcinoma lines was downregulated by at least two-fold as compared to the control HMEC cell line.

The expression of SEQ ID NO:73 was downregulated by at least two-fold in C3A liver cell cultures, as compared to normal liver cells.

The expression of SEQ ID NO:76 was downregulated by at least two-fold in lung cancer tissue as compared to normal lung tissue.

These experiments indicate that SEQ ID NO:71, SEQ ID NO:73, and SEQ ID NO:76 exhibited significant differential expression patterns using microarray techniques. Therefore, in various embodiments, SEQ ID NO:71, SEQ ID NO:73, and SEQ ID NO:76 can be used for one or more of the following: i) monitoring treatment of receptors and membrane-associated disorders and related diseases and conditions, including cancers, ii) diagnostic assays for receptors and membrane-associated disorders and related diseases and conditions, including cancers, and iii) developing therapeutics and/or other treatments for receptors and membrane-associated disorders and related diseases and conditions, including cancers.

For example, SEQ ID NO:82 and SEQ ID NO:86-87 showed differential expression in certain breast carcinoma cell lines versus primary mammary epithelial cells as determined by microarray analysis. The gene expression profile of a primary mammary epithelial cell line, HMEC, was compared to the gene expression profiles of breast carcinoma lines at different stages of tumor progression. Cell lines compared included: a) MCF7, a nonmalignant breast adenocarcinoma cell line isolated from the pleural effusion of a 69-year-old female; b) T-47D, a breast carcinoma cell line isolated from a pleural effusion obtained from a 54-year-old female with an infiltrating ductal carcinoma of the breast; c) Sk-BR-3, a breast adenocarcinoma cell line isolated from a malignant pleural effusion of a 43-year-old female; d) BT-20, a breast carcinoma cell line derived in vitro from tumor mass isolated from a 74-year-old female; e) MDA-mb-435S, a spindle shaped strain that evolved from the parent line (435) isolated from the pleural effusion of a 31-year-old female with metastatic, ductal adenocarcinoma of the breast; and f) MDA-mb-231, a metastatic breast tumor cell line derived from the pleural effusion of a 51-year-old female with metastatic breast carcinoma. The microarray experiments showed that the expression of SEQ ID NO:82 was increased by at least two fold in cells from BT20 breast carcinoma cell line relative to cells from the primary mammary epithelial cell line, HMEC. The expression of SEQ ID NO:86 was decreased by at least two fold in cells from MDA-mb435S breast carcinoma cell line relative to cells from the primary mammary epithelial cell line, HMEC. The expression of SEQ ID NO:87 was increased by at least two fold in cells from MDA-mb-435S breast carcinoma cell line relative to cells from the primary mammary epithelial cell line, HMEC. Therefore, in various embodiments, SEQ ID NO:82 and SEQ ID NO:86-87 can be used for one or more of the following: i) monitoring treatment of breast cancer, ii) diagnostic assays for breast cancer, and iii) developing therapeutics and/or other treatments for breast cancer.

SEQ ID NO:82 also showed differential expression in certain prostate carcinoma cell lines versus normal prostate epithelial cells as determined by microarray analysis. Three prostate carcinoma cell lines, DU 145, LNCaP, and PC-3 were included in the experiments. DU 145 was isolated from a metastatic site in the brain of a 69 year old male with widespread metastatic prostate carcinoma. DU 145 has no detectable sensitivity to hormones; forms colonies in semi-solid medium; is only weekly positive for acid phosphatase; and cells are negative for prostate specific antigen (PSA). LNCaP is a prostate carcinoma cell line isolated from a lymph node biopsy of a 50 year old male with metastatic prostate carcinoma. LNCaP expresses PSA, produces prostate acid phosphatase, and expresses androgen receptors. PC-3, a prostate adenocarcinoma cell line, was isolated from a metastatic site in the bone of a 62 year old male with grade IV prostate adenocarcinoma. The normal epithelial cell line, PrEC, is a primary prostate epithelial cell line isolated from a normal donor. In one experiment, the expression of cDNAs from the prostate carcinoma cell lines representing various stages of prostate tumor progression were compared with that of the normal prostate epithelial cells under the same culture conditions. The expression of cDNAs from the prostate carcinoma cell lines grown under optimal conditions (in the presence of growth factors and nutrients) were compared to that of the normal prostate epithelial cells grown under restrictive conditions (in the absence of growth factors and hormones). The experiment showed that the expression of SEQ ID NO:82 was increased by at least two fold in PC-3 and DU145 prostate carcinoma lines grown under optimal conditions relative to PrECs grown under restrictive conditions. Therefore, in various embodiments, SEQ ID NO:82 can be used for one or more of the following: i) monitoring treatment of prostate cancer, ii) diagnostic assays for prostate cancer, and iii) developing therapeutics and/or other treatments for prostate cancer.

In an alternative example, SEQ ID NO:87 also showed differential expression in certain prostate carcinoma cell lines versus normal prostate epithelial cells as determined by microarray analysis. The prostate carcinoma cell lines include CA-HPV-10, DU 145, LNCAP, and PC-3. CA-HPV-7 was derived from cells from a 63 year old male with prostate adenocarcinoma and was transformed by transfection with HPV18 DNA. DU 145 was isolated from a metastatic site in the brain of a 69 year old male with widespread metastatic prostate carcinoma. DU 145 has no detectable sensitivity to hormones; forms colonies in semi-solid medium; is only weakly positive for acid phosphatase; and cells are negative for prostate specific antigen (PSA). LNCAP is a prostate carcinoma cell line isolated from a lymph node biopsy of a 50 year old male with metastatic prostate carcinoma. LNCaP expresses PSA, produces prostate acid phosphatase, and expresses androgen receptors. PC-3, a prostate adenocarcinoma cell line, was isolated from a metastatic site in the bone of a 62 year old male with grade IV prostate adenocarcinoma. The normal epithelial cell line, PZ-HPV-7 was derived from epithelial cells cultured from normal tissue from the peripheral zone of the prostate. The PZ-HPV-7 cells were transformed by transfection with HPV18. The microarray experiments showed that the expression of SEQ ID NO:87 was decreased by at least two fold in two out of four prostate carcinoma lines (DU 145 and LNCaP) relative to cells from the normal prostate epithelial cell line, PZ-HPV-7. Therefore, in various embodiments, SEQ ID NO:87 can be used for one or more of the following: i) monitoring treatment of prostate cancer, ii) diagnostic assays for prostate cancer, and iii) developing therapeutics and/or other treatments for prostate cancer.

Furthermore, the expression of SEQ ID NO:87, as determined by microarray analysis, was increased by at least two fold in sigmoid colon tissues relative to normal sigmoid colon tissues. The sigmoid colon tumor tissue which originated from a metastatic gastric sarcoma (stromal tumor) was harvested from a 48 year old female donor. The normal sigmoid colon tissue was harvested from grossly uninvolved sigmoid colon tissue of the same donor. Therefore, in various embodiments, SEQ ID NO:87 can be used for one or more of the following: i) monitoring treatment of colon cancer, ii) diagnostic assays for colon cancer, and iii) developing therapeutics and/or other treatments for colon cancer. In addition, the expression of SEQ ID NO:87, as determined by microarray analysis, was increased by at least two fold in Tangier disease-derived fibroblasts relative to normal fibroblasts. Both types of cells were cultured in the presence of cholesterol and compared with the same cell type in the absence of cholesterol. The human fibroblasts were obtained from skin transplants from both normal subjects and two patients with homozygous Tangier disease. Cell lines were immortalized by transfection with human papillomavirus 16 genes E6 and E7 and a neomycin resistance selectable marker. TD derived cells are deficient in an assay of apoA-I mediated tritiated cholesterol efflux. Therefore, in various embodiments, SEQ ID NO:87 can be used for one or more of the following: i) monitoring treatment of Tangier disease, ii) diagnostic assays for Tangier disease, and iii) developing therapeutics and/or other treatments for Tangier disease.

In yet another example, the expression of SEQ ID NO:87 was increased by at least two-fold in treated human adipocytes from obese and normal donors when compared to non-treated adipocytes from the same donors. The normal human primary subcutaneous preadipocytes were isolated from adipose tissue of a 28-year-old healthy female with a body mass index (BMI) of 23.59. The obese human primary subcutaneous preadipocytes were isolated from adipose tissue of a 40-year-old healthy female with a body mass index (BMI) of 32.47. The preadipocytes were cultured and induced to differentiate into adipocytes by culturing them in the differentiation medium containing the active components, PPAR-γ agonist and human insulin. Human preadipocytes were treated with human insulin and PPAR-γ agonist for three days and subsequently were switched to medium containing insulin for 24 hours, 48 hours, 4 days, 8 days or 15 days before the cells were collected for analysis. Differentiated adipocytes were compared to untreated preadipocytes maintained in culture in the absence of inducing agents. Between 80% and 90% of the preadipocytes finally differentiated to adipocytes as observed under phase contrast microscope. The experiments showed that at two out of five time points (8 and 15 days), the expression of SEQ ID NO:87 was increased by at least two-fold in normal adipocytes, and at four out of five time points (48 hours, 4, 8, and 15 days), the expression of SEQ ID NO:87 was increased by at least 2 fold in human adipocytes from obese donors. Therefore, in various embodiments, SEQ ID NO:87 can be used for one or more of the following: i) monitoring treatment of diabetes mellitus and other disorders, such as obesity, hypertension, and atherosclerosis, ii) diagnostic assays for diabetes mellitus and other disorders, such as obesity, hypertension, and atherosclerosis, and iii) developing therapeutics and/or other treatments for diabetes mellitus and other disorders, such as obesity, hypertension, and atherosclerosis.

For example, SEQ ID NO:89 and SEQ ID NO:93 showed differential expression in certain breast carcinoma cell lines versus a non-malignant mammary epithelial cell line as determined by microarray analysis. The non-malignant mammary epithelial cell line, MCF10A, was isolated from a 36-year-old female with fibrocystic breast disease. The breast carcinoma cell lines include BT20, a breast carcinoma cell line derived in vitro from cells emigrating out of thin slices of a tumor mass isolated from a 74-year-old female; MCF7, a breast adenocarcinoma cell line derived from the pleural effusion of a 69-year-old female; MDA-mb-231, a metastatic breast tumor cell line derived from the pleural effusion of a 51-year-old female with metastatic breast carcinoma; SkBR3, a breast adenocarcinoma cell line isolated from a malignant pleural effusion of a 43-year-old female; and T47D, a breast carcinoma cell line derived from a pleural effusion from a 54-year-old female with an infiltrating ductal carcinoma of the breast. All cell cultures were propagated in a chemically-defined, serum-free medium, H14, according to the supplier's recommendations and grown to 70-80% confluence prior to RNA isolation. The microarray experiments showed that the expression of SEQ ID NO:89 was increased by at least two fold in MCF7 breast carcinoma line relative to non-malignant mammary epithelial cells. The expression of SEQ ID NO:93 was increased by at least two fold in T47D breast carcinoma line relative to non-malignant mammary epithelial cells. Therefore, in various embodiments, SEQ ID NO:89 and SEQ ID NO:93 can be used for one or more of the following: i) monitoring treatment of breast cancer, ii) diagnostic assays for breast cancer, and iii) developing therapeutics and/or other treatments for breast cancer.

In an alternative example, SEQ ID NO:89 showed differential expression in lung squamous carcinoma tissues versus normal lung tissues as determined by microarray analysis. In two separate experiments, the expression of SEQ ID NO:89 was increased by at least two fold in lung squamous carcinoma tissues relative to grossly uninvolved normal lung tissue from the same donors. The normal and tumorous lung tissues were isolated from a 75 year old female and a 68 year old female. Therefore, in various embodiments, SEQ ID NO:89 can be used for one or more of the following: i) monitoring treatment of lung cancer, ii) diagnostic assays for lung cancer, and iii) developing therapeutics and/or other treatments for lung cancer.

In yet another example, SEQ ID NO:93 showed differential expression in certain prostate carcinoma cell lines versus normal prostate epithelial cells as determined by microarray analysis. Three prostate carcinoma cell lines, DU 145, LNCAP, and PC-3 were included in the experiments. DU 145 was isolated from a metastatic site in the brain of a 69 year old male with widespread metastatic prostate carcinoma. DU 145 has no detectable sensitivity to hormones; forms colonies in semi-solid medium; is only weekly positive for acid phosphatase; and cells are negative for prostate specific antigen (PSA). LNCAP is a prostate carcinoma cell line isolated from a lymph node biopsy of a 50 year old male with metastatic prostate carcinoma. LNCaP expresses PSA, produces prostate acid phosphatase, and expresses androgen receptors. PC-3, a prostate adenocarcinoma cell line, was isolated from a metastatic site in the bone of a 62 year old male with grade IV prostate adenocircinoma. The normal epithelial cell line, PrEC, is a primary prostate epithelial cell line isolated from a normal donor. In one experiment, the expression of cDNAs from the prostate carcinoma cell lines representing various stages of prostate tumor progression were compared with that of the normal prostate epithelial cells under the same culture conditions. The expression of cDNAs from the prostate carcinoma cell lines were compared to that of the normal prostate epithelial cells grown under the same conditions (in the absence of growth factors and hormones). The experiment showed that the expression of SEQ ID NO:93 was increased by at least two fold in DU145 prostate carcinoma lines grown under optimal conditions relative to PrECs grown under restrictive conditions. Therefore, in various embodiments, SEQ ID NO:93 can be used for one or more of the following: i) monitoring treatment of prostate cancer, ii) diagnostic assays for prostate cancer, and iii) developing therapeutics and/or other treatments for prostate cancer.

XII. Complementary Polynucleotides

Sequences complementary to the REMAP-encoding sequences, or any parts thereof, are used to detect, decrease, or inhibit expression of naturally occurring REMAP. Although use of oligonucleotides comprising from about 15 to 30 base pairs is described, essentially the same procedure is used with smaller or with larger sequence fragments. Appropriate oligonucleotides are designed using OLIGO 4.06 software (National Biosciences) and the coding sequence of REMAP. To inhibit transcription, a complementary oligonucleotide is designed from the most unique 5′ sequence and used to prevent promoter binding to the coding sequence. To inhibit translation, a complementary oligonucleotide is designed to prevent ribosomal binding to the REMAP-encoding transcript.

XIII. Expression of REMAP

Expression and purification of REMAP is achieved using bacterial or virus-based expression systems. For expression of REMAP in bacteria, cDNA is subcloned into an appropriate vector containing an antibiotic resistance gene and an inducible promoter that directs high levels of cDNA transcription. Examples of such promoters include, but are not limited to, the trp-lac (tac) hybrid promoter and the T5 or T7 bacteriophage promoter in conjunction with the lac operator regulatory element. Recombinant vectors are transformed into suitable bacterial hosts, e.g., BL21(DE3). Antibiotic resistant bacteria express REMAP upon induction with isopropyl beta-D-thiogalactopyranoside (IPTG). Expression of REMAP in eukaryotic cells is achieved by infecting insect or mammalian cell lines with recombinant Autographica californica nuclear polyhedrosis virus (AcMNPV), commonly known as baculovirus. The nonessential polyhedrin gene of baculovirus is replaced with cDNA encoding REMAP by either homologous recombination or bacterial-mediated transposition involving transfer plasmid intermediates. Viral infectivity is maintained and the strong polyhedrin promoter drives high levels of cDNA transcription. Recombinant baculovirus is used to infect Spodoptera frugiperda (Sf9) insect cells in most cases, or human hepatocytes, in some cases. Infection of the latter requires additional genetic modifications to baculovinis (Engelhard, E. K. et al. (1994) Proc. Natl. Acad. Sci. USA 91:3224-3227; Sandig, V. et al. (1996) Hum. Gene Ther. 7:1937-1945).

In most expression systems, REMAP is synthesized as a fusion protein with, e.g., glutathione S-transferase (GST) or a peptide epitope tag, such as FLAG or 6-His, permitting rapid, single-step, affinity-based purification of recombinant fusion protein from crude cell lysates. GST, a 26-kilodalton enzyme from Schistosoma japonicum, enables the purification of fusion proteins on immobilized glutathione under conditions that maintain protein activity and antigenicity (Amersham Biosciences). Following purification, the GST moiety can be proteolytically cleaved from REMAP at specifically engineered sites. FLAG, an 8-amino acid peptide, enables immunoaffinity purification using commercially available monoclonal and polyclonal anti-FLAG antibodies (Eastman Kodak). 6-His, a stretch of six consecutive histidine residues, enables purification on metal-chelate resins (QIAGEN). Methods for protein expression and purification are discussed in Ausubel et al. (supra, ch. 10 and 16). Purified REMAP obtained by these methods can be used directly in the assays shown in Examples XVII, XVIII, and XIX, where applicable.

XIV. Functional Assays

REMAP function is assessed by expressing the sequences encoding REMAP at physiologically elevated levels in mammalian cell culture systems. cDNA is subcloned into a mammalian expression vector containing a strong promoter that drives high levels of cDNA expression. Vectors of choice include PCMV SPORT plasmid (Invitrogen, Carlsbad Calif.) and PCR3.1 plasmid (Invitrogen), both of which contain the cytomegalovirus promoter. 5-10 μg of recombinant vector are transiently transfected into a human cell line, for example, an endothelial or hematopoietic cell line, using either liposome formulations or electroporation. 1-2 μg of an additional plasmid containing sequences encoding a marker protein are co-transfected. Expression of a marker protein provides a means to distinguish transfected cells from nontransfected cells and is a reliable predictor of cDNA expression from the recombinant vector. Marker proteins of choice include, e.g., Green Fluorescent Protein (GFP; Clontech), CD64, or a CD64-GFP fusion protein. Flow cytometry (FCM), an automated, laser optics-based technique, is used to identify transfected cells expressing GFP or CD64-GFP and to evaluate the apoptotic state of the cells and other cellular properties. FCM detects and quantifies the uptake of fluorescent molecules that diagnose events preceding or coincident with cell death. These events include changes in nuclear DNA content as measured by staining of DNA with propidium iodide; changes in cell size and granularity as measured by forward light scatter and 90 degree side light scatter; down-regulation of DNA synthesis as measured by decrease in bromodeoxyuridine uptake; alterations in expression of cell surface and intracellular proteins as measured by reactivity with specific antibodies; and alterations in plasma membrane composition as measured by the binding of fluorescein-conjugated Annexin V protein to the cell surface. Methods in flow cytometry are discussed in Ormerod, M. G. (1994; Flow Cytometry, Oxford, New York N.Y.).

The influence of REMAP on gene expression can be assessed using highly purified populations of cells transfected with sequences encoding REMAP and either CD64 or CD64-GFP. CD64 and CD64-GFP are expressed on the surface of transfected cells and bind to conserved regions of human immunoglobulin G (IgG). Transfected cells are efficiently separated from nontransfected cells using magnetic beads coated with either human IgG or antibody against CD64 (DYNAL, Lake Success N.Y.). mRNA can be purified from the cells using methods well known by those of skill in the art. Expression of mRNA encoding REMAP and other genes of interest can be analyzed by northern analysis or microarray techniques.

XV. Production of REMAP Specific Antibodies

REMAP substantially purified using polyacrylamide gel electrophoresis (PAGE; see, e.g., Harrington, M. G. (1990) Methods Enzymol. 182:488-495), or other purification techniques, is used to immunize animals (e.g., rabbits, mice, etc.) and to produce antibodies using standard protocols.

Alternatively, the REMAP amino acid sequence is analyzed using LASERGENE software (DNASTAR) to determine regions of high immunogenicity, and a corresponding oligopeptide is synthesized and used to raise antibodies by means known to those of skill in the art. Methods for selection of appropriate epitopes, such as those near the C-terminus or in hydrophilic regions are well described in the art (Ausubel et al., supra, ch. 11).

Typically, oligopeptides of about 15 residues in length are synthesized using an ABI 431A peptide synthesizer (Applied Biosystems) using FMOC chemistry and coupled to KLH (Sigma-Aldrich, St. Louis Mo.) by reaction with N-maleidobenzoyl-N-hydroxysuccinimide ester (MBS) to increase immunogenicity (Ausubel et al., supra). Rabbits are immunized with the oligopeptide-KLH complex in complete Freund's adjuvant. Resulting antisera are tested for antipeptide and anti-REMAP activity by, for example, binding the peptide or REMAP to a substrate, blocking with 1% BSA, reacting with rabbit antisera, washing, and reacting with radio-iodinated goat anti-rabbit IgG.

XVI. Purification of Naturally Occurring REMAP Using Specific Antibodies

Naturally occurring or recombinant REMAP is substantially purified by immunoaffinity chromatography using antibodies specific for REMAP. An immunoaffinity column is constructed by covalently coupling anti-REMAP antibody to an activated chromatographic resin, such as CNBr-activated SEPHAROSE (Amersham Biosciences). After the coupling, the resin is blocked and washed according to the manufacturer's instructions.

Media containing REMAP are passed over the immunoaffinity column, and the column is washed under conditions that allow the preferential absorbance of REMAP (e.g., high ionic strength buffers in the presence of detergent). The column is eluted under conditions that disrupt antibody/REMAP binding (e.g., a buffer of pH 2 to pH 3, or a high concentration of a chaotrope, such as urea or thiocyanate ion), and REMAP is collected.

XVII. Identification of Molecules Which Interact with REMAP

REMAP, or biologically active fragments thereof, are labeled with 1′ Bolton-Hunter reagent (Bolton, A. E. and W. M. Hunter (1973) Biochem. J. 133:529-539). Candidate molecules previously arrayed in the wells of a multi-well plate are incubated with the labeled REMAP, washed, and any wells with labeled REMAP complex are assayed. Data obtained using different concentrations of REMAP are used to calculate values for the number, affinity, and association of REMAP with the candidate molecules.

Alternatively, molecules interacting with REMAP are analyzed using the yeast two-hybrid system as described in Fields, S. and O. Song (1989; Nature 340:245-246), or using commercially available kits based on the two-hybrid system, such as the MATCHMAKER system (Clontech).

REMAP may also be used in the PATHCALLING process (CuraGen Corp., New Haven Conn.) which employs the yeast two-hybrid system in a high-throughput manner to determine all interactions between the proteins encoded by two large libraries of genes (Nandabalan, K. et al. (2000) U.S. Pat. No. 6,057,101).

XVIII. Demonstration of REMAP Activity

An assay for REMAP activity measures the expression of REMAP on the cell surface. cDNA encoding REMAP is transfected into an appropriate mammalian cell line. Cell surface proteins are labeled with biotin as described (de la Fuente, M. A. et al. (1997) Blood 90:2398-2405). Immunoprecipitations are performed using REMAP-specific antibodies, and immunoprecipitated samples are analyzed using sodium dodecyl sulfate polyacrylamide gel electrophoresis (SDS-PAGE) and immunoblotting techniques. The ratio of labeled immunoprecipitant to unlabeled immunoprecipitant is proportional to the amount of REMAP expressed on the cell surface.

In the alternative, an assay for REMAP activity is based on a prototypical assay for ligand/receptor-mediated modulation of cell proliferation. This assay measures the rate of DNA synthesis in Swiss mouse 3T3 cells. A plasmid containing polynucleotides encoding REMAP is added to quiescent 3T3 cultured cells using transfection methods well known in the art. The transiently transfected cells are then incubated in the presence of [³H]thymidine, a radioactive DNA precursor molecule. Varying amounts of REMAP ligand are then added to the cultured cells. Incorporation of [³H]thymidine into acid-precipitable DNA is measured over an appropriate time interval using a radioisotope counter, and the amount incorporated is directly proportional to the amount of newly synthesized DNA. A linear dose-response curve over at least a hundred-fold REMAP ligand concentration range is indicative of receptor activity. One unit of activity per milliliter is defined as the concentration of REMAP producing a 50% response level, where 100% represents maximal incorporation of [³H]thymidine into acid-precipitable DNA (McKay, I. and I. Leigh, eds. (1993) Growth Factors: A Practical Approach, Oxford University Press, New York N.Y., p. 73.)

In a further alternative, the assay for REMAP activity is based upon the ability of GPCR family proteins to modulate G protein-activated second messenger signal transduction pathways (e.g., cAMP; Gaudin, P. et al. (1998) J. Biol. Chem. 273:4990-4996). A plasmid encoding full length REMAP is transfected into a mammalian cell line (e.g., Chinese hamster ovary (CHO) or human embryonic kidney (BFK-293) cell lines) using methods well-known in the art. Transfected cells are grown in 12-well trays in culture medium for 48 hours, then the culture medium is discarded, and the attached cells are gently washed with PBS. The cells are then incubated in culture medium with or without ligand for 30 minutes, then the medium is removed and cells lysed by treatment with 1 M perchloric acid. The cAMP levels in the lysate are measured by radioimmunoassay using methods well-known in the art. Changes in the levels of cAMP in the lysate from cells exposed to ligand compared to those without ligand are proportional to the amount of REMAP present in the transfected cells.

To measure changes in inositol phosphate levels, the cells are grown in 24-well plates containing 1×10⁵ cells/well and incubated with inositol-free media and [³H]myoinositol, 2 μCi/well, for 48 hr. The culture medium is removed, and the cells washed with buffer containing 10 mM LiCl followed by addition of ligand. The reaction is stopped by addition of perchloric acid. Inositol phosphates are extracted and separated on Dowex AG1-X8 (Bio-Rad) anion exchange resin, and the total labeled inositol phosphates counted by liquid scintillation. Changes in the levels of labeled inositol phosphate from cells exposed to ligand compared to those without ligand are proportional to the amount of REMAP present in the transfected cells.

In a further alternative, the ion conductance capacity of REMAP is demonstrated using an electrophysiological assay. REMAP is expressed by transforming a mammalian cell line such as COS7, HeLa or CHO with a eukaryotic expression vector encoding REMAP. Eukaryotic expression vectors are commercially available, and the techniques to introduce them into cells are well known to those skilled in the art. A small amount of a second plasmid, which expresses any one of a number of marker genes such as β-galactosidase, is co-transformed into the cells in order to allow rapid identification of those cells which have taken up and expressed the foreign DNA. The cells are incubated for 48-72 hours after transformation under conditions appropriate for the cell line to allow expression and accumulation of REMAP and β-galactosidase. Transformed cells expressing β-galactosidase are stained blue when a suitable colorimetric substrate is added to the culture media under conditions that are well known in the art. Stained cells are tested for differences in membrane conductance due to various ions by electrophysiological techniques that are well known in the art. Untransformed cells, and/or cells transformed with either vector sequences alone or β-galactosidase sequences alone, are used as controls and tested in parallel. The contribution of REMAP to cation or anion conductance can be shown by incubating the cells using antibodies specific for either REMAP. The respective antibodies will bind to the extracellular side of REMAP, thereby blocking the pore in the ion channel, and the associated conductance.

In a further alternative, REMAP transport activity is assayed by measuring uptake of labeled substrates into Xenopus laevis oocytes. Oocytes at stages V and VI are injected with REMAP mRNA (10 ng per oocyte) and incubated for 3 days at 18° C. in OR2 medium (82.5 mM NaCl, 2.5 mM KCl, 1 mM CaCl₂, 1 mM MgCl₂, 1 mM Na₂HPO₄, 5 mM Hepes, 3.8 mM NaOH, 50 μg/ml gentamycin, pH 7.8) to allow expression of REMAP protein. Oocytes are then transferred to standard uptake medium (100 mM NaCl, 2 mM KCl, 1 mM CaCl, 1 mM MgCl₂, 10 mM Hepes/Tris pH 7.5). Uptake of various substrates (e.g., amino acids, sugars, drugs, and neurotransmitters) is initiated by adding a ³H substrate to the oocytes. After incubating for 30 minutes, uptake is terminated by washing the oocytes three times in Na⁺-free medium, measuring the incorporated ³H, and comparing with controls. REMAP activity is proportional to the level of internalized ³H substrate.

In a further alternative, REMAP protein kinase (PK) activity is measured by phosphorylation of a protein substrate using gamma-labeled [³²P]-ATP and quantitation of the incorporated radioactivity using a gamma radioisotope counter. REMAP is incubated with the protein substrate, [³²P]-ATP, and an appropriate kinase buffer. The ³²P incorporated into the product is separated from free [³²P]-ATP by electrophoresis and the incorporated ³²P is counted. The amount of ³²P recovered is proportional to the PK activity of REMAP in the assay. A determination of the specific amino acid residue phosphorylated is made by phosphoamino acid analysis of the hydrolyzed protein.

Further, adenylyl cylcase activity of REMAP is demonstrated by the ability to convert ATP to cAMP (Mittal, C. K. (1986) Methods Enzymol. 132:422-428). In this assay REMAP is incubated with the substrate [α-³²P]ATP, following which the excess substrate is separated from the product cyclic [³²P] AMP. REMAP activity is determined in 12×75 mm disposable culture tubes containing 5 μl of 0.6 M Tris-HCl, pH 7.5, 5 μl of 0.2 M MgCl₂, 5 μl of 150 mM creatine phosphate containing 3 units of creatine phosphokinase, 5 μl of 4.0 mM 1-methyl-3-isobutylxanthine, 5 μl of 20 mM cAMP, 5 μl 20 mM dithiothreitol, 5 μl of 10 mM ATP, 10 μl [α-³²P]ATP (2-4×10⁶ cpm), and water in a total volume of 100 μl. The reaction mixture is prewarmed to 30° C. The reaction is initiated by adding REMAP to the prewarmed reaction mixture. After 10-15 minutes of incubation at 30° C., the reaction is terminated by adding 25 μl of 30% ice-cold trichloroacetic acid (TCA). Zero-time incubations and reactions incubated in the absence of REMAP are used as negative controls. Products are separated by ion exchange chromatography, and cyclic [³²P] AMP is quantified using a P-radioisotope counter. The REMAP activity is proportional to the amount of cyclic [³²P] AMP formed in the reaction. XIX. Identification of REMAP Ligands REMAP is expressed in a eukaryotic cell line such as CHO (Chinese Hamster Ovary) or HEK (Human Embryonic Kidney) 293 which have a good history of GPCR expression and which contain a wide range of G-proteins allowing for functional coupling of the expressed REMAP to downstream effectors. The transformed cells are assayed for activation of the expressed receptors in the presence of candidate ligands. Activity is measured by changes in intracellular second messengers, such as cyclic AMP or Ca²⁺. These may be measured directly using standard methods well known in the art, or by the use of reporter gene assays in which a luminescent protein (e.g. firefly luciferase or green fluorescent protein) is under the transcriptional control of a promoter responsive to the stimulation of protein kinase C by the activated receptor (Milligan, G. et al. (1996) Trends Pharmacol. Sci. 17:235-237). Assay technologies are available for both of these second messenger systems to allow high throughput readout in multi-well plate format, such as the adenylyl cyclase activation FlashPlate Assay (NEN Life Sciences Products), or fluorescent Ca²⁺ indicators such as Fluo-4 AM (Molecular Probes) in combination with the FLIPR fluorimetric plate reading system (Molecular Devices). In cases where the physiologically relevant second messenger pathway is not known, REMAP may be coexpressed with the G-proteins G_{α15/16} which have been demonstrated to couple to a wide range of G-proteins (Offermanns, S. and M. I. Simon (1995) J. Biol. Chem. 270:15175-15180), in order to funnel the signal transduction of the REMAP through a pathway involving phospholipase C and Ca²⁺ mobilization. Alternatively, REMAP may be expressed in engineered yeast systems which lack endogenous GPCRs, thus providing the advantage of a null background for REMAP activation screening. These yeast systems substitute a human GPCR and G_{α} protein for the corresponding components of the endogenous yeast pheromone receptor pathway. Downstream signaling pathways are also modified so that the normal yeast response to the signal is converted to positive growth on selective media or to reporter gene expression (Broach, J. R. and J. Thorner (1996) Nature 384 (supp.):14-16). The receptors are screened against putative ligands including known GPCR ligands and other naturally occurring bioactive molecules. Biological extracts from tissues, biological fluids and cell supernatants are also screened.

Various modifications and variations of the described compositions, methods, and systems of the invention will be apparent to those skilled in the art without departing from the scope and spirit of the invention. It will be appreciated that the invention provides novel and useful proteins, and their encoding polynucleotides, which can be used in the drug discovery process, as well as methods for using these compositions for the detection, diagnosis, and treatment of diseases and conditions. Although the invention has been described in connection with certain embodiments, it should be understood that the invention as claimed should not be unduly limited to such specific embodiments. Nor should the description of such embodiments be considered exhaustive or limit the invention to the precise forms disclosed. Furthermore, elements from one embodiment can be readily recombined with elements from one or more other embodiments. Such combinations can form a number of embodiments within the scope of the invention. It is intended that the scope of the invention be defined by the following claims and their equivalents.

TABLE 1

| | | Incyte | | Incyte | |
|---|---|---|---|---|---|
| Incyte | Polypeptide | Polypeptide | Polynucleotide | Polynucleotide | Incyte Full Length |
| Project ID | SEQ ID NO: | ID | SEQ ID NO: | ID | Clones |
| 3356677 | 1 | 3356677CD1 | 48 | 3356677CB1 | |
| 7481665 | 2 | 7481665CD1 | 49 | 7481665CB1 | |
| 3563859 | 3 | 3563859CD1 | 50 | 3563859CB1 | |
| 2588884 | 4 | 2588884CD1 | 51 | 2588884CB1 | 2588884CA2 |
| 7503422 | 5 | 7503422CD1 | 52 | 7503422CB1 | |
| 7503424 | 6 | 7503424CD1 | 53 | 7503424CB1 | |
| 7503571 | 7 | 7503571CD1 | 54 | 7503571CB1 | 5946903CA2, 71826507CA2 |
| 7505722 | 8 | 7505722CD1 | 55 | 7505722CB1 | |
| 7505798 | 9 | 7505798CD1 | 56 | 7505798CB1 | 90085569CA2 |
| 7505847 | 10 | 7505847CD1 | 57 | 7505847CB1 | |
| 7505862 | 11 | 7505862CD1 | 58 | 7505862CB1 | 973101CA2 |
| 7762537 | 12 | 7762537CD1 | 59 | 7762537CB1 | |
| 90033462 | 13 | 90033462CD1 | 60 | 90033462CB1 | 90033462CA2, 90033478CA2, |
| | | | | | 90033494CA2 |
| 1644869 | 14 | 1644869CD1 | 61 | 1644869CB1 | 90171183CA2 |
| 6288712 | 15 | 6288712CD1 | 62 | 6288712CB1 | 6288712CA2, 6717974CA2, |
| | | | | | 7207246CA2, 90166685CA2, |
| | | | | | 90166693CA2 |
| 71830156 | 16 | 71830156CD1 | 63 | 71830156CB1 | 6246040CA2 |
| 7505044 | 17 | 7505044CD1 | 64 | 7505044CB1 | 90179613CA2, 90179637CA2, |
| | | | | | 90179713CA2 |
| 7505086 | 18 | 7505086CD1 | 65 | 7505086CB1 | |
| 7505784 | 19 | 7505784CD1 | 66 | 7505784CB1 | |
| 7505813 | 20 | 7505813CD1 | 67 | 7505813CB1 | 90182824CA2 |
| 7505873 | 21 | 7505873CD1 | 68 | 7505873CB1 | 90060871CA2 |
| 7505881 | 22 | 7505881CD1 | 69 | 7505881CB1 | |
| 7503510 | 23 | 7503510CD1 | 70 | 7503510CB1 | |
| 7714715 | 24 | 7714715CD1 | 71 | 7714715CB1 | 90197262CA2, 90197362CA2, |
| | | | | | 90197386CA2 |
| 7506032 | 25 | 7506032CD1 | 72 | 7506032CB1 | 90187946CA2, 90188319CA2, |
| | | | | | 90188476CA2 |
| 7506034 | 26 | 7506034CD1 | 73 | 7506034CB1 | 90059536CA2 |
| 7506100 | 27 | 7506100CD1 | 74 | 7506100CB1 | 7628458CA2 |
| 1743113 | 28 | 1743113CD1 | 75 | 1743113CB1 | 90197420CA2 |
| 7505144 | 29 | 7505144CD1 | 76 | 7505144CB1 | |
| 7506132 | 30 | 7506132CD1 | 77 | 7506132CB1 | |
| 8142016 | 31 | 8142016CD1 | 78 | 8142016CB1 | |
| 7506135 | 32 | 7506135CD1 | 79 | 7506135CB1 | |
| 90086301 | 33 | 90086301CD1 | 80 | 90086301CB1 | 90086149CA2 |
| 7487373 | 34 | 7487373CD1 | 81 | 7487373CB1 | |
| 7506228 | 35 | 7506228CD1 | 82 | 7506228CB1 | 6913184CA2 |
| 7506194 | 36 | 7506194CD1 | 83 | 7506194CB1 | |
| 7506434 | 37 | 7506434CD1 | 84 | 7506434CB1 | 90117366CA2 |
| 7490974 | 38 | 7490974CD1 | 85 | 7490974CB1 | |
| 7506224 | 39 | 7506224CD1 | 86 | 7506224CB1 | |
| 7506280 | 40 | 7506280CD1 | 87 | 7506280CB1 | |
| 7508326 | 41 | 7508326CD1 | 88 | 7508326CB1 | 1288849CA2 |
| 7506370 | 42 | 7506370CD1 | 89 | 7506370CB1 | 7276685CA2 |
| 6312989 | 43 | 6312989CD1 | 90 | 6312989CB1 | |
| 7501108 | 44 | 7501108CD1 | 91 | 7501108CB1 | 90213787CA2 |
| 7507581 | 45 | 7507581CD1 | 92 | 7507581CB1 | |
| 7506361 | 46 | 7506361CD1 | 93 | 7506361CB1 | |
| 7509211 | 47 | 7509211CD1 | 94 | 7509211CB1 | |

TABLE 2

| Poly- | Incyte | GenBank | | |
|---|---|---|---|---|
| peptide | Poly- | ID NO: | | |
| SEQ | peptide | or PROTEOME | Probability | |
| ID NO: | ID | ID NO: | Score | Annotation |
| 2 | 7481665CD1 | 336694\|OGT | 2.1E−23 | [Homo sapiens][Transferase; Small molecule-binding protein] |
| | | | | [Nuclear; Cytoplasmic] O-linked N-acetylglucosamine |
| | | | | transferase (UDP-N-acetylglucosamine: polypeptide-N- |
| | | | | acetylglucosaminyltransferase), enzyme that functions in O-glycosyl |
| | | | | ation, may be involved in insulin secretion and glucose |
| | | | | homeostasis, may play a role in signal transduction |
| 3 | 3563859CD1 | g14017773 | 1.0E−169 | [Mus musculus] Cg10671-like |
| | | | | Yawata, M., et al. (2001) Immunogenetics 53: 119-129 |
| | | | | Nucleotide sequence analysis of the approximately 35-kb segment |
| | | | | containing interferon-gamma-inducible mouse proteasome activator |
| | | | | genes |
| 4 | 2588884CD1 | g4468344 | 3.8E−70 | [Homo sapiens] LOX1 |
| | 2588884CD1 | 344658\|OLR1 | 3.3E−71 | [Homo sapiens][Receptor (protein translocation)][Plasma |
| | | | | membrane] Lectin-like oxidized low density lipoprotein receptor, |
| | | | | member of the C-type lectin receptor family involved in degradation |
| | | | | of oxidized low density lipoprotein by vascular endothelial cells, |
| | | | | associated with atherosclerosis |
| 5 | 7503422CD1 | g562106 | 2.6E−99 | [Homo sapiens] dlk gene product |
| | | | | Lee, Y. L., et al. (1995) Biochim. Biophys. Acta 1261: 223-232 |
| | | | | dlk, pG2 and Pref-1 mRNAs encode similar proteins belonging to the |
| | | | | EGF-like superfamily. Identification of polymorphic variants of |
| | | | | this RNA |
| | | 335048\|DLK1 | 1.2E−99 | [Homo sapiens][Inhibitor or repressor][Secretory |
| | | | | vesicles; Cytoplasmic; Extracellular (excluding cell wall)] |
| | | | | Preadipocyte factor (fetal antigen 1), a member of the epidermal |
| | | | | growth factor-like family and putative homolog of Drosophila |
| | | | | homeotic proteins Delta and Notch, inhibits adipocyte |
| | | | | differentiation and may function in neuroendocrine differentiation |
| | | 342754\|RTN1 | 3.2E−31 | [Homo sapiens][Ligand][Endoplasmic reticulum; |
| | | | | Cytoplasmic] Member of the reticulon family of endoplasmic |
| | | | | reticulum proteins, has strong similarity to murine Dll1, which is |
| | | | | likely involved in cell-cell communication during somitogenesis |
| | | | | and development of the nervous system |
| 6 | 7503424CD1 | g562106 | 1.8E−102 | [Homo sapiens] dlk gene product |
| | | | | Lee, Y. L., et al. (1995) Biochim. Biophys. Acta 1261: 223-232 |
| | | | | dlk, pG2 and Pref-1 mRNAs encode similar proteins belonging to the |
| | | | | EGF-like superfamily. Identification of polymorphic variants of |
| | | | | this RNA |
| | | 335048\|DLK1 | 4.9E−102 | [Homo sapiens][Inhibitor or repressor][Secretory |
| | | | | vesicles; Cytoplasmic; Extracellular (excluding cell wall)] |
| | | | | Preadipocyte factor (fetal antigen 1), a member of the epidermal |
| | | | | growth factor-like family and putative homolog of Drosophila |
| | | | | homeotic proteins Delta and Notch, inhibits adipocyte |
| | | | | differentiation and may function in neuroendocrine differentiation |
| | | 342754\|RTN1 | 3.8E−42 | [Homo sapiens][Ligand][Endoplasmic reticulum; |
| | | | | Cytoplasmic] Member of the reticulon family of endoplasmic |
| | | | | reticulum proteins, has strong similarity to murine Dll1, which is |
| | | | | likely involved in cell-cell communication during somitogenesis |
| | | | | and development of the nervous system |
| 7 | 7503571CD1 | g2587054 | 5.1E−59 | [Homo sapiens] putative tetraspan transmembrane protein L6H |
| | | 338550\|TM4SF5 | 4.5E−60 | [Homo sapiens][Plasma membrane] Transmembrane 4 |
| | | | | superfamily member 5, member of the TM4 superfamily which includes |
| | | | | proteins with four putative transmembrane domains, highly |
| | | | | expressed in tumor cells |
| | | 341368\|TM4SF4 | 1.0E−37 | [Homo sapiens][Plasma membrane] Transmembrane 4 |
| | | | | superfamily member 4, member of the tetraspan membrane protein |
| | | | | superfamily that mediates density-associated inhibition of cell |
| | | | | proliferation |
| | | 365785\|TM4SF1 | 4.3E−23 | [Homo sapiens][Plasma membrane] L6 antigen, member of |
| | | | | the transmembrane 4 superfamily (TM4SF), a cell surface tumor |
| | | | | antigen that is highly expressed in lung, breast, colon, and ovarian |
| | | | | carcinomas |
| 8 | 7505722CD1 | g180098 | 6.0E−11 | [Homo sapiens] differentiation antigen |
| | | | | Simmons, D. and Seed, B. (1988) J. Immunol. 141: 2797-2800 |
| | | | | Isolation of a cDNA encoding CD33, a differentiation antigen of |
| | | | | myeloid progenitor cells |
| 8 | | 334524\|CD33 | 5.2E−12 | [Homo sapiens][Adhesin/agglutinin; Inhibitor or repressor; |
| | | | | Receptor (signalling); Small molecule-binding protein][Plasma |
| | | | | membrane] Myeloid cell antigen, a sialoadhesin that may mediate |
| | | | | cell-to-cell adhesion, acts as an inhibitory receptor that inhibits |
| | | | | the proliferation of normal and leukemic myeloid cells, expressed |
| | | | | only in cells of myelomonocytic lineage |
| 9 | 7505798CD1 | 559592 | 5.4E−66 | [Homo sapiens] Protein of unknown function, has a region of |
| | | \|DJ167A19.1 | | weak similarity to a region of SLC22A4, which is a proton/organic |
| | | | | cation transporter |
| 13 | 90033462CD1 | 728144\|K07H8.2 | 1.2E−27 | [Caenorhabditis elegans] Protein of unknown function, has |
| | | | | strong similarity toC. elegans ZK 185.2 and a divalent cation |
| | | | | transporter domain |
| | | | | Jiang, M. et al. (2001) Genome-wide analysis of developmental and |
| | | | | sex-regulated gene expression profiles in Caenorhabditis elegans. |
| | | | | Proc. Natl. Acad. Sci. USA 98: 218-223 |
| | | 253821\|ZK1053.6 | 1.2E−12 | [Caenorhabditis elegans] Protein of unknown function, has |
| | | | | moderate similarity to C. elegans T20D4.5 and a divalent cation |
| | | | | transporter domain |
| | | | | Bateman, A. et al. (1999) Pfam 3.1: 1313 multiple alignments and |
| | | | | profile HMMs match the majority of proteins. Nucleic Acids Res. |
| | | | | 27: 260-262 |
| 14 | 1644869CD1 | g7209574 | 4.7E−239 | [Homo sapiens] LAK-4p |
| 15 | 6288712CD1 | 84929330 | 8.4E−27 | [Homo sapiens] hypoxia-inducbile gene 1 |
| 16 | 71830156CD1 | g6434902 | 2.5E−121 | [Homo sapiens] tetraspanin TM4-B |
| | | | | Puls, K. L. et al. (1999) The molecular characterisation of a |
| | | | | novel tetraspanin protein, TM4-B(1). Biochim. Biophys. Acta |
| | | | | 1447: 93-99 |
| | | 429028\|TM4-B | 2.2E−122 | [Homo sapiens] Tetraspanin TM4-B, member of the tetraspanin |
| | | | | superfamily, closely homolgous to TSPAN-1 and widely expressed |
| | | | | Puls, K. L. et al. (1999) The molecular characterisation of a |
| | | | | novel tetraspanin protein, TM4-B(1). Biochim. Biophys. Acta |
| | | | | 1447: 93-99 |
| 16 | | 608978\|Tm4sf | 5.7E−21 | [Mus musculus] Transmembrane 4 superfamily member 6, member |
| | | | | of the tetraspanin family, may be involved in cell adhesion, |
| | | | | migration, and proliferation |
| | | | | Todd, S. C. et al. (1998) Sequences and expression of six new |
| | | | | members of the tetraspanin/TM4SF family. Biochim Biophys Acta |
| | | | | 1399: 101-104 |
| 17 | 7505044CD1 | g180098 | 2.5E−124 | [Homo sapiens] differentiation antigen |
| | | | | Simmons, D. and Seed, B. (1988) Isolation of a cDNA encoding CD33, |
| | | | | a differentiation antigen of myeloid progenitor cells. J. Immunol. |
| | | | | 141: 2797-2800 |
| | | 334524\|CD33 | 2.2E−125 | [Homo sapiens][Adhesin/agglutinin; Inhibitor orrepressor; |
| | | | | Receptor (signalling); Small molecule-binding protein][Plasma |
| | | | | membrane] Myeloid cell antigen, a sialoadhesin that may mediate |
| | | | | cell-to-cell adhesion, acts as an inhibitory receptor that |
| | | | | inhibits the proliferation of normal and leukemic myeloid cells, |
| | | | | expressed only in cells of myelomonocytic lineage |
| | | | | Vitale, C. et al. (1999) Engagement of p75/AIRM1 or CD33 inhibits |
| | | | | the proliferation of normal or leukemic myeloid cells. Proc. Natl. |
| | | | | Acad. Sci. USA 96: 15091-15096 |
| | | 704267\|Siglece | 3.0E−48 | [Mus musculus] Sialic acid-binding immunoglobulin-like |
| | | | | lectin E, recruits protein tyrosine phosphatases SHP-1 and SHP-2, |
| | | | | which inhibit immunoreceptor signal transduction, may function as |
| | | | | an inhibitory receptor in hematopoietic cells and the immune system |
| | | | | Yu, Z. et al. (2001) mSiglec-E, a novel mouse CD33-related |
| | | | | siglec (sialic acid-binding immunoglobulin-like lectin) that |
| | | | | recruits Src homology 2 (SH2)-domain-containing protein tyrosine |
| | | | | phosphatases SHP-1 and SHP-2. Biochem. J. 353: 483-492 |
| 18 | 7505086CD1 | g183391 | 0.0 | [Homo sapiens] granule membrane protein-140 (GMP-140) |
| | | | | precursor |
| | | | | Johnston, G. I. et al. (1989) Cloning of GMP-140, a granule |
| | | | | membrane protein of platelets and endothelium: Sequence similarity |
| | | | | to proteins involved in cell adhesion and inflammation. |
| | | | | Cell 56: 1033-1044 |
| 18 | | 337948\|SELP | 0.0 | [Homo sapiens][Adhesin/agglutinin; Receptor (signalling); |
| | | | | Small molecule-binding protein] |
| | | | | [Secretory vesicles; Cytoplasmic; Plasma membrane] P- |
| | | | | selectin (granule membrane protein 140 kD), adhesion molecule, |
| | | | | recruits leukocytes to blood vessel endothelium during inflammation |
| | | | | and monocytes to artherosclerotic sites; elevated plasma and |
| | | | | platelet levels are associated with hypercholesteremia |
| | | | | Johnston, G. I. et al. (1990) Structure of the human gene |
| | | | | encoding granule membrane protein-140, a member of the selectin |
| | | | | family of adhesion receptors for leukocytes. J. Biol. |
| | | | | Chem. 265: 21381-21385 |
| | | 585795\|Selp | 1.6E−229 | [Mus musculus][Adhesin/agglutinin; Receptor (signalling); |
| | | | | Small molecule-binding protein] P-selectin (platelet selectin), |
| | | | | adhesion molecule that recruits monocytes to sites of inflammation, |
| | | | | involved in leukocyte rolling, may play a role in tumor metastasis; |
| | | | | human SELP is associated with hypercholesteremia |
| | | | | Weller, A. et al. (1992) Cloning of the mouse endothelial |
| | | | | selectins. Expression of both E- and P- selectin is inducible by |
| | | | | tumor necrosis factor alpha. J. Biol. Chem. 267: 15176-15183 |
| 19 | 7505784CD1 | g3152701 | 2.2E−89 | [Homo sapiens] tetraspan NET-1 |
| | | | | Serru, V. et al. (2000) Sequence and expression of seven new |
| | | | | tetraspans. Biochim. Biophys. Acta 1478: 159-163 |
| | | 342880\|TSPAN-1 | 5.0E−90 | [Homo sapiens] Tetraspan 1, member of the tetraspanin |
| | | | | transmembrane 4 (TM4SF) superfamily, may regulate cell |
| | | | | proliferation, differentiation, adhesion, and migration |
| | | | | Todd, S. C. et al. (1998) Sequences and expression of six new |
| | | | | members of the tetraspanin/TM4SF family. Biochim. Biophys. |
| | | | | Acta 1399: 101-104 |
| 19 | | 608746\|Tm4sf2 | 7.4E−16 | [Mus musculus][Plasma membrane] Member of the |
| | | | | transmembrane 4 superfamily (TM4SF) that may play a role in |
| | | | | neuronal functioning, expressed on immature T cells and in the |
| | | | | brain; mutations in the corresponding human gene are associated |
| | | | | with mental retardation Zemni, R. et al. (2000) A new gene |
| | | | | involved in X-linked mental retardation identified by analysis of |
| | | | | an X; 2 balanced translocation. Nat. Genet. 24: 167-170 |
| 20 | 7505813CD1 | g29794 | 3.3E−133 | [Homo sapiens] CD37 (AA 1-244) |
| | | | | Classon, B. J. et al. (1989) The primary structure of the human |
| | | | | leukocyte antigen CD37, a species homologue of the rat MRC OX-44 |
| | | | | antigen. J. Exp. Med. 169: 1497-1502 |
| | | 334532\|CD37 | 2.9E−134 | [Homo sapiens][Plasma membrane] CD37 leukocyte surface |
| | | | | antigen, member of the transmembrane 4 superfamily of |
| | | | | glycoproteins with four predicted transmembrane domains; mutation |
| | | | | in mouse Cd37 results in compromised humoral immune response |
| | | | | Virtaneva, K. I. et al. (1993) The genes for CD37, CD53, and R2, |
| | | | | all members of a novel gene family, are located on different |
| | | | | chromosomes. Immunogenetics 37: 46146-5 |
| | | 583747\|Cd37 | 1.1E−106 | [Mus musculus][Plasma membrane] Cd37 leukocyte surface |
| | | | | antigen, member of the transmembrane 4 superfamily of |
| | | | | glycoproteins with four predicted transmembrane domains; mutation |
| | | | | results in compromised humoral immune response |
| | | | | Tomlinson, M. G. and Wright, M. D. (1996) Characterisation of |
| | | | | mouse CD37: cDNA and genomic cloning. Mol. Immunol. 33: 867-872 |
| 21 | 7505873CD1 | g4206155 | 6.5E−235 | [Homo sapiens] Mcd4p homolog |
| | | | | Gaynor, E. C. et al. (1999) MCD4 encodes a conserved endoplasmic |
| | | | | reticulum membrane protein essential for |
| | | | | glycosylphosphatidylinositol anchor synthesis in yeast. Mol. Biol. |
| | | | | Cell 10: 627-648 |
| 21 | | 428848\|PIGN | 5.7E−236 | [Homo sapiens][Transferase][Endoplasmic reticulum; |
| | | | | Cytoplasmic] Phosphatidylinositol glycan class N, a putative |
| | | | | endoplasmicreticulum protein that may act in GPI anchor synthesis, |
| | | | | has multiple transmembrane domains, and contains sequence motifs |
| | | | | found in phosphodiesterases and pyrophosphatases |
| | | | | Gaynor, E. C. et al. (1999) MCD4 encodes a conserved endoplasmic |
| | | | | reticulum membrane protein essential for |
| | | | | glycosylphosphatidylinositol anchor synthesis in yeast. Mol. Biol. |
| | | | | Cell 10: 627-648 |
| | | 587247\|Pign | 3.7E−221 | [Mus musculus][Transferase][Endoplasmic reticulum; |
| | | | | Cytoplasmic] Phosphatidylinositol glycan class N, transfers |
| | | | | phosphoethanol amine to the first mannose of |
| | | | | glycosylphosphatidylinositol anchors |
| | | | | Hong, Y. et al. (1999) Pig-n, a Mammalian Homologue of Yeast |
| | | | | Mcd4p, Is Involved in Transferring Phosphoethanolamine to the |
| | | | | First Mannose of the Glycosylphosphatidylinositol. J. Biol. |
| | | | | Chem. 274: 35099-35106 |
| 22 | 7505881CD1 | g11559250 | 4.6E−104 | [Homo sapiens] MS4A7 |
| | | | | Ishibashi, K. et al. (2001) Identification of a new multigene |
| | | | | four-transmembrane family (MS4A) related to CD20, HTm4 and beta |
| | | | | subunit of the high-affinity IgE receptor. Gene 264: 87-93 |
| | | 663409\|MS4A7 | 4.0E−105 | [Homo sapiens][Plasma membrane] Member of a four |
| | | | | transmembrane domain family, has similarity to B-cell-specific |
| | | | | antigen CD20, hematopoietic-cell-specific protein HTm4, and high |
| | | | | affinity IgE receptor beta chain |
| | | | | Ishibashi, K. et al. (2001) Identification of a new multigene |
| | | | | four-transmembrane family (MS4A) related to CD20, HTm4 and beta |
| | | | | subunit of the high-affinity IgE receptor. Gene 264: 87-93. |
| 23 | 7503510CD1 | g13537355 | 0.0 | [Homo sapiens] membrane glycoprotein LIG-1. |
| 23 | | 319126\|Img | 0.0 | [Mus musculus][Plasma membrane] Integral membrane |
| | | | | glycoprotein, a member of the leucine-rich repeat and |
| | | | | immunoglobulin superfamilies, may function as a glial cell- |
| | | | | specific adhesion molecule or receptor, may be involved in |
| | | | | neuroglial differentiation and development. |
| | | | | Suzuki, Y. et al. (1996) J. Biol. Chem. 271: 22522-22527. |
| | | 346594\|KIAA0806 | 5.3E−174 | [Homo sapiens] Protein with high similarity to murine Mm.944 |
| | | | | (LIG-1), which may function as an adhesion molecule or glial |
| | | | | receptor, contains leucine-rich repeats, a leucine-rich repeat |
| | | | | C-terminal cysteine-rich domain, and an immunoglobulin (Ig) |
| | | | | domain. |
| | | | | Nagase, T. et al. (1998) DNA Res. 5: 277-286. |
| 24 | 7714715CD1 | g4877582 | 1.7E−19 | [Homo sapiens] lipoma HMGIC fusion partner. |
| | | | | Petit, M.M. et al. (1999) Genomics 57: 438-441. |
| | | 346708\|LHFPL2 | 6.7E−105 | [Homo sapiens] Protein with low similarity to LHFP, which is |
| | | | | a member of the LHFP-like family and whose corresponding gene is |
| | | | | fused to HMGIC in lipoma cells. |
| | | | | Nagase, T. et al. (1996) DNA Res. 3: 321-329. |
| | | 342584\|LHFP | 1.5E−20 | [Homo sapiens] Lipoma HMGIC fusion partner, a member of a |
| | | | | family of high mobility group isoform C translocation partners; |
| | | | | corresponding gene is fused to HMGIC in lipomas with t(12; 13) |
| | | | | and may have a role in the etiology of lipomas. |
| | | | | Petit, M. M. R. et al. (1996) Genomics 36: 118-129. |
| 25 | 7506032CD1 | g14250557 | 1.1E−57 | [Homo sapiens] CGI-78 protein. |
| 26 | 7506034CD1 | g18032261 | 0.0 | transmembrane protein H4 [Homo sapiens] |
| 27 | 7506100CD1 | g4097253 | 9.8E−49 | [Homo sapiens] calcitonin gene-related peptide receptor |
| | | | | component protein. |
| | | | | Luebke, A. E. et al. (1996) Proc. Natl. Acad. Sci. U.S.A. |
| | | | | 93: 3455-3460. |
| 27 | | 569030\|CGRP-RCP | 8.7E−50 | [Homo sapiens][Receptor (signalling)] Calcitonin gene- |
| | | | | related peptide (CALCA) receptor component protein, associates |
| | | | | with CGRP receptor (CALCRL) to facilitate CGRP-mediated signalling, |
| | | | | associated with the acrosome and predicted to play a role in |
| | | | | reproduction. |
| | | | | Luebke, A. E. et al. (1996) supra |
| | | 325296\|Crcp | 8.6E−43 | [Mus musculus][Activator][Plasma membrane; |
| | | | | Unspecified membrane] Calcitonin gene-related peptide(CALCA) |
| | | | | receptor component protein, associates with the CGRP receptor |
| | | | | (Calcrl) to modulate CGRP-mediated inhibition of myometrial smooth |
| | | | | muscle contractions, mediates CGRP- and adrenomedullin receptor- |
| | | | | mediated signalling. |
| | | | | Luebke, A. E. et al. (1996) supra |
| 28 | 1743113CD1 | g3138977 | 8.3E−13 | [Caenorhabditis elegans] odorant response protein ODR-4. |
| | | | | Dwyer, N. D. et al. (1998) Cell 93: 455-466. |
| | | 714615\|odr-4 | 7.4E−14 | [Caenorhabditis elegans][Unknown][Golgi; Endoplasmic |
| | | | | reticulum; Axon; Cell body (soma); Other vesicles of the |
| | | | | secretory/endocytic pathways; Dendrite] Membrane-associated |
| | | | | protein involved in localization of odorant receptors to olfactory |
| | | | | neuron cilia. |
| 29 | 7505144CD1 | g1655592 | 2.9E−95 | [Homo sapiens] folate receptor. |
| | | | | Page, S. T. et al. (1993) J. Mol. Biol. 229: 1175-1183. |
| | | 335362\|FOLR2 | 5.4E−96 | [Homo sapiens] [Small molecule-binding protein] |
| | | | | [Unspecified membrane] Placental folate-binding protein |
| | | | | (folate receptor beta). |
| | | | | Prasad, P. D. et al. (1994) Biochim. Biophys. Acta 1223: 71-75. |
| 29 | | 335364\|FOLR3 | 3.9E−77 | [Homo sapiens][Receptor (signalling); Small molecule- |
| | | | | binding protein][Unspecified membrane] Folate receptor 3 |
| | | | | (gamma), one of a family of folate receptors that includes FOLR1 |
| | | | | and FOLR2, binds folic acid, primarily a secreted protein due to |
| | | | | lack of an efficient signal for glycosylphosphatidylinositol |
| | | | | anchor modification. |
| | | | | Shen, F. et al. (1994) Biochemistry 33: 1209-1215. |
| 30 | 7506132CD1 | g6759605 | 2.3E−96 | [Rattus norvegicus] Tspan-2 protein. |
| | | | | Birling, M. C. et al. (1999) J. Neurochem. 73: 2600-2608. |
| | | 658310 | 2.1E−97 | [Rattus norvegicus] Tetraspan 2, member of the transmembrane |
| | | Tspan-2 | | 4 superfamily of proteins, has four putative transmembrane |
| | | | | domains, and may play a role in central nervous system development |
| | | | | and nerve ensheathment. |
| | | | | Todd, S. C. et al. (1998) Biochim. Biophys. Acta 1399: 101-104. |
| | | 342882\|TSPAN-2 | 1.9E−96 | [Homo sapiens][Unspecified membrane] Tetraspan 2, |
| | | | | member of the transmembrane 4 superfamily of proteins, has four |
| | | | | putative transmembrane domains, and may play a role in cell |
| | | | | migration, proliferation, and adhesion. |
| | | | | Todd, S. C. et al. supra. |
| 31 | 8142016CD1 | g14209832 | 1.1E−276 | [Homo sapiens] transmembrane mucin MUC13. |
| | | | | Williams, S. J. et al. (2001) J. Biol. Chem. 276: 18327-18336. |
| | | 331012\|Rn.10719 | 3.2E−107 | [Rattus norvegicus] Protein with high similarity to lymphoc |
| | | | | yte antigen 64 (murine Ly64), which has serine/threonine-rich |
| | | | | tandem repeats and EGF-like cysteine-rich repeats, may regulate |
| | | | | cellular responses to IL-3, and is highly expressed in primary |
| | | | | myeloid progenitor cells. |
| | | 320822\|Ly64 | 1.0E−101 | [Mus musculus][Plasma membrane] Lymphocyte antigen 64, |
| | | | | has serine/threonine-rich tandem repeats and epidermal growth |
| | | | | factor-like cysteine-rich repeats, may regulate cellular |
| | | | | responses to IL-3, highly expressed in primary myeloid |
| | | | | progenitor cells. |
| | | | | Roshak, A. K. et al. (1999) J. Leukoc. Biol. 65: 43-49. |
| 32 | 7506135CD1 | g2564916 | 2.0E−277 | [Homo sapiens] cote1. |
| | | | | Winfield, S. L. et al. (1997) Genome Res. 7: 1020-1026. |
| 33 | 90086301CD1 | g183650 | 1.5E−70 | [Homo sapiens] gastrin releasing peptide receptor |
| | | | | (Corjay, M. H. et al. (1991) J. Biol. Chem. 266: 18771-18779.) |
| | | 342098\|GRPR | 1.3E−71 | [Homo sapiens][Receptor (signalling)][Plasma |
| | | | | membrane] Gastrin-releasing peptide receptor, a G-protein |
| | | | | coupled receptor, expressed in a variety of lung carcinomas |
| | | | | (Cardona, C. et al. (1992) Biochem J. 281: 115-120; Saurin, |
| | | | | J. C. et al. (1999) Cancer Res. 59: 962-967.) |
| | | 583125\|Grpr | 1.1E−56 | [Mus musculus][Receptor (signalling)][Plasma |
| | | | | membrane] Gastrin-releasing peptide receptor (bombesin |
| | | | | receptor), a G-protein coupled receptor, promotes growth of |
| | | | | fibroblasts, has strong similarity to human GRPR, which is |
| | | | | expressed in a variety of lung carcinomas |
| | | | | (Spindel, E. R. et al. (1990) Mol. Endocrinol. 4: 1956-1963; |
| | | | | King, K. A. et al. (1995) Proc. Natl. Acad. Sci. USA 92: |
| | | | | 4357-4361.) |
| 34 | 7487373CD1 | g11908217 | 9.7E−168 | [Homo sapiens] HOR5'Beta6 |
| | | | | (Bulger, M. et al. (2000) Proc. Natl. Acad. Sci. U.S.A. |
| | | | | 97: 14560-14565.) |
| | | 418919\|Olfr64 | 1.4E−143 | [Mus musculus][Receptor (signalling)][Plasma |
| | | | | membrane] Member of the rhodopsin family of G-protein coupled |
| | | | | receptors (GPCR), has low similarity to Olfr49, which is a |
| | | | | member of the G-protein coupled receptor family, has a likely |
| | | | | role in the olfactory response |
| | | 418920\|Olfr65 | 6.7E−121 | [Mus musculus][Receptor (signalling)][Plasma |
| | | | | membrane] Member of the rhodopsin family of G-protein coupled |
| | | | | receptors (GPCR), has low similarity to human OR2F1, which is a |
| | | | | member of a family of nasal epithelial G protein coupled seven- |
| | | | | transmembrane receptors that are involved in olfactory |
| | | | | transduction |
| 35 | 7506228CD1 | g5114049 | 2.3E−152 | [Homo sapiens] flotillin |
| | | | | (Zhang, Q. H. et al. (2000) Genome Res. 10: 1546-1560.) |
| 35 | | 342462\|FLOT1 | 2.6E−153 | [Homo sapiens][Plasma membrane] Flotillin 1, a caveola |
| | | | | e associated protein that may be involved in formation of lipid |
| | | | | rafts, which have been implicated in numerous cellular processes |
| | | | | including signal transduction; production is increased in senile |
| | | | | plaques in Alzheimer brain |
| | | | | (Kokubo, H. et al. (2000) Neurosci. Lett. 290: 93-96; |
| | | | | Edgar, A. J., and Polak, J. M. (2001)Int. J. Biochem. Cell Biol. |
| | | | | 33: 53-64.) |
| | | 320626\|Flot1 | 3.5E−149 | [Mus musculus][Plasma membrane] Flotillin 1, involved |
| | | | | in localizing proteins to caveolae and in phagocyte formation; |
| | | | | human FLOT1 is associated with Alzheimer's disease |
| | | | | (Bickel, P. E. et al. (1997) J. Biol. Chem. 272: 13793-13802; |
| | | | | Dermine, J. F. et al. (2001) J. Biol. Chem. 276: 18507-18512.) |
| 36 | 7506194CD1 | g9049783 | 0.0 | [Homo sapiens] adenylyl cyclase type VI |
| | | | | (Wicker, R. et al. (2000) Biochim. Biophys. Acta 1493: |
| | | | | 279-283.) |
| | | 613267\|ADCY6 | 2.0E−246 | [Homo sapiens][Lyase] Adenylyl cyclase type V, an ATP- |
| | | | | pyrophosphate lyase that converts ATP to cAMP, activity is |
| | | | | inhibited by calcium |
| | | | | (Wicker, R. et al. (2000) supra; Cooper, D. M. et al. (1994) |
| | | | | Biochem. J. 297: 437-440.) |
| | | 324218\|Adcy6 | 9.7E−223 | [Mus musculus][Lyase][Plasma membrane] Adenylate |
| | | | | cyclase type VI, an ATP-pyrophosphate lyase that converts ATP to |
| | | | | cAMP, activity is stimulated by forskolin and inhibited by |
| | | | | calcium; improves cardiac function |
| | | | | (Marjamaki, A. et al. (1997) J. Biol. Chem. 272: 16466-16473; |
| | | | | Roth, D. M. et al. (1999) Circulation 99: 3099-3102.) |
| 37 | 7506434CD1 | g292039 | 4.7E−12 | [Homo sapiens] GABA-alpha receptor beta-3 subunit |
| | | | | (Kirkness, E. F. and Fraser, C. M. (1993) J. Biol. Chem. |
| | | | | 268: 4420-4428.) |
| 37 | | 591053\|Gabrb3 | 8.1E−12 | [Rattus norvegicus][Channel (passive transporter); |
| | | | | Receptor (signalling); Transporter][Plasma membrane] |
| | | | | Beta 3 subunit of the gamma-amino butyric acid A receptor, which |
| | | | | is a chloride channel and the major inhibitory neurotransmitter |
| | | | | receptor in the brain; deletions in human GABRB3 are implicated |
| | | | | in Angelman syndrome |
| | | | | (Klausberger, T. et al. (2001) J. Biol. Chem. 276: 16024-16032.) |
| | | 323602\|Gabrb3 | 8.1E−12 | [Mus musculus][Channel (passive transporter); Receptor |
| | | | | (signalling); Transporter][Plasma membrane] Beta 3 |
| | | | | subunit of the gamma-amino butyric acid A receptor, which is a |
| | | | | chloride channel and the major inhibitory neurotransmitter |
| | | | | receptor in the brain; gene mutations cause cleft palate and |
| | | | | seizures. Human GABRB3 is associated with Angelman syndrome |
| | | | | (Uusi-Oukari, M. et al. (2000) Mol. Cell Neurosci. 16: 34-41; |
| | | | | DeLorey, T. M. et al. (1998) J. Neurosci. 18: 8505-8514.) |
| 38 | 7490974CD1 | g3874991 | 1.4E−35 | [Caenorhabditis elegans] UNC-93 protein |
| | | | | (Ainscough R. et al. (1998) Science 282: 2012-2018.) |
| | | 299537\|Hs.22033 | 1.4E−127 | [Homo sapiens] Protein which has weak similarity to a |
| | | | | region of C. elegans unc-93, which |
| | | | | is a putative membrane protein involved in muscle action |
| | | 241126\|unc-93 | 1.2E−36 | [Caenorhabditis elegans] Putative membrane protein involved |
| | | | | in muscle action |
| | | | | (Levin, J. Z., and Horvitz, H. R. (1992) J. Cell Biol. |
| | | | | 117: 143-155). |
| 39 | 7506224CD1 | g11596110 | 5.4E−279 | [Homo sapiens] transmembrane protein vezatin |
| | | | | (Kussel-Andermann, P. et al. (2000) EMBO J. 19: 6020-6029.) |
| | | 598206\|VEZATIN | 1.2E−281 | [Homo sapiens] Protein that interacts with the FERM domain |
| | | | | of MYO7A, has similarity to mouse Vezatin |
| | | | | (Kussel-Andermann, P. et al. (2000) supra.) |
| 40 | 7506280CD1 | g3746652 | 2.9E−81 | [Homo sapiens] JWA protein |
| 40 | | 664973\|Gtrap3-18 | 1.8E−77 | [Rattus norvegicus] Glutamate transporter EAAC1-interacting |
| | | | | protein, binds to the intracellular region of the excitatory |
| | | | | glutamate carrier EAAC1 (Slc1a1), reducing the affinity of |
| | | | | EEAC1 for its substrate and thereby reducing glutamate transport |
| | | | | (Lin, C. I. et al. (2001) Nature 410: 84-88.) |
| | | 746509\|Arl6ip5 | 4.2E−76 | [Mus musculus] Protein that binds to ARL-6, which is a |
| | | | | member of the ADP-ribosylation factor-like family |
| | | | | (Ingley, E. et al. (1999) FEBS Lett. 459: 69-74.) |
| 41 | 7508326CD1 | g13436206 | 8.70E−189 | [Homo sapiens] Similar to G protein gamma 3 linked gene |
| | | 731479\|MGC4694 | 7.5E−190 | [Homo sapiens] Protein which has strong similarity to |
| | | | | uncharacterized G protein gamma 3 linked gene (mouse Gng31g), |
| | | | | mutations in the human GNG3LG (BSCL2) gene are associated with |
| | | | | Congenital generalized lipodystrophy or Berardinelli Seip |
| | | | | syndrome (BSCL) |
| | | 587933\|Gng3lg | 1.1E−160 | [Mus musculus] G protein gamma 3 linked gene, inhibits growth |
| | | | | when expressed in E. coli; mutations in the human GNG3LG |
| | | | | (BSCL2) gene result in Congenital generalized lipodystrophy, or |
| | | | | Berardinelli Seip syndrome (BSCL) |
| | | | | (Magre, J. et al. (2001) Nat. Genet. 28: 365-370; Downes, |
| | | | | G. B. et al. (1998) Genomics 53: 220-230.) |
| 42 | 7506370CD1 | g6941999 | 9.9E−90 | [Mus musculus] MMTV receptor variant 1 |
| | | | | (Golovkina, T. V. et al. (1998) J. Virol. 72: 3066-3071.) |
| 43 | 6312989CD1 | g2564916 | 3.0E−263 | [Homo sapiens] cote1 |
| | | | | (Winfield, S. L. et al. (1997) Genome Res. 7: 1020-1026.) |
| 44 | 7501108CD1 | g7242876 | 1.6E−105 | [Mus musculus] kidney predominant protein |
| 45 | 7507581CD1 | g6014681 | 1.3E−62 | [Drosophila melanogaster] F protein |
| | | 731651\|FLJ14466 | 7.4E−124 | [Homo sapiens] Protein which has high similarity to |
| | | | | uncharacterized CAP-binding protein complex interacting protein |
| | | | | 2 (human CBCIP2) |
| 46 | 7506361CD1 | g3211722 | 0.0 | [Homo sapiens] lamin B receptor homolog TM7SF2; ANG1 |
| | | | | (Lemmens, I. H. et al. (1998) Genomics 49: 437-442.) |
| 46 | | 338558\|TM7SF2 | 0.0 | [Homo sapiens][Endoplasmic reticulum; Cytoplasmic; |
| | | | | Plasma membrane] Transmembrane 7 superfamily member 2, a |
| | | | | member of the lamin B receptor-sterol reductase family of |
| | | | | proteins, contains seven putative C-terminal transmembrane |
| | | | | domains, localizes exclusively to the endoplasmic reticulum |
| | | | | (Lemmens, I. H. et al. (1998) supra; Holmer, L. et al. (1998) |
| | | | | Genomics 54: 469-476.) |
| | | 336232\|LBR | 5.0E−95 | [Homo sapiens][DNA-binding protein][Nuclear; |
| | | | | Nuclear matrix] Lamin B receptor, a nuclear envelope |
| | | | | inner membrane protein, may mediate interaction between |
| | | | | chromatin and the nuclear envelope |
| | | | | (Steen, R. L., and Collas, P. (2001) J. Cell Biol. |
| | | | | 153: 621-626; Haraguchi, T. et al. (2000) J. |
| | | | | Cell Sci. 113: 779-794.) |
| 47 | 7509211CD1 | g2564916 | 0.0 | [Homo sapiens] cote1 |
| | | | | (Winfield, S. L. et al. (1997) Genome Res. 7: 1020-1026.) |

TABLE 3

| SEQ | Incyte | Amino | Potential | Potential | | Analytical |
|---|---|---|---|---|---|---|
| ID | Polypeptide | Acid | Phosphorylation | Glycosylation | Signature Sequences, | Methods |
| NO: | ID | Residues | Sites | Sites | Domains and Motifs | and Databases |
| 1 | 3356677CD1 | 414 | S122 S355 S397 | N3 N40 N228 | signal_cleavage: M1-G60 | SPSCAN |
| | | | T244 T260 | | | |
| | | | | | Signal Peptide: M38-G60 | HMMER |
| | | | | | Cytosolic domains: T70-Y89, | TMHMMER |
| | | | | | M150-Q188, T244-P254, | |
| | | | | | P325-L414 | |
| | | | | | Transmembrane domains: | |
| | | | | | W47-L69, I90-G112, | |
| | | | | | Y127-I149, F189-F206, | |
| | | | | | L221-F243, V255-I277, | |
| | | | | | A302-F324 | |
| | | | | | Non-cytosolic domains: | |
| | | | | | M1-P46, D113-C126, | |
| | | | | | G207-Y220, L278-A301 | |
| | | | | | PHOTOSYSTEM II PROTEIN P | BLIMPS_{—} |
| | | | | | PD02346: G165-G207, | PRODOM |
| | | | | | D211-L249, P342-A378 | |
| 2 | 7481665CD1 | 836 | S130 S196 S211 | N76 | signal_cleavage: M22-S78 | SPSCAN |
| | | | S252 S409 S528 | | | |
| | | | S616 S621 S669 | | | |
| | | | S716 S765 S795 | | | |
| | | | T218 T302 T412 | | | |
| | | | T650 Y458 Y490 | | | |
| | | | Y559 | | | |
| | | | | | Signal Peptide: M22-A52 | HMMER |
| | | | | | TPR Domain: A437-H470 | HMMER_PFAM |
| | | | | | L705-D738, S570-H603, | |
| | | | | | H604-A637, N502-F535, | |
| | | | | | S743-Q776, E638-Q670, | |
| | | | | | A471-H501, A777-S810, | |
| | | | | | A536-S569 | |
| 2 | | | | | Cytosolic domains: T24-G34, | TMHMMER |
| | | | | | K111-P221, T287-M306, | |
| | | | | | V362-T387 | |
| | | | | | Transmembrane domains: | |
| | | | | | F4-Y23, L35-V57, | |
| | | | | | F88-V110, F222-M244, | |
| | | | | | I264-V286, R307-F329, | |
| | | | | | V339-V361, W388-W410 | |
| | | | | | Non-cytosolic domains: | |
| | | | | | M1-P3, G58-S87, | |
| | | | | | G245-Y263, K330-L338, | |
| | | | | | K411-T836 | |
| | | | | | Leucine zipper pattern: | MOTIFS |
| | | | | | L216-L237, L806-L827 | |
| 3 | 3563859CD1 | 401 | S260 S396 S397 | | signal_cleavage: M1-G43 | SPSCAN |
| | | | T25 T224 T230 | | | |
| | | | T356 | | | |
| | | | | | Cytosolic domains: S149-C160, | TMHMMER |
| | | | | | A223-F297, H353-K358 | |
| | | | | | Transmembrane domains: | |
| | | | | | I126-G148, L161-V183, | |
| | | | | | S203-L222, L298-G320, | |
| | | | | | V330-F352, V359-W381 | |
| | | | | | Non-cytosolic domains: | |
| | | | | | M1-R125, N184-N202, | |
| | | | | | L321-L329, S382-N401 | |
| 4 | 2588884CD1 | 181 | S86 S98 S154 T2 | N73 N139 | Cytosolic domain: M1-P33 | TMHMMER |
| | | | T10 T133 | | Transmembrane domain: W34-L56 | |
| | | | | | Non-cytosolic domain: G57-I181 | |
| | | | | | Adipokinetic hormone family | BLIMPS_{—} |
| | | | | | proteins BL00256: Q28-C36 | BLOCKS |
| | | | | | LECTIN-LIKE OXIDIZED LDL | BLAST_PRODOM |
| | | | | | RECEPTOR LECTIN PD031175: | |
| | | | | | T52-E132 | |
| | | | | | RECEPTOR OXIDIZED | BLAST_PRODOM |
| | | | | | LIPOPROTEIN LECTIN-LIKE | |
| | | | | | LDL LECTIN ENDOTHELIAL | |
| | | | | | FOR LOW DENSITY PD020742: | |
| | | | | | M1-V51 | |
| | | | | | Leucine zipper pattern: | MOTIFS |
| | | | | | L60-L81 | |
| 5 | 7503422CD1 | 249 | S103 S221 T2 T126 | | signal_cleavage: M1-G23 | SPSCAN |
| | | | | | Signal Peptide: M1-G18, | HMMER |
| | | | | | M1-S20, M1-G23, | |
| | | | | | M1-A24, M1-C26 | |
| | | | | | EGF-like domain: C114-C150, | HMMER_PFAM |
| | | | | | C57-C85, C26-C54 | |
| | | | | | Cytosolic domain: N193-I249 | TMHMMER |
| | | | | | Transmembrane domain: I170-L192 | |
| | | | | | Non-cytosolic domain: M1-A169 | |
| | | | | | Laminin-type EGF-like (LE) | BLIMPS_{—} |
| | | | | | domain proteins BL01248: | BLOCKS |
| | | | | | C45-C57 | |
| | | | | | Type II EGF-like signature | BLIMPS_{—} |
| | | | | | PR00010: D88-N99, G124-E131, | PRINTS |
| | | | | | S135-F145, S146-I152 | |
| | | | | | NADH-ubiquinone/ | BLIMPS_PFAM |
| | | | | | plastoquinone oxidoreductase | |
| | | | | | chain 6 PF00499: I174-N193 | |
| | | | | | GLYCOPROTEIN PREADIPOCYTE | BLAST_PRODOM |
| | | | | | DELTA-LIKE PROTEIN PRECURSOR | |
| | | | | | DLK CONTAINS: FETAL ANTIGEN FA1 | |
| | | | | | PD012424: C163-I249 | |
| | | | | | GLYCOPROTEIN PREADIPOCYTE | BLAST_PRODOM |
| | | | | | DELTA-LIKE PRECURSOR FACTOR | |
| | | | | | PROTEIN DLK PREF1 | |
| | | | | | ADIPOCYTE DIFFERENTIATION | |
| | | | | | PD150292: F17-C54 | |
| | | | | | PROTEIN GLYCOPROTEIN | BLAST_PRODOM |
| | | | | | EGF-LIKE DOMAIN TRANSMEMBRANE | |
| | | | | | PRECURSOR REPEAT | |
| | | | | | SIGNAL RECEPTOR SIMILAR | |
| | | | | | PD004979: C54-N159 | |
| 5 | | | | | EGF DM00003 | BLAST_DOMO |
| | | | | | P80370\|18-70: G18-P71 | |
| | | | | | Q07645\|107-174: C92-S155 | |
| | | | | | P80370\|129-169: G112-V153 | |
| | | | | | Q07645\|18-66: G18-L67 | |
| | | | | | EGF-like domain signature 1: | MOTIFS |
| | | | | | C43-C54, C74-C85, | |
| | | | | | C139-C150 | |
| | | | | | EGF-like domain signature 2: | MOTIFS |
| | | | | | C43-C54, C74-C85, | |
| | | | | | C139-C150 | |
| 6 | 7503424CD1 | 289 | S105 S120 S194 | N100 | signal_cleavage: M1-G23 | SPSCAN |
| | | | S261 T2 T143 | | | |
| | | | T184 | | | |
| | | | | | Signal Peptide: M1-G18, | HMMER |
| | | | | | M1-S20, M1-G23, M1-A24, | |
| | | | | | M1-C26 | |
| | | | | | EGF-like domain: C92-C124, | HMMER_PFAM |
| | | | | | C131-C167, C57-C85, | |
| | | | | | C26-C54 | |
| | | | | | Cytosolic domain: N234-I289 | TMHMMER |
| | | | | | Transmembrane domain: I211-L233 | |
| | | | | | Non-cytosolic domain: M1-A210 | |
| | | | | | Type II EGF-like signature | BLIMPS_{—} |
| | | | | | PR00010: D88-N99, G109-Y119, | PRINTS |
| | | | | | S163-I169 | |
| | | | | | NADH-ubiquinone/plastoquinone | BLIMPS_PFAM |
| | | | | | oxidoreductase chain 6 PF00499: | |
| | | | | | I215-N234 | |
| | | | | | GLYCOPROTEIN PREADIPOCYTE | BLAST_PRODOM |
| | | | | | DELTA-LIKEPROTEIN PRECURSOR | |
| | | | | | DLK CONTAINS: FETAL ANTIGEN | |
| | | | | | FA1 PD012424: L198-I289 | |
| 6 | | | | | GLYCOPROTEIN PREADIPOCYTE | BLAST_PRODOM |
| | | | | | DELTA-LIKE PRECURSOR FACTOR | |
| | | | | | PROTEIN DLK PREF1 ADIPOCYTE | |
| | | | | | DIFFERENTIATION PD150292: | |
| | | | | | F17-C54 | |
| | | | | | PROTEIN GLYCOPROTEIN | BLAST_PRODOM |
| | | | | | EGF-LIKE DOMAIN TRANSMEMBRANE | |
| | | | | | PRECURSOR REPEAT SIGNAL RECEPTOR | |
| | | | | | SIMILAR PD004979: C85-N176 | |
| | | | | | GLYCOPROTEIN METAL BINDING | BLAST_PRODOM |
| | | | | | CHELATION METAL THIOLATE CLUSTER | |
| | | | | | REPEAT INTEGRIN PRECURSOR CELL | |
| | | | | | ADHESION PD000782: C37-P160 | |
| | | | | | EGF DM00003 | BLAST_DOMO |
| | | | | | P80370\|72-127: G72-D128 | |
| | | | | | Q07645\|107-174: D107-S172 | |
| | | | | | P80370\|129-169: G129-V170 | |
| | | | | | P80370\|18-70: G18-P71 | |
| | | | | | EGF-like domain signature 1: | MOTIFS |
| | | | | | C43-C54, C74-C85, | |
| | | | | | C113-C124.C156-C167 | |
| | | | | | EGF-like domain signature 2: | MOTIFS |
| | | | | | C43-C54, C74-C85, | |
| | | | | | C113-C124, C156-C167 | |
| 7 | 7503571CD1 | 170 | S139 T3 T113 | N111 N128 | Signal Peptide: M1-A26 | HMMER |
| | | | | N152 | | |
| | | | | | TRANSMEMBRANE PROTEIN SIGNAL | BLAST_PRODOM |
| | | | | | ANCHOR GLYCOPROTEIN ANTIGEN L6 | |
| | | | | | TETRASPAN MEMBRANE MM3 TUMOR- | |
| | | | | | ASSOCIATED PD011179: M60-Q166, | |
| | | | | | M1-M93 | |
| 7 | | | | | SIGNAL-ANCHOR TRANSMEMBRANE | BLAST_DOMO |
| | | | | | DM04739 | |
| | | | | | P48230\|1-201: G55-R163, | |
| | | | | | M1-L61 | |
| | | | | | A53399\|1-202: M60-Q166, | |
| | | | | | M1-M93 | |
| | | | | | P30408\|1-201: M60-Q166, | |
| | | | | | M1-M93 | |
| 8 | 7505722CD1 | 328 | S126 S213 S308 | N172 N312 | signal_cleavage: M1-S19 | SPSCAN |
| | | | T23 | | | |
| | | | | | Signal Peptide: M1-P16, | HMMER |
| | | | | | M1-S19, M1-V21, M1-T23 | |
| | | | | | Immunoglobulin domain: | HMMER_PFAM |
| | | | | | G57-V144, C187-A239 | |
| | | | | | Cytosolic domain: A284-P328 | TMHMMER |
| | | | | | Transmembrane domain: A261-A283 | |
| | | | | | Non-cytosolic domain: M1-G260 | |
| | | | | | CELL PRECURSOR GLYCOPROTEIN | BLAST_PRODOM |
| | | | | | TRANSMEMBRANE SIGNAL | |
| | | | | | IMMUNOGLOBULIN FOLD ADHESION | |
| | | | | | ALTERNATIVE SPLICING PD005007: | |
| | | | | | W44-G201 | |
| 9 | 7505798CD1 | 287 | S3 S29 S60 T8 T28 | N111 | Cytosolic domains: M1-P95, | TMHMMER |
| | | | T169 | | R156-P167, R223-L228 | |
| | | | | | Transmembrane domains: F96-Q118, | |
| | | | | | V133-L155, | |
| | | | | | Y168-L190, W200-V222, | |
| | | | | | V229-C248 | |
| | | | | | Non-cytosolic domains: R119-K132, | |
| | | | | | W191-Q199, | |
| | | | | | K249-S287 | |
| 10 | 7505847CD1 | 300 | S55 S119 S149 | N172 | signal_cleavage: M1-G30 | SPSCAN |
| | | | S157 S166 S211 | | | |
| | | | S240 S282 T54 T77 | | | |
| | | | T99 T297 Y105 | | | |
| 10 | | | | | Signal Peptide: M1-G30, | HMMER |
| | | | | | M1-A25 | |
| | | | | | Cytosolic domains: M1-G8, | TMHMMER |
| | | | | | S195-R300 | |
| | | | | | Transmembrane domains: A9-W31, | |
| | | | | | N172-L194 | |
| | | | | | Non-cytosolic domain: N32-C171 | |
| 11 | 7505862CD1 | 297 | S151 S187 T116 | N142 | Cytosolic domains: M1-D20, | TMHMMER |
| | | | | | L81-Q159 | |
| | | | | | Transmembrane domains: V21-A43, | |
| | | | | | L58-I80, L160-L179 | |
| | | | | | Non-cytosolic domains: F44-G57, | |
| | | | | | K180-S297 | |
| | | | | | Leucine zipper pattern: L165-L186 | MOTIFS |
| 12 | 7762537CD1 | 200 | T10 | N164 | Cytosolic domain: A140-S166 | TMHMMER |
| | | | | | Transmembrane domains: Y117-K139, | |
| | | | | | F167-I189 | |
| | | | | | Non-cytosolic domains: M1-D116, | |
| | | | | | A190-P200 | |
| | | | | | Cell attachment sequence: | MOTIFS |
| | | | | | R149-D151 | |
| 13 | 90033462CD1 | 282 | S8 S35 S77 S88 | N78 | Cytosolic domains: M1-G157, | TMHMMER |
| | | | S106 S137 T10 T27 | | R238-P249 | |
| | | | T148 T194 Y115 | | Transmembrane domains: I158-V180, | |
| | | | | | A215-W237, Y250-L272 | |
| | | | | | Non-cytosolic domains: L181-L214, | |
| | | | | | I273-D282 | |
| | | | | | Leucine zipper pattern: L251-L272 | MOTIFS |
| 14 | 1644869CD1 | 805 | S23 S112 S127 | N103 N312 | Cytosolic domains: K235-T254, | TMHMMER |
| | | | S171 S177 S321 | | M362-A432, L493-R512, | |
| | | | S365 S369 S397 | | E576-E595, Q674-T722 | |
| | | | S420 S575 S631 | | Transmembrane domains: A212-L234, | |
| | | | S773 T14 T45 T78 | | L255-F277, M339-S361, | |
| | | | T138 T184 T289 | | V433-F455, A470-V492, | |
| | | | T329 T392 T689 | | N513-G530, L553-S575, | |
| | | | T788 Y598 Y691 | | L596-I618, V651-W673, | |
| | | | | | F723-G745 | |
| | | | | | Non-cytosolic domains: M1-L211, | |
| | | | | | P278-N338, S456-E469, | |
| | | | | | R531-F552, I619-T650, | |
| | | | | | Q746-A805 | |
| | | | | | LAK4P PD129199: G485-A805 | BLAST_PRODOM |
| | | | | | Leucine zipper pattern: | MOTIFS |
| | | | | | L213-L234, L475-L496 | |
| 14 | | | | | Binding-protein-dependent | MOTIFS |
| | | | | | transport systems inner | |
| | | | | | membrane comp. sign: M362-K390 | |
| 15 | 6288712CD1 | 96 | S7 S76 S86 | N84 | signal_cleavage: M1-A38 | SPSCAN |
| | | | | | Cytosolic domain: K43-S53 | TMHMMER |
| | | | | | Transmembrane domains: A23-Y42, | |
| | | | | | L54-S76 | |
| | | | | | Non-cytosolic domains: M1-E22, | |
| | | | | | C77-L96 | |
| 16 | 71830156CD1 | 244 | S10 S165 T126 | N158 | signal_cleavage: M1-G26 | SPSCAN |
| | | | Y140 | | | |
| | | | | | Tetraspanin family: K12-L243 | HMMER_PFAM |
| | | | | | Cytosolic domains: M1-K12, | TMHMMER |
| | | | | | G78-L88, K242-G244 | |
| | | | | | Transmembrane domains: K13-G35, | |
| | | | | | L55-Y77, F89-F111, | |
| | | | | | S219-I241 | |
| | | | | | Non-cytosolic domains: G36-L54, | |
| | | | | | F112-L218 | |
| | | | | | Transmembrane 4 family proteins | BLIMPS_{—} |
| | | | | | BL00421: S9-I27, L60-L98, | BLOCKS |
| | | | | | V147-N158, Y175-C180, | |
| | | | | | Q214-L243 | |
| | | | | | Transmembrane 4 family signature: | PROFILESCAN |
| | | | | | L54-V107 | |
| | | | | | Transmembrane four family | BLIMPS_{—} |
| | | | | | signature PR00259: K13-G36, | PRINTS |
| | | | | | L54-T80, K81-L109, | |
| | | | | | T217-L243 | |
| | | | | | TRANSMEMBRANE GLYCOPROTEIN | BLAST_PRODOM |
| | | | | | SIGNAL ANCHOR PROTEIN ANTIGEN | |
| | | | | | MEMBRANE PHOTORECEPTOR VISION | |
| | | | | | CD9 CELL | |
| | | | | | PD000920: K12-F167, S187-L239 | |
| | | | | | TRANSMEMBRANE 4 FAMILY DM00947 | BLAST_DOMO |
| | | | | | \|P41732\|2-238: P7-L239 | |
| | | | | | \|P48509\|8-250: L11-L243 | |
| | | | | | \|P27701\|1-258: K12-L239 | |
| | | | | | \|P08962\|1-232: L14-L234 | |
| 17 | 7505044CD1 | 237 | S121 S180 T15 T73 | N33 N82 N103 | signal_cleavage: M1-T15 | SPSCAN |
| | | | T96 T157 T227 | N170 | | |
| | | | | | Immunoglobulin domain: G29-V87 | HMMER_PFAM |
| | | | | | Cytosolic domain: K156-Q237 | TMHMMER |
| | | | | | Transmembrane domain: G133-V155 | |
| | | | | | Non-cytosolic domain: M1-H132 | |
| | | | | | OB BINDING MYELOID CELL | BLAST_PRODOM |
| | | | | | SURFACE ANTIGEN CD33 PRECURSOR | |
| | | | | | GP67 GLYCOPROTEIN PD015772: | |
| | | | | | K123-T236 | |
| | | | | | CELL PRECURSOR GLYCOPROTEIN | BLAST_PRODOM |
| | | | | | TRANSMEMBRANE SIGNAL | |
| | | | | | IMMUNOGLOBULIN FOLD ADHESION | |
| | | | | | ALTERNATIVE SPLICING PD005007: | |
| | | | | | L14-Q101 | |
| 18 | 7505086CD1 | 790 | S47 S56 S162 S240 | N54 N98 N180 | signal_cleavage: M1-N35 | SPSCAN |
| | | | S278 S291 S349 | N212 N219 | | |
| | | | S402 S413 S531 | N272 N411 | | |
| | | | S547 S557 S619 | N460 N518 | | |
| | | | S685 T106 T251 | N665 N716 | | |
| | | | T477 T588 T628 | N723 N741 | | |
| | | | T760 | | | |
| | | | | | EGF-like domain: C163-C194 | HMMER_PFAM |
| | | | | | Lectin C-type domain: K49-T160 | HMMER_PFAM |
| | | | | | Sushi domain (SCR repeat): | HMMER_PFAM |
| | | | | | C262-C319, C448-C505, | |
| | | | | | C572-C629, C324-C381, | |
| | | | | | C386-C443, C200-C257, | |
| | | | | | C642-C699, C510-C567, | |
| | | | | | C704-C761 | |
| | | | | | C-type lectin domain signature | PROFILESCAN |
| | | | | | and profile: G105-G179 | |
| 18 | | | | | Selectin superfamily complement- | BLIMPS_{—} |
| | | | | | binding repeat signature PR00343: | PRINTS |
| | | | | | C200-N219, F220-S227, | |
| | | | | | D232-W250, T437-Q447 | |
| | | | | | PSELECTIN GLYCOPROTEIN LECTIN | BLAST_PRODOM |
| | | | | | PRECURSOR GRANULE MEMBRANE | |
| | | | | | PROTEIN GMP140 PADGEM CD62P | |
| | | | | | PD009117: M1-R57 | |
| | | | | | PROTEIN F36H2.3A F36H2.3B | BLAST_PRODOM |
| | | | | | Sushi domain PD004794: | |
| | | | | | G558-G765, S162-A701, | |
| | | | | | L446-C761, S472-A785 | |
| | | | | | PRECURSOR GLYCOPROTEIN LECTIN | BLAST_PRODOM |
| | | | | | LSELECTIN ADHESION LEUKOCYTE | |
| | | | | | ENDOTHELIAL CELL MOLECULE | |
| | | | | | TRANSMEMBRANE EGF-LIKE PD151850: | |
| | | | | | Y159-E199 | |
| | | | | | COMPLEMENT REGULATORY PROTEIN | BLAST_PRODOM |
| | | | | | PD060257: G486-K703, | |
| | | | | | I267-A507 | |
| | | | | | SUSHI REPEAT DM04887 | BLAST_DOMO |
| | | | | | \|P16581\|1-609: | |
| | | | | | K37-A569, C337-Q762 | |
| | | | | | \|P33730\|1-610: | |
| | | | | | S21-A569, C381-F786 | |
| | | | | | \|P27113\|1-551: K37-Q506, | |
| | | | | | C381-F732, Q444-D763 | |
| | | | | | C-TYPE LECTIN DM00035\| | BLAST_DOMO |
| | | | | | P16109\|31-153: S31-K154 | |
| | | | | | C-type lectin domain | MOTIFS |
| | | | | | signature: C131-C158 | |
| | | | | | EGF-like domain signature 1: | MOTIFS |
| | | | | | C183-C194 | |
| | | | | | EGF-like domain signature 2: | MOTIFS |
| | | | | | C183-C194 | |
| 19 | 7505784CD1 | 172 | S63 T43 T74 T87 | N72 N85 N109 | signal_cleavage: M1-A34 | SPSCAN |
| | | | T113 | N115 | | |
| 19 | | | | | Signal Peptide: M11-A34, | HMMER |
| | | | | | M10-A34, M11-A38, | |
| | | | | | M11-A30 | |
| | | | | | Tetraspanin family: L16-L171 | HMMER_PFAM |
| | | | | | Cytosolic domains: M1-M11, | TMHMMER |
| | | | | | K58-T145 | |
| | | | | | Transmembrane domains: I12-A30, | |
| | | | | | A35-I57, V146-Y168 | |
| | | | | | Non-cytosolic domains: E31-A34, | |
| | | | | | C169-Q172 | |
| | | | | | Transmembrane 4 family proteins | BLIMPS_{—} |
| | | | | | BL00421: S5-I23, T74-N85, | BLOCKS |
| | | | | | N142-L171 | |
| | | | | | Transmembrane four family | BLIMPS_{—} |
| | | | | | signature PR00259: M11-L39, | PRINTS |
| | | | | | T145-L171 | |
| | | | | | TRANSMEMBRANE 4 FAMILY DM00947 | BLAST_DOMO |
| | | | | | \|P41732\|2-238: | |
| | | | | | L16-L167, I7-L26 | |
| | | | | | \|P19075\|1-236: | |
| | | | | | L16-L171, I7-L26 | |
| | | | | | IP48509\|8-250: N15-Q172 | |
| 20 | 7505813CD1 | 253 | S2 S118 T110 T132 | N170 N183 | Signal Peptide: M1-G26 | HMMER |
| | | | T137 T191 Y246 | N188 | | |
| | | | | | Tetraspanin family: K12-E228, | HMMER_PFAM |
| | | | | | M232-L242 | |
| | | | | | Cytosolic domains: M1-K12, | TMHMMER |
| | | | | | K81-L86 | |
| | | | | | Transmembrane domains: Y13-I38, | |
| | | | | | S58-L80, L87-S109 | |
| | | | | | Non-cytosolic domains: D39-W57, | |
| | | | | | T110-R253 | |
| | | | | | Transmembrane 4 family proteins | BLIMPS_{—} |
| | | | | | BL00421: S9-S27, V60-F98, | BLOCKS |
| | | | | | V146-Y157, V177-Y182, | |
| | | | | | R209-L238 | |
| | | | | | Transmembrane 4 family | PROFILESCAN |
| | | | | | signature: V52-L107 | |
| | | | | | Transmembrane four family | BLIMPS_{—} |
| | | | | | signature PR00259: Y13-I36, | PRINTS |
| | | | | | L54-L80, K81-S109, | |
| | | | | | I216-L242 | |
| 20 | | | | | TRANSMEMBRANE GLYCOPROTEIN | BLAST_PRODOM |
| | | | | | SIGNAL ANCHOR PROTEIN ANTIGEN | |
| | | | | | MEMBRANE PHOTORECEPTOR VISION | |
| | | | | | CD9 CELL PD000920: K12-F162 | |
| | | | | | TRANSMEMBRANE 4 FAMILY | BLAST_DOMO |
| | | | | | DM00947 | |
| | | | | | \|P11049\|3-280: A3-L242, | |
| | | | | | E228-R253 | |
| | | | | | \|P31053\|3-272: A3-L242, | |
| | | | | | E228-V245 | |
| | | | | | \|P27701\|1-258: S6-I192, | |
| | | | | | E228-L242 | |
| | | | | | \|I49561\|1-266: C7-K195 | |
| | | | | | Leucine zipper pattern: | MOTIFS |
| | | | | | L73-L94 | |
| | | | | | Transmembrane 4 family | MOTIFS |
| | | | | | signature: G65-L87 | |
| 21 | 7505873CD1 | 431 | S18 S223 T172 | N128 N192 | signal_cleavage: M1-F16 | SPSCAN |
| | | | T376 T416 T417 | N350 N391 | | |
| | | | | | Signal Peptide: M32-A58 | HMMER |
| | | | | | Cytosolic domains: | TMHMMER |
| | | | | | M1-M1, T416-M431 | |
| | | | | | Transmembrane domains: | |
| | | | | | L2-F24, S393-L415 | |
| | | | | | Non-cytosolic domain: | |
| | | | | | T25-I392 | |
| | | | | | PROTEIN CHROMOSOME ORF | BLAST_PRODOM |
| | | | | | YLL031C INTERGENIC REGION | |
| | | | | | TRANSMEMBRANE C27A12.9 XII | |
| | | | | | READING PD008858: | |
| | | | | | P40-K374, L7-D57 | |
| 22 | 7505881CD1 | 206 | S105 S109 S151 | | Cytosolic domains: M1-T46, | TMHMMER |
| | | | S201 T15 | | K107-I206 | |
| | | | | | Transmembrane domains: | |
| | | | | | V47-A69, M84-G106 | |
| | | | | | Non-cytosolic domain: | |
| | | | | | P70-L83 | |
| 23 | 7503510CD1 | 694 | S81 S270 S326 | N74 N150 N246 | signal_cleavage: M1-A33 | SPSCAN |
| | | | S356 S379 S403 | N292 N318 | | |
| | | | S473 S588 S681 | | | |
| | | | T192 T214 T320 | | | |
| | | | T370 T499 T538 | | | |
| | | | T604 T609 T690 | | | |
| | | | | | Signal Peptide: M1-A33, | HMMER |
| | | | | | M1-A34 | |
| | | | | | Leucine Rich Repeat: | HMMER_PFAM |
| | | | | | S332-R355, S236-S259, | |
| | | | | | K308-S331, S356-S379, | |
| | | | | | S189-P211, G407-K430, | |
| | | | | | R212-N235, N93-S114, | |
| | | | | | W69-P92, K260-T283, | |
| | | | | | S383-E406, P164-S187, | |
| | | | | | S140-P163, A284-Q307, | |
| | | | | | H116-K136 | |
| | | | | | Leucine rich repeat | HMMER_PFAM |
| | | | | | C-terminal domain: | |
| | | | | | D440-D490 | |
| | | | | | Leucine rich repeat | HMMER_PFAM |
| | | | | | N-terminal domain: | |
| | | | | | P40-P67 | |
| | | | | | Immunoglobulin domain: | HMMER_PFAM |
| | | | | | G509-I579, T613-A671 | |
| | | | | | MEMBRANE GLYCOPROTEIN | BLAST_PRODOM |
| | | | | | MEMBRANE PD172109: | |
| | | | | | D491-F583 | |
| | | | | | CODED FOR BY C ELEGANS | BLAST_PRODOM |
| | | | | | CDNA YK6G3.3 SIMILARITY | |
| | | | | | MULTIPLE LEUCINE-RICH | |
| | | | | | PD037237: L432-I610 | |
| | | | | | MEMBRANE GLYCOPROTEIN | BLAST_PRODOM |
| | | | | | MEMBRANE PD165826: | |
| | | | | | E29-T70 | |
| | | | | | Leucine zipper pattern: | MOTIFS |
| | | | | | L52-L73, L59-L80 | |
| 24 | 7714715CD1 | 228 | S88 S211 T7 T209 | | signal_cleavage: M1-A23 | SPSCAN |
| | | | | | Signal Peptide: M11-A31, | HMMER |
| | | | | | M1-A23, M1-A31 | |
| 24 | | | | | Cytosolic domains: M1-L12, | TMHMMER |
| | | | | | T121-S131, Q204-L228 | |
| | | | | | Transmembrane domains: | |
| | | | | | W13-I35, Q98-F120, | |
| | | | | | I132-Y154, L181-A203 | |
| | | | | | Non-cytosolic domains: | |
| | | | | | G36-W97, P155-S180 | |
| 25 | 7506032CD1 | 216 | S98 S112 S193 | | Cytosolic domains: M1-A4, | TMHMMER |
| | | | S196 T67 | | M55-K69, D138-K143, | |
| | | | | | A190-R216 | |
| | | | | | Transmembrane domains: | |
| | | | | | V5-T27, I32-F54, | |
| | | | | | Y70-Y91, L115-F137, | |
| | | | | | K144-F163, Y167-A189 | |
| | | | | | Non-cytosolic domains: | |
| | | | | | E28-R31, K92-R114, | |
| | | | | | I164-S166 | |
| | | | | | VF36H2L.1 PROTEIN PD129998: | BLAST_PRODOM |
| | | | | | C9-I126, L116-S196 | |
| | | | | | Prokaryotic Lipoprotein: | MOTIFS |
| | | | | | T184-C194 | |
| 26 | 7506034CD1 | 359 | S186 S261 S322 | N63 N180 | DHHC zinc finger domain: | HMMER_PFAM |
| | | | T182 Y198 | | E90-G154 | |
| | | | | | Cytosolic domains: M1-R18, | TMHMMER |
| | | | | | N74-T145 | |
| | | | | | Transmembrane domains: | |
| | | | | | L19-D41, T51-F73, | |
| | | | | | L146-T168 | |
| | | | | | Non-cytosolic domains: | |
| | | | | | S42-H50, Q169-R359 | |
| | | | | | M18.8 PROTEIN PD182183: | BLAST_PRODOM |
| | | | | | K193-E355, Y138-E190, | |
| | | | | | L148-Y171 | |
| | | | | | PROTEIN CHROMOSOME | BLAST_PRODOM |
| | | | | | C ELEGANS TRANSMEMBRANE | |
| | | | | | ZK757.1 ANK REPEAT | |
| | | | | | SIMILARITY REGION PD003041: | |
| | | | | | L98-L148 | |
| | | | | | YOR034C; MEMBRANE; DM05142\| | BLAST_DOMO |
| | | | | | Q09701\|316-569: | |
| | | | | | S55-Q169 | |
| 27 | 7506100CD1 | 115 | S12 S42 T16 T46 | | Cytidine and deoxycytidylate | BLIMPS_{—} |
| | | | T60 T103 | | deaminases zinc-binding | BLOCKS |
| | | | | | regions BL00903: Y19-Q28 | |
| 27 | | | | | PROTEIN COMPONENT CGRP- | BLAST_PRODOM |
| | | | | | RECEPTOR CALCITONIN GENE- | |
| | | | | | RELATED PEPTIDE RECEPTOR | |
| | | | | | M106.3 CHROMOSOME II | |
| | | | | | PD021274: Y14-A115, | |
| | | | | | M1-S21 | |
| 28 | 1743113CD1 | 454 | S36 S133 S143 | N126 | signal_cleavage: M1-S36 | SPSCAN |
| | | | S160 S240 S241 | | | |
| | | | S293 S335 S366 | | | |
| | | | T96 T128 T144 | | | |
| | | | T312 T411 Y168 | | | |
| | | | Y220 | | | |
| | | | | | Cytosolic domain: D454-D454 | TMHMMER |
| | | | | | Transmembrane domain: | |
| | | | | | N431-S453 | |
| | | | | | Non-cytosolic domain: | |
| | | | | | M1-Q430 | |
| | | | | | ODORANT RESPONSE PROTEIN | BLAST_PRODOM |
| | | | | | ODR4 COSMID | |
| | | | | | Y102E9 PD042765: L15-A441 | |
| 29 | 7505144CD1 | 251 | S37 S134 S186 | N132 N191 | signal_cleavage: M1-A38 | SPSCAN |
| | | | S196 T81 T89 | | | |
| | | | T162 | | | |
| | | | | | Signal Peptide: M23-A38, | HMMER |
| | | | | | M23-D40, M18-A38, | |
| | | | | | M18-R41, M1-A38, | |
| | | | | | M23-A38 | |
| | | | | | Folate receptor family: | HMMER_PFAM |
| | | | | | W22-A242 | |
| | | | | | PROTEIN FOLATE RECEPTOR | BLAST_PRODOM |
| | | | | | GLYCOPROTEIN PRECURSOR SIGNAL | |
| | | | | | FOLATE-BINDING MEMBRANE GPI | |
| | | | | | ANCHOR MULTIGENE | |
| | | | | | PD006906: S37-W173, | |
| | | | | | C146-G233 | |
| | | | | | FOLATE BINDING PROTEIN | BLAST_PRODOM |
| | | | | | PD113535: M1-C36 | |
| 29 | | | | | FOLATE-BINDING PROTEIN | BLAST_DOMO |
| | | | | | DM02165 | |
| | | | | | \|P14207\|2-254: | |
| | | | | | V19-S206, C146-G251 | |
| | | | | | \|P41439\|2-242: | |
| | | | | | W20-S206, C146-G233 | |
| | | | | | \|P15328\|22-256: | |
| | | | | | R41-S175, C146-L250 | |
| | | | | | \|P02702\|1-221: | |
| | | | | | R41-S206, L124-A220 | |
| 30 | 7506132CD1 | 193 | S46 T122 Y56 | N139 | Signal Peptide: M1-A31, | HMMER |
| | | | | | M1-A28 | |
| | | | | | Tetraspanin family: K12-G153, | HMMER_PFAM |
| | | | | | I173-I186 | |
| | | | | | Cytosolic domains: M1-K12, | TMHMMER |
| | | | | | C78-L88 | |
| | | | | | Transmembrane domains: Y13-W35, | |
| | | | | | F55-C77, G89-I111 | |
| | | | | | Non-cytosolic domains: F36-Y54, | |
| | | | | | G112-I193 | |
| | | | | | Transmembrane 4 family | BLIMPS_{—} |
| | | | | | proteins BL00421: R9-S27, | BLOCKS |
| | | | | | V61-F99, F145-S156 | |
| | | | | | Transmembrane 4 family | PROFILESCAN |
| | | | | | signature: F55-F108 | |
| | | | | | Transmembrane four family | BLIMPS_{—} |
| | | | | | signature PR00259: Y13-F36, | PRINTS |
| | | | | | F55-M81, R82-F110, | |
| | | | | | V160-I186 | |
| | | | | | TRANSMEMBRANE GLYCOPROTEIN | BLAST_PRODOM |
| | | | | | SIGNAL ANCHOR PROTEIN ANTIGEN | |
| | | | | | MEMBRANE PHOTORECEPTOR VISION | |
| | | | | | CD9 CELL PD000920: K12-G153, | |
| | | | | | K172-I186 | |
| | | | | | TRANSMEMBRANE 4 FAMILY | BLAST_DOMO |
| | | | | | DM00947 | |
| | | | | | \|P30932\|1-220: | |
| | | | | | R5-L169, I173-S189 | |
| | | | | | \|I49589\|2-221: | |
| | | | | | R5-L169, I173-S189 | |
| | | | | | \|P18582\|2-232: | |
| | | | | | G6-S156, I173-S189 | |
| | | | | | \|P19075\|1-236: | |
| | | | | | C10-G153, K172-N188 | |
| | | | | | Prokaryotic Lipoprotein: | MOTIFS |
| | | | | | A67-C77 | |
| | | | | | Transmembrane 4 family | MOTIFS |
| | | | | | signature: G66-L88 | |
| 31 | 8142016CD1 | 529 | S4 S142 S157 S288 | N168 N186 | signal_cleavage: M1-N37 | SPSCAN |
| | | | S313 S381 S428 | N210 N223 | | |
| | | | T15 T46 T119 | N301 N349 | | |
| | | | T164 T226 T240 | N466 | | |
| | | | T311 T318 T320 | | | |
| | | | T340 T351 T461 | | | |
| | | | | | Signal Peptide: M18-V32, | HMMER |
| | | | | | M18-A35, M18-N37, | |
| | | | | | M18-G39, M18-S41, | |
| | | | | | M18-Q38 | |
| | | | | | SEA domain: K229-Y345 | HMMER_PFAM |
| | | | | | Cytosolic domain: T461-Y529 | TMHMMER |
| | | | | | Transmembrane domain: | |
| | | | | | L438-V460 | |
| | | | | | Non-cytosolic domain: | |
| | | | | | M1-Q437 | |
| | | | | | CELL SURFACE ANTIGEN 114/A10 | BLAST_PRODOM |
| | | | | | PRECURSOR GLYCOPROTEIN | |
| | | | | | SIGNAL EGF-LIKE DOMAIN | |
| | | | | | REPEAT PD040348: R290-Y529 | |
| | | | | | PROTEIN PRECURSOR GLYCOPROTEIN | BLAST_PRODOM |
| | | | | | SIGNAL REPEAT ANTIGEN SURFACE | |
| | | | | | MEROZOITE CELL TRANSMEMBRANE | |
| | | | | | PD000546: T25-T184, | |
| | | | | | T47-N192 | |
| | | | | | EGF-like domain signature 2: | MOTIFS |
| | | | | | C214-C227, C406-C420 | |
| 32 | 7506135CD1 | 573 | S4 S8 S17 S63 | N174 | Cytosolic domain: S56-S75 | TMHMMER |
| | | | S164 S223 S247 | | Transmembrane domains: A33-A55, | |
| | | | S260 S310 S369 | | N76-V98 | |
| | | | S396 S427 S447 | | Non-cytosolic domains: M1-Q32, | |
| | | | S479 S500 S534 | | C99-L573 | |
| | | | S543 S549 T13 T59 | | | |
| | | | T139 T205 T365 | | | |
| | | | T404 | | | |
| | | | | | COTE1 TRANSMEMBRANE PROTEIN | BLAST_PRODOM |
| | | | | | PD146399: S67-L573, M1-L137 | |
| | | | | | Cell attachment sequence: | MOTIFS |
| | | | | | R199-D201 | |
| 33 | 90086301CD1 | 232 | S70 S145 S158 | N20 N58 N157 | 7 transmembrane receptor | HMMER_PFAM |
| | | | S176 S205 T67 | | (rhodopsin family): G57-R138 | |
| | | | T171 | | | |
| | | | | | Cytosolic domains: I62-N73, | TMHMMER |
| | | | | | A136-F232 | |
| | | | | | Transmembrane domains: I39-L61, | |
| | | | | | V74-V96, I116-S135 | |
| | | | | | Non-cytosolic domains: M1-G38, | |
| | | | | | D97-L115 | |
| | | | | | Bombesin receptor signature | BLIMPS_{—} |
| | | | | | PR00358: C93-F108, | PRINTS |
| | | | | | K114-T130 | |
| | | | | | Gastrin-releasing peptide | BLIMPS_{—} |
| | | | | | PR00640: N4-C19, | PRINTS |
| | | | | | C19-D33 | |
| | | | | | tRNA synthetases class I | BLIMPS_PFAM |
| | | | | | PF00587: G225-F232 | |
| | | | | | TRANSCRIPTION PROTEIN DNA | BLIMPS_{—} |
| | | | | | PD02448: I8-P44, | PRODOM |
| | | | | | A153-K178, G203-H226 | |
| | | | | | GASTRIN-RELEASING PEPTIDE | BLAST_PRODOM |
| | | | | | RECEPTOR GRPR GRP-PREFERRING | |
| | | | | | BOMBESIN G-PROTEIN COUPLED | |
| | | | | | TRANSMEMBRANEGLYCOPROTEIN | |
| | | | | | PD019393: M1-S70 | |
| 33 | | | | | G-PROTEIN COUPLED RECEPTORS | BLAST_DOMO |
| | | | | | DM00013\|P30550\|34-337: | |
| | | | | | W34-K140 | |
| | | | | | DM00013\|P28336\|37-339: | |
| | | | | | V42-R138 | |
| | | | | | DM00013\|P47751\|40-344: | |
| | | | | | P37-R138 | |
| | | | | | DM00013\|P35371\|41-345: | |
| | | | | | P37-K140 | |
| 34 | 7487373CD1 | 312 | S106 S228 S291 | N4 N40 | 7 transmembrane receptor | HMMER_PFAM |
| | | | T273 | | (rhodopsin family): G39-Y290 | |
| | | | | | Cytosolic domains: I47-M57, | TMHMMER |
| | | | | | A123-K141, K221-C239, | |
| | | | | | K293-A312 | |
| | | | | | Transmembrane domains: | |
| | | | | | W24-L46, Y58-L80, | |
| | | | | | Y100-I122, I142-F164, | |
| | | | | | V198-L220, V240-F262, | |
| | | | | | I272-I292 | |
| | | | | | Non-cytosolic domains: | |
| | | | | | M1-H23, G81-A99, | |
| | | | | | P165-P197, G263-H271 | |
| | | | | | G-protein coupled receptors | BLIMPS_{—} |
| | | | | | proteins BL00237: R88-P127, | BLOCKS |
| | | | | | V205-Y216, A233-I259, | |
| | | | | | P282-Q298 | |
| | | | | | Olfactory receptor signature | BLIMPS_{—} |
| | | | | | PR00245: M57-T78, A175-D189, | PRINTS |
| | | | | | L236-V251 | |
| | | | | | RECEPTOR OLFACTORY PROTEIN | BLAST_PRODOM |
| | | | | | RECEPTOR-LIKE G-PROTEIN | |
| | | | | | COUPLED TRANSMEMBRANE | |
| | | | | | GLYCOPROTEIN MULTIGENE | |
| | | | | | FAMILY PD000921: Y166-I243 | |
| | | | | | PUTATIVE G-PROTEIN COUPLED | BLAST_PRODOM |
| | | | | | RECEPTOR RA1C PD170483: | |
| | | | | | I246-L307 | |
| | | | | | G-PROTEIN COUPLED RECEPTORS | BLAST_DOMO |
| | | | | | DM00013\|G45774\|18-309: | |
| | | | | | P16-R303 | |
| | | | | | DM00013\|P23273\|18-306: | |
| | | | | | H22-L304 | |
| | | | | | DM00013\|P30954\|29-316: | |
| | | | | | I25-R303 | |
| | | | | | DM00013\|P23269\|15-304: | |
| | | | | | P16-L304 | |
| 35 | 7506228CD1 | 379 | S52 S157 S267 | | Band 7 protein family | BLIMPS_{—} |
| | | | T102 T181 Y112 | | proteins | BLOCKS |
| | | | Y190 Y244 | | BL01270: R40-K78, Q70-H107, | |
| | | | | | D108-K136 | |
| | | | | | FLOTILLIN 1 GROWTH | BLAST_PRODOM |
| | | | | | ASSOCIATED PROTEIN SURFACE | |
| | | | | | ANTIGEN FLOTILLIN EPIDERMAL | |
| | | | | | FLOTILLIN 2 | |
| | | | | | PD022875: K118-Q215 | |
| | | | | | FLOTILLIN 1 FLOTILLIN GROWTH | BLAST_PRODOM |
| | | | | | ASSOCIATED PROTEIN PD151185: | |
| | | | | | S321-A379 | |
| | | | | | SURFACE ANTIGEN GROWTH | BLAST_PRODOM |
| | | | | | ASSOCIATED PROTEIN EPIDERMAL | |
| | | | | | FLOTILLIN 2 | |
| | | | | | FLOTILLIN 1 PD011251: | |
| | | | | | K196-A296 | |
| | | | | | PROTEIN FLOTILLIN 1 GROWTH | BLAST_PRODOM |
| | | | | | ASSOCIATED SURFACE ANTIGEN | |
| | | | | | FLOTILLIN GLGBGBSB INTERGENIC | |
| | | | | | REGION TRANSMEMBRANE | |
| | | | | | PD150046: M1-D127 | |
| 36 | 7506194CD1 | 453 | S28 S114 S133 | N148 N397 | Adenylate and Guanylate | HMMER_PFAM |
| | | | S202 S313 S399 | | cyclase catalytic domain: | |
| | | | S400 T33 T40 | | H349-G449 | |
| | | | Y445 | | | |
| | | | | | Cytosolic domains: M1-S151, | TMHMMER |
| | | | | | C198-M208, P254-A259, | |
| | | | | | E311-S453 | |
| | | | | | Transmembrane domains: | |
| | | | | | L152-A174, P178-V197, | |
| | | | | | W209-D231, S236-L253, | |
| | | | | | A260-L278, Q288-A310 | |
| | | | | | Non-cytosolic domains: | |
| | | | | | R175-Q177, P232-P235, | |
| | | | | | N279-K287 | |
| | | | | | Glucose-6-phosphate | BLIMPS_{—} |
| | | | | | dehydrogenase proteins | BLOCKS |
| | | | | | BL00069: R280-I302, | |
| | | | | | L184-L219 | |
| 36 | | | | | Guanylate cyclases proteins | BLIMPS_{—} |
| | | | | | BL00452: A379-L421, | BLOCKS |
| | | | | | R431-F446 | |
| | | | | | Guanylate cyclases signature: | PROFILESCAN |
| | | | | | E360-A423 | |
| | | | | | CYCLASE TYPE ADENYLYL LYASE | BLAST_PRODOM |
| | | | | | ADENYLATE ATP PYROPHOSPHATE | |
| | | | | | LYASE CAMP SYNTHESIS | |
| | | | | | TRANSMEMBRANE PD009574: | |
| | | | | | C118-R233 | |
| | | | | | CYCLASE TYPE VI ADENYLYL | BLAST_PRODOM |
| | | | | | LYASE ADENYLATE ATP | |
| | | | | | PYROPHOSPHATE LYASE CA2 + | |
| | | | | | INHIBITABLE CAMP PD016570: | |
| | | | | | M1-A58, R62-S117 | |
| | | | | | CYCLASE LYASE ADENYLYL | BLAST_PRODOM |
| | | | | | ADENYLATE TYPE ATP PYRO- | |
| | | | | | PHOSPHATE LYASE CAMP | |
| | | | | | SYNTHESIS TRANSMEMBRANE | |
| | | | | | PD003877: S234-N359 | |
| | | | | | CYCLASE LYASE SYNTHESIS | BLAST_PRODOM |
| | | | | | TRANSMEMBRANE ADENYLATE | |
| | | | | | ADENYLYL GLYCOPROTEIN ATP | |
| | | | | | PYROPHOSPHATE LYASE CAMP | |
| | | | | | PD000360: V350-G449 | |
| | | | | | GUANYLATE CYCLASE CATALYTIC | BLAST_DOMO |
| | | | | | DOMAIN | |
| | | | | | DM02293\|P30804\|91-260: | |
| | | | | | E92-S263 | |
| | | | | | GUANYLATE CYCLASES | BLAST_DOMO |
| | | | | | DM00173\|P30804\|262-536: | |
| | | | | | G264-N359, N365-E429 | |
| | | | | | DM00173\|S41603\|357-632: | |
| | | | | | G264-N359, V350-G432 | |
| | | | | | DM00173\|P30803\|276-551: | |
| | | | | | G264-N359, V350-G432 | |
| 36 | | | | | Cell attachment sequence: | MOTIFS |
| | | | | | R280-D282 | |
| | | | | | Guanylate cyclases | MOTIFS |
| | | | | | signature: G380-D403 | |
| 37 | 7506434CD1 | 36 | S27 | | signal_cleavage: M1-N29 | SPSCAN |
| | | | | | Signal Peptide: M1-S27 | HMMER |
| | | | | | Cytosolic domain: M1-G6 | TMHMMER |
| | | | | | Transmembrane domain: G7-N29 | |
| | | | | | Non-cytosolic domain: D30-G36 | |
| | | | | | LIF/OSM family signature: | PROFILESCAN |
| | | | | | M1-W35, M1-A34, | |
| | | | | | M1-S33, M1-G36, M1-P31 | |
| 38 | 7490974CD1 | 398 | S4 S52 S71 S173 | | Signal Peptide: M1-A21, | HMMER |
| | | | S241 T189 T258 | | M1-G23, M1-G24, M1-S27 | |
| | | | | | Cytosolic domains: M1-R6, | TMHMMER |
| | | | | | Q164-R197, K253-T258, | |
| | | | | | G357-M398 | |
| | | | | | Transmembrane domains: N7-Q26, | |
| | | | | | V141-L163, L198-L215, | |
| | | | | | I230-G252, G259-W281, | |
| | | | | | L334-L356 | |
| | | | | | Non-cytosolic domains: | |
| | | | | | S27-L140, S216-G229, | |
| | | | | | R282-A333 | |
| 39 | 7506224CD1 | 750 | S35 S171 S258 | N262 N291 | Cytosolic domain: R153-K750 | TMHMMER |
| | | | S285 S464 S535 | N613 N620 | Transmembrane domain: L130-I152 | |
| | | | S549 S565 S578 | N647 | Non-cytosolic domain: M1-M129 | |
| | | | S588 S597 S611 | | | |
| | | | S619 S630 S648 | | | |
| | | | S667 S692 T26 T38 | | | |
| | | | T160 T166 T235 | | | |
| | | | T474 T615 T625 | | | |
| | | | T684 T725 T730 | | | |
| | | | Y371 Y533 | | | |
| | | | | | Leucine zipper pattern: | MOTIFS |
| | | | | | L403-L424 | |
| 40 | 7506280CD1 | 162 | S18 S112 | | signal_cleavage: M1-A57 | SPSCAN |
| 40 | | | | | Signal Peptide: M40-A57 | HMMER |
| | | | | | Cytosolic domain: A58-M77 | TMHMMER |
| | | | | | Transmembrane domains: L35-A57, | |
| | | | | | V78-I100 | |
| | | | | | Non-cytosolic domains: M1-F34, | |
| | | | | | T101-E162 | |
| | | | | | PROTEIN PRENYLATED RAB | BLAST_PRODOM |
| | | | | | ACCEPTOR F22013.28 F19P19.27 | |
| | | | | | T19C21.15 YIP3 | |
| | | | | | TRANSMEMBRANE JWA PD011145: | |
| | | | | | F34-R114, I5-D28 | |
| | | | | | JM4 PROTEIN, COMPLETE CDS | BLAST_PRODOM |
| | | | | | CLONE IMAGE 546750 AND | |
| | | | | | LLNLC110F1857Q7 RZPD BERLIN | |
| | | | | | PD100903: I29-R114, D2-D25 | |
| | | | | | PROTEIN JM4 COMPLETE CDS | BLAST_PRODOM |
| | | | | | CLONE IMAGE LLNLC110F1857Q7 | |
| | | | | | RZPD BERLIN JWA | |
| | | | | | PD100906: L115-E148 | |
| 41 | 7508326CD1 | 417 | S207 S223 S299 | N152 N261 | Cytosolic domains: M1-Q94, | TMHMMER |
| | | | S308 S332 S337 | | S272-S417 | |
| | | | S344 S355 S370 T3 | | Transmembrane domains: | |
| | | | T136 | | F95-Y117, F249-F271 | |
| | | | | | Non-cytosolic domain: Y118-N248 | |
| | | | | | Leucine zipper pattern: | MOTIFS |
| | | | | | L84-L105, L91-L112 | |
| 42 | 7506370CD1 | 176 | S37 S46 S104 S138 | N32 | signal_cleavage: M1-R39 | SPSCAN |
| | | | Y45 | | | |
| 43 | 6312989CD1 | 579 | S10 S18 S96 S100 | N252 N362 | Cytosolic domains: S148-S159, | TMHMMER |
| | | | S109 S155 S178 | | S208-K263 | |
| | | | S208 S243 S253 | | Transmembrane domains: A125-A147, | |
| | | | S352 S411 S433 | | C160-W179, L185-L207, | |
| | | | S453 S485 S506 | | N264-V286 | |
| | | | S540 S549 S555 | | Non-cytosolic domains: M1-Q124, | |
| | | | T105 T151 T254 | | K180-T184, C287-L579 | |
| | | | T327 T393 | | | |
| 43 | | | | | COTE1 PROTEIN PD146399: M93-L448, | BLAST_PRODOM |
| | | | | | A415-L579, R6-G49 | |
| | | | | | Leucine zipper pattern: | MOTIFS |
| | | | | | L193-L214 | |
| | | | | | Cell attachment sequence: | MOTIFS |
| | | | | | R387-D389 | |
| 44 | 7501108CD1 | 357 | S151 S200 S226 | N65 N95 N134 | signal_cleavage: M1-G35 | SPSCAN |
| | | | S249 S255 S351 | N159 N187 | | |
| | | | | N230 N284 | | |
| | | | | | Signal Peptides: M1-G35, | HMMER |
| | | | | | M1-L34 | |
| | | | | | Cytosolic domains: M1-E6, | TMHMMER |
| | | | | | H346-N357 | |
| | | | | | Transmembrane domains: C7-A29, | |
| | | | | | G323-L345 | |
| | | | | | Non-cytosolic domain: P30-L322 | |
| | | | | | Leucine zipper pattern: | MOTIFS |
| | | | | | L322-L343 | |
| 45 | 7507581CD1 | 301 | S12 S27 S30 S34 | N223 | Cytosolic domains: S141-L174, | TMHMMER |
| | | | S59 S65 S75 S89 | | R259-A301 | |
| | | | S159 S163 S263 | | Transmembrane domains: G118-I140, | |
| | | | T51 T212 T266 | | A175-V197, A236-Y258 | |
| | | | | | Non-cytosolic domains: M1-P117, | |
| | | | | | K198-A235 | |
| | | | | | 32.0 KD PROTEIN IN CHROMOSOME | BLAST_PRODOM |
| | | | | | III TRANSMEMBRANE PD128096: | |
| | | | | | L81-F199, A235-L286 | |
| | | | | | Cell attachment sequence: | MOTIFS |
| | | | | | R289-D291 | |
| 46 | 7506361CD1 | 562 | S44 S91 S117 S123 | | Ergosterol biosynthesis | HMMER_PFAM |
| | | | S148 T65 T84 | | ERG4/ERG24 family: T149-C516 | |
| | | | T380 T440 T523 | | | |
| | | | | | Cytosolic domains: M1-R231, | TMHMMER |
| | | | | | M284-G388, T487-P562 | |
| | | | | | Transmembrane domains: | |
| | | | | | Y232-G254, L264-Y283, | |
| | | | | | F389-F411, M464-F486 | |
| | | | | | Non-cytosolic domains: L255-M263, | |
| | | | | | L412-I463 | |
| 46 | | | | | Ergosterol biosynthesis | BLIMPS_{—} |
| | | | | | ERG4/ERG24 family proteins | BLOCKS |
| | | | | | BL01017: S300-N314, P315-L340, | |
| | | | | | L366-F411, L466-T518 | |
| | | | | | LAMIN B RECEPTOR HOMOLOG | BLAST_PRODOM |
| | | | | | TM7SF2 | |
| | | | | | PD178440: A16-A217 | |
| | | | | | PD167876: T505-P562 | |
| | | | | | REDUCTASE STEROL TRANS- | BLAST_PRODOM |
| | | | | | MEMBRANE OXIDOREDUCTASE | |
| | | | | | BIOSYNTHESIS C14 | |
| | | | | | C14REDUCTASE PROTEIN | |
| | | | | | LAMIN B | |
| | | | | | PD004179: G215-K443, | |
| | | | | | P416-L478, G473-R503 | |
| | | | | | ERGOSTEROL BIOSYNTHESIS | BLAST_DOMO |
| | | | | | ERG4/ERG24 FAMILY DM01860 | |
| | | | | | \|A53616\|196-614: | |
| | | | | | Y210-I430, L433-W507 | |
| | | | | | \|P23913\|190-607: | |
| | | | | | Y210-K443, L433-W507 | |
| | | | | | \|JC4057\|3-423: | |
| | | | | | A219-K443, L433-R503 | |
| | | | | | \|P38670\|11-489: | |
| | | | | | E220-G420, I430-S475, | |
| | | | | | Y480-R503 | |
| | | | | | Ergosterol biosynthesis | MOTIFS |
| | | | | | ERG4/ERG24 family signature | |
| | | | | | 1: G301-R316 | |
| 47 | 7509211CD1 | 651 | S4 S8 S17 S63 S97 | N141 N251 | Cytosolic domains: M1-Q32, | TMHMMER |
| | | | S132 S142 S241 | | C98-K152 | |
| | | | S300 S324 S337 | | Transmembrane domains: A33-A55, | |
| | | | S387 S446 S473 | | S75-S97, N153-V175 | |
| | | | S504 S524 S556 | | Non-cytosolic domains: S56-F74, | |
| | | | S578 S612 S621 | | C176-L651 | |
| | | | S627 T13 T59 | | | |
| | | | T143 T216 T282 | | | |
| | | | T442 T481 | | | |
| 47 | | | | | COTE1 PROTEIN PD146399: | BLAST_PRODOM |
| | | | | | M1-Y237, F51-L651 | |
| | | | | | Leucine zipper pattern: | MOTIFS |
| | | | | | L82-L103 | |
| | | | | | Cell attachment sequence: | MOTIFS |
| | | | | | R276-D278 | |

TABLE 4

| Polynucleotide | |
|---|---|
| SEQ ID NO: / | |
| Incyte ID/Sequence | |
| Length | Sequence Fragments |
| 48/3356677CB1/2061 | 1-290, 1-716, 1-1083, 11-515, 11-572, 95-290, 149-223, |
| | 196-529, 384-773, 395-665, 411-715, 431-718, 431-866, 455-549, |
| | 503-715, 548-1139, 628-858, 744-1142, 781-936, 781-1031, |
| | 884-1245, 1061-1712, 1164-1712, 1259-1466, 1332-2019, 1343-1987, |
| | 1403-1987, 1442-2038, 1529-1728, 1608-2046, 1608-2061, 1623-2045, |
| | 1635-2040, 1639-2045 |
| 49/7481665CB1/2649 | 1-578, 93-514, 93-518, 93-574, 93-651, 111-212, 270-1098, |
| | 648-1367, 648-1383, 677-1370, 687-930, 687-953, 687-1007, |
| | 687-1010, 687-1161, 687-1193, 687-1196, 687-1209, 687-1292, |
| | 687-1381, 687-1391, 687-1394, 712-1391, 748-1391, 793-1098, |
| | 793-1391, 794-1391, 810-913, 820-1391, 859-1453, 1239-1845, |
| | 1239-1852, 1239-1947, 1254-1822, 1281-1700, 1281-1889, 1281-1913, |
| | 1281-1941, 1281-1966, 1281-1971, 1282-1542, 1285-1766, 1290-1960, |
| | 1315-1391, 1318-1392, 1440-1954, 1490-2196, 1533-2310, 1583-2154, |
| | 1782-2418, 1853-2085, 1864-2372, 1882-2453, 1912-2148, 1921-2258, |
| | 1944-2484, 1990-2649, 2025-2282, 2055-2318, 2069-2628, 2084-2505, |
| | 2093-2431, 2093-2455, 2097-2624, 2121-2406 |
| 50/3563859CB1/1528 | 1-287, 1-391, 1-462, 1-1500, 281-863, 481-1500, 570-1248, |
| | 584-816, 593-922, 663-942, 666-975, 666-1143, 682-974, 687-1484, |
| | 697-901, 708-994, 709-889, 714-959, 714-1340, 749-915, 766-1500, |
| | 769-1026, 792-863, 854-1487, 961-1272, 1039-1507, 1043-1297, |
| | 1043-1528, 1056-1317, 1056-1451, 1056-1517, 1058-1502, 1068-1500, |
| | 1096-1361, 1096-1452, 1096-1500, 1096-1511, 1134-1420, 1156-1498, |
| | 1180-1498, 1189-1528, 1190-1528, 1198-1500, 1211-1500, 1253-1519, |
| | 1273-1500, 1273-1513, 1319-1518, 1366-1500 |
| 51/2588884CB1/1469 | 1-850, 532-697, 532-735, 532-786, 532-1052, 532-1150, 532-1198, |
| | 532-1469, 534-768, 534-993, 536-778, 536-1211, 538-743, 539-962, |
| | 542-1003, 548-779, 569-833, 587-812, 644-788, 687-1369, 693-962, |
| | 721-1294, 1237-1469 |
| 53/7503424CB1/1464 | 1-504, 1-537, 2-293, 3-164, 4-313, 5-292, 5-516, 5-677, |
| | 12-560, 14-259, 15-237, 15-254, 15-1260, 16-507, 16-540, |
| | 16-675, 17-287, 17-308, 17-646, 17-655, 19-671, 21-251, |
| | 21-640, 51-331, 77-667, 87-321, 88-341, 88-359, 100-355, |
| | 105-355, 105-369, 112-341, 112-362, 112-631, 114-628, 137-351, |
| | 146-388, 146-677, 168-277, 171-423, 175-431, 176-432, 190-400, |
| | 190-437, 208-497, 215-450, 216-495, 216-498, 223-468, 223-598, |
| | 232-453, 232-489, 234-499, 239-425, 246-537, 246-552, 247-513, |
| | 252-476, 252-525, 271-545, 273-525, 280-519, 280-550, 281-521, |
| | 283-552, 297-580, 339-626, 340-586, 346-557, 346-596, 351-620, |
| | 356-565, 366-604, 368-669, 375-640, 377-637, 379-587, 395-668, |
| | 409-624, 409-629, 423-671, 428-648, 428-670, 428-677, 432-673, |
| | 434-677, 435-675, 435-677, 439-677, 468-677, 478-623, 496-669, |
| | 514-677, 537-635, 551-677, 637-1251, 716-840, 716-913, 716-948, |
| | 716-949, 716-954, 716-1037, 716-1041, 716-1200, 716-1227, |
| | 716-1237, 716-1248, 716-1260, 716-1272, 717-1089, 718-931, |
| | 719-1207, 720-1192, 724-1282, 725-924, 726-1291, 728-1053, |
| | 729-960, 729-992, 732-1156, 734-887, 735-1023, 748-1015, |
| | 752-950, 752-1009, 752-1212, 753-1219, 755-975, 756-1268, |
| | 757-994, 757-1021, 759-1044, 759-1111, 761-969, 761-1010, |
| | 761-1013, 761-1016, 762-996, 762-1020, 762-1024, 762-1032, |
| | 762-1033, 764-979, 769-1033, 769-1052, 769-1271, 772-1275, |
| | 775-1017, 780-983, 780-1006, 780-1064, 780-1201, |
| | 783-1120, 784-1120, 784-1273, 785-1016, 786-1258, 788-1223, |
| | 790-1055, 791-1077, 791-1231, 791-1273, 791-1276, 792-1256, |
| | 794-1053, 795-1035, 795-1039, 799-1256, 800-1274, 806-1084, |
| | 808-1275, 812-1077, 812-1100, 813-1031, 815-1027, 815-1276, |
| | 816-1276, 821-1264, 822-1026, 822-1094, 825-1041, 831-1097, |
| | 832-1257, 833-1260, 835-1072, 842-1277, 843-1080, 844-1220, |
| | 851-1042, 851-1088, 854-1080, 855-1082, 856-1087, 860-1195, |
| | 860-1316, 861-1060, 865-1014, 866-1325, 867-1190, 868-1101, |
| | 868-1121, 869-1274, 870-1081, 873-1185, 876-1118, 876-1131, |
| | 876-1142, 876-1143, 878-1110, 878-1121, 879-1096, 879-1106, |
| | 879-1144, 879-1148, 880-1154, 881-1106, 883-1099, 885-1260, |
| | 890-1121, 892-1110, 892-1114, 892-1127, 897-1175, 900-1132, |
| | 900-1260, 901-1162, 908-1096, 911-1288, 919-1156, 919-1163, |
| | 919-1188, 921-1168, 921-1179, 921-1192, 921-1194, 924-1272, |
| | 925-1257, 926-1183, 927-1201, 927-1223, 927-1270, 928-1144, |
| | 937-1072, 937-1257, 940-1188, 941-1173, 941-1257, 945-1292, |
| | 946-1258, 949-1208, 949-1257, 955-1257, 955-1277, 956-1251, |
| | 956-1260, 956-1276, 958-1229, |
| | 960-1272, 968-1100, 968-1171, 968-1206, 971-1272, 973-1272, |
| | 974-1256, 975-1201, 977-1111, 977-1208, 977-1218, 979-1198, |
| | 981-1257, 985-1223, 986-1306, 988-1260, 993-1235, 993-1246, |
| | 994-1271, 996-1260, 1003-1268, 1003-1277, 1004-1234, 1005-1247, |
| | 1007-1141, 1007-1240, 1008-1250, 1008-1291, 1009-1280, 1010-1220, |
| | 1015-1246, 1017-1171, 1017-1276, 1021-1250, 1021-1274, 1023-1275, |
| | 1024-1260, 1027-1256, 1027-1270, 1028-1200, 1028-1222, 1028-1288, |
| | 1034-1270, 1038-1157, 1046-1301, 1048-1260, 1052-1272, 1055-1295, |
| | 1058-1120, 1058-1273, 1058-1291, 1064-1289, 1066-1272, |
| | 1067-1272, 1067-1288, 1072-1260, 1072-1279, 1073-1222, 1075-1246, |
| | 1075-1276, 1078-1302, 1083-1314, 1089-1326, 1091-1313, 1091-1334, |
| | 1098-1243, 1099-1288, 1102-1294, 1104-1279, 1113-1340, 1113-1345, |
| | 1138-1241, 1138-1278, 1138-1321, 1139-1247, 1141-1274, 1141-1310, |
| | 1144-1236, 1144-1391, 1145-1337, 1149-1331, 1156-1179, 1161-1271, |
| | 1163-1271, 1164-1260, 1165-1271, 1177-1312, 1179-1302, 1184-1320, |
| | 1186-1271, 1189-1260, 1193-1260, 1196-1464, 1209-1246 |
| 54/7503571CB1/657 | 1-165, 1-194, 1-296, 1-348, 1-516, 1-573, 1-574, 1-634, |
| | 1-640, 4-627, 12-194, 126-639, 194-392, 194-573, 194-639, |
| | 196-627, 198-573, 199-573, 199-646, 201-649, 208-628, 229-627, |
| | 336-626, 362-626, 366-573, 369-657, 370-571, 401-631, 512-573, |
| | 512-638, 512-657, 587-624, 587-625, 587-626 |
| 55/7505722CB1/1513 | 1-596, 1-618, 16-730, 18-497, 19-553, 93-525, 130-525, |
| | 253-1008, 330-986, 384-768, 549-1253, 550-1179, 791-1513, |
| | 973-1372 |
| 56/7505798CB1/1026 | 1-278, 1-336, 1-342, 3-589, 8-195, 15-259, 18-275, 20-1026, |
| | 23-276, 25-301, 66-342, 68-346, 71-294, 85-345, 92-315, |
| | 276-1014, 286-546, 325-942, 331-355, 336-998, 349-554, 349-622, |
| | 349-624, 349-829, 350-981, 358-873, 370-690, 378-557, 389-873, |
| | 391-909, 394-961, 406-612, 409-684, 409-691, 415-667, 435-962, |
| | 460-701, 465-814, 479-787, 483-775, 509-752, 521-950, 548-808, |
| | 552-783, 598-857, 601-821, 612-868, 628-874, 657-961, 660-961, |
| | 671-967, 695-1010, 715-961, 716-1026, 729-935, 790-916, 846-1026 |
| 57/7505847CB1/1895 | 1-285, 4-541, 7-1876, 10-776, 10-830, 10-831, 10-858, |
| | 10-863, 10-867, 16-708, 22-277, 22-671, 38-554, 48-675, |
| | 49-366, 52-322, 54-818, 55-656, 60-314, 60-741, 63-695, |
| | 65-627, 66-650, 70-329, 71-332, 71-679, 72-287, 72-330, |
| | 73-498, 73-516, 73-746, 75-328, 76-375, 77-320, 77-321, |
| | 77-343, 78-310, 78-362, 78-860, 79-336, 79-635, 79-684, |
| | 81-311, 81-327, 81-686, 81-710, 81-719, 82-343, 83-355, |
| | 83-368, 84-333, 84-355, 84-360, 84-573, 85-587, 85-840, |
| | 86-332, 86-788, 87-333, 87-735, 89-290, 89-333, 89-624, |
| | 89-712, 90-864, 91-369, 92-375, 92-668, 95-318, 95-338, |
| | 95-343, 95-385, 95-388, 95-392, 95-470, 95-833, 96-348, |
| | 96-420, 96-526, 97-361, 97-395, 98-343, 98-362, 98-704, |
| | 99-287, 99-355, 100-340, 100-674, 100-719, 103-317, 103-420, |
| | 106-370, 106-402, 106-728, 106-814, 107-648, 109-667, 112-334, |
| | 115-366, 116-617, 121-682, 125-383, 128-389, 140-385, 148-419, |
| | 161-801, 163-392, 168-448, 170-774, 171-639, 171-731, 180-422, |
| | 182-434, 184-438, 185-705, 187-835, 188-438, 190-694, 190-776, |
| | 193-741, |
| | 196-457, 214-421, 222-507, 235-818, 242-500, 257-469, 263-535, |
| | 264-857, 266-511, 280-520, 301-819, 311-705, 313-511, 323-513, |
| | 330-614, 335-838, 336-581, 342-609, 345-597, 355-622, 356-820, |
| | 376-649, 386-864, 388-611, 389-647, 390-649, 394-661, 394-844, |
| | 411-511, 425-709, 425-714, 432-670, 432-699, 433-547, 434-688, |
| | 452-772, 452-818, 456-697, 461-730, 470-867, 472-723, 478-593, |
| | 481-743, 499-722, 500-731, 507-716, 509-783, 516-735, 520-731, |
| | 520-738, 525-826, 527-691, 529-739, 545-831, 576-859, 683-858, |
| | 771-981, 771-1062, 884-1154, 900-1542, 903-1157, 918-1550, |
| | 1012-1160, 1041-1283, 1083-1825, 1125-1576, 1243-1499, 1256-1499, |
| | 1256-1505, 1265-1525, 1289-1579, 1299-1579, 1429-1879, 1433-1669, |
| | 1435-1699, 1445-1861, 1456-1708, 1470-1732, 1475-1725, 1475-1880, |
| | 1509-1724, 1512-1880, 1531-1895, 1538-1878, 1545-1862, 1566-1879, |
| | 1572-1846, 1596-1872, 1666-1827, 1669-1765, 1669-1890, 1670-1869, |
| | 1694-1887, 1696-1825, 1696-1850, 1711-1825, 1731-1880, 1741-1863, |
| | 1777-1895, 1818-1895 |
| 58/7505862CB1/1723 | 1-244, 1-1709, 1-1717, 47-674, 91-377, 91-403, 91-644, |
| | 263-509, 356-616, 373-640, 391-608, 398-674, 433-674, 439-537, |
| | 477-634, 478-671, 518-991, 543-1092, 588-1110, 670-856, 670-990, |
| | 670-1107, 670-1191, 672-891, 682-1136, 684-1149, 686-930, |
| | 688-1234, 695-940, 699-870, 712-1398, 724-1001, 728-1172, |
| | 740-882, 749-1011, 755-1314, 770-1350, 777-1006, 777-1239, |
| | 777-1356, 778-1326, 779-1284, 790-1006, 794-1068, 809-1104, |
| | 810-1449, 816-1542, 826-1016, 826-1162, 832-1097, 833-1308, |
| | 862-1012, 893-1699, 903-1144, 943-1605, 949-1220, 956-1251, |
| | 982-1275, 998-1219, 1005-1224, 1014-1280, 1023-1277, 1030-1232, |
| | 1032-1603, 1034-1694, 1072-1301, 1074-1692, 1076-1380, 1083-1664, |
| | 1088-1671, 1098-1536, 1108-1249, 1114-1397, 1128-1669, 1130-1333, |
| | 1133-1413, 1135-1723, 1136-1579, |
| | 1142-1598, 1145-1626, 1160-1717, 1164-1595, 1182-1457, 1190-1646, |
| | 1191-1481, 1200-1655, 1200-1656, 1200-1723, 1201-1696, 1210-1721, |
| | 1216-1497, 1224-1696, 1226-1679, 1229-1721, 1230-1721, 1238-1702, |
| | 1244-1721, 1251-1702, 1265-1721, 1266-1505, 1273-1651, 1274-1705, |
| | 1274-1708, 1274-1721, 1275-1705, 1286-1702, 1286-1721, 1287-1516, |
| | 1289-1721, 1298-1702, 1302-1561, 1304-1702, 1306-1711, 1306-1721, |
| | 1307-1709, 1319-1702, 1319-1712, 1322-1709, 1324-1647, 1329-1710, |
| | 1331-1708, 1335-1706, 1347-1708, 1349-1702, 1355-1705, 1358-1605, |
| | 1358-1690, 1358-1706, 1364-1718, 1366-1637, 1370-1708, 1371-1708, |
| | 1376-1668, 1379-1659, 1388-1716, 1399-1653, 1418-1707, 1421-1702, |
| | 1421-1707, 1423-1708, 1424-1702, 1429-1559, 1442-1619, 1448-1601, |
| | 1462-1696, 1483-1702, 1512-1703, 1519-1702, 1552-1716, 1563-1721 |
| 59/7762537CB1/1714 | 1-314, 105-405, 318-591, 327-946, 330-588, 344-586, 421-757, |
| | 421-876, 421-942, 429-1042, 441-1047, 481-1172, 495-1171, |
| | 558-799, 579-866, 628-921, 679-946, 679-1120, 684-945, 685-1094, |
| | 695-1305, 698-1419, 704-972, 706-1113, 738-1009, 812-1423, |
| | 821-1149, 824-1079, 824-1106, 827-1079, 832-1119, 841-1163, |
| | 860-1060, 860-1147, 912-1295, 912-1529, 924-1037, 925-1209, |
| | 929-1449, 934-1294, 946-1156, 975-1692, 982-1607, 984-1152, |
| | 1010-1280, 1040-1694, 1045-1297, 1049-1313, 1059-1714, 1086-1678, |
| | 1105-1679, 1127-1702, 1128-1691, 1140-1668, 1148-1642, 1148-1703, |
| | 1151-1560, 1162-1440, 1209-1455, 1209-1679, 1209-1694, 1210-1336, |
| | 1213-1703, 1223-1481, 1228-1707, 1228-1714, 1247-1702, 1248-1702, |
| | 1252-1707, 1256-1702, 1291-1699, 1330-1703, 1341-1713, 1355-1702, |
| | 1357-1598, 1383-1543, 1386-1667, 1395-1629, 1395-1688, 1395-1702, |
| | 1395-1714, 1442-1648, 1442-1666, 1453-1702, 1463-1703, 1472-1610, |
| | 1485-1702, 1487-1702, 1496-1703, 1538-1714, 1548-1714, 1550-1703, |
| | 1550-1714, 1599-1714 |
| 60/90033462CB1/1199 | 1-629, 9-474, 105-783, 106-600, 110-990, 110-1068, 111-934, |
| | 114-727, 114-824, 114-831, 114-836, 114-837, 114-844, 114-907, |
| | 114-909, 114-930, 114-935, 114-953, 114-954, 114-1032, 319-1193, |
| | 368-1193, 371-1199, 433-1199, 457-1199, 469-1199 |
| 61/1644869CB1/4572 | 1-674, 131-374, 131-403, 132-402, 137-716, 140-550, 140-704, |
| | 140-749, 140-782, 197-558, 216-378, 216-992, 246-651, 248-537, |
| | 459-1097, 565-1140, 701-1287, 761-1267, 786-1319, 786-1391, |
| | 796-1428, 915-1702, 917-1557, 1004-1296, 1052-1717, 1103-1722, |
| | 1128-1759, 1366-1996, 1406-1878, 1422-2052, 1426-1717, 1620-2179, |
| | 1636-2238, 1750-2003, 1763-2439, 1852-2067, 1859-2441, 1908-2129, |
| | 1988-2548, 2052-2316, 2056-2566, 2076-2695, 2080-2662, 2084-2756, |
| | 2087-2652, 2211-2844, 2271-2478, 2271-2645, 2271-2696, 2388-3142, |
| | 2389-2947, 2447-3040, 2540-2805, 2580-3164, 2630-3164, 2655-2938, |
| | 2679-2937, 2681-2906, 2681-3148, 2690-3155, 2707-3235, 2713-3321, |
| | 2730-2994, 2730-3309, 2797-3424, 2798-3047, 2802-3489, 2815-3041, |
| | 2815-3230, 2820-3319, 2822-3068, 2825-3430, 2835-3081, 2858-3398, |
| | 2883-3418, 2884-3136, 2902-3609, 2929-3595, 2945-3184, 2948-3384, |
| | 2971-3602, |
| | 2975-3234, 2977-3079, 2977-3086, 3017-3693, 3030-3268, 3063-3323, |
| | 3078-3674, 3090-3522, 3141-3378, 3141-3565, 3141-3760, 3142-3562, |
| | 3142-3805, 3156-3657, 3180-4497, 3189-3421, 3190-3689, 3190-3730, |
| | 3197-3697, 3203-3580, 3208-3331, 3210-3475, 3216-3404, 3216-3471, |
| | 3216-3692, 3216-3735, 3247-3711, 3271-3984, 3276-3481, 3279-4012, |
| | 3285-3550, 3313-3909, 3334-3827, 3389-3909, 3465-3731, 3499-3740, |
| | 3547-3791, 3577-3832, 3585-3879, 3599-4098, 3620-4131, 3630-4237, |
| | 3630-4248, 3639-3944, 3644-4326, 3651-3902, 3653-4096, 3659-4403, |
| | 3666-4119, 3667-4104, 3680-4304, 3703-4367, 3710-4208, 3733-4418, |
| | 3734-4213, 3740-4206, 3759-3988, 3777-4255, 3784-4030, 3790-4498, |
| | 3794-3991, 3794-4234, 3795-4050, 3805-4132, 3817-4073, 3817-4188, |
| | 3829-4307, 3842-4451, 3851-4128, 3854-4436, 3856-4230, 3860-4341, |
| | 3863-4429, 3866-4130, 3866-4138, 3866-4317, 3874-4541, |
| | 3879-4139, 3881-4355, 3881-4414, 3886-4498, 3897-4150, 3897-4170, |
| | 3907-4174, 3913-4534, 3923-4160, 3926-4176, 3934-4535, 3936-4194, |
| | 3945-4367, 3952-4440, 3953-4564, 3961-4545, 3965-4506, 3975-4113, |
| | 3987-4269, 3993-4546, 4005-4531, 4012-4542, 4026-4562, 4027-4569, |
| | 4043-4547, 4043-4561, 4046-4382, 4059-4568, 4073-4546, 4078-4281, |
| | 4078-4558, 4078-4564, 4088-4552, 4089-4536, 4090-4551, 4091-4548, |
| | 4091-4558, 4103-4566, 4113-4402, 4116-4546, 4128-4504, 4128-4552, |
| | 4130-4557, 4131-4546, 4131-4552, 4132-4402, 4135-4547, 4135-4572, |
| | 4136-4568, 4139-4345, 4139-4367, 4139-4514, 4139-4552, 4139-4553, |
| | 4139-4561, 4146-4377, 4148-4366, 4155-4515, 4158-4552, 4168-4343, |
| | 4172-4567, 4174-4552, 4179-4516, 4179-4552, 4184-4552, 4186-4552, |
| | 4191-4473, 4195-4449, 4196-4552, 4196-4553, 4199-4552, 4205-4546, |
| | 4221-4433, 4225-4505, 4228-4438, 4235-4508, 4236-4384, 4256-4534, |
| | 4257-4572, 4271-4539, 4271-4545, 4271-4568, 4285-4492, 4328-4513, |
| | 4329-4547, 4329-4558, 4338-4499, 4452-4552 |
| 62/6288712CB1/2044 | 1-216, 49-326, 152-762, 554-1136, 558-1354, 559-1071, 814-1108, |
| | 814-1240, 814-1377, 814-1425, 814-1496, 1187-1925, 1281-2013, |
| | 1438-2044, 1576-1832, 1576-2044 |
| 63/71830156CB1/1300 | 1-471, 1-516, 1-540, 1-550, 1-569, 1-601, 1-625, 1-626, |
| | 1-690, 1-782, 1-874, 1-982, 8-716, 30-282, 142-751, 192-659, |
| | 211-835, 222-785, 343-995, 384-1003, 385-996, 389-970, 391-949, |
| | 434-981, 434-987, 517-981, 524-997, 545-977, 548-982, 558-995, |
| | 594-981, 609-986, 700-965, 715-1300, 723-981, 723-996 |
| 64/7505044CB1/1081 | 1-269, 1-545, 1-1076, 1-1078, 24-258, 39-289, 56-237, |
| | 104-651, 145-651, 155-761, 177-954, 231-482, 300-561, 301-891, |
| | 346-843, 364-1010, 411-1055, 458-1045, 476-1053, 485-1034, |
| | 486-799, 564-711, 564-1009, 567-1050, 592-1069, 594-1060, |
| | 595-879, 602-739, 611-879, 614-879, 624-879, 629-1076, 655-879, |
| | 661-879, 663-879, 673-961, 732-982, 732-1044, 732-1065, 780-1052, |
| | 807-1081 |
| 65/7505086CB1/2899 | 1-596, 16-130, 16-310, 16-349, 16-466, 16-577, 16-776, |
| | 16-2899, 19-640, 23-306, 108-686, 270-614, 285-826, 293-889, |
| | 350-1132, 410-988, 427-944, 460-996, 463-1012, 495-1037, |
| | 557-1206, 559-1287, 635-917, 655-1204, 704-1301, 727-1309, |
| | 783-1293, 842-1418, 856-1067, 880-1498, 910-1591, 970-1506, |
| | 999-1629, 1000-1692, 1029-1604, 1206-1470, 1216-1923, 1222-1842, |
| | 1228-1854, 1247-1928, 1255-1615, 1273-1855, 1314-1959, 1387-1640, |
| | 1392-2000, 1411-2106, 1454-2078, 1460-1928, 1465-1720, 1510-2174, |
| | 1516-2209, 1518-2128, 1525-1702, 1525-2013, 1525-2027, 1525-2067, |
| | 1525-2081, 1525-2088, 1525-2142, 1525-2291, 1534-2253, 1541-2225, |
| | 1562-2171, 1566-1837, 1570-2142, 1573-2106, 1580-2251, 1592-2244, |
| | 1599-2215, 1600-2150, 1606-2288, 1646-2267, 1662-1913, 1702-2241, |
| | 1757-2034, 1762-2321, 1800-2280, 1819-2228, 1832-2166, 1928-2270, |
| | 2010-2251, 2016-2134, 2054-2281, 2054-2310, 2102-2292, 2185-2455, |
| | 2185-2660, 2185-2676, 2185-2692, 2185-2704, 2185-2723, 2185-2729, |
| | 2203-2321, 2319-2876, 2368-2896, 2384-2851, 2415-2696, 2415-2839, |
| | 2415-2860, 2426-2890, 2428-2527, 2428-2894, 2452-2697, 2578-2808, |
| | 2614-2894, 2639-2899 |
| 66/7505784CB1/880 | 1-192, 1-193, 16-151, 16-184, 16-741, 18-163, 20-302, |
| | 21-120, 21-197, 22-116, 22-179, 66-329, 200-367, 200-708, |
| | 212-504, 218-738, 229-336, 229-500, 243-528, 243-778, 251-534, |
| | 252-591, 253-461, 253-489, 254-435, 254-487, 254-504, 255-403, |
| | 255-464, 255-532, 258-499, 259-469, 265-510, 266-536, 266-537, |
| | 268-486, 276-755, 276-759, 276-804, 276-806, 302-531, 302-571, |
| | 306-557, 316-476, 320-590, 326-743, 326-802, 329-743, 340-554, |
| | 340-588, 343-609, 359-586, 360-648, 365-632, 366-742, 383-880, |
| | 391-679, 391-688, 402-679, 409-686, 412-678, 415-701, 419-677, |
| | 419-691, 424-569, 430-690, 431-689, 438-698, 451-715, 455-729, |
| | 456-707, 458-745, 459-747, 520-657, 538-713, 586-644, 593-866, |
| | 613-879, 616-749 |
| 67/7505813CB1/1172 | 1-625, 1-1164, 18-512, 18-797, 23-272, 29-521, 32-307, |
| | 38-642, 39-788, 41-293, 41-342, 43-333, 43-775, 45-596, |
| | 45-599, 45-665, 45-674, 45-759, 51-611, 55-184, 56-588, |
| | 64-617, 71-376, 75-548, 77-719, 82-643, 88-642, 113-617, |
| | 129-291, 153-555, 155-439, 166-688, 170-410, 175-410, 184-463, |
| | 190-745, 193-444, 194-489, 194-490, 194-511, 194-722, 197-323, |
| | 199-430, 199-483, 204-458, 205-477, 209-444, 232-508, 252-880, |
| | 258-513, 259-449, 264-546, 264-561, 278-517, 278-630, 308-557, |
| | 308-583, 309-794, 322-788, 329-605, 329-624, 346-530, 346-640, |
| | 359-595, 370-877, 392-583, 398-629, 398-643, 398-666, 398-690, |
| | 399-660, 401-612, 403-712, 411-703, 414-731, 416-583, 423-669, |
| | 455-580, 463-761, 471-659, 472-709, 487-707, 489-748, 491-744, |
| | 500-758, 507-773, 510-608, 510-789, 517-722, 521-746, 524-786, |
| | 528-700, |
| | 553-788, 554-801, 580-785, 695-1165, 737-1155, 773-1168, |
| | 797-1032, 798-993, 798-1022, 798-1040, 798-1043, 798-1087, |
| | 798-1153, 798-1156, 798-1168, 798-1169, 798-1172, 801-1158, |
| | 802-1050, 810-1060, 824-1153, 831-1098, 832-1160, 837-1154, |
| | 846-1153, 847-1168, 861-1153, 863-1149, 865-1154, 871-1158, |
| | 875-1153, 880-1153, 881-1153, 882-1153, 883-1154, 884-1154, |
| | 886-1118, 893-1115, 893-1160, 894-1153, 895-1153, 897-1157, |
| | 900-1107, 904-1145, 911-1167, 912-1151, 914-1124, 917-1169, |
| | 919-1164, 930-1095, 930-1171, 946-1162, 946-1171, 947-1153, |
| | 951-1153, 952-1158, 961-1163, 969-1165, 971-1169, 975-1153, |
| | 975-1154, 975-1167, 978-1132, 983-1172, 987-1153, 1006-1153, |
| | 1019-1169, 1057-1153 |
| 68/7505873CB1/1998 | 1-205, 1-241, 1-463, 1-669, 1-686, 1-1974, 3-678, 125-777, |
| | 125-813, 125-837, 125-844, 125-871, 125-872, 125-879, 125-882, |
| | 125-885, 125-890, 125-917, 125-918, 125-957, 125-958, 125-960, |
| | 125-1004, 153-529, 337-752, 426-1074, 455-771, 491-1090, |
| | 571-829, 571-832, 571-976, 571-1061, 571-1125, 582-1037, |
| | 584-1250, 592-1206, 639-889, 639-1150, 667-1304, 830-1691, |
| | 841-1269, 856-1694, 900-1691, 938-1694, 954-1691, 955-1696, |
| | 1156-1264, 1156-1338, 1339-1963, 1346-1973, 1368-1919, 1375-1964, |
| | 1379-1699, 1384-1680, 1406-1649, 1416-1626, 1417-1995, 1443-1974, |
| | 1464-1960, 1466-1974, 1468-1914, 1471-1974, 1474-1953, 1477-1830, |
| | 1484-1956, 1495-1989, 1516-1872, 1525-1845, 1547-1974, 1556-1982, |
| | 1566-1973, 1573-1998, 1584-1976, 1597-1850, 1614-1877, 1646-1973, |
| | 1655-1895, 1655-1974, 1692-1947, 1693-1954, 1710-1933, 1715-1974, |
| | 1728-1954, 1824-1988 |
| 69/7505881CB1/919 | 1-222, 8-209, 8-212, 8-276, 8-912, 11-283, 51-313, 54-318, |
| | 54-328, 54-353, 55-260, 57-309, 58-302, 58-669, 63-682, |
| | 64-657, 65-586, 67-300, 68-304, 68-317, 72-273, 77-331, |
| | 77-341, 77-431, 77-695, 77-716, 78-319, 82-262, 82-370, |
| | 220-538, 254-660, 294-423, 299-570, 423-684, 465-663, 466-919, |
| | 498-725, 505-682, 539-703, 549-907, 703-910, 827-919 |
| 70/7503510CB1/3858 | 1-3820, 1-3831, 124-403, 124-474, 124-511, 124-523, 124-529, |
| | 124-547, 124-576, 124-595, 124-612, 124-621, 126-398, 127-861, |
| | 132-583, 132-615, 132-628, 132-645, 132-689, 132-698, 132-713, |
| | 132-718, 132-724, 132-731, 132-777, 132-778, 132-813, 132-852, |
| | 142-594, 142-609, 155-583, 171-360, 181-663, 197-433, 242-482, |
| | 252-357, 252-360, 257-561, 260-911, 338-865, 352-1026, 366-478, |
| | 390-656, 436-671, 436-839, 473-882, 514-959, 564-806, 689-1241, |
| | 697-1249, 813-1482, 833-1469, 917-1569, 945-1339, 946-1492, |
| | 973-1329, 1036-1523, 1101-1765, 1107-1727, 1114-1501, 1148-1426, |
| | 1176-1430, 1185-1684, 1215-1465, 1216-1818, 1217-1466, 1221-1883, |
| | 1250-1811, 1266-1814, 1273-1508, 1293-1875, 1416-1880, 1468-1929, |
| | 1487-1561, 1494-1922, 1494-1929, 1512-1691, 1530-1811, 1876-2447, |
| | 1994-2485, |
| | 1995-2634, 2058-2336, 2096-2345, 2109-2346, 2111-2348, 2111-2633, |
| | 2123-2357, 2127-2403, 2140-2335, 2149-2353, 2152-2434, 2161-2402, |
| | 2163-2393, 2171-2434, 2171-2437, 2225-2499, 2307-2431, |
| | 2417-3021, 2420-3002, 2472-2733, 2481-2735, 2496-2761, 2498-2750, |
| | 2503-2834, 2520-3178, 2523-3292, 2524-2785, 2525-2799, 2532-2801, |
| | 2537-3054, 2544-2792, 2545-2803, 2545-2821, 2553-2817, 2574-2847, |
| | 2575-2869, 2575-3027, 2576-2790, 2581-2633, 2584-2824, |
| | 2585-2883, 2597-2996, 2599-2835, 2599-2847, 2599-3188, 2600-2824, |
| | 2606-2891, 2618-2858, 2620-2849, 2632-3146, 2640-3258, 2642-2925, |
| | 2642-3266, 2654-2873, 2654-2878, 2656-2984, 2657-2947, 2669-2747, |
| | 2680-2971, 2683-2768, 2683-2948, 2683-2965, 2683-3023, |
| | 2683-3290, 2689-2885, 2689-2923, 2689-2949, 2709-3063, 2709-3119, |
| | 2712-3461, 2723-3201, 2727-3009, 2727-3325, 2729-3209, 2731-3436, |
| | 2741-2949, 2741-3402, 2744-3025, 2744-3216, 2744-3274, 2755-2948, |
| | 2762-2970, 2778-3333, 2779-3019, 2779-3036, |
| | 2781-2993, 2791-3074, 2795-3317, 2797-3353, 2800-3050, 2801-3091, |
| | 2808-3081, 2810-3066, 2810-3375, 2812-3194, 2812-3206, 2822-3042, |
| | 2848-3154, 2854-3307, 2855-3116, 2856-3112, 2858-3115, 2861-3014, |
| | 2862-3106, 2878-3437, 2879-3266, 2888-3173, 2897-3514, 2911-3259, |
| | 2913-3191, 2914-3353, 2916-3179, 2923-3148, 2943-3236, 2949-3173, |
| | 2950-3531, 2954-3456, 2956-3200, 2956-3637, 2966-3225, 2966-3260, |
| | 2966-3261, 2969-3459, 2969-3603, 2970-3157, 2970-3184, 2972-3242, |
| | 2973-3484, 2979-3241, 2982-3232, 2988-3621, 2999-3223, 3001-3377, |
| | 3001-3444, 3007-3275, 3012-3273, 3015-3775, 3017-3328, 3018-3273, |
| | 3019-3305, 3033-3251, 3037-3749, 3042-3329, 3045-3259, 3045-3262, |
| | 3045-3263, 3045-3281, 3045-3446, 3045-3461, 3048-3298, 3049-3341, |
| | 3056-3312, 3061-3263, 3061-3486, 3063-3308, 3071-3366, 3072-3288, |
| | 3072-3327, 3072-3336, 3072-3342, 3072-3343, 3074-3306, 3075-3166, |
| | 3079-3288, 3079-3515, 3081-3342, 3083-3386, 3085-3379, 3088-3359, |
| | 3094-3702, 3097-3472, 3104-3769, 3108-3364, 3122-3406, 3123-3388, |
| | 3129-3367, 3129-3776, 3130-3686, 3131-3388, |
| | 3139-3352, 3139-3439, 3139-3814, 3141-3445, 3141-3747, 3144-3406, |
| | 3144-3408, 3144-3426, 3144-3677, 3144-3805, 3148-3371, 3154-3447, |
| | 3160-3693, 3166-3728, 3166-3772, 3166-3804, 3168-3430, 3172-3429, |
| | 3172-3451, 3172-3817, 3172-3846, 3174-3389, 3174-3394, 3174-3441, |
| | 3176-3702, 3184-3603, 3187-3482, 3190-3466, 3191-3421, 3193-3673, |
| | 3194-3465, 3200-3453, 3200-3497, 3212-3496, 3216-3779, 3225-3498, |
| | 3226-3783, 3227-3465, 3227-3817, 3228-3538, 3232-3470, 3235-3527, |
| | 3235-3804, 3235-3844, 3236-3487, 3237-3819, 3242-3819, 3244-3765, |
| | 3246-3715, 3250-3788, 3251-3516, 3252-3530, 3256-3491, 3256-3528, |
| | 3256-3539, 3256-3558, 3258-3500, 3261-3480, 3261-3815, 3269-3558, |
| | 3270-3566, 3274-3564, 3276-3508, 3279-3836, 3283-3838, 3285-3834, |
| | 3293-3627, 3311-3820, 3313-3852, 3315-3517, 3322-3819, 3330-3812, |
| | 3331-3600, 3331-3806, 3331-3819, 3331-3828, 3332-3504, 3333-3599, |
| | 3334-3580, 3338-3771, 3338-3843, 3341-3777, 3344-3826, 3347-3813, |
| | 3348-3819, 3353-3631, 3364-3827, 3371-3819, 3372-3817, 3372-3821, |
| | 3374-3819, 3376-3819, 3377-3823, |
| | 3380-3819, 3384-3760, 3385-3823, 3387-3727, 3391-3819, 3391-3833, |
| | 3392-3837, 3393-3812, 3395-3858, 3399-3819, 3401-3821, 3402-3813, |
| | 3402-3819, 3406-3825, 3407-3821, 3410-3825, 3410-3844, 3411-3825, |
| | 3411-3848, 3412-3812, 3412-3819, 3413-3819, 3415-3819, 3416-3819, |
| | 3417-3828, 3422-3819, 3426-3819, 3428-3822, 3430-3670, 3434-3654, |
| | 3434-3754, 3434-3782, 3434-3806, 3434-3815, 3436-3682, 3439-3722, |
| | 3447-3823, 3448-3819, 3453-3825, 3456-3699, 3460-3820, 3463-3819, |
| | 3463-3822, 3464-3738, 3468-3810, 3469-3819, 3472-3819, 3473-3823, |
| | 3475-3819, 3485-3738, 3492-3813, 3495-3819, 3496-3722, 3497-3766, |
| | 3499-3820, 3500-3819, 3501-3813, 3507-3721, 3507-3809, 3507-3810, |
| | 3507-3817, 3510-3796, 3516-3826, 3518-3619, 3518-3804, 3523-3800, |
| | 3528-3818, 3530-3809, 3534-3820, 3548-3785, 3570-3846, 3575-3820, |
| | 3600-3820, 3608-3819, 3610-3824, 3621-3849, 3623-3784, 3623-3822, |
| | 3623-3850, 3627-3716, 3640-3819, 3642-3820, 3656-3843, 3659-3803, |
| | 3676-3819, 3682-3813, 3697-3819, 3712-3819, 3714-3819, 3736-3813, |
| | 3736-3850, 3750-3819 |
| 71/7714715CB1/4992 | 1-714, 26-4978, 42-321, 42-341, 90-589, 504-1001, 528-1039, |
| | 650-1222, 791-1406, 803-1333, 1133-1425, 1134-1437, 1277-1726, |
| | 1304-1562, 1417-1956, 1575-1995, 1725-2219, 1845-2305, 1975-2600, |
| | 1982-2316, 2040-2676, 2052-2504, 2062-2310, 2106-2707, 2151-2566, |
| | 2234-2543, 2259-2424, 2297-2547, 2363-2950, 2384-2895, 2384-2896, |
| | 2418-2895, 2466-2779, 2466-2986, 2504-2696, 2505-3120, 2507-2807, |
| | 2552-2798, 2606-3083, 2608-3033, 2613-3255, 2670-2960, 2702-2880, |
| | 2723-3216, 2729-3008, 2729-3010, 2799-2859, 2802-3241, 2836-3094, |
| | 2866-3128, 2890-3107, 2890-3418, 2909-3210, 2938-3533, 2979-3354, |
| | 3039-3525, 3108-3757, 3161-3465, 3171-3448, 3174-3381, 3189-3319, |
| | 3202-3702, 3223-3804, 3225-3792, 3240-3507, 3264-3714, 3271-3469, |
| | 3322-3534, 3346-3629, 3350-3762, 3370-3612, 3370-3635, 3380-3787, |
| | 3412-3822, 3432-3717, 3442-3634, 3535-3765, 3536-3794, 3536-3846, |
| | 3542-4022, 3550-3814, 3560-4191, 3581-3873, 3584-4022, 3595-3792, |
| | 3609-3865, 3613-4059, |
| | 3614-3824, 3614-3885, 3626-3871, 3630-3924, 3632-3837, 3632-4159, |
| | 3669-4141, 3670-4330, 3675-3974, 3676-3936, 3676-4168, 3699-3728, |
| | 3706-3951, 3717-4194, 3737-3986, 3743-3983, 3772-4203, 3813-4028, |
| | 3828-4064, 3833-4103, 3852-4174, 3860-4090, 3874-4110, 3891-4120, |
| | 3917-4509, 3938-4207, 3938-4359, 3938-4511, 3940-4480, 3945-4167, |
| | 3953-4183, 3953-4496, 3973-4248, 3995-4201, 3995-4232, 3995-4250, |
| | 3996-4269, 4005-4258, 4007-4288, 4020-4478, 4025-4262, 4029-4275, |
| | 4030-4320, 4033-4238, 4033-4275, 4050-4309, 4053-4294, 4055-4316, |
| | 4062-4322, 4070-4344, 4070-4354, 4089-4248, 4094-4276, 4094-4622, |
| | 4139-4387, 4156-4387, 4168-4426, 4168-4594, 4177-4316, 4186-4476, |
| | 4188-4476, 4191-4417, 4243-4467, 4248-4460, 4265-4557, 4274-4545, |
| | 4274-4551, 4342-4543, 4361-4555, 4511-4969, 4514-4767, 4516-4992, |
| | 4542-4963, 4544-4968, 4548-4990, 4573-4962, 4580-4774, 4591-4855, |
| | 4598-4963, 4599-4968, 4619-4962, 4620-4962, 4622-4962, 4648-4962, |
| | 4649-4935, 4652-4963, 4660-4963, 4663-4965, 4677-4963, 4778-4963, |
| | 4817-4963 |
| 72/7506032CB1/791 | 1-240, 1-298, 1-790, 5-303, 8-295, 8-480, 8-498, 8-509, |
| | 8-601, 8-624, 8-781, 9-291, 13-243, 13-273, 13-363, 14-363, |
| | 19-237, 37-340, 129-360, 163-363, 235-781, 251-631, 253-781, |
| | 265-781, 319-779, 360-779, 363-774, 363-779, 363-781, 363-791, |
| | 369-772, 370-781, 376-781, 404-781, 428-504, 433-791, 435-694, |
| | 435-758, 437-781, 494-778, 494-781, 504-647, 514-781, 516-781, |
| | 517-711, 533-781, 549-775, 551-777, 564-778, 603-781, 606-681, |
| | 650-781 |
| 73/7506034CB1/2036 | 1-552, 8-705, 13-335, 13-454, 17-307, 17-656, 19-274, 21-275, |
| | 22-279, 25-322, 25-2036, 28-268, 28-524, 29-210, 30-282, |
| | 32-345, 33-299, 34-325, 35-290, 35-307, 35-426, 35-488, |
| | 36-821, 37-239, 37-257, 37-306, 37-625, 40-564, 42-579, |
| | 45-499, 45-614, 49-326, 49-605, 51-320, 51-654, 56-355, |
| | 56-430, 61-714, 62-542, 62-690, 65-318, 77-674, 88-512, |
| | 107-599, 128-431, 135-232, 161-772, 162-364, 173-804, 174-450, |
| | 176-606, 180-665, 187-676, 194-711, 201-820, 202-852, 215-644, |
| | 218-649, 218-758, 218-827, 218-838, 254-816, 254-913, 254-937, |
| | 254-1016, 259-915, 267-713, 269-713, 311-914, 314-574, |
| | 314-784, 337-618, 337-633, 351-620, 370-622, 372-616, 391-647, |
| | 399-656, 427-545, 469-664, 475-712, 505-919, 531-654, 710-964, |
| | 711-909, 711-974, 711-1117, 711-1169, 714-964, 746-964, |
| | 964-1161, 964-1210, 964-1516, |
| | 975-1596, 1003-1554, 1024-1253, 1038-1265, 1058-1512, 1061-1625, |
| | 1067-1635, 1073-1981, 1090-1340, 1090-1341, 1121-1360, 1130-1408, |
| | 1150-1980, 1151-1980, 1168-1980, 1171-1610, 1182-1980, 1184-1981, |
| | 1186-1456, 1187-1785, 1190-1472, 1195-1460, 1195-1787, 1202-1485, |
| | 1208-1394, 1209-1978, 1211-1606, 1226-1499, 1227-1980, 1247-1507, |
| | 1249-1468, 1249-1698, 1250-1538, 1259-1500, 1259-1506, 1260-1563, |
| | 1262-1408, 1276-1545, 1278-1604, 1279-1609, 1280-1529, 1281-1461, |
| | 1293-1978, 1295-1978, 1315-1957, 1351-1846, 1352-1455, 1354-1951, |
| | 1362-1398, 1372-2028, 1373-1621, 1373-1626, 1373-1628, 1373-1632, |
| | 1373-1654, 1374-1646, 1383-1957, 1393-1870, 1393-2006, 1394-1633, |
| | 1396-2016, 1399-1793, 1410-2016, 1411-1647, 1417-2018, 1420-1922, |
| | 1436-2036, 1447-1865, 1452-2012, 1453-1739, 1453-1856, 1453-1963, |
| | 1453-2027, 1454-1990, 1470-1600, 1470-2014, 1472-2036, |
| | 1486-1737, 1486-1779, 1500-1712, 1514-2034, 1515-1775, 1528-2036, |
| | 1540-2036, 1543-1991, 1547-2012, 1548-1818, 1548-1823, 1557-2033, |
| | 1560-2008, 1564-2036, 1566-2031, 1570-2024, 1570-2026, 1574-2027, |
| | 1574-2035, 1582-2029, 1585-2015, 1589-1841, 1589-2027, 1591-2028, |
| | 1595-1981, 1596-2029, 1600-1833, 1601-2029, 1606-1936, 1606-2036, |
| | 1608-2036, 1611-2025, 1613-1866, 1613-2031, 1614-2036, 1615-2026, |
| | 1619-2036, 1620-2028, 1620-2032, 1624-2023, 1626-2027, 1627-2028, |
| | 1629-2025, 1629-2027, 1629-2029, 1629-2036, 1630-2027, 1632-2025, |
| | 1633-2036, 1639-2030, 1647-2036, 1650-2027, 1653-2023, 1657-2025, |
| | 1658-2036, 1659-2027, 1660-2036, 1661-2024, 1664-2025, 1668-2027, |
| | 1677-1907, 1677-2019, 1690-1919, 1691-1960, 1693-2036, 1694-1933, |
| | 1702-2027, 1703-2033, 1706-2028, 1712-2028, 1713-2027, 1716-2025, |
| | 1719-2029, 1720-2025, 1749-2027, 1750-2031, 1753-2029, 1763-2027, |
| | 1781-2031, 1785-2027, 1789-2029, 1791-2025, 1795-2036, 1797-2027, |
| | 1805-2029, 1808-2029, 1843-2027, 1851-2024, 1940-2036, 1977-2027 |
| 74/7506100CB1/1100 | 1-669, 17-1088, 32-294, 77-705, 101-555, 153-573, 153-692, |
| | 153-839, 156-502, 156-545, 161-408, 182-633, 204-769, 205-638, |
| | 209-845, 224-624, 242-518, 335-909, 371-913, 385-1078, 387-886, |
| | 393-513, 397-1011, 399-641, 420-1034, 422-1052, 426-1077, |
| | 427-1034, 430-907, 432-959, 433-725, 433-847, 439-843, 443-682, |
| | 443-958, 461-1045, 461-1054, 485-1100, 491-1019, 636-894, |
| | 685-1074, 733-1100, 836-1088 |
| 75/1743113CB1/2043 | 1-272, 35-311, 35-330, 53-630, 55-324, 71-339, 75-306, 75-374, |
| | 75-651, 81-308, 81-313, 81-321, 81-360, 81-594, 83-343, |
| | 428-1646, 438-1082, 534-980, 575-881, 575-1037, 575-1077, |
| | 575-1120, 636-788, 663-1279, 679-924, 702-991, 736-957, |
| | 738-1264, 825-1499, 925-1112, 965-1197, 965-1243, 967-1164, |
| | 1024-1311, 1024-1324, 1053-1296, 1068-1712, 1085-1340, 1101-1710, |
| | 1111-1384, 1200-1649, 1200-1789, 1202-1462, 1236-1511, 1255-1666, |
| | 1278-1912, 1301-1893, 1382-1724, 1383-1566, 1394-1727, 1437-1681, |
| | 1445-1719, 1473-2000, 1490-1677, 1518-1709, 1531-2043, 1554-1709, |
| | 1554-1716, 1569-1724, 1569-1916, 1608-1953, 1638-1883, 1638-1908, |
| | 1652-1899, 1769-1979 |
| 76/7505144CB1/1128 | 1-1107, 46-264, 46-496, 57-446, 59-318, 59-673, 61-126, |
| | 61-299, 61-656, 62-314, 68-342, 72-310, 73-441, 73-506, |
| | 73-535, 73-675, 74-368, 74-675, 76-383, 101-961, 186-393, |
| | 186-568, 231-334, 268-705, 281-705, 289-658, 340-675, 369-582, |
| | 377-602, 377-643, 390-605, 409-675, 411-705, 420-675, 446-646, |
| | 447-657, 452-639, 458-664, 462-672, 466-675, 484-611, 484-675, |
| | 495-654, 502-675, 503-639, 611-1022, 713-1083, 737-1123, |
| | 745-1005, 745-1040, 745-1104, 745-1125, 745-1127, 745-1128, |
| | 746-1073, 748-1011, 754-1058, 754-1065, 754-1076, 754-1084, |
| | 757-1008, 763-959, 763-988, 763-1005, 763-1041, 763-1049, |
| | 763-1050, 763-1070, 763-1075, 763-1076, 763-1087, 767-1077, |
| | 768-1101, 788-917, 788-1013, 795-949, 795-1052, 795-1078, |
| | 795-1085, 799-1075, 803-1075, 822-1086, 829-1005, 830-1031, |
| | 830-1110, 833-1083, 836-1083, 840-1107, 844-1086, 846-1008, |
| | 862-1102, 873-1110, 874-1071, 874-1109, 875-1088, 881-1110, |
| | 893-1082, 898-1083 |
| 77/7506132CB1/871 | 1-579, 27-871, 28-305, 29-260, 39-218, 39-285, 39-317, |
| | 43-478, 43-512, 51-372, 51-491, 53-341, 53-557, 54-324, |
| | 55-341, 60-295, 110-275, 189-475, 252-545, 261-513, 288-513, |
| | 317-755, 318-772, 342-586, 556-840, 675-871 |
| 78/8142016CB1/2300 | 1-467, 34-467, 212-833, 214-426, 214-432, 214-472, 214-796, |
| | 214-1849, 420-988, 430-482, 445-482, 478-515, 478-520, 478-530, |
| | 481-530, 493-799, 493-1062, 554-838, 554-1173, 590-817, 626-1280, |
| | 671-984, 676-969, 710-1232, 716-974, 791-1339, 834-1148, |
| | 835-1023, 835-1429, 835-1435, 835-1444, 835-1459, 835-1473, |
| | 835-1480, 835-1630, 916-1240, 928-1205, 930-1466, 937-1426, |
| | 956-1227, 974-1469, 997-1598, 1072-1508, 1073-1537, 1074-1508, |
| | 1078-1552, 1083-1585, 1089-1760, 1125-1656, 1137-1770, 1150-1859, |
| | 1152-1952, 1160-1698, 1170-1835, 1171-1425, 1174-2018, 1182-1944, |
| | 1190-1435, 1192-1479, 1196-1887, 1206-1771, 1233-1719, 1233-1744, |
| | 1242-1919, 1258-1818, 1258-1867, 1264-1513, |
| | 1268-1520, 1270-1879, 1272-1528, 1289-1563, 1294-1611, 1294-1772, |
| | 1294-2061, 1294-2065, 1299-2025, 1314-2165, 1322-1565, 1344-1599, |
| | 1367-1611, 1380-1861, 1385-1993, 1388-1811, 1390-2222, 1391-1620, |
| | 1391-1699, 1391-1861, 1391-1978, 1391-2015, 1391-2035, 1391-2085, |
| | 1391-2096, 1391-2109, 1391-2123, 1391-2228, 1423-1671, 1429-2300, |
| | 1435-1712, 1461-1981, 1463-1658, 1463-2033, 1470-1736, 1470-1995, |
| | 1472-2066, 1481-1735, 1512-1788, 1525-1728, 1525-1745, 1525-1783, |
| | 1525-2078, 1526-1745, 1552-2112, 1556-1843, 1561-1838, 1572-1827, |
| | 1573-1745, 1580-2007, 1581-1745, 1587-1842, 1587-2078, 1594-1835, |
| | 1600-1840, 1604-1890, 1613-1806, 1642-1866 |
| 79/7506135CB1/2903 | 1-293, 11-188, 15-116, 17-469, 19-2882, 23-150, 23-236, |
| | 24-192, 24-211, 26-227, 45-227, 50-254, 56-225, 56-233, |
| | 61-239, 68-222, 74-228, 87-232, 302-914, 303-910, 389-661, |
| | 665-1279, 821-993, 929-1465, 932-1393, 942-1356, 992-1435, |
| | 1037-1647, 1037-1659, 1044-1655, 1056-1539, 1095-1649, 1095-1652, |
| | 1095-1710, 1109-1383, 1206-1764, 1229-1519, 1230-1605, 1257-1519, |
| | 1280-1786, 1291-1649, 1372-1616, 1378-1928, 1412-1665, 1429-1497, |
| | 1447-1659, 1459-1892, 1472-1649, 1531-1792, 1621-1937, 1723-1976, |
| | 1737-2420, 1738-1975, 1757-2414, 1779-2391, 1789-2035, 1789-2065, |
| | 1789-2407, 1789-2420, 1795-2384, 1804-2073, 1804-2305, 1831-2607, |
| | 1836-2455, 1841-2082, 1844-2061, 1845-2209, |
| | 1846-2444, 1856-2106, 1856-2409, 1857-2098, 1859-2156, 1860-2269, |
| | 1881-2607, 1888-2174, 1888-2412, 1890-2138, 1890-2153, 1891-2609, |
| | 1937-2186, 1943-2163, 1943-2546, 1953-2192, 1953-2203, 1953-2205, |
| | 1953-2233, 1962-2207, 2012-2264, 2020-2506, 2021-2252, 2025-2854, |
| | 2038-2296, 2038-2366, 2048-2314, 2053-2277, 2055-2621, 2067-2327, |
| | 2093-2235, 2093-2308, 2099-2374, 2099-2376, 2101-2819, 2122-2812, |
| | 2125-2823, 2142-2492, 2142-2709, 2142-2786, 2163-2817, 2179-2515, |
| | 2183-2631, 2190-2892, 2208-2784, 2216-2801, 2230-2664, 2248-2892, |
| | 2252-2879, 2253-2835, 2266-2523, 2272-2879, 2279-2859, 2280-2516, |
| | 2300-2572, 2312-2875, 2314-2874, 2315-2732, 2331-2618, 2334-2840, |
| | 2335-2874, 2337-2735, 2347-2541, 2360-2875, 2361-2829, 2361-2872, |
| | 2362-2636, 2380-2891, |
| | 2382-2882, 2404-2903, 2410-2696, 2416-2892, 2422-2704, 2422-2710, |
| | 2422-2723, 2422-2727, 2427-2903, 2429-2883, 2430-2700, 2435-2882, |
| | 2441-2891, 2441-2892, 2448-2883, 2456-2883, 2460-2883, 2460-2892, |
| | 2461-2903, 2462-2823, 2463-2883, 2465-2903, 2470-2708, 2470-2892, |
| | 2472-2880, 2472-2897, 2491-2853, 2499-2730, 2508-2891, 2512-2892, |
| | 2514-2883, 2515-2810, 2515-2892, 2524-2900, 2524-2903, 2529-2785, |
| | 2529-2867, 2529-2883, 2534-2896, 2540-2891, 2549-2897, 2550-2892, |
| | 2557-2903, 2596-2814, 2596-2879, 2596-2899, 2606-2903, 2612-2892, |
| | 2619-2892, 2619-2899, 2626-2888, 2629-2883, 2639-2890, 2643-2892, |
| | 2650-2892, 2656-2879, 2716-2892, 2717-2892, 2721-2892, 2733-2881, |
| | 2774-2891, 2810-2886, 2815-2891, 2815-2903 |
| 80/90086301CB1/1480 | 1-909, 1-1480, 669-1480 |
| 81/7487373CB1/1498 | 1-768, 560-1498 |
| 82/7506228CB1/1788 | 1-429, 48-429, 60-429, 99-338, 100-429, 104-429, 109-338, |
| | 113-425, 118-429, 119-429, 121-347, 128-1761, 133-417, 139-308, |
| | 171-336, 175-368, 185-780, 202-422, 206-886, 209-429, 212-429, |
| | 246-429, 267-429, 276-429, 426-640, 426-664, 426-687, 426-688, |
| | 426-694, 426-919, 426-920, 426-929, 427-564, 427-653, 427-656, |
| | 427-662, 427-664, 427-667, 427-686, 427-696, 427-717, 427-727, |
| | 427-743, 427-765, 427-778, 427-1026, 427-1089, 428-694, 430-710, |
| | 431-719, 434-890, 440-954, 442-737, 445-749, 446-709, 447-624, |
| | 447-702, 450-1087, 451-733, 455-704, 460-1003, 462-704, 464-701, |
| | 464-706, 464-718, 464-733, 464-734, 465-995, 466-890, 468-761, |
| | 468-1109, 468-1160, 471-979, 474-719, 477-760, 478-681, 481-723, |
| | 481-733, 482-1260, 491-767, 491-890, 492-1386, 493-752, 493-1216, |
| | 497-769, 502-798, 504-763, 509-727, 509-916, 510-769, 517-1103, |
| | 523-766, 523-779, 523-789, 523-995, 523-1119, 523-1124, |
| | 524-795, 529-636, 530-794, 530-820, 530-1128, 530-1174, 534-784, |
| | 534-1191, 539-807, 539-832, 539-898, 539-1150, 540-807, 541-1185, |
| | 545-1021, 547-789, 547-1064, 548-683, 548-774, 548-804, 550-809, |
| | 550-825, 553-748, 555-1021, 556-1168, 561-837, 562-894, 564-852, |
| | 564-1186, 570-753, 570-1269, 572-864, 572-895, 572-1125, 573-695, |
| | 573-737, 573-852, 573-921, 573-1108, 574-799, 574-838, 574-857, |
| | 574-899, 574-921, 574-938, 575-849, 576-817, 576-1132, 577-816, |
| | 577-1040, 578-814, 578-862, 583-843, 585-847, 585-1070, 589-1261, |
| | 590-822, 590-1021, 591-857, 591-1217, 595-884, 601-825, 601-864, |
| | 602-845, 602-869, 602-890, 603-880, 604-870, 604-877, 604-893, |
| | 609-728, 610-849, 610-865, 613-1160, 620-865, 620-1243, 620-1477, |
| | 622-1249, 623-1229, 626-904, 626-922, 626-1246, 628-893, 630-925, |
| | 631-859, 631-888, 632-1103, 634-768, 634-885, 642-893, |
| | 643-887, 643-908, 648-850, 649-889, 653-1270, 656-924, 659-935, |
| | 662-1242, 663-939, 668-899, 668-924, 671-872, 671-1142, 671-1156, |
| | 673-874, 673-924, |
| | 674-1247, 675-937, 676-1521, 682-1239, 684-1393, 689-1068, |
| | 690-1130, 691-757, 693-993, 693-1230, 694-994, 698-961, |
| | 701-934, 702-922, 702-943, 702-1323, 707-898, 707-1011, |
| | 707-1306, 707-1324, 711-959, 711-1298, 713-1016, 715-971, |
| | 715-1249, 715-1398, 719-1202, 720-976, 724-928, 729-954, |
| | 729-1136, 735-980, 735-983, 735-1411, 739-1202, 746-1027, |
| | 747-983, 749-1360, 752-1592, 753-1221, 753-1420, 757-1604, |
| | 758-1004, 758-1058, 760-1246, 761-964, 761-1008, 761-1360, |
| | 762-1320, 764-1050, 769-1230, 771-905, 771-1032, 772-1221, |
| | 774-1026, 776-1202, 782-1386, 784-1362, 789-1330, 792-1204, |
| | 795-1108, 795-1327, 800-1328, 805-1245, 806-1411, 806-1418, |
| | 809-1065, 811-1382, 812-1364, 812-1383, 812-1402, 812-1418, |
| | 814-1097, 827-1065, 827-1097, 827-1368, 829-1473, 829-1571, |
| | 830-1100, 833-1387, 846-1482, 846-1582, 846-1598, 848-1073, |
| | 849-1420, 851-1051, 853-1134, 853-1551, 854-1068, 854-1125, |
| | 854-1324, 855-1094, 855-1638, 856-1062, 856-1526, 857-1093, |
| | 861-1148, 866-1493, 867-1382, 867-1409, 873-1166, 873-1277, |
| | 875-1136, 875-1140, 876-1638, 878-1604, 881-1174, 881-1641, |
| | 883-1100, 883-1148, 883-1214, 884-1069, 884-1076, 884-1150, |
| | 886-981, 887-1171, 888-1010, 889-1596, 893-1593, 894-1122, |
| | 896-1171, 896-1560, 896-1640, 897-1382, 899-1163, 905-1591, |
| | 908-1183, 908-1208, 909-1153, 909-1164, 909-1587, 909-1600, |
| | 910-1182, 911-1149, 911-1170, 912-1472, 914-1122, 914-1359, |
| | 915-1217, 923-1592, 934-1580, 935-1214, 936-1474, 936-1591, |
| | 940-1569, 940-1643, 942-1200, 942-1591, 945-1594, 948-1289, |
| | 949-1191, 950-1555, 952-1257, 956-1275, 956-1535, 960-1174, |
| | 961-1378, 962-1638, 965-1642, 966-1230, 970-1688, 973-1276, |
| | 975-1226, 980-1231, 980-1234, 982-1385, 982-1591, 982-1594, |
| | 983-1228, 989-1239, 991-1606, 996-1220, 999-1243, 1000-1266, |
| | 1000-1588, 1002-1254, 1003-1312, 1003-1585, 1004-1170, 1004-1270, |
| | 1004-1289, 1004-1528, 1004-1541, 1004-1583, 1004-1593, 1004-1600, |
| | 1004-1601, 1004-1630, 1004-1641, 1005-1176, 1005-1231, 1005-1264, |
| | 1005-1274, 1005-1306, 1005-1334, 1005-1366, 1005-1491, 1005-1544, |
| | 1005-1589, 1005-1593, 1007-1345, 1008-1248, 1008-1560, 1011-1600, |
| | 1012-1602, 1017-1536, 1021-1594, 1023-1257, |
| | 1023-1604, 1025-1600, 1026-1120, 1026-1594, 1026-1644, 1028-1178, |
| | 1030-1644, 1031-1231, 1032-1526, 1033-1590, 1034-1635, 1037-1304, |
| | 1038-1516, 1038-1534, 1038-1628, 1039-1310, 1039-1385, 1041-1303, |
| | 1042-1289, 1044-1586, 1045-1647, 1048-1243, 1049-1336, 1051-1564, |
| | 1052-1608, 1058-1644, 1062-1301, 1068-1324, 1070-1644, 1079-1644, |
| | 1082-1305, 1082-1606, 1082-1651, 1088-1373, 1088-1644, 1089-1644, |
| | 1091-1536, 1103-1406, 1104-1338, 1104-1343, 1104-1650, 1105-1250, |
| | 1113-1435, 1114-1246, 1114-1247, 1118-1371, 1118-1644, 1119-1367, |
| | 1119-1643, 1119-1644, 1123-1568, 1125-1644, 1131-1363, 1132-1644, |
| | 1139-1572, 1141-1719, 1143-1379, 1146-1644, 1148-1353, 1155-1482, |
| | 1155-1513, 1159-1270, 1162-1363, 1163-1591, 1164-1427, 1164-1447, |
| | 1165-1471, 1167-1414, 1167-1644, 1168-1426, 1169-1404, 1173-1389, |
| | 1174-1642, 1174-1643, 1175-1644, 1176-1644, 1179-1635, 1184-1644, |
| | 1185-1628, 1186-1641, 1186-1644, 1187-1484, 1188-1642, 1190-1644, |
| | 1191-1644, 1193-1472, 1193-1641, 1196-1552, 1196-1644, 1199-1644, |
| | 1200-1635, 1204-1382, 1204-1552, 1205-1472, |
| | 1360-1643, 1360-1644, 1362-1620, 1363-1644, 1366-1644, 1367-1641, |
| | 1367-1643, 1368-1420, 1369-1556, 1369-1578, 1370-1645, 1374-1637, |
| | 1374-1644, 1374-1737, 1376-1639, 1376-1761, 1379-1644, 1381-1644, |
| | 1385-1737, 1386-1745, 1387-1570, 1387-1643, 1387-1644, 1388-1644, |
| | 1390-1644, 1391-1643, 1393-1644, 1396-1644, 1399-1644, 1400-1528, |
| | 1401-1643, 1404-1644, 1405-1741, 1408-1676, 1410-1644, 1411-1644, |
| | 1412-1639, 1412-1644, 1412-1650, 1413-1644, 1414-1644, 1419-1581, |
| | 1420-1644, 1421-1644, 1422-1644, 1423-1608, 1424-1644, 1425-1644, |
| | 1426-1758, 1428-1644, 1429-1638, 1430-1644, 1431-1644, 1433-1644, |
| | 1434-1643, 1434-1644, 1442-1577, 1445-1644, 1449-1586, 1449-1640, |
| | 1449-1641, 1451-1644, 1458-1644, 1463-1644, 1464-1761, 1469-1557, |
| | 1469-1644, 1476-1644, 1482-1644, 1488-1644, 1489-1645, 1497-1643, |
| | 1505-1643, 1506-1644, 1508-1644, 1510-1643, 1511-1644, 1512-1644, |
| | 1514-1745, 1532-1644, 1554-1643, 1554-1788, 1564-1657, 1567-1644, |
| | 1573-1644, 1577-1644, 1590-1644, 1597-165 |
| 83/7506194CB1/1386 | 1-644, 25-1386, 102-751, 109-748, 297-727, 739-1011, 1023-1152, |
| | 1023-1187, 1023-1228, 1023-1280, 1023-1353, 1023-1378, 1023-1385, |
| | 1146-1229, 1164-1366 |
| 84/7506434CB1/1885 | 1-675, 1-1885, 255-494, 255-1043, 284-499, 286-557, 391-835, |
| | 503-1402, 512-968, 527-784, 527-848, 557-1403, 564-1404, |
| | 571-1401, 602-1395, 603-1392, 604-1401, 644-1402, 649-1402, |
| | 667-1401, 673-1401, 700-1401, 752-1401, 773-1136, 983-1476, |
| | 993-1251, 1031-1386, 1052-1390, 1063-1390, 1064-1386, 1064-1390, |
| | 1078-1385, 1089-1404, 1164-1432, 1216-1390, 1224-1404, 1227-1416, |
| | 1255-1717, 1280-1390, 1380-1641, 1417-1853, 1418-1885 |
| 85/7490974CB1/1788 | 1-104, 1-342, 109-428, 109-438, 109-446, 190-428, 388-1209, |
| | 472-687, 486-1060, 725-1431, 955-1787, 1000-1787, 1018-1787, |
| | 1092-1787, 1093-1787, 1101-1573, 1143-1704, 1151-1788, 1252-1788, |
| | 1272-1783, 1318-1788, 1324-1785, 1332-1748, 1332-1788, 1333-1779, |
| | 1340-1780, 1340-1787, 1342-1785, 1351-1656, 1351-1672, 1414-1788, |
| | 1437-1779, 1442-1713, 1442-1788, 1463-1783 |
| 86/7506224CB1/2786 | 1-200, 2-200, 7-860, 8-2776, 24-782, 33-200, 201-609, |
| | 307-794, 324-747, 326-860, 395-971, 423-627, 472-1170, 529-785, |
| | 604-828, 611-652, 653-717, 656-902, 687-865, 735-1425, 795-1425, |
| | 803-1433, 819-1309, 819-1324, 822-1425, 825-1425, 829-1425, |
| | 842-1105, 842-1288, 842-1318, 842-1367, 842-1435, 842-1455, |
| | 842-1461, 858-1425, 863-1016, 863-1260, 863-1360, 863-1421, |
| | 863-1435, 863-1445, 866-1534, 868-1151, 883-1426, 884-1425, |
| | 905-1183, 905-1184, 910-1425, 926-1620, 947-1217, 950-1284, |
| | 955-1425, 956-1590, 957-1502, 961-1236, 998-1273, 1030-1639, |
| | 1048-1595, 1058-1508, 1070-1259, 1077-1371, 1079-1321, 1081-1306, |
| | 1127-1824, 1134-1425, 1148-1717, 1151-1425, 1159-1425, 1166-1433, |
| | 1169-1425, 1173-1425, 1175-1629, 1192-1460, 1194-1884, 1200-1884, |
| | 1201-1869, 1221-1845, |
| | 1225-1871, 1233-1863, 1242-1481, 1242-1745, 1244-1870, 1246-1825, |
| | 1255-1798, 1256-1370, 1256-1799, 1274-1509, 1276-1863, 1289-1872, |
| | 1295-1917, 1298-1917, 1323-1770, 1336-1919, 1345-2003, 1345-2049, |
| | 1347-2043, 1359-1937, 1364-1662, 1371-1959, 1382-1620, 1385-1908, |
| | 1392-1804, 1392-1833, 1393-1983, 1397-1988, 1403-1798, 1418-1686, |
| | 1418-1855, 1418-1884, 1423-1992, 1423-1995, 1425-2113, 1434-1637, |
| | 1434-1641, 1434-1674, 1434-1703, 1434-2015, 1434-2082, 1435-1955, |
| | 1458-1746, 1458-1773, 1465-1871, 1467-2120, 1468-2081, 1469-1727, |
| | 1469-1728, 1469-1734, 1475-1806, 1476-1742, 1488-1753, 1491-1792, |
| | 1499-2047, 1510-2159, 1514-1763, 1514-1764, 1514-1951, 1514-2091, |
| | 1518-1908, 1520-2076, 1520-2169, 1521-2178, 1526-1971, 1535-2151, |
| | 1535-2191, 1535-2211, 1563-2004, 1566-1819, 1586-2146, 1590-2230, |
| | 1591-2181, 1591-2209, 1597-2235, 1603-1853, 1613-1759, 1617-1875, |
| | 1623-2245, 1627-2161, 1628-2174, 1647-2300, 1650-1939, 1652-1904, |
| | 1653-2249, 1661-1849, 1667-2315, 1690-2316, 1692-1960, 1692-1963, |
| | 1696-2221, 1696-2271, 1697-2243, 1699-1967, |
| | 1702-2258, 1707-1924, 1713-2348, 1719-2307, 1720-1887, 1734-2249, |
| | 1748-2063, 1755-1983, 1769-2423, 1772-2206, 1772-2247, 1772-2298, |
| | 1772-2348, 1772-2356, 1776-2234, 1780-2323, 1783-1967, 1790-2068, |
| | 1793-2465, 1798-1966, 1799-2237, 1803-1995, 1803-2405, 1806-2135, |
| | 1815-2490, 1816-2488, 1819-2445, 1822-2085, 1822-2091, 1824-2233, |
| | 1825-2095, 1825-2125, 1826-2101, 1828-2277, 1834-2404, 1844-2527, |
| | 1845-2401, 1847-2244, 1849-2323, 1853-2265, 1855-2470, 1856-2108, |
| | 1856-2121, 1856-2421, 1859-2112, 1859-2115, 1859-2119, 1866-2422, |
| | 1866-2456, 1870-2499, 1877-2224, 1877-2352, 1877-2458, 1890-2160, |
| | 1890-2316, 1890-2476, 1892-2473, 1894-2371, 1895-2159, 1895-2160, |
| | 1900-2514, 1906-2077, 1924-2360, 1924-2435, 1928-2116, 1937-2198, |
| | 1939-2150, 1948-2563, 1965-2231, 1969-2076, 1970-2517, 1973-2528, |
| | 1986-2205, 1995-2528, 1998-2253, 2011-2523, 2026-2233, 2026-2323, |
| | 2035-2310, 2036-2708, 2064-2528, 2073-2762, 2074-2243, 2075-2576, |
| | 2087-2321, 2097-2242, 2100-2355, 2107-2528, 2111-2762, 2120-2712, |
| | 2125-2687, 2129-2346, 2134-2776, |
| | 2135-2528, 2139-2528, 2148-2446, 2152-2778, 2168-2786, 2169-2774, |
| | 2170-2778, 2171-2774, 2172-2431, 2188-2766, 2191-2619, 2191-2778, |
| | 2216-2435, 2217-2481, 2220-2527, 2240-2778, 2250-2778, 2258-2778, |
| | 2264-2526, 2280-2778, 2284-2539, 2290-2591, 2292-2778, 2300-2576, |
| | 2303-2778, 2304-2778, 2308-2778, 2309-2774, 2309-2778, 2312-2778, |
| | 2313-2778, 2319-2778, 2321-2778, 2322-2778, 2324-2777, 2325-2577, |
| | 2325-2778, 2328-2718, 2329-2771, 2331-2642, 2331-2778, 2332-2778, |
| | 2333-2778, 2343-2778, 2347-2778, 2350-2778, 2351-2617, 2351-2778, |
| | 2354-2778, 2356-2527, 2356-2528, 2359-2778, 2362-2651, 2363-2778, |
| | 2366-2778, 2369-2778, 2371-2778, 2380-2778, 2383-2778, 2384-2778, |
| | 2389-2778, 2390-2778, 2396-2668, 2399-2688, 2400-2778, 2401-2778, |
| | 2404-2778, 2408-2682, 2409-2778, 2415-2778, 2416-2778, 2421-2735, |
| | 2422-2778, 2426-2778, 2453-2778, 2467-2759, 2471-2778, 2473-2778, |
| | 2481-2685, 2483-2778, 2495-2778, 2500-2778, 2507-2778, 2526-2778, |
| | 2530-2778, 2546-2778, 2547-2778, 2558-2778, 2566-2778, 2602-2778, |
| | 2605-2778, 2623-2778, 2689-2777 |
| 87/7506280CB1/2020 | 1-2018, 85-347, 85-696, 184-278, 184-406, 184-420, 184-421, |
| | 184-450, 184-497, 184-864, 185-374, 185-734, 195-473, 196-786, |
| | 197-321, 198-443, 201-641, 201-664, 210-493, 211-456, 222-569, |
| | 223-670, 232-456, 232-470, 232-651, 234-491, 249-509, 249-523, |
| | 249-729, 249-840, 254-461, 256-531, 259-857, 266-512, 266-906, |
| | 269-734, 272-556, 273-525, 277-859, 281-865, 283-489, 293-619, |
| | 304-571, 312-741, 312-855, 314-580, 315-441, 315-585, 319-889, |
| | 329-587, 331-617, 336-908, 341-842, 342-569, 343-639, 343-807, |
| | 348-519, 355-884, 362-864, 364-886, 369-628, 373-638, 373-811, |
| | 376-510, 376-651, 376-677, 377-924, 378-629, 379-746, 392-618, |
| | 394-584, 401-881, 414-899, 416-675, 419-706, 420-698, 420-930, |
| | 420-983, 431-787, 431-1075, 435-661, 443-923, 444-606, 444-1027, |
| | 445-715, 451-690, 453-727, 456-867, 457-932, 458-932, 461-1328, |
| | 465-710, 470-725, 471-867, 475-756, 476-1059, 476-1121, 477-676, |
| | 477-869, |
| | 477-989, 478-864, 479-781, 488-703, 490-712, 490-726, 497-743, |
| | 502-757, 506-922, 514-1022, 517-797, 518-801, 518-1102, 526-698, |
| | 526-815, 529-811, 534-758, 534-802, 535-1020, 545-767, 545-840, |
| | 548-712, 549-788, 550-793, 550-806, 552-839, 557-922, 559-806, |
| | 566-1283, 567-955, 579-857, 583-922, 584-932, 587-844, 587-988, |
| | 588-849, 590-1052, 590-1284, 591-856, 591-869, 591-871, 595-922, |
| | 599-927, 606-869, 607-977, 609-883, 610-839, 614-1297, 616-922, |
| | 617-1199, 622-901, 623-890, 632-922, 633-899, 635-869, 636-879, |
| | 640-914, 642-1267, 645-888, 648-894, 648-971, 649-1217, 656-900, |
| | 663-953, 663-1282, 663-1297, 665-895, 674-933, 676-982, 683-936, |
| | 688-907, 688-916, 702-1259, 704-952, 707-902, 708-960, 716-972, |
| | 724-974, 725-973, 725-1324, 726-1165, 727-932, 738-903, 738-921, |
| | 740-1019, 741-918, 741-1287, 742-1024, 745-1109, 746-1009, |
| | 747-1036, 748-1020, 750-988, 752-1003, 753-1001, 754-1226, |
| | 756-1022, 756-1025, 756-1027, 759-881, 759-1021, 759-1035, |
| | 764-1006, 764-1044, 766-1391, 771-1023, 771-1032, 771-1317, |
| | 772-1193, 776-932, |
| | 779-982, 780-976, 804-1037, 808-1057, 808-1069, 808-1089, |
| | 809-1130, 812-1055, 813-1342, 827-1351, 830-1351, 832-1091, |
| | 837-1342, 837-1351, 842-1008, 842-1064, 843-1131, 848-1096, |
| | 862-1121, 866-1311, 869-1143, 870-1166, 871-1002, 871-1163, |
| | 872-1320, 873-1178, 873-1351, 875-1325, 876-1351, 879-1178, |
| | 881-1351, 884-1185, 884-1320, 884-1340, 886-1157, 886-1340, |
| | 890-1167, 897-1132, 899-1341, 901-1162, 903-1129, 908-1143, |
| | 908-1190, 913-1351, 917-1309, 918-1351, 922-1177, 922-1351, |
| | 923-1306, 923-1350, 927-1229, 928-1146, 928-1295, 929-1350, |
| | 936-1351, 937-1351, 938-1351, 940-1351, 941-1351, 945-1101, |
| | 949-1200, 949-1241, 951-1253, 959-1316, 962-1336, 962-1350, |
| | 966-1351, 967-1351, 973-1251, 973-1351, 974-1202, 974-1351, |
| | 976-1297, 979-1351, 981-1320, 992-1240, 992-1245, 995-1320, |
| | 1001-1189, 1002-1320, 1007-1351, 1011-1234, 1060-1291, 1067-1743, |
| | 1076-1363, 1086-1648, 1088-1328, 1106-1368, 1106-1374, 1111-1325, |
| | 1120-1321, 1127-2017, 1130-1351, 1136-1410, 1140-1813, 1156-1324, |
| | 1156-1410, 1162-1390, 1167-1717, 1193-1958, 1205-1811, 1235-1733, |
| | 1251-1962, 1259-1371, 1271-2006, 1283-1961, |
| | 1296-1875, 1306-1999, 1308-1620, 1308-1854, 1326-1987, 1335-1586, |
| | 1336-1608, 1338-1949, 1341-1773, 1345-1507, 1353-1570, 1353-1574, |
| | 1360-1608, 1361-1611, 1364-1578, 1364-1617, 1365-1837, 1367-1725, |
| | 1384-2018, 1389-1571, 1391-1602, 1397-1862, 1398-1673, 1409-1826, |
| | 1410-1652, 1410-1656, 1411-1749, 1415-2006, 1416-1679, 1417-1881, |
| | 1422-1646, 1428-1647, 1428-1685, 1429-1668, 1453-1743, 1453-2018, |
| | 1455-1554, 1459-1780, 1460-1758, 1464-1702, 1466-2005, 1469-2005, |
| | 1489-2020, 1491-1748, 1496-1733, 1497-1760, 1500-1749, 1500-1783, |
| | 1500-1801, 1501-1761, 1502-2018, 1503-2018, 1506-2019, 1507-1766, |
| | 1507-1790, 1522-1776, 1522-1783, 1522-2006, 1526-2000, 1527-1799, |
| | 1529-1747, 1533-1798, 1537-2020, 1547-2020, 1548-1791, 1549-1798, |
| | 1551-1759, 1551-1831, 1551-2020, 1553-2020, 1558-1814, 1559-2020, |
| | 1567-1836, 1571-1975, 1574-1787, 1574-1809, 1574-1823, 1574-1849, |
| | 1575-2005, 1576-1882, 1576-1980, 1576-2016, 1578-2019, 1584-2018, |
| | 1584-2020, 1586-1898, 1589-1857, 1590-2020, 1597-1869, |
| | 1599-2019, 1601-2018, 1602-2016, 1605-2018, |
| | 1606-2019, 1606-2020, 1607-2018, 1608-2018, 1609-1925, 1609-2018, |
| | 1610-2018, 1613-2018, 1613-2020, 1614-2018, 1615-2018, 1618-2020, |
| | 1619-1899, 1619-2017, 1619-2018, 1620-2018, 1621-1878, 1622-2020, |
| | 1623-2018, 1625-2018, 1626-2020, 1629-1884, 1629-2016, 1637-1934, |
| | 1638-2014, 1647-1911, 1647-1940, 1655-1914, 1658-1888, 1658-1935, |
| | 1658-1964, 1662-2020, 1671-1748, 1671-1948, 1675-1851, 1675-1890, |
| | 1675-1968, 1679-2018, 1680-2018, 1685-2018, 1686-2018, 1689-1967, |
| | 1689-1998, 1689-2016, 1690-2018, 1691-1896, 1692-2020, 1695-2016, |
| | 1700-1969, 1700-2018, 1700-2020, 1701-2020, 1703-2020, 1707-2018, |
| | 1708-2020, 1710-2016, 1712-1977, 1718-2018, 1722-2018, 1729-1983, |
| | 1730-1946, 1730-1987, 1730-1993, 1730-2016, 1731-1964, 1731-1980, |
| | 1731-2020, 1734-2011, 1737-1998, 1739-2018, 1746-2018, 1746-2020, |
| | 1749-2020, 1752-2020, 1759-1965, 1773-2018, 1774-2019, 1793-2020, |
| | 1815-2018, 1815-2019, 1819-2020, 1821-2020, 1825-2018, 1825-2020, |
| | 1831-2020, 1835-2018, 1839-2018, 1859-2020, 1881-2018, 1883-2016 |
| 88/7508326CB1/1487 | 1-261, 1-591, 1-1471, 147-872, 555-1101, 830-1487 |
| 89/7506370CB1/1120 | 1-286, 3-1120, 250-462, 250-810, 322-544, 322-555, 322-573, |
| | 322-579, 322-583, 322-996, 325-588, 327-946, 336-650, 336-714, |
| | 336-768, 336-776, 340-624, 351-614, 355-497, 355-588, 355-812, |
| | 356-597, 364-628, 368-645, 371-558, 374-853, 374-1106, 376-1088, |
| | 380-683, 381-640, 381-785, 382-898, 384-658, 386-680, 389-646, |
| | 392-1044, 394-627, 397-627, 397-898, 398-998, 399-646, 408-673, |
| | 409-900, 413-1006, 413-1017, 418-565, 418-692, 429-697, 437-679, |
| | 444-650, 444-666, 452-737, 454-752, 461-1036, 465-917, 470-662, |
| | 481-1065, 482-754, 482-997, 502-918, 502-1041, 506-736, 506-761, |
| | 510-996, 511-753, 512-944, 519-1040, 521-775, 535-829, 539-1120, |
| | 550-885, 551-1065, 556-1060, 564-839, 564-846, |
| | 565-1096, 565-1120, 566-1111, 569-1009, 571-862, 571-884, |
| | 571-1057, 573-1107, 577-804, 578-830, 579-1036, 588-1073, |
| | 599-815, 603-890, 603-1092, 605-1043, 605-1120, 609-1120, |
| | 613-1020, 617-908, 618-843, 619-1072, 629-897, 629-1063, |
| | 633-1111, 645-1110, 649-1120, 654-1110, 658-1112, 659-1120, |
| | 667-1021, 668-940, 668-974, 670-874, 670-1046, 673-1115, |
| | 677-1103, 678-1110, 678-1113, 679-1110, 680-1112, 681-1111, |
| | 683-933, 683-944, 683-970, 683-1096, 685-1074, 686-1107, |
| | 687-1111, 689-1038, 689-1111, 693-819, 694-1120, 696-945, |
| | 696-1049, 696-1120, 698-1109, 698-1111, 698-1116, 700-840, |
| | 700-1114, 700-1120, 701-1111, 701-1117, 702-1117, 703-1112, |
| | 703-1120, 704-1093, 706-1111, 710-1115, |
| | 711-1075, 712-1065, 712-1106, 713-900, 713-926, 713-1110, |
| | 713-1120, 714-1111, 715-1120, 716-1111, 720-1112, 721-1114, |
| | 722-1107, 723-1117, 725-1107, 727-1120, 729-1109, 731-1097, |
| | 731-1117, 732-1107, 732-1111, 733-1006, 735-968, 736-1111, |
| | 737-1111, 738-970, 739-1113, 740-1110, 741-989, 747-981, |
| | 747-994, 747-1070, 749-926, 750-1045, 753-1111, 754-1111, |
| | 756-1116, 756-1117, 760-1112, 762-1119, 772-1120, 778-1113, |
| | 781-1111, 781-1112, 785-1053, 785-1111, 785-1114, 786-1113, |
| | 787-1111, 790-1042, 794-1111, 797-1004, 797-1111, 798-1105, |
| | 799-1114, 801-1042, 803-1098, 810-1111, 812-1117, 813-1111, |
| | 815-1020, 815-1111, 816-1111, 820-1114, 822-1111, 823-1052, |
| | 823-1111, 823-1120, 824-1111, 836-1093, 836-1111, 838-1117, |
| | 842-1106, 845-1107, 847-1096, 847-1105, 852-1075, 852-1106, |
| | 852-1111, 852-1112, 852-1118, 852-1120, 858-1120, 871-1117, |
| | 873-1110, 880-1111, 880-1114, 885-1111, 888-1105, 891-1115, |
| | 891-1116, 897-1120, 899-1111, 905-1111, 912-1110, 935-1116, |
| | 950-1110, 952-1120, 981-1116, 985-1120 |
| 91/7501108CB1/1451 | 1-235, 1-286, 1-366, 1-436, 1-509, 1-538, 1-569, 1-615, |
| | 1-1415, 2-267, 2-460, 2-516, 2-521, 3-509, 4-246, 4-263, |
| | 4-271, 4-595, 9-303, 13-129, 13-146, 13-234, 13-259, 13-265, |
| | 13-270, 13-500, 13-558, 13-585, 13-591, 13-595, 13-615, |
| | 14-241, 14-290, 15-538, 15-539, 16-191, 16-206, 16-240, |
| | 16-293, 16-365, 16-509, 16-526, 16-536, 16-544, 16-555, |
| | 16-582, 17-287, 17-292, 17-301, 17-367, 17-375, 17-438, |
| | 17-449, 17-453, 17-468, 17-472, 17-474, 17-500, 17-509, |
| | 17-515, 17-544, 17-564, 17-566, 17-595, 18-254, 18-311, |
| | 20-241, 20-625, 20-726, 20-761, 21-224, 21-254, 21-595, |
| | 23-596, 24-761, 28-761, 29-482, 31-148, 31-300, 31-308, |
| | 32-239, 32-255, 32-262, 32-276, 34-611, 34-752, 43-304, |
| | 43-338, 45-686, 45-716, 49-761, 54-182, 56-722, 62-300, |
| | 71-774, 73-591, 80-763, 83-697, 102-585, 104-171, 113-372, |
| | 114-255, 114-440, 114-557, 114-582, 114-659, 114-679, 114-691, |
| | 114-697, 114-698, 114-711, 114-718, 114-738, 114-746, 123-370, |
| | 130-370, 132-702, 139-423, 166-406, 174-416, 182-411, 187-470, |
| | 188-747, 188-781, 190-755, 191-673, 194-435, 219-447, 222-442, |
| | 226-459, |
| | 243-727, 251-496, 261-805, 266-534, 277-798, 283-516, 285-523, |
| | 287-532, 298-826, 303-790, 314-746, 314-780, 354-578, 355-753, |
| | 356-614, 377-631, 377-657, 393-825, 406-645, 406-669, 406-675, |
| | 406-795, 415-660, 419-645, 432-825, 436-716, 451-750, 453-730, |
| | 468-583, 471-721, 502-707, 509-775, 510-755, 513-816, 517-676, |
| | 518-771, 543-825, 548-790, 559-811, 565-812, 569-825, 617-811, |
| | 773-985, 823-951, 823-1027, 823-1047, 823-1049, 823-1059, |
| | 823-1066, 823-1072, 823-1077, 823-1174, 823-1201, 823-1217, |
| | 823-1320, 823-1322, 823-1332, 823-1337, 823-1377, 823-1403, |
| | 823-1415, 824-1329, 824-1415, 825-1445, 826-1393, 827-1439, |
| | 828-1064, 830-1075, 830-1123, 830-1443, 833-1062, 843-1056, |
| | 848-1403, 859-1062, 859-1129, 867-1413, 869-1448, 880-1077, |
| | 880-1429, 892-1141, 892-1178, 892-1384, 894-985, 894-1091, |
| | 902-1379, 912-1095, 912-1389, 913-1171, 923-1413, 936-1140, |
| | 948-1154, 969-1389, 975-1242, 982-1239, 986-1278, 989-1231, |
| | 1048-1320, 1048-1328, 1073-1301, 1073-1450, 1073-1451, |
| | 1074-1431, 1080-1336, 1089-1376, 1112-1317, 1113-1397, 1126-1377, |
| | 1127-1260, 1146-1359, 1147-1336, 1165-1431, 1174-1405, 1227-1428, |
| | 1247-1400, 1248-1397 |
| 92/7507581CB1/1488 | 1-150, 1-311, 119-707, 123-1488, 144-823, 257-507, 257-674, |
| | 259-1076, 287-532, 322-681, 330-629, 334-784, 343-918, 361-904, |
| | 368-601, 371-917, 405-1035, 432-722, 446-696, 447-672, |
| | 447-1038, 448-1181, 455-686, 467-1101, 480-687, 497-1048, |
| | 501-1204, 502-1048, 526-910, 541-950, 552-846, 570-837, 571-819, |
| | 582-850, 617-1171, 631-838, 633-1324, 640-1174, 662-1431, |
| | 728-972, 744-1151, 752-1048, 754-1016, 803-1487, 811-1469, |
| | 832-1101, 849-1050, 849-1151, 860-1438, 866-1134, 891-1170, |
| | 891-1374, 910-1464, 913-1129, 937-1474, 947-1488, 955-1436, |
| | 962-1359, 962-1410, 973-1332, 985-1465, 1000-1488, 1007-1250, |
| | 1016-1244, 1026-1415, 1027-1389, 1029-1364, 1030-1486, 1045-1326, |
| | 1047-1475, 1051-1393, 1067-1448, 1074-1422, 1081-1475, 1086-1475, |
| | 1097-1475, 1107-1475, 1117-1475, 1132-1403, 1176-1475, 1186-1475, |
| | 1201-1432, 1201-1437, 1201-1466, 1201-1468, 1201-1477, 1207-1475, |
| | 1258-1487, 1266-1475, 1276-1474, 1280-1475, 1280-1479 |
| 93/7506361CB1/1875 | 1-1875, 603-629, 708-1253, 708-1313, 717-1015, 720-1228, |
| | 720-1301, 739-1413, 757-1413, 780-1087, 803-1046, 823-1145, |
| | 823-1272, 823-1294, 823-1326, 823-1396, 825-1107, 825-1410, |
| | 833-1077, 833-1299, 833-1359, 847-1274, 865-1155, 866-1098, |
| | 913-1200, 922-1309, 927-1184, 938-1413, 944-1087, 948-1247, |
| | 971-1287, 971-1324, 978-1222, 980-1114, 984-1258, 986-1092, |
| | 986-1249, 1005-1262, 1006-1257, 1011-1321, 1011-1327, 1011-1328, |
| | 1011-1331, 1018-1327, 1020-1278, 1024-1313, 1037-1279, 1039-1290, |
| | 1050-1272, 1050-1324, 1051-1413, 1052-1309, 1052-1326, 1055-1294, |
| | 1094-1352, 1130-1356, 1130-1377, 1145-1406, 1145-1413, 1157-1391, |
| | 1161-1394, 1164-1393, 1189-1413, 1192-1534, |
| | 1313-1589, 1411-1875, 1412-1621, 1412-1631, 1412-1643, 1412-1645, |
| | 1412-1647, 1412-1650, 1412-1655, 1412-1667, 1412-1677, 1412-1852, |
| | 1412-1869, 1414-1875, 1416-1865, 1437-1868, 1438-1868, 1441-1688, |
| | 1441-1703, 1441-1869, 1442-1869, 1443-1868, 1443-1869, 1459-1873, |
| | 1462-1726, 1463-1869, 1464-1868, 1466-1869, 1468-1868, 1472-1868, |
| | 1473-1865, 1480-1875, 1483-1710, 1485-1859, 1491-1868, 1497-1648, |
| | 1498-1875, 1503-1726, 1503-1857, 1503-1869, 1510-1869, 1511-1738, |
| | 1513-1823, 1513-1868, 1520-1868, 1523-1869, 1531-1868, 1536-1875, |
| | 1566-1797, 1572-1868, 1575-1869, 1593-1869, 1597-1868, 1598-1868, |
| | 1608-1844, 1612-1869, 1617-1813, 1617-1848, 1617-1874, 1622-1869, |
| | 1636-1875, 1655-1875, 1664-1868, 1686-1869, 1703-1869, 1711-1868, |
| | 1714-1873, 1718-1869, 1725-1875, 1738-1875, 1742-1865, 1743-1868, |
| | 1758-1863, 1798-1875 |
| 94/7509211CB1/3153 | 1-229, 17-468, 19-3115, 24-211, 26-226, 45-227, 50-254, |
| | 56-225, 61-245, 68-222, 74-228, 87-232, 127-820, 157-302, |
| | 290-815, 296-820, 303-820, 388-660, 427-820, 515-1186, 530-813, |
| | 530-820, 902-1591, 1159-1695, 1162-1623, 1190-1565, 2279-3072, |
| | 2298-3068, 2385-3073, 2396-3050, 2404-2954, 2449-3034, 2450-2899, |
| | 2466-3105, 2467-2986, 2469-3116, 2481-3130, 2486-3068, 2548-2965, |
| | 2553-3082, 2567-3073, 2570-2968, 2635-3113, 2637-3143, 2642-3071, |
| | 2649-3131, 2654-3104, 2655-2937, 2655-2943, 2655-2956, 2662-3116, |
| | 2664-3116, 2667-3147, 2678-3147, 2693-3127, 2698-3146, 2703-2941, |
| | 2703-3125, 2703-3129, 2705-3125, 2724-3086, 2731-3078, 2732-2963, |
| | 2741-3124, 2745-3126, 2747-3116, 2748-3127, 2756-3085, 2757-3133, |
| | 2762-3018, 2763-3078, 2767-3125, 2773-3124, 2782-3125, 2783-3127, |
| | 2790-3153, 2829-3047, 2829-3118, 2852-3127, 2852-3132, 2859-3121, |
| | 2883-3126, 2893-3122, 2921-3129 |

TABLE 5

| Polynucleotide SEQ | | |
|---|---|---|
| ID NO: | Incyte Project ID: | Representative Library |
| 48 | 3356677CB1 | SINTTMR02 |
| 49 | 7481665CB1 | BRAINOR03 |
| 50 | 3563859CB1 | HEARFET02 |
| 51 | 2588884CB1 | LUNGNOT22 |
| 53 | 7503424CB1 | PGANNOT03 |
| 54 | 7503571CB1 | COLNNOT19 |
| 55 | 7505722CB1 | BONEUNR01 |
| 56 | 7505798CB1 | MIXDTXE01 |
| 57 | 7505847CB1 | HNT2AGT01 |
| 58 | 7505862CB1 | MUSCNOT02 |
| 59 | 7762537CB1 | MENITUT03 |
| 60 | 90033462CB1 | ADRETUE02 |
| 61 | 1644869CB1 | THYRDIE01 |
| 62 | 6288712CB1 | FIBPFEA01 |
| 63 | 71830156CB1 | TESTNOT17 |
| 64 | 7505044CB1 | THYRNOT10 |
| 65 | 7505086CB1 | CONNNOT01 |
| 66 | 7505784CB1 | BRSTTUT03 |
| 67 | 7505813CB1 | PANCTUT02 |
| 68 | 7505873CB1 | KIDETXF04 |
| 69 | 7505881CB1 | SPLNNOT04 |
| 70 | 7503510CB1 | BRSTNOT01 |
| 71 | 7714715CB1 | UTRSTME01 |
| 72 | 7506032CB1 | LATRTUT02 |
| 73 | 7506034CB1 | LIVRNON08 |
| 74 | 7506100CB1 | SKIRNOR01 |
| 75 | 1743113CB1 | PROSTUT12 |
| 76 | 7505144CB1 | LUNGDIS03 |
| 77 | 7506132CB1 | UTRSTDT01 |
| 78 | 8142016CB1 | MIXDTME01 |
| 79 | 7506135CB1 | NERDTDN03 |
| 82 | 7506228CB1 | BRAHNON05 |
| 83 | 7506194CB1 | BRSTTUT08 |
| 84 | 7506434CB1 | PITUNOT01 |
| 85 | 7490974CB1 | LIVRTUN04 |
| 86 | 7506224CB1 | EPIPUNA01 |
| 87 | 7506280CB1 | EOSITXT01 |
| 88 | 7508326CB1 | BRAINOT11 |
| 89 | 7506370CB1 | OVARDIN02 |
| 91 | 7501108CB1 | PROSTUT08 |
| 92 | 7507581CB1 | LUNGAST01 |
| 93 | 7506361CB1 | PKINDNV08 |
| 94 | 7509211CB1 | FIBRTXS07 |

TABLE 6

| Library | Vector | Library Description |
|---|---|---|
| ADRETUE02 | PCDNA2.1 | This 5′ biased random primed library was constructed using |
| | | RNA isolated from right adrenal tumor tissue removed from a 49- |
| | | year-old Caucasian male during unilateral adrenalectomy. |
| | | Pathology indicated adrenal cortical carcinoma comprising nearly |
| | | the entire specimen. The tumor was attached to the adrenal gland |
| | | which showed mild cortical atrophy. The tumor was encapsulated, |
| | | being surrounded by a thin (1-3 mm) rim of connective tissue. |
| | | The patient presented with adrenal cancer, abdominal pain, pyrexia |
| | | of unknown origin, and deficiency anemia. Patient history |
| | | included benign hypertension. Previous surgeries included |
| | | adenotonsillectomy. Patient medications included aspirin, calcium, |
| | | and iron. Family history included atherosclerotic coronary artery |
| | | disease in the mother; cerebrovascular accident and |
| | | atherosclerotic coronary artery disease in the father; and |
| | | benign hypertension in the grandparent(s). |
| BONEUNR01 | PCDNA2.1 | This random primed library was constructed using pooled cDNA |
| | | from two different donors. cDNA was generated using mRNA isolated |
| | | from an untreated MG-63 cell line derived from an osteosarcoma |
| | | tumor removed from a 14-year-old Caucasian male (donor A) and |
| | | using mRNA isolated from sacral bone tumor tissue removed from an |
| | | 18-year-old Caucasian female (donor B) during an exploratory |
| | | laparotomy and soft tissue excision. Pathology indicated giant |
| | | cell tumor of the sacrum in donor B. Donor B's history included |
| | | pelvic joint pain, constipation, urinary incontinence, |
| | | unspecified abdominal/pelvic symptoms, and a pelvic soft tissue |
| | | malignant neoplasm. Family history included prostate cancer in |
| | | donor B. |
| BRAHNON05 | pINCY | This normalized hippocampus tissue library was constructed from |
| | | 1.6 million independent clones from a hippocampus tissue library. |
| | | Starting RNA was made from posterior hippocampus removed from a |
| | | 35-year-old Caucasian male who died from cardiac failure. |
| | | Pathology indicated moderate leptomeningeal fibrosis and multiple |
| | | microinfarctions of the cerebral neocortex. The cerebral hemisphere |
| | | revealed moderate fibrosis of the leptomeninges with focal |
| | | calcifications. There was evidence of shrunken and slightly |
| | | eosinophilic pyramidal neurons throughout the cerebral hemispheres. |
| | | There were small microscopic areas of cavitation with gliosis, |
| | | scattered through the cerebral cortex. Patient history included |
| | | cardiomyopathy, CHF, cardiomegaly, an enlarged spleen and liver. |
| | | Patient medications included simethicone, Lasix, Digoxin, Colace, |
| | | Zantac, captopril, and Vasotec. The library was normalized in |
| | | two rounds using conditions adapted from Soares et al., PNAS |
| | | (1994) 91: 9228 and Bonaldo et al., Genome Research 6 (1996): 791, |
| | | except that a significantly longer (48 hours/round) reannealing |
| | | hybridization was used. |
| BRAINOR03 | PBK-CMV | This random primed library was constructed using pooled cDNA |
| | | from two donors. cDNA was generated using mRNA isolated from |
| | | brain tissue removed from a Caucasian male fetus (donor A) who was |
| | | stillborn with a hypoplastic left heart at 23 weeks' gestation and |
| | | from brain tissue removed from a Caucasian male fetus (donor B), |
| | | who died at 23 weeks' gestation from premature birth. Serologies |
| | | were negative for both donors and family history for donor B |
| | | included diabetes in the mother. |
| BRAINOT11 | pINCY | Library was constructed using RNA isolated from brain tissue |
| | | removed from the right temporal lobe of a 5-year-old Caucasian |
| | | male during a hemispherectomy. Pathology indicated extensive |
| | | polymicrogyria and mild to moderate gliosis (predominantly |
| | | subpial and subcortical), consistent with chronic seizure |
| | | disorder. Family history included a cervical neoplasm. |
| BRSTNOT01 | PBLUESCRIPT | Library was constructed using RNA isolated from the breast |
| | | tissue of a 56-year-old Caucasian female who died in a motor |
| | | vehicle accident. |
| BRSTTUT03 | PSPORT1 | Library was constructed using RNA isolated from breast tumor |
| | | tissue removed from a 58-year-old Caucasian female during a |
| | | unilateral extended simple mastectomy. Pathology indicated |
| | | multicentric invasive grade 4 lobular carcinoma. The mass was |
| | | identified in the upper outer quadrant, and three separate |
| | | nodules were found in the lower outer quadrant of the left |
| | | breast. Patient history included skin cancer, rheumatic heart |
| | | disease, osteoarthritis, and tuberculosis. Family history |
| | | included cerebrovascular disease, coronary artery aneurysm, |
| | | breast cancer, prostate cancer, atherosclerotic coronary artery |
| | | disease, and type I diabetes. |
| BRSTTUT08 | pINCY | Library was constructed using RNA isolated from breast tumor |
| | | tissue removed from a 45-year-old Caucasian female during |
| | | unilateral extended simple mastectomy. Pathology indicated |
| | | invasive nuclear grade 2-3 adenocarcinoma, ductal type, with |
| | | 3 of 23 lymph nodes positive for metastatic disease. Greater than |
| | | 50% of the tumor volume was in situ, both comedo and non-comedo |
| | | types. Immunostains were positive for estrogen/progesterone |
| | | receptors, and uninvolved tissue showed proliferative changes. |
| | | The patient concurrently underwent a total abdominal hysterectomy. |
| | | Patient history included valvuloplasty of mitral valve without |
| | | replacement, rheumatic mitral insufficiency, and rheumatic heart |
| | | disease. Family history included acute myocardial infarction, |
| | | atherosclerotic coronary artery disease, and type II diabetes. |
| COLNNOT19 | pINCY | Library was constructed using RNA isolated from the cecal tissue |
| | | of an 18-year-old Caucasian female. The cecal tissue, along |
| | | with the appendix and ileum tissue, were removed during bowel |
| | | anastomosis. Pathology indicated Crohn's disease of the ileum |
| | | involving 15 cm of the small bowel. |
| CONNNOT01 | pINCY | Library was constructed using RNA isolated from mesentery fat |
| | | tissue obtained from a 71-year-old Caucasian male during a |
| | | partial colectomy and permanent colostomy. Family history |
| | | included atherosclerotic coronary artery disease, myocardial |
| | | infarction, and extrinsic asthma. |
| EOSITXT01 | pINCY | Library was constructed using RNA isolated from eosinophils |
| | | stimulated with IL-5. |
| EPIPUNA01 | PSPORT1 | Library was constructed using RNA isolated from untreated |
| | | prostatic epithelial cell tissue removed from a 17-year-old |
| | | Hispanic male. Serologies were negative. |
| FIBPFEA01 | PSPORT1 | This amplified library was constructed using RNA isolated from |
| | | untreated fibroblasts of the prostate stroma removed from a |
| | | male fetus (Clonetics, Sample #CC-2508) who died after 26 |
| | | weeks' gestation. |
| FIBRTXS07 | pINCY | This subtracted library was constructed using 1.3 million clones |
| | | from a dermal fibroblast library and was subjected to two |
| | | rounds of subtraction hybridization with 2.8 million clones from |
| | | an untreated dermal fibroblast tissue library. The starting |
| | | library for subtraction was constructed using RNA isolated from |
| | | treated dermal fibroblast tissue removed from the breast of a |
| | | 31-year-old Caucasian female. The cells were treated with 9CIS |
| | | retinoic acid. The hybridization probe for subtraction was |
| | | derived from a similarly constructed library from RNA isolated |
| | | from untreated dermal fibroblast tissue from the same donor. |
| | | Subtractive hybridization conditions were based on the |
| | | methodologies of Swaroop et al., NAR (1991) 19: 1954 and Bonaldo, |
| | | et al., Genome Research (1996) 6: 791. |
| HEARFET02 | pINCY | Library was constructed using RNA isolated from heart tissue |
| | | removed from a Caucasian male fetus, who was stillborn with a |
| | | hypoplastic left heart and died at 23 weeks' gestation. |
| HNT2AGT01 | PBLUESCRIPT | Library was constructed at Stratagene (STR937233), using RNA |
| | | isolated from the hNT2 cell line derived from a human |
| | | teratocarcinoma that exhibited properties characteristic of a |
| | | committed neuronal precursor. Cells were treated with retinoic |
| | | acid for 5 weeks and with mitotic inhibitors for two weeks and |
| | | allowed to mature for an additional 4 weeks in conditioned |
| | | medium. |
| KIDETXF04 | PCMV-ICIS | Library was constructed using RNA isolated from a treated, |
| | | transformed embryonal cell line (293-EBNA) derived from kidney |
| | | epithelial tissue. The cells were treated with 5-aza-2′- |
| | | deoxycytidine (5AZA) for 72 hours and Trichostatin A for 24 hours |
| | | and transformed with adenovirus 5 DNA. |
| LATRTUT02 | pINCY | Library was constructed using RNA isolated from a myxoma removed |
| | | from the left atrium of a 43-year-old Caucasian male |
| | | during annuloplasty. Pathology indicated atrial myxoma. Patient |
| | | history included pulmonary insufficiency, acute myocardial |
| | | infarction, atherosclerotic coronary artery disease, |
| | | hyperlipidemia, and tobacco use. Family history included benign |
| | | hypertension, acute myocardial infarction, atherosclerotic |
| | | coronary artery disease, and type II diabetes. |
| LIVRNON08 | pINCY | This normalized library was constructed from 5.7 million |
| | | independent clones from a pooled liver tissue library. Starting |
| | | RNA was made from pooled liver tissue removed from a 4-year-old |
| | | Hispanic male who died from anoxia and a 16 week female fetus |
| | | who died after 16-weeks gestation from anencephaly. Serologies |
| | | were positive for cytolomegalovirus in the 4-year-old. Patient |
| | | history included asthma in the 4-year-old. Family history |
| | | included taking daily prenatal vitamins and mitral valve |
| | | prolapse in the mother of the fetus. The library was normalized |
| | | in 2 rounds using conditions adapted from Soares et al., PNAS |
| | | (1994) 91: 9228 and Bonaldo et al., Genome Research 6 (1996): 791, |
| | | except that a significantly longer (48 hours/round) reannealing |
| | | hybridization was used. |
| LIVRTUN04 | pINCY | This normalized liver tumor cell line library was constructed |
| | | from 1.72 million independent clones from a hepatocyte library. |
| | | Starting RNA was isolated from an untreated C3A hepatocyte cell |
| | | line, which is a derivative of a hepatoblastoma removed |
| | | from a 15-year-old Caucasian male. The library was normalized in |
| | | two rounds using conditions adapted from Soares et al., |
| | | PNAS (1994) 91: 9228-9232 and Bonaldo et al., Genome Research |
| | | (1996) 6: 791, (except that a significantly longer (48 - |
| | | hours/round) reannealing hybridization was used. |
| LUNGAST01 | PSPORT1 | Library was constructed using RNA isolated from the lung tissue |
| | | of a 17-year-old Caucasian male, who died from head trauma. |
| | | Patient history included asthma. |
| LUNGDIS03 | pINCY | Library was constructed using diseased lung tissue. 0.76 million |
| | | clones from a diseased lung tissue library were subjected to |
| | | two rounds of subtraction hybridization with 5.1 million clones |
| | | from a normal lung tissue library. The starting library for |
| | | subtraction was constructed using polyA RNA isolated from |
| | | diseased lung tissue. Patient history included idiopathic |
| | | pulmonary disease. Subtractive hybridization conditions were |
| | | based on the methodologies of Swaroop et al. (1991) Nucleic |
| | | Acids Res. 19: 1954; and Bonaldo et al. Genome Res. (1996) |
| | | 6: 791. |
| LUNGNOT22 | pINCY | Library was constructed using RNA isolated from lung tissue |
| | | removed from a 58-year-old Caucasian female. The tissue sample |
| | | used to construct this library was found to have tumor |
| | | contaminant upon microscopic examination. Pathology for the |
| | | associated tumor tissue indicated a caseating granuloma. Family |
| | | history included congestive heart failure, breast cancer, |
| | | secondary bone cancer, acute myocardial infarction and |
| | | atherosclerotic coronary artery disease. |
| MENITUT03 | pINCY | Library was constructed using RNA isolated from brain meningioma |
| | | tissue removed from a 35-year-old Caucasian female |
| | | during excision of a cerebral meningeal lesion. Pathology |
| | | indicated a benign neoplasm in the right cerebellopontine angle |
| | | of the brain. Patient history included hypothyroidism. Family |
| | | history included myocardial infarction and breast cancer. |
| MIXDTME01 | PBK-CMV | This 5′ biased random primed library was constructed using |
| | | pooled cDNA from five donors. cDNA was generated using |
| | | mRNA isolated from small intestine tissue removed from a |
| | | Caucasian male fetus (donor A), who died at 23 weeks' gestation |
| | | from premature birth; from colon epithelium tissue removed from |
| | | a 13-year-old Caucasian female (donor B) who died from a |
| | | motor vehicle accident; from diseased gallbladder tissue removed |
| | | from a 58-year-old Caucasian female (donor C) during |
| | | cholecystectomy and partial parathyroidectomy; from stomach |
| | | tissue removed from a 68-year-old Caucasian female (donor D) |
| | | during a partial gastrectomy; and from breast skin removed from |
| | | a 71-year-old Caucasian female (donor E) during a unilateral |
| | | extended simple mastectomy. For donor C, pathology indicated |
| | | chronic cholecystitis and cholelithiasis. The patient presented |
| | | with abdominal pain and benign parathyroid neoplasm. Patient |
| | | medications included Capoten, Catapres, Norvasc, Synthroid, |
| | | and Xanax. For donor D, pathology indicated the uninvolved |
| | | stomach tissue showed mild chronic gastritis. Patient medications |
| | | included Prilosec, zidoxin, Metamucil, calcium, and vitamins. |
| | | Donor E presented with malignant breast neoplasm and induratio |
| MIXDTXE01 | PBK-CMV | This 5′ biased random primed library was constructed using |
| | | pooled cDNA from nine donors. cDNA was generated using |
| | | mRNA isolated from Jurkat cell line derived from the T cells of |
| | | a male (donor A), THP-1 cell line derived from the peripheral |
| | | blood of a 1-year-old male (donor B), Daudi cell line derived |
| | | from B-lymphoblasts from a 16-year-old black male (donor C), |
| | | RPMI-1666 cell line derived from lymphoma tissue from a 29-year- |
| | | old Caucasian male (donor D), spleen from a 1-year-old |
| | | Caucasian male (donor E), thymus removed from a 21-year-old |
| | | Caucasian male (donor F) during a thymectomy, lymph node |
| | | from a 42-year-old Caucasian female (donor G), thymus tumor from |
| | | a 56-year-old Caucasian female |
| | | (donor H) during a total thymectomy and PBMC's from a pool of |
| | | donors (donor I). The patients presented with anemia and |
| | | persistent hyperplastic thymus (H). Patient history included |
| | | acute T-cell leukemia (A); acute monocytic leukemia (B); Burkitt's |
| | | lymphoma (C); Hodgkin's disease (D); Bronchitis (E); hydrocele, |
| | | regional enteritis or the small intestine, atopic dermatitis and |
| | | benign neoplasm of the parathyroid (F); heart murmur and cardiac |
| | | arrest (G); and cardiac dysrhythmia and left bundle branch |
| | | block (H). Previous surgeries included an appendectomy and |
| | | parathyroid surgery (F); unspecified heart surgery (G); and a |
| | | normal delivery (H). Family history included benign hypertension |
| | | in the grandparent(s) and coronary artery disease in the |
| | | father of donor F. |
| MUSCNOT02 | PSPORT1 | Library was constructed using RNA isolated from the psoas muscle |
| | | tissue of a 12-year-old Caucasian male. |
| NERDTDN03 | pINCY | This normalized dorsal root ganglion tissue library was |
| | | constructed from 1.05 million independent clones from a dorsal |
| | | root ganglion tissue library. Starting RNA was made from dorsal |
| | | root ganglion tissue removed from the cervical spine of a 32-year- |
| | | old Caucasian male who died from acute pulmonary edema, acute |
| | | bronchopneumonia, bilateral pleural effusions, pericardial |
| | | effusion, and malignant lymphoma (natural killer cell type). The |
| | | patient presented with pyrexia of unknown origin, malaise, |
| | | fatigue, and gastrointestinal bleeding. Patient history included |
| | | probable cytomegalovirus infection, liver congestion, and |
| | | steatosis, splenomegaly, hemorrhagic cystitis, thyroid |
| | | hemorrhage, respiratory failure, pneumonia of the left lung, |
| | | natural killer cell lymphoma of the pharynx, Bell's palsy, and |
| | | tobacco and alcohol abuse. Previous surgeries included |
| | | colonoscopy, closed colon biopsy, adenotonsillectomy, and |
| | | nasopharyngeal endoscopy and biopsy. Patient medications included |
| | | Diflucan (fluconazole), Deltasone (prednisone), hydrocodone, |
| | | Lortab, Alprazolam, |
| | | Reazodone, ProMace-Cytabom, Etoposide, Cisplatin, Cytarabine, |
| | | and dexamethasone. The patient received radiation therapy |
| | | and multiple blood transfusions. The library was normalized in 2 |
| | | rounds using conditions adapted from Soares et al., PNAS |
| | | (1994) 91: 9228-9232 and Bonaldo et al., Genome Research 6 |
| | | (1996): 791, except that a significantly longer (48 hours/round) |
| | | reannealing hybridization was used. |
| OVARDIN02 | pINCY | This normalized ovarian tissue library was constructed from 5.76 |
| | | million independent clones from an ovary library. Starting |
| | | RNA was made from diseased ovarian tissue removed from a 39-year- |
| | | old Caucasian female during total abdominal |
| | | hysterectomy, bilateral salpingo-oophorectomy, dilation |
| | | andcurettage, partial colectomy, incidental appendectomy, and |
| | | temporary colostomy. Pathology indicated the right and left |
| | | adnexa, mesentery and muscularis propria of the sigmoid colon |
| | | were extensively involved by endometriosis. Endometriosis also |
| | | involved the anterior and posterior serosal surfaces of the |
| | | uterus and the cul-de-sac. The endometrium was proliferative. |
| | | Pathology for the associated tumor tissue indicated multiple (3 |
| | | intramural, 1 subserosal) leiomyomata. The patient presented with |
| | | abdominal pain and infertility. Patient history included |
| | | scoliosis. Family history included hyperlipidemia, benign |
| | | hypertension, atherosclerotic coronary artery disease, depressive |
| | | disorder, brain cancer, and type II diabetes. The library was |
| | | normalized in two rounds using conditions adapted from Soares et al., |
| | | PNAS (1994) 91: 9228 and Bonaldo et al., Genome Research 6 (1996): |
| | | 791, except that a significantly longer (48-hours/round) reannealing |
| | | hybridization was used. |
| PANCTUT02 | pINCY | Library was constructed using RNA isolated from pancreatic tumor |
| | | tissue removed from a 45-year-old Caucasian female |
| | | during radical pancreaticoduodenectomy. Pathology indicated a |
| | | grade 4 anaplastic carcinoma. Family history included benign |
| | | hypertension, hyperlipidemia and atherosclerotic coronary artery |
| | | disease. |
| PGANNOT03 | pINCY | Library was constructed using RNA isolated from paraganglionic |
| | | tumor tissue removed from the intra-abdominal region of a |
| | | 46-year-old Caucasian male during exploratory laparotomy. |
| | | Pathology indicated a benign paraganglioma and was associated |
| | | with a grade 2 renal cell carcinoma, clear cell type, which did |
| | | not penetrate the capsule. Surgical margins were negative for |
| | | tumor. |
| PITUNOT01 | PBLUESCRIPT | Library was constructed using RNA obtained from Clontech (CLON |
| | | 6584-2, lot 35278). The RNA was isolated from the |
| | | pituitary glands removed from a pool of 18 male and female |
| | | Caucasian donors, 16 to 70 years old, who died from trauma. |
| PKINDNV08 | PCR2-TOPOTA | Library was constructed using pooled cDNA from different donors. |
| | | cDNA was generated using mRNA isolated from pooled |
| | | skeletal muscle tissue removed from ten 21 to 57-year-old |
| | | Caucasian male and female donors who died from sudden death; |
| | | from pooled thymus tissue removed from nine 18 to 32-year-old |
| | | Caucasian male and female donors who died from sudden |
| | | death; from pooled liver tissue removed from 32 Caucasian male |
| | | and female fetuses who died at 18-24 weeks gestation due to |
| | | spontaneous abortion; from kidney tissue removed from 59 |
| | | Caucasian male and female fetuses who died at 20-33 weeks |
| | | gestation due to spontaneous abortion; and from brain tissue |
| | | removed from a Caucasian male fetus who died at 23 weeks |
| | | gestation due to fetal demise. |
| PROSTUT08 | pINCY | Library was constructed using RNA isolated from prostate tumor |
| | | tissue removed from a 60-year-old Caucasian male during |
| | | radical prostatectomy and regional lymph node excision. |
| | | Pathology indicated an adenocarcinoma (Gleason grade 3 + 4). |
| | | Adenofibromatous hyperplasia was also present. The patient |
| | | presented with elevated prostate specific antigen (PSA). Patient |
| | | history included a kidney cyst, and hematuria. Family history |
| | | included tuberculosis, cerebrovascular disease, and |
| | | arteriosclerotic coronary artery disease. |
| PROSTUT12 | pINCY | Library was constructed using RNA isolated from prostate tumor |
| | | tissue removed from a 65-year-old Caucasian male during a |
| | | radical prostatectomy. Pathology indicated an adenocarcinoma |
| | | (Gleason grade 2 + 2). Adenofibromatous hyperplasia was also |
| | | present. The patient presented with elevated prostate specific |
| | | antigen (PSA). |
| SINTTMR02 | PCDNA2.1 | This random primed library was constructed using RNA isolated |
| | | from small intestine tissue removed from a 59-year-old male. |
| | | Pathology for the matched tumor tissue indicated multiple (9) |
| | | carcinoid tumors, grade 1, in the small bowel. The largest tumor |
| | | was associated with a large mesenteric mass. Multiple convoluted |
| | | segments of bowel were adhered to the tumor. A single (1 of 13) |
| | | regional lymph node was positive for malignancy. The peritoneal |
| | | biopsy indicated focal fat necrosis. |
| SKIRNOR01 | PCDNA2.1 | This random primed library was constructed using RNA isolated |
| | | from skin tissue removed from the breast of a 17-year-old |
| | | Caucasian female during bilateral reduction mammoplasty. Patient |
| | | history included breast hypertrophy. Family history |
| | | included benign hypertension. |
| SPLNNOT04 | pINCY | Library was constructed using RNA isolated from the spleen |
| | | tissue of a 2-year-old Hispanic male, who died from cerebral |
| | | anoxia. Past medical history and serologies were negative. |
| TESTNOT17 | pINCY | Library was constructed from testis tissue removed from a |
| | | 26-year-old Caucasian male who died from head trauma due to a |
| | | motor vehicle accident. Serologies were negative. Patient |
| | | history included a hernia at birth, tobacco use (1½ ppd), |
| | | marijuana use, and daily alcohol use (beer and hard liquor). |
| THYRDIE01 | PCDNA2.1 | This 5′ biased random primed library was constructed using |
| | | RNA isolated from diseased thyroid tissue removed from a 22-year |
| | | old Caucasian female during closed thyroid biopsy, partial |
| | | thyroidectomy, and regional lymph node excision. Pathology |
| | | indicated adenomatous hyperplasia. The patient presented with |
| | | malignant neoplasm of the thyroid. Patient history included |
| | | normal delivery, alcohol abuse, and tobacco abuse. Previous |
| | | surgeries included myringotomy. Patient medications included an |
| | | unspecified type of birth control pills. Family history included |
| | | hyperlipidemia and depressive disorder in the mother; and |
| | | benign hypertension, congestive heart failure, and chronic |
| | | leukemia in the grandparent(s). |
| THYRNOT10 | pINCY | Library was constructed using RNA isolated from the diseased left |
| | | thyroid tissue removed from a 30-year-old Caucasian |
| | | female during a unilateral thyroid lobectomy and parathyroid |
| | | reimplantation. Pathology indicated lymphocytic thyroiditis. |
| UTRSTDT01 | pINCY | Library was constructed using RNA isolated from uterus tissue |
| | | removed from a 46-year-old Caucasian female who died from |
| | | cardiopulmonary arrest. Patient history included liver and |
| | | breast cancer. |
| UTRSTME01 | PCDNA2.1 | This 5′ biased random primed library was constructed using |
| | | RNA isolated from uterus tissue removed from a 49-year-old |
| | | Caucasian female during vaginal hysterectomy and bilateral |
| | | salpingo-oophorectomy. Pathology for the matched tumor tissue |
| | | indicated multiple (6) intramural leiomyomata. The patient |
| | | presented with excessive menstruation, deficiency anemia, and |
| | | dysmenorrhea. Patient history included abdominal pregnancy, |
| | | headache, and chronic obstructive asthma. Previous surgeries |
| | | included hemorrhoidectomy, knee ligament repair, and intranasal |
| | | lesion destruction. Patient medications included Azmacort, |
| | | Proventil, Trazadone, Zostrix HP, iron, Premarin, and vitamin C. |
| | | Family history included alcohol abuse, atherosclerotic |
| | | coronary artery disease, upper lobe lung cancer, and carotid |
| | | endarterectomy in the father; breast fibroadenosis in the |
| | | sibling(s); and acute myocardial infarction, liver cancer, acute |
| | | leukemia, and breast cancer (central) in the grandparent(s). |

TABLE 7

| Program | Description | Reference | Parameter Threshold |
|---|---|---|---|
| ABI FACTURA | A program that removes vector | Applied Biosystems, Foster | |
| | sequences and masks ambiguous | City, CA. | |
| | bases in nucleic acid sequences. | | |
| ABI/PARACEL | A Fast Data Finder useful in | Applied Biosystems, Foster | Mismatch <50% |
| FDF | comparing and annotating amino | City, CA; Paracel Inc., | |
| | acid or nucleic acid sequences. | Pasadena, CA. | |
| ABI | A program that assembles | Applied Biosystems, Foster | |
| AutoAssembler | nucleic acid sequences. | City, CA. | |
| BLAST | A Basic Local Alignment Search | Altschul, S. F. et al. | ESTs: Probability value = |
| | Tool useful in sequence | (1990) J. Mol. Biol. 215: | 1.0E−8 or less |
| | similarity search for amino | 403-410; Altschul, S. F. | Full Length sequences: |
| | acid and nucleic acid sequences. | et al. (1997) Nucleic | Probability value = |
| | BLAST includes five functions: | Acids Res. 25: 3389- | 1.0E−10 or less |
| | blastp, blastn, blastx, tblastn, | 3402. | |
| | and tblastx. | | |
| FASTA | A Pearson and Lipman algorithm | Pearson, W. R. and D. J. | ESTs: fasta E value = |
| | that searches for similarity | Lipman (1988) Proc. Natl. | 1.06E−6 |
| | between a query sequence and | Acad Sci. USA 85: 2444- | Assembled ESTs: fasta |
| | a group of sequences of the | 2448; Pearson, W. R. (1990) | Identity = 95% or greater |
| | same type. FASTA comprises as | Methods Enzymol. 183: | and Match length = 200 |
| | least five functions: fasta, | 63-98; and Smith, T. F. | bases or greater; fastx E |
| | tfasta, fastx, tfastx, and | and M. S. Waterman (1981) | values = 1.0E−8 or less |
| | ssearch. | Adv. Appl. Math. 2: | Full Length sequences: |
| | | 482-489. | fastx score = 100 or |
| | | | greater |
| BLIMPS | A BLocks IMProved Searcher that | Henikoff, S. and J. G. | Probability value = |
| | matches a sequence against | Henikoff (1991) Nucleic | 1.0E−3 or less |
| | those in BLOCKS, PRINTS, DOMO, | Acids Res. 19: 6565- | |
| | PRODOM, and PFAM databases to | 6572; Henikoff, J. G. & S. | |
| | search for gene families, | Henikoff (1996) Methods | |
| | sequence homology, and | Enzymol. 266: 88-105; | |
| | structural fingerprint regions. | and Attwood, T. K. | |
| | | et al. (1997) J. Chem. | |
| | | Inf. Comput. Sci. 37: | |
| | | 417-424. | |
| HMMER | An algorithm for searching a | Krogh, A. et al. (1994) | PFAM, INCY, SMART, or TIGRFAM |
| | query sequence against hidden | J. Mol. Biol. 235: 1501- | hits: Probability value = |
| | Markov model (HMM)-based | 1531; Sonnhammer, E. L. L. | 1.0E−3 or less Signal |
| | databases of protein family | et al. (1988) Nucleic Acids | peptide hits: Score = 0 or |
| | consensus sequences, such as | Res. 26: 320-322; | greater |
| | PFAM, INCY, SMART, and TIGRFAM. | Durbin, R. et al. (1998) Our | |
| | | World View, in a Nutshell, | |
| | | Cambridge Univ. Press, | |
| | | p. 1-350 | |
| ProfileScan | An algorithm that searches | Gribskov, M. et al. (1988) | Normalized quality score ≧ |
| | for structural and sequence | CABIOS 4: 61-66; | GCG-specified “HIGH” |
| | motifs in protein sequences | Gribskov, M. et al. (1989) | value for that particular |
| | that match sequence patterns | Methods Enzymol. 183: 146- | Prosite motif. |
| | defined in Prosite. | 159; Bairoch, A. et al. | Generally, score = |
| | | (1997) Nucleic Acids Res. | 1.4−2.1. |
| | | 25: 217-221. | |
| Phred | A base-calling algorithm that | Ewing, B. et al. (1998) | |
| | examines automated sequencer | Genome Res. 8: 175-185; | |
| | traces with high sensitivity | Ewing, B. and P. Green | |
| | and probability. | (1998) Genome Res. 8: | |
| | | 186-194. | |
| Phrap | A Phils Revised Assembly | Smith, T. F. and M. S. | Score = 120 or greater; |
| | Program including SWAT and | Waterman (1981) Adv. Appl. | Match length = 56 or greater |
| | CrossMatch, programs based on | Math. 2: 482-489; Smith, | |
| | efficient implementation of the | T. F. and M. S. Waterman | |
| | Smith-Waterman algorithm, | (1981) J. Mol. Biol. 147: | |
| | useful in searching sequence | 195-197; and Green, P., | |
| | homology and assembling DNA | University of Washington, | |
| | sequences. | Seattle, WA. | |
| Consed | A graphical tool for viewing | Gordon, D. et al. (1998) | |
| | and editing Phrap assemblies. | Genome Res. 8: 195-202. | |
| SPScan | A weight matrix analysis | Nielson. H. et al. (1997) | Score = 3.5 or greater |
| | program that scans protein | Protein Engineering 10: | |
| | sequences for the presence | 1-6; Claverie, J. M. | |
| | of secretory signal peptides. | and S. Audic (1997) | |
| | | CABIOS 12: 431-439. | |
| TMAP | A program that uses weight | Persson, B. and P. Argos | |
| | matrices to delineate | (1994) J. Mol. Biol. | |
| | transmembrane segments on | 237: 182-192; Persson, | |
| | protein sequences and | B. and P. Argos (1996) | |
| | determine orientation. | Protein Sci. 5: 363-371. | |
| TMHMMER | A program that uses a hidden | Sonnhammer, E. L. et al. | |
| | Markov model (HMM) to delineate | (1998) Proc. Sixth Intl. | |
| | transmembrane segments on | Conf. on Intelligent | |
| | protein sequences and determine | Systems for Mol. Biol., | |
| | orientation. | Glasgow et al., eds., The | |
| | | Am. Assoc. for Artificial | |
| | | Intelligence Press, Menlo | |
| | | Park, CA, pp. 175-182. | |
| Motifs | A program that searches amino | Bairoch, A. et al. (1997) | |
| | acid sequences for patterns | Nucleic Acids Res. 25: | |
| | that matched those defined | 217-221; Wisconsin | |
| | in Prosite. | Package Program Manual, | |
| | | version 9, page M51-59, | |
| | | Genetics Computer Group, | |
| | | Madison, WI. | |

TABLE 8

| SEQ | | | | | | EST | Al- | Al- | | Caucasian | African | Asian | Hispanic |
|---|---|---|---|---|---|---|---|---|---|---|---|---|---|
| ID | | | | EST | CB1 | Al- | lele | lele | Amino | Allele 1 | Allele 1 | Allele 1 | Allele 1 |
| NO: | PID | EST ID | SNP ID | SNP | SNP | lele | 1 | 2 | Acid | frequency | frequency | frequency | frequency |
| 82 | 7506228 | 2112443H1 | SNP00131740 | 98 | 631 | G | A | G | G138 | n/a | n/a | n/a | n/a |
| 82 | 7506228 | 377285H1 | SNP00063812 | 175 | 747 | A | A | G | I177 | n/a | n/a | n/a | n/a |
| 82 | 7506228 | 7123291H1 | SNP00092195 | 288 | 638 | G | G | T | G140 | n/a | n/a | n/a | n/a |
| 82 | 7506228 | 7123291H1 | SNP00113910 | 519 | 407 | C | C | T | I63 | n/a | n/a | n/a | n/a |
| 86 | 7506224 | 056888H1 | SNP00053556 | 3 | 1294 | T | T | C | L421 | n/a | n/a | n/a | n/a |
| 86 | 7506224 | 1234444H1 | SNP00003796 | 127 | 1779 | A | G | A | M583 | n/a | n/a | n/a | n/a |
| 86 | 7506224 | 1293012H1 | SNP00146136 | 194 | 2018 | T | T | C | C662 | n/a | n/a | n/a | n/a |
| 86 | 7506224 | 1353573H1 | SNP00146135 | 101 | 1518 | C | C | T | Q496 | n/a | n/a | n/a | n/a |
| 86 | 7506224 | 1404229H1 | SNP00024078 | 168 | 2697 | T | T | C | non- | n/a | n/a | n/a | n/a |
| | | | | | | | | | coding | | | | |
| 86 | 7506224 | 1893956H1 | SNP00128176 | 33 | 2383 | A | A | G | non- | n/a | n/a | n/a | n/a |
| | | | | | | | | | coding | | | | |
| 86 | 7506224 | 2284385H1 | SNP00024076 | 93 | 1779 | A | G | A | M583 | 0.96 | n/a | n/a | n/a |
| 86 | 7506224 | 2671183H1 | SNP00003795 | 176 | 1385 | C | C | T | H451 | 0.74 | n/a | n/a | n/a |
| 86 | 7506224 | 2671183H1 | SNP00146134 | 127 | 1336 | C | C | T | S435 | n/a | n/a | n/a | n/a |
| 86 | 7506224 | 2939104H1 | SNP00053555 | 203 | 1108 | T | T | C | L359 | n/d | n/d | n/d | n/d |
| 86 | 7506224 | 3471493H1 | SNP00132436 | 122 | 1202 | A | A | G | S390 | n/a | n/a | n/a | n/a |
| 86 | 7506224 | 4433138H1 | SNP00024077 | 92 | 1947 | T | T | G | S639 | 0.95 | n/a | n/a | n/a |
| 86 | 7506224 | 546618H1 | SNP00003842 | 12 | 2230 | A | A | G | D733 | n/a | n/a | n/a | n/a |
| 86 | 7506224 | 6981529H1 | SNP00053554 | 323 | 426 | G | A | G | A132 | 0.93 | 0.91 | n/d | 0.94 |
| 86 | 7506224 | 7262269H1 | SNP00148110 | 147 | 473 | T | T | G | F147 | n/a | n/a | n/a | n/a |
| 88 | 7508326 | 1288671H1 | SNP00006550 | 237 | 1136 | G | G | A | E334 | n/a | n/a | n/a | n/a |
| 88 | 7508326 | 1288671H1 | SNP00073295 | 175 | 1074 | C | C | A | P314 | n/a | n/a | n/a | n/a |
| 88 | 7508326 | 1396918H1 | SNP00029915 | 53 | 1428 | C | C | T | non- | n/a | n/a | n/a | n/a |
| | | | | | | | | | coding | | | | |
| 89 | 7506370 | 1642795H1 | SNP00108006 | 36 | 213 | C | C | T | A61 | n/a | n/a | n/a | n/a |
| 89 | 7506370 | 1642795H1 | SNP00108007 | 69 | 249 | A | A | G | N73 | n/a | n/a | n/a | n/a |
| 89 | 7506370 | 6267662H1 | SNP00108007 | 13 | 285 | A | A | G | N85 | n/a | n/a | n/a | n/a |
| 90 | 6312989 | 1731793H1 | SNP00036588 | 66 | 1749 | C | C | T | P477 | n/d | n/d | n/d | n/d |
| 90 | 6312989 | 2176287H1 | SNP00067594 | 133 | 1966 | C | C | A | S549 | n/a | n/a | n/a | n/a |
| 90 | 6312989 | 2642846H1 | SNP00067595 | 73 | 2096 | A | A | C | non- | n/a | n/a | n/a | n/a |
| | | | | | | | | | coding | | | | |
| 91 | 7501108 | 1345691H1 | SNP00024762 | 175 | 308 | G | A | G | V94 | n/a | n/a | n/a | n/a |
| 91 | 7501108 | 1474830H1 | SNP00024764 | 61 | 818 | G | G | C | V264 | n/a | n/a | n/a | n/a |
| 91 | 7501108 | 1550205H1 | SNP00004160 | 39 | 695 | G | G | A | V223 | 0.25 | n/a | n/a | n/a |
| 91 | 7501108 | 2607354H1 | SNP00155138 | 221 | 1411 | T | T | G | non- | n/a | n/a | n/a | n/a |
| | | | | | | | | | coding | | | | |
| 91 | 7501108 | 2615552H1 | SNP00059640 | 71 | 635 | T | T | C | S203 | 0.28 | 0.12 | 0.27 | 0.28 |
| 91 | 7501108 | 4865334H1 | SNP00004161 | 168 | 1284 | T | T | G | non- | n/a | n/a | n/a | |
| | | | | | | | | | coding | | | | |
| 91 | 7501108 | 7111705H2 | SNP00024764 | 416 | 671 | G | G | C | A215 | n/a | n/a | n/a | n/a |
| 92 | 7507581 | 103570H1 | SNP00024069 | 145 | 956 | C | C | T | S260 | n/a | n/a | n/a | n/a |
| 92 | 7507581 | 1893676H1 | SNP00003837 | 106 | 1202 | G | A | G | non- | n/a | n/a | n/a | n/a |
| | | | | | | | | | coding | | | | |
| 92 | 7507581 | 1893676H1 | SNP00059937 | 172 | 1268 | C | C | T | non- | n/a | n/a | n/a | n/a |
| | | | | | | | | | coding | | | | |
| 92 | 7507581 | 3037648H1 | SNP00003836 | 222 | 975 | T | T | C | T266 | n/a | n/a | n/a | n/a |
| 92 | 7507581 | 3037648H1 | SNP00024068 | 20 | 773 | C | T | C | S199 | n/a | n/a | n/a | n/a |
| 92 | 7507581 | 3329650H1 | SNP00055085 | 142 | 723 | C | C | T | I182 | 0.84 | 0.88 | 0.74 | 0.65 |
| 92 | 7507581 | 3329650H1 | SNP00135947 | 264 | 846 | C | C | T | N223 | n/a | n/a | n/a | n/a |
| 93 | 7506361 | 1275761H1 | SNP00016025 | 133 | 1262 | C | C | A | T381 | n/a | n/a | n/a | n/a |
| 93 | 7506361 | 1368438H1 | SNP00100755 | 160 | 1661 | C | C | A | G514 | n/a | n/a | n/a | n/a |
| 93 | 7506361 | 2092953H1 | SNP00016027 | 213 | 1847 | G | G | A | non- | n/a | n/a | n/a | n/a |
| | | | | | | | | | coding | | | | |
| 93 | 7506361 | 3389302H1 | SNP00067482 | 84 | 798 | G | A | G | E227 | n/a | n/a | n/a | n/a |
| 93 | 7506361 | 5312385H1 | SNP00073522 | 19 | 774 | G | G | C | A219 | n/a | n/a | n/a | n/a |
| 93 | 7506361 | 6835541H1 | SNP00016026 | 159 | 1416 | C | C | T | L433 | n/a | n/a | n/a | n/a |
| 93 | 7506361 | 6835541H1 | SNP00111715 | 127 | 1385 | C | C | A | P422 | n/d | n/a | n/a | n/a |
| 93 | 7506361 | 7963996H1 | SNP00120336 | 202 | 33 | A | G | A | non- | n/a | n/a | n/a | n/a |
| | | | | | | | | | coding | | | | |
| 94 | 7509211 | 1731793H1 | SNP00036588 | 66 | 2238 | C | C | T | P548 | n/d | n/d | n/d | n/d |
| 94 | 7509211 | 2176287H1 | SNP00067594 | 133 | 2458 | C | C | A | S621 | n/a | n/a | n/a | n/a |
| 94 | 7509211 | 2642846H1 | SNP00067595 | 73 | 2588 | A | A | C | non- | n/a | n/a | n/a | n/a |
| | | | | | | | | | coding | | | | |
| 94 | 7509211 | 4000464H1 | SNP00067595 | 2 | 2581 | A | A | C | non- | n/a | n/a | n/a | n/a |
| | | | | | | | | | coding | | | | |
| 94 | 7509211 | 4259369H1 | SNP00067594 | 211 | 2455 | C | C | A | S620 | n/a | n/a | n/a | n/a |
| 94 | 7509211 | 6324551H1 | SNP00067594 | 123 | 2454 | C | C | A | S620 | n/a | n/a | n/a | n/a |
| 94 | 7509211 | 6372396H1 | SNP00067595 | 54 | 2586 | A | A | C | non- | n/a | n/a | n/a | n/a |
| | | | | | | | | | coding | | | | |

1. An isolated polypeptide selected from the group consisting of:
a) a polypeptide comprising an amino acid sequence selected from the group consisting of SEQ ID NO:1-47
b) a polypeptide comprising a naturally occurring amino acid sequence at least 90% identical to an amino acid sequence selected from the group consisting of SEQ ID NO:1-4, SEQ ID NO:6, SEQ ID NO:8, SEQ ID NO:13-15, SEQ ID NO:24, SEQ ID NO:27-28, SEQ ID NO:33-34, SEQ ID NO:37-38, SEQ ID NO:43, and SEQ ID NO:45,
c) a polypeptide comprising a naturally occurring amino acid sequence at least 95% identical to the amino acid sequence of SEQ ID NO:16,
d) a polypeptide comprising a naturally occurring amino acid sequence at least 94% identical to the amino acid sequence of SEQ ID NO:23,
e) a polypeptide comprising a naturally occurring amino acid sequence at least 97% identical to the amino acid sequence of SEQ ID NO:31,
f) a polypeptide comprising a naturally occurring amino acid sequence at least 96% identical to the amino acid sequence of SEQ ID NO:42,
g) a polypeptide consisting essentially of a naturally occurring amino acid sequence at least 90% identical to an amino acid sequence selected from the group consisting of SEQ ID NO:5, SEQ ID NO:7, SEQ ID NO:9-11, SEQ ID NO:17-22, SEQ ID NO:25-26, SEQ ID NO:29-30, SEQ ID NO:32, SEQ ID NO:35-36, SEQ ID NO:39-41, SEQ ID NO:44, and SEQ ID NO:4647,
h) a biologically active fragment of a polypeptide having an amino acid sequence selected from the group consisting of SEQ ID NO:1-47, and
i) an immunogenic fragment of a polypeptide having an amino acid sequence selected from the group consisting of SEQ ID NO:1-47.

2. An isolated polypeptide of claim 1 comprising an amino acid sequence selected from the group consisting of SEQ ID NO:1-47.

3. An isolated polynucleotide encoding a polypeptide of claim 1.

4. An isolated polynucleotide encoding a polypeptide of claim 2.

5. An isolated polynucleotide of claim 4 comprising a polynucleotide sequence selected from the group consisting of SEQ ID NO:48-94.

6. A recombinant polynucleotide comprising a promoter sequence operably linked to a polynucleotide of claim 3.

7. A cell transformed with a recombinant polynucleotide of claim 6.

8. (canceled)

9. A method of producing a polypeptide of claim 1, the method comprising:
a) culturing a cell under conditions suitable for expression of the polypeptide, wherein said cell is transformed with a recombinant polynucleotide, and said recombinant polynucleotide comprises a promoter sequence operably linked to a polynucleotide encoding the polypeptide of claim 1, and
b) recovering the polypeptide so expressed.

10. (canceled)

11. An isolated antibody which specifically binds to a polypeptide of claim 1.

12. An isolated polynucleotide selected from the group consisting of:
a) a polynucleotide comprising a polynucleotide sequence selected from the group consisting of SEQ ID NO:48-94,
b) a polynucleotide comprising a naturally occurring polynucleotide sequence at least 90% identical to a polynucleotide sequence selected from the group consisting of SEQ ID NO:48-63, SEQ ID NO:65-81, SEQ ID NO:85, SEQ ID NO:90, and SEQ ID NO:94,
c) a polynucleotide comprising a naturally occurring polynucleotide sequence at least 94% identical to a polynucleotide sequence of SEQ ID NO:64,
d) a polynucleotide comprising a naturally occurring polynucleotide sequence at least 97% identical to the polynucleotide sequence of SEQ ID NO:82,
e) a polynucleotide comprising a naturally occurring polynucleotide sequence at least 99% identical to an polynucleotide sequence selected from the group consisting of SEQ ID NO:83-84,
f) a polynucleotide comprising a naturally occurring polynucleotide sequence at least 92% identical to the polynucleotide sequence of SEQ ID NO:92,
g) a polynucleotide consisting essentially of a naturally occurring polynucleotide sequence at least 90% identical to a polynucleotide sequence selected from the group consisting of SEQ ID NO:86-89, SEQ ID NO:91, and SEQ ID NO:93,
h) a polynucleotide complementary to a polynucleotide of a),
i) a polynucleotide complementary to a polynucleotide of b),
j) a polynucleotide complementary to a polynucleotide of c),
k) a polynucleotide complementary to a polynucleotide of d),
l) a polynucleotide complementary to a polynucleotide of e),
m) a polynucleotide complementary to a polynucleotide of f),
n) a polynucleotide complementary to a polynucleotide of g), and
o) an RNA equivalent of a)-n).

13. (canceled)

14. A method of detecting a target polynucleotide in a sample, said target polynucleotide having a sequence of a polynucleotide of claim 12, the method comprising:
a) hybridizing the sample with a probe comprising at least 20 contiguous nucleotides comprising a sequence complementary to said target polynucleotide in the sample, and which probe specifically hybridizes to said target polynucleotide, under conditions whereby a hybridization complex is formed between said probe and said target polynucleotide or fragments thereof, and
b) detecting the presence or absence of said hybridization complex, and, optionally, if present, the amount thereof.

15. (canceled)

16. A method of detecting a target polynucleotide in a sample, said target polynucleotide having a sequence of a polynucleotide of claim 12, the method comprising:
a) amplifying said target polynucleotide or fragment thereof using polymerase chain reaction amplification, and
b) detecting the presence or absence of said amplified target polynucleotide or fragment thereof, and, optionally, if present, the amount thereof.

17. A composition comprising a polypeptide of claim 1 and a pharmaceutically acceptable excipient.

18. (canceled)

19. (canceled)

20. A method of screening a compound for effectiveness as an agonist of a polypeptide of claim 1, the method comprising:
a) exposing a sample comprising a polypeptide of claim 1 to a compound, and
b) detecting agonist activity in the sample.

21. (canceled)

22. (canceled)

23. A method of screening a compound for effectiveness as an antagonist of a polypeptide of claim 1, the method comprising:
a) exposing a sample comprising a polypeptide of claim 1 to a compound, and
b) detecting antagonist activity in the sample.

24. (canceled)

25. (canceled)

26. A method of screening for a compound that specifically binds to the polypeptide of claim 1, the method comprising:
a) combining the polypeptide of claim 1 with at least one test compound under suitable conditions, and
b) detecting binding of the polypeptide of claim 1 to the test compound, thereby identifying a compound that specifically binds to the polypeptide of claim 1.

27. A method of screening for a compound that modulates the activity of the polypeptide of claim 1, the method comprising:
a) combining the polypeptide of claim 1 with at least one test compound under conditions permissive for the activity of the polypeptide of claim 1,
b) assessing the activity of the polypeptide of claim 1 in the presence of the test compound, and
c) comparing the activity of the polypeptide of claim 1 in the presence of the test compound with the activity of the polypeptide of claim 1 in the absence of the test compound, wherein a change in the activity of the polypeptide of claim 1 in the presence of the test compound is indicative of a compound that modulates the activity of the polypeptide of claim 1.

28. (canceled)

29. A method of assessing toxicity of a test compound, the method comprising:
a) treating a biological sample containing nucleic acids with the test compound,
b) hybridizing the nucleic acids of the treated biological sample with a probe comprising at least 20 contiguous nucleotides of a polynucleotide of claim 12 under conditions whereby a specific hybridization complex is formed between said probe and a target polynucleotide in the biological sample, said target polynucleotide comprising a polynucleotide sequence of a polynucleotide of claim 12 or fragment thereof,
c) quantifying the amount of hybridization complex, and
d) comparing the amount of hybridization complex in the treated biological sample with the amount of hybridization complex in an untreated biological sample, wherein a difference in the amount of hybridization complex in the treated biological sample is indicative of toxicity of the test compound.

30. (canceled)

31. The antibody of claim 11, wherein the antibody is:
a) a chimeric antibody,
b) a single chain antibody,
c) a Fab fragment,
d) a F(ab′)₂ fragment, or
e) a humanized antibody.

32-45. (canceled)

46. A microarray wherein at least one element of the microarray is a polynucleotide of claim 13.

47-149. (canceled)
